(19) United States
(12) Patent Application Publication
(10) Pub. No.: US 2005/0137220 A1
(43) Pub. Date: Jun. 23, 2005

(54) Beta-carboline compounds and analogues thereof as mitogen-activated protein kinase-activated protein kinase-2 inhibitors

(75) Inventors: David R. Anderson, Lake St. Louis, MO (US); Ingrid P. Buchler, South University City, MO (US); Shridhar G. Hegde, Ballwin, MO (US); Matthew W. Mahoney, St. Peters, MO (US); Marvin J. Meyers, St. Charles, MO (US); David B. Reitz, Chesterfield, MO (US); John I. Trujillo, St. Peters, MO (US); William F. Vernier, Oceanside, CA (US); Kun K. Wu, Chesterfield, MO (US)

Correspondence Address: Charles E. Dunlap;Nelson Mullins Riley & Scarborough, LLP, 17th Floor, 1320 Main Street, Columbia, SC, 29201, US

(73) Assignee: Pharmacia Corporation, Chesterfield, MO (US)

(21) Appl. No.: 10/895,187

(22) Filed: Jul. 20, 2004

Related U.S. Application Data
(60) Provisional application No. 60/489,410, filed on Jul. 23, 2003.

(51) Int. Cl. A61K 31/4745; A61K 31/407

(52) U.S. Cl. 514/291; 514/411

(57) ABSTRACT

A method is described for inhibiting mitogen activated protein kinase-activated protein kinase-2 in a subject in need of such inhibition, where the method involves administering to the subject a beta-carboline MK-2 inhibiting compound, or a pharmaceutically acceptable salt thereof.

### CROSS-REFERENCE TO RELATED PATENTS AND PATENT APPLICATIONS

This application is related to and claims the priority benefit of U.S. Patent Application Ser. No. 60/489,410 filed Jul. 23, 2003, which is incorporated by reference herein in its entirety.

### BACKGROUND OF THE INVENTION

1) Field of the Invention

The present invention relates to beta-carboline compounds and analogues thereof and their use as inhibitors of mitogen-activated protein kinase-activated protein kinase-2 (MK-2), and also to a method for preventing or treating a disease or disorder that can be treated or prevented by modulating the activity of MK-2 in a subject, and to pharmaceutical compositions and kits that include these MK-2 inhibitors.

2) Description of the Related Art

Mitogen-activated protein kinases (MAPKs) are members of conserved signal transduction pathways that activate transcription factors, translation factors and other target molecules in response to a variety of extracellular signals. MAPKs are activated by phosphorylation at a dual phosphorylation motif with the sequence Thr-X-Tyr by mitogen-activated protein kinase kinases (MAPKKs).

In higher eukaryotes, the physiological role of MAPK signaling has been correlated with cellular events such as proliferation, oncogenesis, development and differentiation. Accordingly, the ability to regulate signal transduction via these pathways could lead to the development of treatments and preventive therapies for human diseases associated with MAPK signaling, such as inflammatory diseases, autoimmune diseases and cancer.

In mammalian cells, three parallel MAPK pathways have been described. The best characterized pathway leads to the activation of the extracellular-signal-regulated kinase (ERK). Less well understood are the signal transduction pathways leading to the activation of the cJun N-terminal kinase (JNK) and the p38 MAPK. See e.g., Davis, Trends Biochem. Sci. 19:470-473 (1994).

The p38 MAPK pathway is potentially activated by a wide variety of stresses and cellular insults. These stresses and cellular insults include heat shock, UV irradiation, inflammatory cytokines (such as TNF and IL-1), tunicamycin, chemotherapeutic drugs (i.e., cisplatinum), anisomycin, sorbitol/hyperosmolarity, gamma irradiation, sodium arsenite, and ischaemia. See, Ono, K., et al., Cellular Signalling 12, 1-13 (2000). Activation of the p38 pathway is involved in (1) production of proinflammatory cytokines, such as TNF-α; (2) induction of enzymes, such as Cox-2; (3) expression of an intracellular enzyme, such as iNOS, which plays an important role in the regulation of oxidation; (4) induction of adherent proteins, such as VCAM-1 and many other inflammatory-related molecules. Furthermore, the p38 pathway functions as a regulator in the proliferation and differentiation of cells of the immune system. See Ono, K., et al., Id. at 7.

The p38 kinase is an upstream kinase of mitogen-activated protein kinase-activated protein kinase-2 (MAPKAP kinase-2 or MK-2). See Freshney, N. W., et al., J. Cell, 78:1039-1049 (1994). MK-2 is a protein that appears to be predominantly regulated by p38 in cells. Indeed, MK-2 was the first substrate of p38α to be identified. For example, in vitro phosphorylation of MK-2 by p38α activates MK-2. The substrates that MK-2 acts upon, in turn, include heat shock protein 27, lymphocyte-specific protein 1 (LAP1), cAMP response element-binding protein (CREB), ATF1, serum response factor (SRF), and tyrosine hydroxylase. The substrate of MK-2 that has been best characterized is small heat shock protein 27 (hsp27).

The role of the p38 pathway in inflammatory-related diseases has been studied in several animal models. The pyridinyl imidazole compound SB203580 has been shown to be a specific inhibitor of p38 in vivo, and also has been shown to inhibit activation of MK-2. See Rouse, J., et al., Cell, 78:1027-1037 (1994); Cuenda, A., et al., Biochem. J., 333: 11-15 (1998), as well as a MAP kinase homologue termed reactivating kinase (RK). See Cuenda, A., et al., FEBS Lett. 364(2): 229-233 (1995). Inhibition of p38 by SB203580 can reduce mortality in a murine model of endotoxin-induced shock and inhibit the development of mouse collagen-induced arthritis and rat adjuvant arthritis. See e.g., Badger, A. M., et al., J. Pharmacol Exp. Ther. 279:1453-1461 (1996). Another p38 inhibitor that has been utilized in an animal model that is believed to be more potent than SB203580 in its inhibitory effect on p38 is SB 220025. A recent animal study has demonstrated that SB 220025 caused a significant dose-dependent decrease in vascular density of granulomas in laboratory rats. See Jackson, J. R., et al., J. Pharmacol. Exp. Ther. 284:687-692 (1998). The results of these animal studies indicated that p38, or the components of the p38 pathway, can be useful therapeutic targets for the prevention or treatment of inflammatory disease.

Due to its integral role in the p38 signaling pathway, MK-2 has been used as a monitor for measuring the level of activation in the pathway. Because of its downstream location in the pathway, relative to p38, MK-2 has been measured as a more convenient, albeit indirect, method of assessing p38 activation. However, so far, research efforts exploring therapeutic strategies associated with the modulation of this pathway have focused mainly on the inhibition of p38 kinase.

Several compounds that inhibit the activity of p38 kinase have been described in U.S. Pat. Nos. 6,046,208, 6,251,914, and 6,335,340. These compounds have been suggested to be useful for the treatment of CSBP/RK/p38 kinase mediated disease. Commercial efforts to apply p38 inhibitors have centered around two p38 inhibitors, the pyridinylimidazole inhibitor SKF 86002, and the 2,4,5 triaryl imidazole inhibitor SB203580. See Lee, J. C., et al., Immunopharmacology 47: 185-192 (2000).

Compounds possessing a similar structure have also been investigated as potential p38 inhibitors. Indeed, p38 MSP kinase's role in various disease states has been elucidated through the use of inhibitors. Kotlyarov, A. et al. introduced a targeted mutation into a mouse MK-2 gene, resulting in MK-2-deficient mice. It was shown that mice lacking MK-2 possessed increased stress resistance and survived LPS-induced endotoxic shock better than MK-2⁺ mice. The authors concluded that MK-2 was an essential component in the inflammatory response that regulates biosynthesis of TNFα at a post-transcriptional level. See Kotlyarov, A. et al., Nat. Cell Biol. 1(2): 94-97 (1999).

More recently, Lehner, M. D., et al. reported that MK-2-deficient mice showed increased susceptibility to Listeria monocytogenes infection, and concluded that MK-2 had an essential role in host defense against intracellular bacteria, probably via regulation of TNF and IFN-gamma production required for activation of antibacterial effector mechanisms. See Lehner, M. D., et al., J. Immunol. 168(9): 4667-4673 (2002).

The location of MK-2 in the p38 signaling pathway at a point that is downstream of p38 offers the potential that MK-2 could act as a focal point for modulating the pathway without affecting as many substrates as would the regulation of an enzyme further upstream in the signaling cascade—such as p38 MAP kinase.

A class of compounds that have not heretofore been described as capable of modulating MK-2 enzyme activity are the beta-carboline class of compounds. Beta-carbolines are tricyclic heterocyclic structures with specific side groups attached at various points, differentiating members of this class of compound. See Pari, K., et al., J. Biol. Chem. 275(4): 2455-2462 (2000). The skeleton ring structure of beta-carbolines consists of an indole group attached to a cyclic amine. See U.S. Pat. No. 5,256,533 to Jones, et al.

Historically, beta-carboline compounds have shown a variety of pharmacological actions and have been variously evaluated as cholecystokinin antagonists, benzodiazepine antagonists, sedatives, anti-epileptics, appetite suppressants, anti-cancer agents, anti-convulsants, cardiovascular disorder treatments, anti-depressants, anti-malarial agents and anti-psychotics. See e.g., U.S. Pat. No. 6,069,150 to Spinelli, et al. To date, however, beta-carbolines have not been described as effective for treating TNFα-mediated inflammatory diseases or disorders.

Accordingly, it would be useful to provide compounds and methods that could serve to modulate the activity of MK-2—in particular, to act as inhibitors of MK-2 activity. Such compounds and methods would be useful for the provision of benefits similar to p38 MAP kinase inhibitors, which benefits include the prevention and treatment of diseases and disorders that can be prevented or treated by modulating the activity of MK-2. It would be even more useful to provide MK-2 inhibitors having improved potency and reduced undesirable side effects, relative to p38 inhibitors.

### SUMMARY OF THE INVENTION

Briefly, therefore the present invention is directed to a novel method for treating or preventing a disease or disorder in a subject, which disease or disorder is one that can be treated or prevented by inhibiting the activity of MK-2, the method comprising administering to the subject a compound, or a salt, isomer, or prodrug thereof, wherein the compound has the structure according to formula I: 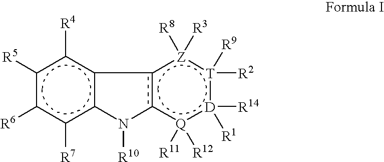
wherein:
Q, D, T and Z are selected from carbon, sulfur, or nitrogen;
when Q is sulfur, R¹² is oxo, and R¹¹ is optionally oxo or is absent;
when T is sulfur, R² is oxo, and R⁹ is optionally oxo or is absent;
when Z is nitrogen, D is nitrogen, and R² and R¹² are oxo;
R¹ is selected from H, alkyl, alkenyl, alkynyl, allyl, acetyl, amino, alkoxy, hydroxy, oxo, carboxy, allylalkyl, benzyl, 2-ethylalkyl, haloacetyl, t-butyldialkylsilyloxyalkyl, alkoxycarbonylaminoalkyl, haloalkylbenzyl, haloalkylcarbonyl, alkylcarbonyl, alkoxycarbonyl, arylcarbonyl, benzyloxycarbonyl, arylalkenylamino, alkoxyheterocyclylcarbonyl, haloarylcarbonyl, or substituted or unsubstituted alkyl, which, if substituted, has one or more substituent groups selected from H, halo, amino, aryl, or heteroaryl;
   R² is selected from H, alkyl, alkoxy, hydroxy, carbonyl, oxo, benzyl, hydroxyalkyl, dialkylamino, carboxyl, amino, halo, alkoxycarbonyl, alkoxycarbonyl-R¹⁵, aminoalkyl, haloalkyl, hydroxyalkylbenzyl, haloalkylbenzyl, arylalkenylamino, haloalkoxybenzyl, cyanoalkyl, heterocyclylalkyl, arylalkyl-R¹⁵, alkyl-SO₂—R¹⁵, substituted or unsubstituted aryl, which, if substituted, has one or more substituent groups selected from H, alkyl, amino, nitro, halo, haloalkyl, or hydroxyalkyl, or substituted or unsubstituted alkyl, which, if substituted, has one or more substituent groups selected from H, halo, amino, aryl, or heteroaryl;
   R³ is selected from H, oxo, alkyl, alkoxy, amino, halo, haloalkyl, hydroxy, carbonyl, alkoxycarbonyl, benzyl, aminoalkyl, hydroxyalkyl, dialkylamino, arylalkenylamino, carboxy, haloalkyl, hydroxyalkylbenzyl, haloalkylbenzyl, haloalkoxybenzyl, heterocyclylalkyl, substituted or unsubstituted aryl, which, if substituted, has one or more substituent groups selected from H, alkyl, amino, nitro, halo, haloalkyl, or hydroxyalkyl, or substituted or unsubstituted alkyl, which, if substituted, has one or more substituent groups selected from H, halo, amino, aryl, or heteroaryl, or R¹ and R³ optionally join to form an alkyl bridge between the ring atoms to which R¹ and R³ are attached, or R² and R³ optionally join to form a cycloalkyl ring;
   R⁴, R⁶ and R⁷ are independently selected from H, alkyl, benzyl, halo, haloalkyl, alkoxy, haloalkoxy, carboxyl, CO₂—R¹⁵, carboxyl, nitro, amino, alkylamino, dialkylamino, benzyloxy, hydrazinocarbonyl, alkoxycarbonyl, alkoxycarbonylheterocyclylcarbonyl, hydroxy, or substituted or unsubstituted 6-membered heterocyclylcarbonyl, which, if substituted, has one or more substituent groups selected from H or alkoxycarbonyl, or R⁶ and R⁷ optionally join to form a heteroaryl ring;
   R⁵ is selected from H, alkyl, alkenyl, alkylthio, hydroxy, carboxyl, halo, alkoxy, benzylalkyl, benzylalkoxy, arylalkyl, nitro, alkoxycarbonylalkoxy, alkylthio, benzyloxy, arylalkynyl, arylcarbonylalkoxy, haloalkoxy, haloalkyl, arylalkoxy, haloarylalkoxy, benzyloxoalkoxy, alkoxycarbonylbenzyloxy, alkoxycarbonyl, alkoxyoxoalkoxy, alkoxyoxoarylalkoxy, halobenzyloxy, heterocyclylalkoxy, dialkylaminooxoalkoxy, aminocarbonyl, arylalkynyl, hydrazinocarbonyl, carbonylalkoxy, alkylsulfonylbenzyloxy, CO₂—R¹⁵, carbamoyl, naphthylalkoxy, alkoxyaryloxy, alkoxycarbonylheterocyclylalkoxy, haloalkylbenzyloxy, alkoxyacetylaminoacetyloxy, acetoxy, cycloalkylalkoxy, acetyloxy, morpholinylcarbonyl, alkoxyalkoxy, or R⁴ and R⁵ optionally join to form a heterocyclic ring;
   R⁸ and R⁹ are independently optionally present and if present, are independently selected from H, oxo, carbonitrile, alkyl, or is optionally absent, or R³ and R⁸ optionally join to form a cycloalkyl ring;
   R¹⁰ is selected from H, alkyl, benzyl, aryl, or hydroxyalkyl;
   R¹¹ is optionally present and if present, is selected from H, oxo, alkyl, halobenzyl, benzyl, haloalkyl, 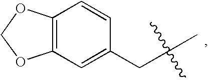
   alkoxycarbonyl, carboxyalkyl, benzodioxinylalkyl, substituted or unsubstituted aryl or arylalkyl, which, if substituted, have one or more substituent groups selected from H, alkyl, or alkoxy;
   R¹² is optionally present and if present, is selected from H, ═S, oxo, alkyl, carbonyl-R¹³, carboxyl-R¹³, ketone, cyano, cyanoalkyl, alkoxycarbonyl, aminoalkyl, alkoxycarbonylalkyl, carbamyl, hydroxycarbamyl, haloalkyl, heterocyclyl, alkylacetate, heterocyclylalkyl, alkoxycarbonylalkyl, hydroxyalkyl, dihydroxyalkyl, cyanoalkyl, ═N—OR¹⁵, ═N—N(R¹⁵)₂, or hydroxyamino, or R¹ and R¹² optionally join to form a substituted or unsubstituted 5 or 6-membered heteroaryl ring, which, if substituted, has one or more substituent groups selected from H, alkyl, oxo, or alkoxycarbonylalkyl, or R¹¹ and R¹² optionally join to form a ring system having the structure: 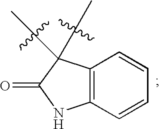
   R¹³ is optionally present and if present, is selected from H, alkyl, alkoxy, amino, alkynyl, allyl, alkenyl, hydrazine, benzyl, glycine, allylalkyl, haloalkyl, hydroxyalkyl, hydroxyamino, dihydroxyalkyl, alkoxyalkyl, alkoxyamino, alkylthioalkyl, cycloalkyl, cyclohexylalkyl, amino, alkylamino, benzylalkyl, benzylamino, benzyloxycarbonylalkylamino, dialkylamino, adamantyl, haloalkoxybenzyl, alkoxyaminoalkyl, haloalkylarylalkyl, heterocyclylalkyl, alkoxyalkylamino, arylthioalkyl, substituted or unsubstituted aryl or arylalkyl, which, if substituted, have one or more substituent groups selected from H, alkyl, alkoxy, amino, nitro, halo, haloalkyl, or hydroxyalkyl;
   R¹⁴ is optionally present and if present, is selected from H or alkyl; and
   R¹⁵ is optionally present and if present, is selected from H, alkyl, alkoxy, or aminoalkyl.

The present invention is also directed to a novel method of modulating the activity of MK-2 in a subject in need of such modulation, the method comprising administering to the subject the MK-2 inhibiting compound having the structure according to formula I: 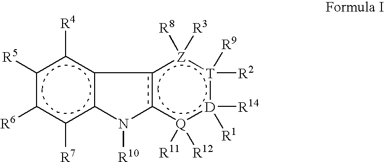
wherein:
Q, D, T and Z are selected from carbon, sulfur, or nitrogen;
when Q is sulfur, R¹² is oxo, and R¹¹ is optionally oxo or is absent;
when T is sulfur, R² is oxo, and R⁹ is optionally oxo or is absent;
when Z is nitrogen, D is nitrogen, and R² and R¹² are oxo;
R¹ is selected from H, alkyl, alkenyl, alkynyl, allyl, acetyl, amino, alkoxy, hydroxy, oxo, carboxy, allylalkyl, benzyl, 2-ethylalkyl, haloacetyl, t-butyldialkylsilyloxyalkyl, alkoxycarbonylaminoalkyl, haloalkylbenzyl, haloalkylcarbonyl, alkylcarbonyl, alkoxycarbonyl, arylcarbonyl, benzyloxycarbonyl, arylalkenylamino, alkoxyheterocyclylcarbonyl, haloarylcarbonyl, or substituted or unsubstituted alkyl, which, if substituted, has one or more substituent groups selected from H, halo, amino, aryl, or heteroaryl;
R² is selected from H, alkyl, alkoxy, hydroxy, carbonyl, oxo, benzyl, hydroxyalkyl, dialkylamino, carboxyl, amino, halo, alkoxycarbonyl, alkoxycarbonyl-R¹⁵, aminoalkyl, haloalkyl, hydroxyalkylbenzyl, haloalkylbenzyl, arylalkenylamino, haloalkoxybenzyl, cyanoalkyl, heterocyclylalkyl, arylalkyl-R¹⁵, alkyl-SO₂—R¹⁵, substituted or unsubstituted aryl, which, if substituted, has one or more substituent groups selected from H, alkyl, amino, nitro, halo, haloalkyl, or hydroxyalkyl, or substituted or unsubstituted alkyl, which, if substituted, has one or more substituent groups selected from H, halo, amino, aryl, or heteroaryl;
R³ is selected from H, oxo, alkyl, alkoxy, amino, halo, haloalkyl, hydroxy, carbonyl, alkoxycarbonyl, benzyl, aminoalkyl, hydroxyalkyl, dialkylamino, arylalkenylamino, carboxy, haloalkyl, hydroxyalkylbenzyl, haloalkylbenzyl, haloalkoxybenzyl, heterocyclylalkyl, substituted or unsubstituted aryl, which, if substituted, has one or more substituent groups selected from H, alkyl, amino, nitro, halo, haloalkyl, or hydroxyalkyl, or substituted or unsubstituted alkyl, which, if substituted, has one or more substituent groups selected from H, halo, amino, aryl, or heteroaryl, or R¹ and R³ optionally join to form an alkyl bridge between the ring atoms to which R¹ and R³ are attached, or R² and R³ optionally join to form a cycloalkyl ring;
R⁴, R⁶ and R⁷ are independently selected from H, alkyl, benzyl, halo, haloalkyl, alkoxy, haloalkoxy, carboxyl, CO₂—R¹⁵, carboxyl, nitro, amino, alkylamino, dialkylamino, benzyloxy, hydrazinocarbonyl, alkoxycarbonyl, alkoxycarbonylheterocyclylcarbonyl, hydroxy, or substituted or unsubstituted 6-membered heterocyclylcarbonyl, which, if substituted, has one or more substituent groups selected from H or alkoxycarbonyl, or R⁶ and R⁷ optionally join to form a heteroaryl ring;
R⁵ is selected from H, alkyl, alkenyl, alkylthio, hydroxy, carboxyl, halo, alkoxy, benzylalkyl, benzylalkoxy, arylalkyl, nitro, alkoxycarbonylalkoxy, alkylthio, benzyloxy, arylalkynyl, arylcarbonylalkoxy, haloalkoxy, haloalkyl, arylalkoxy, haloarylalkoxy, benzyloxoalkoxy, alkoxycarbonylbenzyloxy, alkoxycarbonyl, alkoxyoxoalkoxy, alkoxyoxoarylalkoxy, halobenzyloxy, heterocyclylalkoxy, dialkylaminooxoalkoxy, aminocarbonyl, arylalkynyl, hydrazinocarbonyl, carbonylalkoxy, alkylsulfonylbenzyloxy, CO₂—R¹⁵, carbamoyl, naphthylalkoxy, alkoxyaryloxy, alkoxycarbonylheterocyclylalkoxy, haloalkylbenzyloxy, alkoxyacetylaminoacetyloxy, acetoxy, cycloalkylalkoxy, acetyloxy, morpholinylcarbonyl, alkoxyalkoxy, or R⁴ and R⁵ optionally join to form a heterocyclic ring;
R⁸ and R⁹ are independently optionally present and if present, are independently selected from H, oxo, carbonitrile, alkyl, or is optionally absent, or R³ and R⁸ optionally join to form a cycloalkyl ring;
R¹⁰ is selected from H, alkyl, benzyl, aryl, or hydroxyalkyl;
R¹¹ is optionally present and if present, is selected from H, oxo, alkyl, halobenzyl, benzyl, haloalkyl, 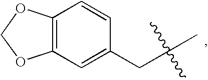
alkoxycarbonyl, carboxyalkyl, benzodioxinylalkyl, substituted or unsubstituted aryl or arylalkyl, which, if substituted, have one or more substituent groups selected from H, alkyl, or alkoxy;
R¹² is optionally present and if present, is selected from H, ═S, oxo, alkyl, carbonyl-R¹³, carboxyl-R¹³, ketone, cyano, cyanoalkyl, alkoxycarbonyl, aminoalkyl, alkoxycarbonylalkyl, carbamyl, hydroxycarbamyl, haloalkyl, heterocyclyl, alkylacetate, heterocyclylalkyl, alkoxycarbonylalkyl, hydroxyalkyl, dihydroxyalkyl, cyanoalkyl, ═N—OR¹⁵, ═N—N(R¹⁵)₂, or hydroxyamino, or R¹ and R¹² optionally join to form a substituted or unsubstituted 5 or 6-membered heteroaryl ring, which, if substituted, has one or more substituent groups selected from H, alkyl, oxo, or alkoxycarbonylalkyl, or R¹¹ and R¹² optionally join to form a ring system having the structure: 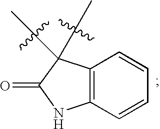
R¹³ is optionally present and if present, is selected from H, alkyl, alkoxy, amino, alkynyl, allyl, alkenyl, hydrazine, benzyl, glycine, allylalkyl, haloalkyl, hydroxyalkyl, hydroxyamino, dihydroxyalkyl, alkoxyalkyl, alkoxyamino, alkylthioalkyl, cycloalkyl, cyclohexylalkyl, amino, alkylamino, benzylalkyl, benzylamino, benzyloxycarbonylalkylamino, dialkylamino, adamantyl, haloalkoxybenzyl, alkoxyaminoalkyl, haloalkylarylalkyl, heterocyclylalkyl, alkoxyalkylamino, arylthioalkyl, substituted or unsubstituted aryl or arylalkyl, which, if substituted, have one or more substituent groups selected from H, alkyl, alkoxy, amino, nitro, halo, haloalkyl, or hydroxyalkyl;
R¹⁴ is optionally present and if present, is selected from H or alkyl; and
R¹⁵ is optionally present and if present, is selected from H, alkyl, alkoxy, or aminoalkyl.

The present invention is also directed to a novel method of inhibiting MK-2 activity in a cell, the method comprising contacting the cell with a MK-2 inhibiting compound having the structure according to formula I: 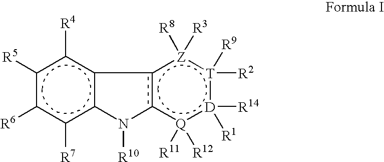
wherein:
Q, D, T and Z are selected from carbon, sulfur, or nitrogen;
when Q is sulfur, R¹² is oxo, and R¹¹ is optionally oxo or is absent;
when T is sulfur, R² is oxo, and R⁹ is optionally oxo or is absent;
when Z is nitrogen, D is nitrogen, and R² and R¹² are oxo;
R¹ is selected from H, alkyl, alkenyl, alkynyl, allyl, acetyl, amino, alkoxy, hydroxy, oxo, carboxy, allylalkyl, benzyl, 2-ethylalkyl, haloacetyl, t-butyldialkylsilyloxyalkyl, alkoxycarbonylaminoalkyl, haloalkylbenzyl, haloalkylcarbonyl, alkylcarbonyl, alkoxycarbonyl, arylcarbonyl, benzyloxycarbonyl, arylalkenylamino, alkoxyheterocyclylcarbonyl, haloarylcarbonyl, or substituted or unsubstituted alkyl, which, if substituted, has one or more substituent groups selected from H, halo, amino, aryl, or heteroaryl;
R² is selected from H, alkyl, alkoxy, hydroxy, carbonyl, oxo, benzyl, hydroxyalkyl, dialkylamino, carboxyl, amino, halo, alkoxycarbonyl, alkoxycarbonyl-R¹⁵, aminoalkyl, haloalkyl, hydroxyalkylbenzyl, haloalkylbenzyl, arylalkenylamino, haloalkoxybenzyl, cyanoalkyl, heterocyclylalkyl, arylalkyl-R¹⁵, alkyl-SO₂—R¹⁵, substituted or unsubstituted aryl, which, if substituted, has one or more substituent groups selected from H, alkyl, amino, nitro, halo, haloalkyl, or hydroxyalkyl, or substituted or unsubstituted alkyl, which, if substituted, has one or more substituent groups selected from H, halo, amino, aryl, or heteroaryl;
R³ is selected from H, oxo, alkyl, alkoxy, amino, halo, haloalkyl, hydroxy, carbonyl, alkoxycarbonyl, benzyl, aminoalkyl, hydroxyalkyl, dialkylamino, arylalkenylamino, carboxy, haloalkyl, hydroxyalkylbenzyl, haloalkylbenzyl, haloalkoxybenzyl, heterocyclylalkyl, substituted or unsubstituted aryl, which, if substituted, has one or more substituent groups selected from H, alkyl, amino, nitro, halo, haloalkyl, or hydroxyalkyl, or substituted or unsubstituted alkyl, which, if substituted, has one or more substituent groups selected from H, halo, amino, aryl, or heteroaryl, or R¹ and R³ optionally join to form an alkyl bridge between the ring atoms to which R¹ and R³ are attached, or R² and R³ optionally join to form a cycloalkyl ring;
R⁴, R⁶ and R⁷ are independently selected from H, alkyl, benzyl, halo, haloalkyl, alkoxy, haloalkoxy, carboxyl, CO₂—R¹⁵, carboxyl, nitro, amino, alkylamino, dialkylamino, benzyloxy, hydrazinocarbonyl, alkoxycarbonyl, alkoxycarbonylheterocyclylcarbonyl, hydroxy, or substituted or unsubstituted 6-membered heterocyclylcarbonyl, which, if substituted, has one or more substituent groups selected from H or alkoxycarbonyl, or R⁶ and R⁷ optionally join to form a heteroaryl ring;
R⁵ is selected from H, alkyl, alkenyl, alkylthio, hydroxy, carboxyl, halo, alkoxy, benzylalkyl, benzylalkoxy, arylalkyl, nitro, alkoxycarbonylalkoxy, alkylthio, benzyloxy, arylalkynyl, arylcarbonylalkoxy, haloalkoxy, haloalkyl, arylalkoxy, haloarylalkoxy, benzyloxoalkoxy, alkoxycarbonylbenzyloxy, alkoxycarbonyl, alkoxyoxoalkoxy, alkoxyoxoarylalkoxy, halobenzyloxy, heterocyclylalkoxy, dialkylaminooxoalkoxy, aminocarbonyl, arylalkynyl, hydrazinocarbonyl, carbonylalkoxy, alkylsulfonylbenzyloxy, CO₂—R¹⁵, carbamoyl, naphthylalkoxy, alkoxyaryloxy, alkoxycarbonylheterocyclylalkoxy, haloalkylbenzyloxy, alkoxyacetylaminoacetyloxy, acetoxy, cycloalkylalkoxy, acetyloxy, morpholinylcarbonyl, alkoxyalkoxy, or R⁴ and R⁵ optionally join to form a heterocyclic ring;
R⁸ and R⁹ are independently optionally present and if present, are independently selected from H, oxo, carbonitrile, alkyl, or is optionally absent, or R³ and R⁸ optionally join to form a cycloalkyl ring;
R¹⁰ is selected from H, alkyl, benzyl, aryl, or hydroxyalkyl;
R¹¹ is optionally present and if present, is selected from H, oxo, alkyl, halobenzyl, benzyl, haloalkyl, 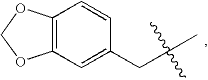
alkoxycarbonyl, carboxyalkyl, benzodioxinylalkyl, substituted or unsubstituted aryl or arylalkyl, which, if substituted, have one or more substituent groups selected from H, alkyl, or alkoxy;
R¹² is optionally present and if present, is selected from H, ═S, oxo, alkyl, carbonyl-R¹³, carboxyl-R¹³, ketone, cyano, cyanoalkyl, alkoxycarbonyl, aminoalkyl, alkoxycarbonylalkyl, carbamyl, hydroxycarbamyl, haloalkyl, heterocyclyl, alkylacetate, heterocyclylalkyl, alkoxycarbonylalkyl, hydroxyalkyl, dihydroxyalkyl, cyanoalkyl, ═N—OR¹⁵, ═N—N(R¹⁵)₂, or hydroxyamino, or R¹ and R¹² optionally join to form a substituted or unsubstituted 5 or 6-membered heteroaryl ring, which, if substituted, has one or more substituent groups selected from H, alkyl, oxo, or alkoxycarbonylalkyl, or R¹¹ and R¹² optionally join to form a ring system having the structure: 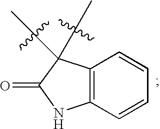
R¹³ is optionally present and if present, is selected from H, alkyl, alkoxy, amino, alkynyl, allyl, alkenyl, hydrazine, benzyl, glycine, allylalkyl, haloalkyl, hydroxyalkyl, hydroxyamino, dihydroxyalkyl, alkoxyalkyl, alkoxyamino, alkylthioalkyl, cycloalkyl, cyclohexylalkyl, amino, alkylamino, benzylalkyl, benzylamino, benzyloxycarbonylalkylamino, dialkylamino, adamantyl, haloalkoxybenzyl, alkoxyaminoalkyl, haloalkylarylalkyl, heterocyclylalkyl, alkoxyalkylamino, arylthioalkyl, substituted or unsubstituted aryl or arylalkyl, which, if substituted, have one or more substituent groups selected from H, alkyl, alkoxy, amino, nitro, halo, haloalkyl, or hydroxyalkyl;
R¹⁴ is optionally present and if present, is selected from H or alkyl; and
R¹⁵ is optionally present and if present, is selected from H, alkyl, alkoxy, or aminoalkyl.

The present invention is also directed to a novel compound, or a pharmaceutically acceptable salt or isomer thereof, the compound having the structure according to formula I: 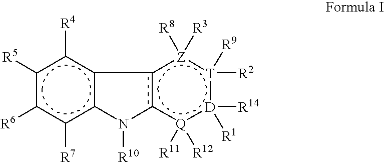
wherein:
Q, D, T and Z are selected from carbon, sulfur, or nitrogen;
when Q is sulfur, R¹² is oxo, and R¹¹ is optionally oxo or is absent;
when T is sulfur, R² is oxo, and R⁹ is optionally oxo or is absent;
when Z is nitrogen, D is nitrogen, and R² and R¹² are oxo;
R¹ is selected from H, alkyl, alkenyl, alkynyl, allyl, acetyl, amino, alkoxy, hydroxy, oxo, carboxy, allylalkyl, benzyl, 2-ethylalkyl, haloacetyl, t-butyldialkylsilyloxyalkyl, alkoxycarbonylaminoalkyl, haloalkylbenzyl, haloalkylcarbonyl, alkylcarbonyl, alkoxycarbonyl, arylcarbonyl, benzyloxycarbonyl, arylalkenylamino, alkoxyheterocyclylcarbonyl, haloarylcarbonyl, or substituted or unsubstituted alkyl, which, if substituted, has one or more substituent groups selected from H, halo, amino, aryl, or heteroaryl;
R² is selected from H, alkyl, alkoxy, hydroxy, carbonyl, oxo, benzyl, hydroxyalkyl, dialkylamino, carboxyl, amino, halo, alkoxycarbonyl, alkoxycarbonyl-R¹⁵, aminoalkyl, haloalkyl, hydroxyalkylbenzyl, haloalkylbenzyl, arylalkenylamino, haloalkoxybenzyl, cyanoalkyl, heterocyclylalkyl, arylalkyl-R¹⁵, alkyl-SO₂—R¹⁵, substituted or unsubstituted aryl, which, if substituted, has one or more substituent groups selected from H, alkyl, amino, nitro, halo, haloalkyl, or hydroxyalkyl, or substituted or unsubstituted alkyl, which, if substituted, has one or more substituent groups selected from H, halo, amino, aryl, or heteroaryl;
R³ is selected from H, oxo, alkyl, alkoxy, amino, halo, haloalkyl, hydroxy, carbonyl, alkoxycarbonyl, benzyl, aminoalkyl, hydroxyalkyl, dialkylamino, arylalkenylamino, carboxy, haloalkyl, hydroxyalkylbenzyl, haloalkylbenzyl, haloalkoxybenzyl, heterocyclylalkyl, substituted or unsubstituted aryl, which, if substituted, has one or more substituent groups selected from H, alkyl, amino, nitro, halo, haloalkyl, or hydroxyalkyl, or substituted or unsubstituted alkyl, which, if substituted, has one or more substituent groups selected from H, halo, amino, aryl, or heteroaryl, or R¹ and R³ optionally join to form an alkyl bridge between the ring atoms to which R¹ and R³ are attached, or R² and R³ optionally join to form a cycloalkyl ring;
R⁴, R⁶ and R⁷ are independently selected from H, alkyl, benzyl, halo, haloalkyl, alkoxy, haloalkoxy, carboxyl, CO₂—R¹⁵, carboxyl, nitro, amino, alkylamino, dialkylamino, benzyloxy, hydrazinocarbonyl, alkoxycarbonyl, alkoxycarbonylheterocyclylcarbonyl, hydroxy, or substituted or unsubstituted 6-membered heterocyclylcarbonyl, which, if substituted, has one or more substituent groups selected from H or alkoxycarbonyl, or R⁶ and R⁷ optionally join to form a heteroaryl ring;
R⁵ is selected from H, alkyl, alkenyl, alkylthio, hydroxy, carboxyl, halo, alkoxy, benzylalkyl, benzylalkoxy, arylalkyl, nitro, alkoxycarbonylalkoxy, alkylthio, benzyloxy, arylalkynyl, arylcarbonylalkoxy, haloalkoxy, haloalkyl, arylalkoxy, haloarylalkoxy, benzyloxoalkoxy, alkoxycarbonylbenzyloxy, alkoxycarbonyl, alkoxyoxoalkoxy, alkoxyoxoarylalkoxy, halobenzyloxy, heterocyclylalkoxy, dialkylaminooxoalkoxy, aminocarbonyl, arylalkynyl, hydrazinocarbonyl, carbonylalkoxy, alkylsulfonylbenzyloxy, CO₂—R¹⁵, carbamoyl, naphthylalkoxy, alkoxyaryloxy, alkoxycarbonylheterocyclylalkoxy, haloalkylbenzyloxy, alkoxyacetylaminoacetyloxy, acetoxy, cycloalkylalkoxy, acetyloxy, morpholinylcarbonyl, alkoxyalkoxy, or R⁴ and R⁵ optionally join to form a heterocyclic ring;
R⁸ and R⁹ are independently optionally present and if present, are independently selected from H, oxo, carbonitrile, alkyl, or is optionally absent, or R³ and R⁸ optionally join to form a cycloalkyl ring;
R¹⁰ is selected from H, alkyl, benzyl, aryl, or hydroxyalkyl;
R¹¹ is optionally present and if present, is selected from H, oxo, alkyl, halobenzyl, benzyl, haloalkyl, 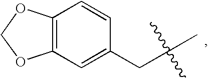
alkoxycarbonyl, carboxyalkyl, benzodioxinylalkyl, substituted or unsubstituted aryl or arylalkyl, which, if substituted, have one or more substituent groups selected from H, alkyl, or alkoxy;
R¹² is optionally present and if present, is selected from H, ═S, oxo, alkyl, carbonyl-R¹³, carboxyl-R¹³, ketone, cyano, cyanoalkyl, alkoxycarbonyl, aminoalkyl, alkoxycarbonylalkyl, carbamyl, hydroxycarbamyl, haloalkyl, heterocyclyl, alkylacetate, heterocyclylalkyl, alkoxycarbonylalkyl, hydroxyalkyl, dihydroxyalkyl, cyanoalkyl, ═N—OR¹⁵, ═N—N(R¹⁵)₂, or hydroxyamino, or R¹ and R¹² optionally join to form a substituted or unsubstituted 5 or 6-membered heteroaryl ring, which, if substituted, has one or more substituent groups selected from H, alkyl, oxo, or alkoxycarbonylalkyl, or R¹¹ and R¹² optionally join to form a ring system having the structure: 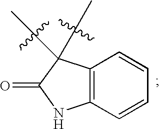
R¹³ is optionally present and if present, is selected from H, alkyl, alkoxy, amino, alkynyl, allyl, alkenyl, hydrazine, benzyl, glycine, allylalkyl, haloalkyl, hydroxyalkyl, hydroxyamino, dihydroxyalkyl, alkoxyalkyl, alkoxyamino, alkylthioalkyl, cycloalkyl, cyclohexylalkyl, amino, alkylamino, benzylalkyl, benzylamino, benzyloxycarbonylalkylamino, dialkylamino, adamantyl, haloalkoxybenzyl, alkoxyaminoalkyl, haloalkylarylalkyl, heterocyclylalkyl, alkoxyalkylamino, arylthioalkyl, substituted or unsubstituted aryl or arylalkyl, which, if substituted, have one or more substituent groups selected from H, alkyl, alkoxy, amino, nitro, halo, haloalkyl, or hydroxyalkyl;
R¹⁴ is optionally present and if present, is selected from H or alkyl; and
R¹⁵ is optionally present and if present, is selected from H, alkyl, alkoxy, or aminoalkyl.

The present invention is also directed to a novel method for treating or preventing a disease or disorder in a subject, which disease or disorder is one that can be treated or prevented by inhibiting the activity of MK-2, the method comprising administering to the subject a compound, or a salt, isomer, or prodrug thereof, wherein the compound has the structure according to formula IV: 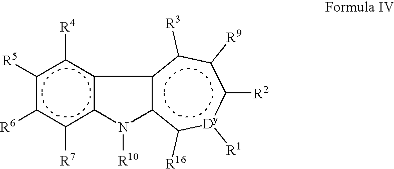
wherein:
D^{y} is selected from carbon or nitrogen;
R¹, R², R³ and R⁹ are independently selected from H, alkyl, alkoxy, carbonitrile, oxo, arylalkenylamino, or amino;
R⁴, R⁶ and R⁷ are independently selected from H, alkyl, alkoxy, halo, carboxyl, nitro, or substituted or unsubstituted 6-membered heteroarylcarbonyl, which, if substituted, have one or more substituent groups selected from H or alkoxycarbonyl;
R⁵ is selected from H or alkoxy;
R¹⁰ is selected from H, alkyl, or aryl; and
R¹⁶ is selected from H, oxo, CO₂H, or oxime.

The present invention is also directed to a novel method for treating or preventing a disease or disorder in a subject, which disease or disorder is one that can be treated or prevented by inhibiting the activity of MK-2, the method comprising administering to the subject a compound, or a pharmaceutically acceptable salt or prodrug thereof, wherein the compound has the structure according to formula V: 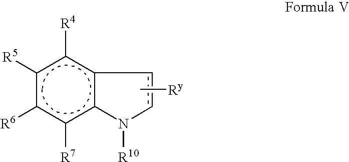
wherein:
R^{y} is optionally fused with the pyrrole ring and is selected from a substituted or unsubstituted 5-7 membered heterocyclic ring or a substituted or unsubstituted 5-7 membered cycloalkyl ring, which, if substituted, have one or more substituent groups selected from H, alkyl, alkoxy, carbonitrile, oxo, arylalkenylamino, halo, oxime, or amino;
R⁴, R⁶ and R⁷ are independently selected from H, alkyl, alkoxy, halo, carboxyl, nitro, or substituted or unsubstituted 6-membered heteroarylcarbonyl, which, if substituted, have one or more substituent groups selected from H or alkoxycarbonyl;
R⁵ is selected from H or alkoxy; and
R¹⁰ is selected from H, alkyl, or aryl.

Among the several advantages found to be achieved by the present invention, therefore, may be noted the provision of a method that could serve to modulate the activity of MK-2—in particular, to inhibit MK-2 activity, and the provision of a method for the prevention and treatment of diseases and disorders that can be prevented or treated by the modulation of MK-2.

### BRIEF DESCRIPTION OF THE DRAWINGS

FIG. 1 is a graph showing paw thickness as a function of time from day 0 to day 7 for MK2 (+/+) and MK2 (−/−) mice, which have received serum injection; and

FIG. 2 is a bar chart showing paw thickness at seven days after injection for normal mice, MK2 (+/+) mice receiving serum, MK2 (−/−) mice receiving serum, and MK2 (+/+) mice receiving serum and anti-TNF antibody.

### DETAILED DESCRIPTION OF THE PREFERRED EMBODIMENTS

In accordance with the present invention, it has been discovered that certain beta-carboline compounds, and analogues of such compounds, can inhibit the activity of mitogen-activated protein kinase-activated protein kinase-2 (which may herein be termed MAPKAP-2, or MK-2). Many of these compounds exhibit their inhibitory effect at low concentrations—having in vitro MK-2 inhibition IC₅₀ values of under 20.0 μM, and with some having IC₅₀ values of under about 1.0 μM, and even as low as about 0.05 μM.

Because MK-2 is an essential component in the inflammatory response that regulates TNFα biosynthesis at a post-transcriptional level, inhibition of MK-2 activity leads to a concomitant reduction in TNFα production. Accordingly, these compounds can be potent and effective drugs for use in methods to prevent or treat diseases and disorders that are mediated by TNFα. For example, they can be used for the prevention or treatment of arthritis.

Preferred MK-2 inhibitors of the present invention include a compound, or a pharmaceutically acceptable salt, isomer, stereoisomer or enantiomer thereof, the compound having the structure shown in formula I: 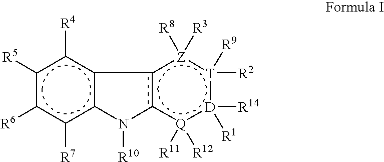
wherein:
Q, D, T and Z are selected from carbon, sulfur, or nitrogen;
when Q is sulfur, R¹² is oxo, and R¹¹ is optionally oxo or is absent;
when T is sulfur, R² is oxo, and R⁹ is optionally oxo or is absent;
when Z is nitrogen, D is nitrogen, and R² and R¹² are oxo;
R¹ is selected from H, alkyl, alkenyl, alkynyl, allyl, acetyl, amino, alkoxy, hydroxy, oxo, carboxy, allylalkyl, benzyl, 2-ethylalkyl, haloacetyl, t-butyldialkylsilyloxyalkyl, alkoxycarbonylaminoalkyl, haloalkylbenzyl, haloalkylcarbonyl, alkylcarbonyl, alkoxycarbonyl, arylcarbonyl, benzyloxycarbonyl, arylalkenylamino, alkoxyheterocyclylcarbonyl, haloarylcarbonyl, or substituted or unsubstituted alkyl, which, if substituted, has one or more substituent groups selected from H, halo, amino, aryl, or heteroaryl;
R² is selected from H, alkyl, alkoxy, hydroxy, carbonyl, oxo, benzyl, hydroxyalkyl, dialkylamino, carboxyl, amino, halo, alkoxycarbonyl, alkoxycarbonyl-R¹⁵, aminoalkyl, haloalkyl, hydroxyalkylbenzyl, haloalkylbenzyl, arylalkenylamino, haloalkoxybenzyl, cyanoalkyl, heterocyclylalkyl, arylalkyl-R¹⁵, alkyl-SO₂—R¹⁵, substituted or unsubstituted aryl, which, if substituted, has one or more substituent groups selected from H, alkyl, amino, nitro, halo, haloalkyl, or hydroxyalkyl, or substituted or unsubstituted alkyl, which, if substituted, has one or more substituent groups selected from H, halo, amino, aryl, or heteroaryl;
R³ is selected from H, oxo, alkyl, alkoxy, amino, halo, haloalkyl, hydroxy, carbonyl, alkoxycarbonyl, benzyl, aminoalkyl, hydroxyalkyl, dialkylamino, arylalkenylamino, carboxy, haloalkyl, hydroxyalkylbenzyl, haloalkylbenzyl, haloalkoxybenzyl, heterocyclylalkyl, substituted or unsubstituted aryl, which, if substituted, has one or more substituent groups selected from H, alkyl, amino, nitro, halo, haloalkyl, or hydroxyalkyl, or substituted or unsubstituted alkyl, which, if substituted, has one or more substituent groups selected from H, halo, amino, aryl, or heteroaryl, or R¹ and R³ optionally join to form an alkyl bridge between the ring atoms to which R¹ and R³ are attached, or R² and R³ optionally join to form a cycloalkyl ring;
R⁴, R⁶ and R⁷ are independently selected from H, alkyl, benzyl, halo, haloalkyl, alkoxy, haloalkoxy, carboxyl, CO₂—R¹⁵, carboxyl, nitro, amino, alkylamino, dialkylamino, benzyloxy, hydrazinocarbonyl, alkoxycarbonyl, alkoxycarbonylheterocyclylcarbonyl, hydroxy, or substituted or unsubstituted 6-membered heterocyclylcarbonyl, which, if substituted, has one or more substituent groups selected from H or alkoxycarbonyl, or R⁶ and R⁷ optionally join to form a heteroaryl ring;
R⁵ is selected from H, alkyl, alkenyl, alkylthio, hydroxy, carboxyl, halo, alkoxy, benzylalkyl, benzylalkoxy, arylalkyl, nitro, alkoxycarbonylalkoxy, alkylthio, benzyloxy, arylalkynyl, arylcarbonylalkoxy, haloalkoxy, haloalkyl, arylalkoxy, haloarylalkoxy, benzyloxoalkoxy, alkoxycarbonylbenzyloxy, alkoxycarbonyl, alkoxyoxoalkoxy, alkoxyoxoarylalkoxy, halobenzyloxy, heterocyclylalkoxy, dialkylaminooxoalkoxy, aminocarbonyl, arylalkynyl, hydrazinocarbonyl, carbonylalkoxy, alkylsulfonylbenzyloxy, CO₂—R¹⁵, carbamoyl, naphthylalkoxy, alkoxyaryloxy, alkoxycarbonylheterocyclylalkoxy, haloalkylbenzyloxy, alkoxyacetylaminoacetyloxy, acetoxy, cycloalkylalkoxy, acetyloxy, morpholinylcarbonyl, alkoxyalkoxy, or R⁴ and R⁵ optionally join to form a heterocyclic ring;
R⁸ and R⁹ are independently optionally present and if present, are independently selected from H, oxo, carbonitrile, alkyl, or is optionally absent, or R³ and R⁸ optionally join to form a cycloalkyl ring;
R¹⁰ is selected from H, alkyl, benzyl, aryl, or hydroxyalkyl;
R¹¹ is optionally present and if present, is selected from H, oxo, alkyl, halobenzyl, benzyl, haloalkyl, 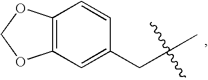
alkoxycarbonyl, carboxyalkyl, benzodioxinylalkyl, substituted or unsubstituted aryl or arylalkyl, which, if substituted, have one or more substituent groups selected from H, alkyl, or alkoxy;
R¹² is optionally present and if present, is selected from H, ═S, oxo, alkyl, carbonyl-R¹³, carboxyl-R¹³, ketone, cyano, cyanoalkyl, alkoxycarbonyl, aminoalkyl, alkoxycarbonylalkyl, carbamyl, hydroxycarbamyl, haloalkyl, heterocyclyl, alkylacetate, heterocyclylalkyl, alkoxycarbonylalkyl, hydroxyalkyl, dihydroxyalkyl, cyanoalkyl, ═N—OR¹⁵, ═N—N(R¹⁵)₂, or hydroxyamino, or R¹ and R¹² optionally join to form a substituted or unsubstituted 5 or 6-membered heteroaryl ring, which, if substituted, has one or more substituent groups selected from H, alkyl, oxo, or alkoxycarbonylalkyl, or R¹¹ and R¹² optionally join to form a ring system having the structure: 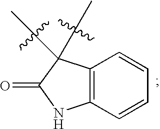
R¹³ is optionally present and if present, is selected from H, alkyl, alkoxy, amino, alkynyl, allyl, alkenyl, hydrazine, benzyl, glycine, allylalkyl, haloalkyl, hydroxyalkyl, hydroxyamino, dihydroxyalkyl, alkoxyalkyl, alkoxyamino, alkylthioalkyl, cycloalkyl, cyclohexylalkyl, amino, alkylamino, benzylalkyl, benzylamino, benzyloxycarbonylalkylamino, dialkylamino, adamantyl, haloalkoxybenzyl, alkoxyaminoalkyl, haloalkylarylalkyl, heterocyclylalkyl, alkoxyalkylamino, arylthioalkyl, substituted or unsubstituted aryl or arylalkyl, which, if substituted, have one or more substituent groups selected from H, alkyl, alkoxy, amino, nitro, halo, haloalkyl, or hydroxyalkyl;
R¹⁴ is optionally present and if present, is selected from H or alkyl; and
R¹⁵ is optionally present and if present, is selected from H, alkyl, alkoxy, or aminoalkyl.

The compounds that are described above include beta-carboline compounds and certain analogues and/or derivatives of beta-carboline compounds, all of which are capable of inhibiting MK-2. As a group, these compounds may be referred to herein as “beta-carboline MK-2 inhibitors”, or “beta-carboline MK-2 inhibiting compounds”.

As shown above, ring substituent groups that join to form additional ring structures adjacent the substituted ring can be described with reference to chemical formulas that show wavy lines cut through the ring to which the substituents are joined, rather than across the bond joining the substituent group to the ring. Accordingly, the partial ring that is shown is the ring to which the substituent groups are shown as being bonded in the general formula.

As used herein, the term “alkyl”, alone or in combination, means an acyclic alkyl radical, linear or branched, which, unless otherwise noted, preferably contains from 1 to about 10 carbon atoms and more preferably contains from 1 to about 6 carbon atoms. “Alkyl” also encompasses cyclic alkyl radicals containing from 3 to about 7 carbon atoms, preferably from 3 to 5 carbon atoms. The alkyl radicals can be optionally substituted with groups as defined below. Examples of such alkyl radicals include methyl, ethyl, chloroethyl, hydroxyethyl, n-propyl, isopropyl, n-butyl, cyanobutyl, isobutyl, sec-butyl, tert-butyl, pentyl, aminopentyl, iso-amyl, hexyl, octyl, and the like.

The term “alkenyl” refers to an unsaturated, acyclic hydrocarbon radical, linear or branched, in so much as it contains at least one double bond. Unless otherwise noted, such radicals preferably contain from 2 to about 6 carbon atoms, preferably from 2 to about 4 carbon atoms, more preferably from 2 to about 3 carbon atoms. The alkenyl radicals may be optionally substituted with groups as defined below. Examples of suitable alkenyl radicals include propenyl, 2-chloropropylenyl, buten-1yl, isobutenyl, penten-1yl, 2-methylbuten-1-yl, 3-methylbuten-1-yl, hexen-1-yl, 3-hydroxyhexen-1-yl, hepten-1-yl, octen-1-yl, and the like.

The term “alkoxy” includes linear or branched oxy-containing radicals, each of which has, unless otherwise noted, alkyl portions of 1 to about 6 carbon atoms, preferably 1 to about 4 carbon atoms, such as methoxy, ethoxy, propoxy, isopropoxy, isobutoxy radicals, and the like.

The term “alkoxyalkyl” also embraces alkyl radicals having one or more alkoxy radicals attached to the alkyl radical, that is, to form monoalkoxyalkyl and dialkoxyalkyl radicals. Examples of such radicals include methoxyalkyls, ethoxyalkyls, propoxyalkyls, isopropoxyalkyls, butoxyalkyls, tert-butoxyalkyls, and the like. The “alkoxy” radicals may be further substituted with one or more halo atoms, such as fluoro, chloro, or bromo, to provide “haloalkoxy” radicals. Examples of such radicals include fluoromethoxy, chloromethoxy, trifluoromethoxy, difluoromethoxy, trifluoroethoxy, fluoroethoxy, tetrafluoroethoxy, pentafluoroethoxy, fluoropropoxy, and the like.

The term “alkylthio” embraces radicals containing a linear or branched alkyl radical, preferably, unless otherwise noted, of from 1 to about 6 carbon atoms, attached to a divalent sulfur atom. An example of “lower alkylthio”, is methylthio (CH₃—S—).

The term “alkylthioalkyl” embraces alkylthio radicals, attached to an alkyl group. An example of such radicals is methylthiomethyl.

The term “heterocyclyl” means a saturated or unsaturated mono- or multi-ring carbocycle wherein one or more carbon atoms is replaced by N, S, P, or O. This includes, for example, structures such as: 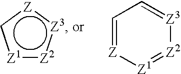
where Z, Z¹, Z², or Z³ is C, S, P, O, or N, with the proviso that one of Z, Z¹, Z², or Z³ is other than carbon, but is not O or S when attached to another Z atom by a double bond or when attached to another O or S atom. Furthermore, the optional substituents are understood to be attached to Z, Z¹, Z², or Z³ only when each is C. The term “heterocycle” also includes fully saturated ring structures, such as piperazinyl, dioxanyl, tetrahydrofuranyl, oxiranyl, aziridinyl, morpholinyl, pyrrolidinyl, piperidinyl, thiazolidinyl, and others.

As used herein, and known to one of ordinary skill in the art, the use of a circular designation, whether a solid circle or a dashed circle, within a cyclic structure is meant to encompass the appropriate double bonding character, if any, between the cyclic ring atoms, regardless of whether the ring atoms are C, S, P, O, or N. Likewise, the use of a dashed line that is adjacent to a solid line or bond is meant to encompass the appropriate double bonding character, if any, between the two ring atoms that are indicated as bonded with either a single bond or a double bond.

The term “heteroaryl” means a fully unsaturated heterocycle, which can include, but is not limited to, furyl, thenyl, pyrryl, imidazolyl, pyrazolyl, pyridyl, thiazolyl, quinolinyl, isoquinolinyl, benzothienyl, and indolyl.

In either, “heterocyclyl” or “heteroaryl”, the point of attachment to the molecule of interest can be at the heteroatom or elsewhere within the ring.

The term “cycloalkyl” means a mono- or multi-ringed carbocycle wherein each ring contains three to about seven carbon atoms, preferably three to about six carbon atoms, and more preferably three to about five carbon atoms. Examples include radicals, such as cyclopropyl, cyclobutyl, cyclopentyl, cyclohexyl, cycloalkenyl, and cycloheptyl. The term “cycloalkyl” additionally encompasses spiro systems wherein the cycloalkyl ring has a carbon ring atom in common with the seven-membered heterocyclic ring of the benzothiepine.

The term “oxo” means a doubly-bonded oxygen.

The term “aryl” means a fully unsaturated mono- or multi-ring carbocycle, including, but not limited to, substituted or unsubstituted phenyl, naphthyl, or anthracenyl.

As used herein, “halo” or “halogen” means fluorine, chlorine, bromine, iodine, or astatine.

As used herein, “organic halide” means a compound having fluorine, chlorine, bromine, iodine, or astatine covalently coupled with an alkyl, alkenyl, alkynyl, alkoxy, aralkyl, aryl, carbonyl, cycloalkyl, benzyl, phenyl, alicyclic or heterocyclic group.

As used herein, the terms “carboxy” or “carboxyl” or “carboxylic acid” refers to a functional group that consists of a carbon atom joined to an oxygen atom by a double bond and to a hydroxyl group, OH, by a single bond.

As used herein, the term “carbamoyl” refers to a carbonyl group covalently bonded at the oxo carbon to an amino group.

As used herein, the term “hydroxamate” refers to a carbonyl group covalently bonded at the oxo carbon to an amino group, wherein the amino group is in turn bonded to a hydroxyl group.

The term “oxime” means a radical comprising ═NOH.

The present beta-carboline MK-2 inhibitors inhibit the activity of the MK-2 enzyme. When it is said that a subject compound inhibits MK-2, it is meant that the MK-2 enzymatic activity is lower in the presence of the compound than it is under the same conditions in the absence of such compound. One method of expressing the potency of a compound as an MK-2 inhibitor is to measure the “IC₅₀” value of the compound. The IC₅₀ value of an MK-2 inhibitor is the concentration of the compound that is required to decrease the MK-2 enzymatic activity by one-half. Accordingly, a compound having a lower IC₅₀ value is considered to be a more potent inhibitor than a compound having a higher IC₅₀ value.

Examples of beta-carboline MK-2 inhibitors that are suitable for purposes of the present invention include, but are not limited to, those compounds described in Table 1 that follows. Where a particular beta-carboline compound has multiple MK-2 values, it is to be understood that the additional MK-2 values are replicate IC₅₀ experiments and should not be construed as limiting the present invention.

TABLE 1
Examples of beta-carboline MK-2 Inhibitors

| | | | | TNF |
|---|---|---|---|---|
| | | | | Release |
| | | | MK-2 | Assay |
| | | | Avg. | Avg. |
| | | | IC50 | IC50 |
| No. | Structure^{a} | Compound Name(s)^{b} | (μM)^{c} | (μM)^{c} |
| 1 | 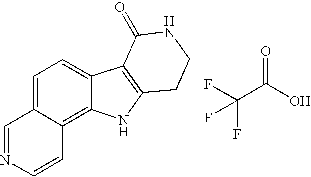 | 8,9,10,11-tetrahydro-7H-py- rido[3′,4′:4,5]pyrrolo[2,3-f]isoquinolin-7-one trifluoroacetate | 0.0408 | 0.845 |
| 2 | 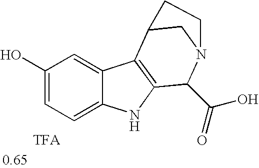 | 7-hydroxy-3,4,5,10-tetrahydro-1H-2,5-methano- azepino[3,4-b]indole-1-carboxylic acid trifluoroacetate | 0.27 | >100 |
| | | | 0.274 | 42.6 |
| 3 | 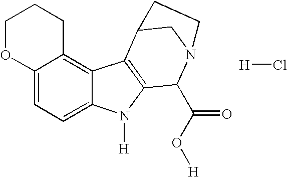 | 2,3,8,10,11,12-hexahydro-1H,7H-9,12-methano- azepino[3,4-b]pyrano[3,2-e]indole-8-car- boxylic acid | 0.283 | >100 |
| 4 | 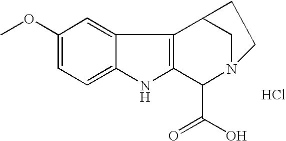 | 7-methoxy-3,4,5,10-tetrahydro-1H-2,5-meth- anoazepino[3,4-b]indole-1-carboxylic acid hydrochloride | 0.367 | >100 |
| | | | 0.398 | >100 |
| | | | | 34.6 |
| 5 | 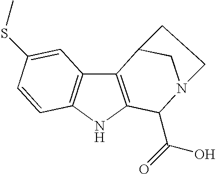 | 7-(methylthio)-3,4,5,10-tetra- hydro-1H-2,5-methano- azepino[3,4-b]indole-1-carboxylic acid | 0.513 | >100 |
| 6 | 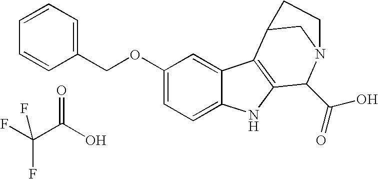 | 7-(benzyloxy)-3,4,5,10-tetra- hydro-1H-2,5-methano- azepino[3,4-b]indole-1-carboxylic acid trifluoroacetate | 0.654 | >100 |
| 7 | 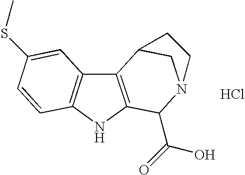 | 7-(methylthio)-3,4,5,10-tetra- hydro-1H-2,5-methano- azepino[3,4-b]indole-1-carboxylic acid hydrochloride | 0.79 | >100 |
| 8 | 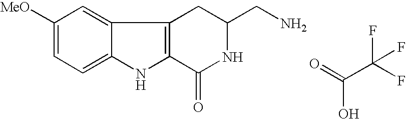 | 3-(aminomethyl)-6-meth- oxy-2,3,4,9-tetra- hydro-1H-beta-carbolin-1-one trifluroacetate | 0.835 | 20.1 |
| 9 | 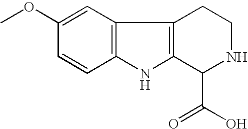 | (1R)-6-methoxy-2,3,4,9-tetra- hydro-1H-beta-car- boline-1-carboxylic acid | 0.997 | >100 |
| 10 | 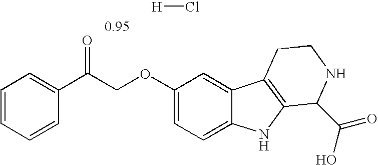 | 6-(2-oxo-2-phenylethoxy)-2,3,4,9-tetra- hydro-1H-beta-carbo- line-1-carboxylic acid hydrochloride | 1.08 | >100 |
| 11 | 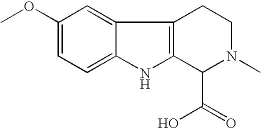 | 6-methoxy-2-methyl-2,3,4,9-tet- rahydro-1H-beta-car- boline-1-carboxylic acid | 1.23 | 78.2 |
| 12 | 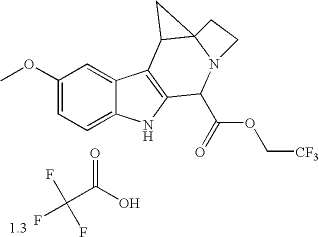 | 2,2,2-trifluoroethyl 7-methoxy-3,4,5,10-tetra- hydro-1H-2,5-methanoazepino[3,4-b]in- dole-1-carboxylate trifluoroacetate | 1.26 | 8.62 |
| 13 | 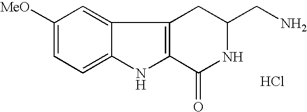 | 3-(aminomethyl)-6-methoxy-2,3,4,9-tetra- hydro-1H-beta-carbolin-1-one hydrochloride | 1.32 | 20.8 |
| 14 | 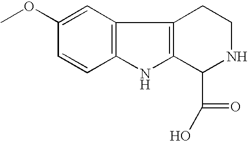 | 6-methoxy-2,3,4,9-tetrahydro-1H-beta-car- boline-1-carboxylic acid | 1.5 | >100 |
| 15 | 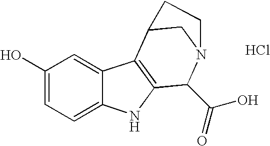 | 7-hydroxy-3,4,5,10-tetrahydro-1H-2,5-methano- azepino[3,4-b]indole-1-carboxylic acid hydrochloride | 1.53 | |
| 16 | 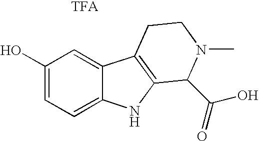 | 6-hydroxy-2-methyl-2,3,4,9-tetra- hydro-1H-beta-car- boline-1-carboxylic acid trifluoroacetate | 1.74 | >100 |
| 17 | 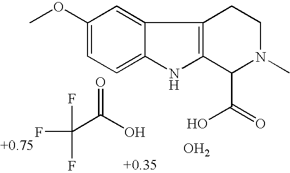 | 6-methoxy-2-methyl-2,3,4,9-tetra- hydro-1H-beta-car- boline-1-carboxylic acid trifluoroacetate hydrate | 1.78 | 25.5 |
| 18 | 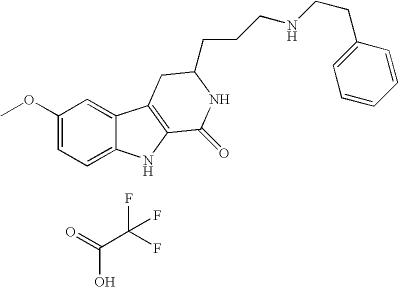 | 6-methoxy-3-{3-[(2-phenyleth- yl)amino]propyl}-2,3,4,9-tetra- hydro-1H-beta-carbolin-1-one trifluoroacetate | 2.03 | 23.7 |
| 19 | 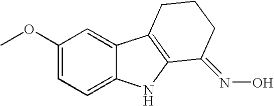 | (1E)-6-methoxy-2,3,4,9-tetrahydro-1H-car- bazol-1-one oxime | 2.1 | 1.89 |
| 20 | 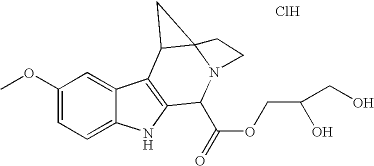 | 2,3-dihydroxypropyl 7-methoxy-3,4,5,10-tetra- hydro-1H-2,5-methanoazepino[3,4-b]in- dole-1-carboxylate hydrochloride | 2.17 | >100 |
| 21 | 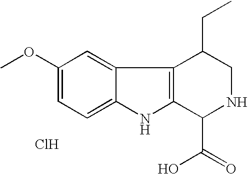 | 4-ethyl-6-methoxy-2,3,4,9-tetra- hydro-1H-beta-car- boline-1-carboxylic acid hydrochloride | 2.27 | >100 |
| 22 | 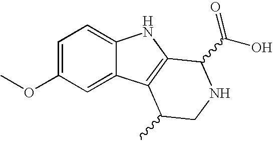 | 6-methoxy-4-methyl-2,3,4,9-tetra- hydro-1H-beta-car- boline-1-carboxylic acid | 2.62 | 92.2 |
| 23 | 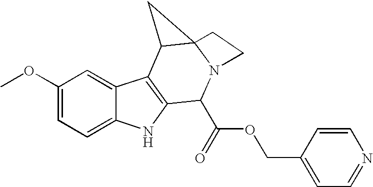 | pyridin-4-ylmethyl 7-meth- oxy-3,4,5,10-tetra- hydro-1H-2,5-methanoazepino[3,4-b]in- dole-1-carboxylate | 2.86 | >100 |
| 24 | 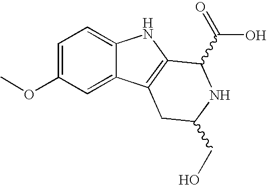 | 3-(hydroxymethyl)-6-methoxy-2,3,4,9-tetra- hydro-1H-beta-carboline-1-carboxylic acid | 3.03 | >100 |
| 25 | 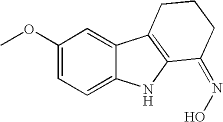 | (1Z)-6-methoxy-2,3,4,9-tetrahydro-1H-car- bazol-1-one oxime | 3.1 | 2.18 |
| 26 | 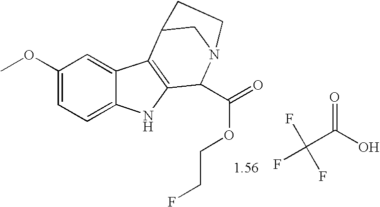 | 2-fluoroethyl 7-meth- oxy-3,4,5,10-tetrahydro-1H-2,5-meth- anoazepino[3,4-b]indole-1-car- boxylate trifluoroacetate | 3.18 | 2.04 |
| 27 | 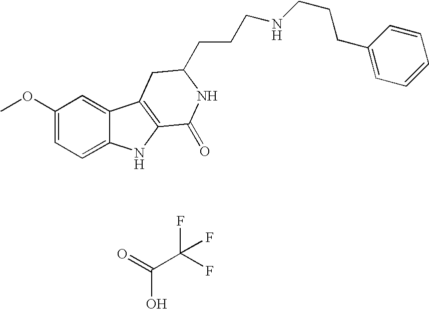 | 6-methoxy-3-{3-[(3-phenyl- propyl)amino]propyl}-2,3,4,9-tetra- hydro-1H-beta-car- bolin-1-one trifluoroacetate | 3.24 | 8.54 |
| 28 | 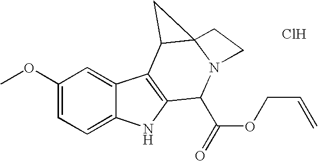 | allyl 7-methoxy-3,4,5,10-tetra- hydro-1H-2,5-meth- anoazepino[3,4-b]indole-1-carboxylate hydrochloride | 3.28 | 14.5 |
| 29 | 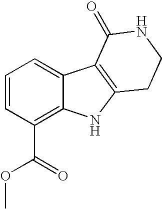 | methyl 1-oxo-2,3,4,5-tetra- hydro-1H-pyrido[4,3-b]in- dole-6-carboxylate | 3.52 | 42.4 |
| 30 | 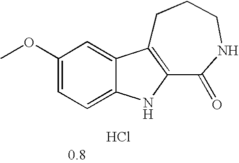 | 7-methoxy-3,4,5,10-tetrahydroazepino[3,4-b]in- dol-1(2H)-one hydrochloride | 3.6 | 2.66 |
| 31 | 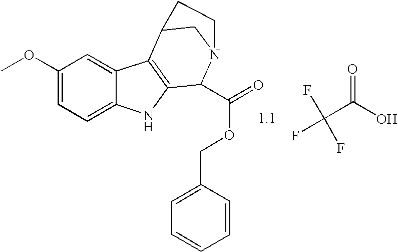 | benzyl 7-meth- oxy-3,4,5,10-tetrahydro-1H-2,5-meth- anoazepino[3,4-b]indole-1-carboxylate trifluoroacetate | 4.6 | 46.2 |
| 32 | 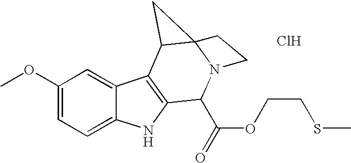 | 2-(methylthio)ethyl 7-methoxy-3,4,5,10-tetra- hydro-1H-2,5-methanoazepino[3,4-b]in- dole-1-carboxylate hydrochloride | 4.4 | 29.9 |
| 33 | 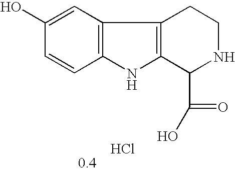 | 6-hydroxy-2,3,4,9-tetrahydro-1H-beta-car- boline-1-carboxylic acid hydrochloride | 4.51 | >100 |
| 34 | 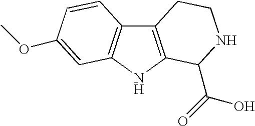 | 7-methoxy-2,3,4,9-tetrahydro-1H-beta-car- boline-1-carboxylic acid | 4.52 | >100 |
| 35 | 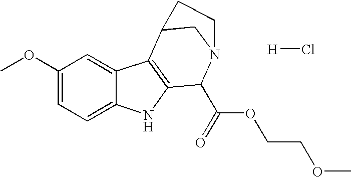 | 2-methoxyethyl 7-methoxy-3,4,5,10-tetra- hydro-1H-2,5-methanoazepino[3,4-b]in- dole-1-carboxylate hydrochloride | 4.72 | 93.2 |
| 36 | 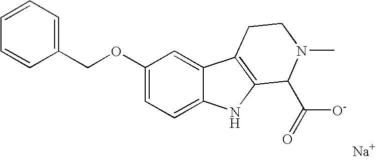 | sodium 6-(benzyloxy)-2-methyl-2,3,4,9-tetra- hydro-1H-beta-carboline-1-carboxylate | 4.83 | >100 |
| | | | 4.96 | 3.58 |
| 37 | 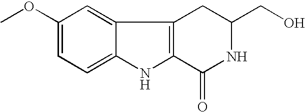 | 3-(hydroxymethyl)-6-methoxy-2,3,4,9-tetra- hydro-1H-beta-carbolin-1-one | 5.03 | >100 |
| 38 | 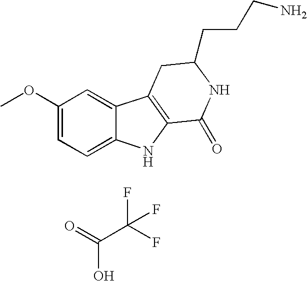 | 3-(3-aminopropyl)-6-methoxy-2,3,4,9-tetra- hydro-1H-beta-carbolin-1-one trifluoroacetate | 5.5 | 36.7 |
| 39 | 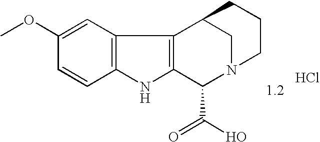 | trans-8-meth- oxy-1,3,4,5,6,11-hexahydro-2,6-methano- azocino[3,4-b]indole-1-carboxylic acid hydrochloride | 5.07 | >100 |
| | | | | 25 |
| 40 | 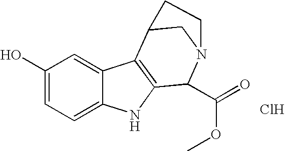 | methyl 7-hydroxy-3,4,5,10-tetra- hydro-1H-2,5-methano- azepino[3,4-b]indole-1-carboxylate hydrochloride | 5.38 | 25.5 |
| 41 | 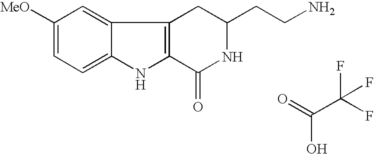 | 3-(2-aminoethyl)-6-methoxy-2,3,4,9-tetra- hydro-1H-beta-carbolin-1-one trifluoroacetate | 5.54 | >100 |
| 42 | 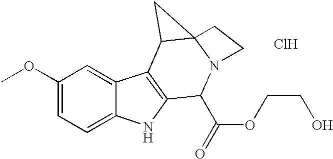 | 2-hydroxyethyl 7-meth- oxy-3,4,5,10-tetrahydro-1H-2,5-meth- anoazepino[3,4-b]indole-1-car- boxylate hydrochloride | 5.64 | >100 |
| 43 | 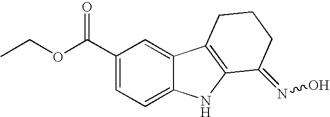 | ethyl 1-(hydroxy- imino)-2,3,4,9-tetrahydro-1H-car- bazole-6-carboxylate | 5.67 | 8.77 |
| 44 | 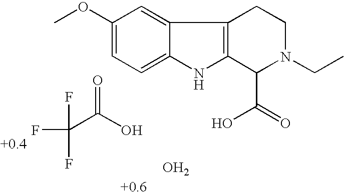 | 2-ethyl-6-methoxy-2,3,4,9-tetra- hydro-1H-beta-car- boline-1-carboxylic acid trifluoroacetate hydrate | 5.79 | >100 |
| 45 | 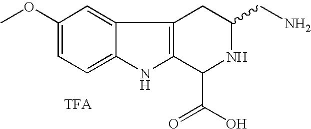 | 3-(aminomethyl)-6-methoxy-2,3,4,9-tetra- hydro-1H-beta-carboline-1-carboxylic acid trifluoroacetate | 5.86 | >100 |
| 46 | 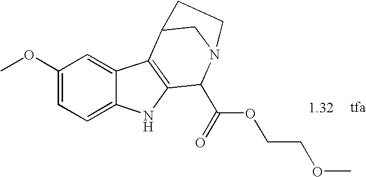 | 2-methoxyethyl 7-methoxy-3,4,5,10-tetra- hydro-1H-2,5-methanoazepino[3,4-b]in- dole-1-carboxylate trifluoroacetate | 6.49 | >100 |
| 47 | 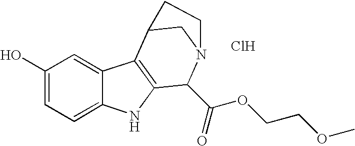 | 2-methoxyethyl 7-hy- droxy-3,4,5,10-tetrahydro-1H-2,5-meth- anoazepino[3,4-b]indole-1-car- boxylate hydrochloride | 6.88 | >100 |
| 48 | 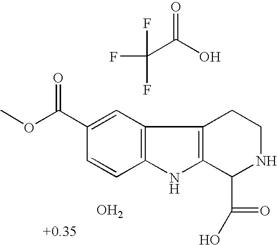 | 6-(methoxycarbonyl)-2,3,4,9-tetra- hydro-1H-beta-car- boline-1-carboxylic acid trifluoroacetate hydrate | 6.88 | >100 |
| 49 | 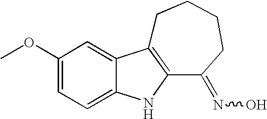 | 2-methoxy-7,8,9,10-tetra- hydrocyclohepta[b]indol-6(5H)-one oxime | 6.9 | 2.89 |

| | | | | TNF |
|---|---|---|---|---|
| | | | | Release |
| | | | MK-2 | Assay |
| | | | Avg. | Avg. |
| | | | IC50 | IC50 |
| No. | Structure^{a} | Compound Name(s)^{b} | (μM)^{c} | (μM)^{c} |
| 50 | 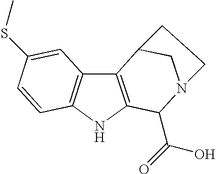 | | 7.01 | 15.3 |
| 51 | 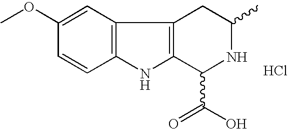 | 6-methoxy-3-methyl-2,3,4,9-tetrahydro-1H-beta-car- boline-1-carboxylic acid | 7.09 | >100 |
| 52 | 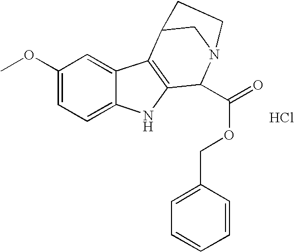 | benzyl 7-methoxy-3,4,5,10-tetrahydro-1H-2,5-meth- anoazepino[3,4-b]indole-1-carboxylate hydrochloride | 7.11 | 20.4 |
| 53 | 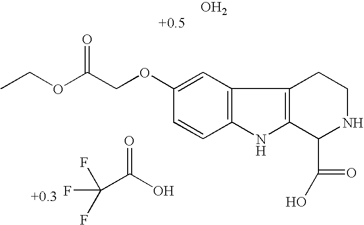 | 6-(2-ethoxy-2-oxoeth- oxy)-2,3,4,9-tetrahydro-1H-beta-car- boline-1-carboxylic acid trifluoroacetate hydrate | 8 | >100 |
| 54 | 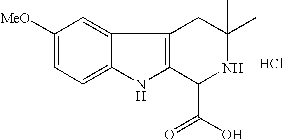 | 6-methoxy-3,3-dimeth- yl-2,3,4,9-tetrahydro-1H-beta-car- boline-1-carboxylic acid | 8.39 | >100 |
| 55 | 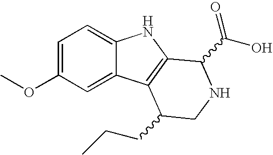 | 6-methoxy-4-propyl-2,3,4,9-tetrahydro-1H-beta-car- boline-1-carboxylic acid | 8.47 | >100 |
| 56 | 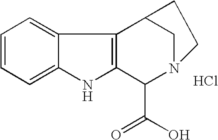 | 3,4,5,10-tetrahydro-1H-2,5-meth- anoazepino[3,4-b]indole-1-carboxylic acid hydrochloride | 9.26 | |
| 57 | 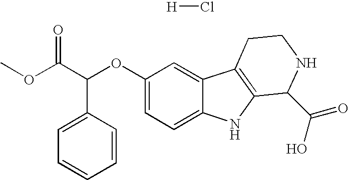 | 6-(2-methoxy-2-oxo-1-phenylethoxy)-2,3,4,9-tetra- hydro-1H-beta-carboline-1-carboxylic acid | 9.45 | 97.3 |
| 58 | 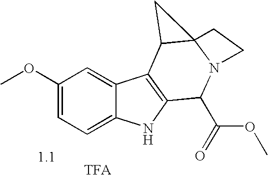 | methyl 7-methoxy-3,4,5,10-tetrahydro-1H-2,5-meth- anoazepino[3,4-b]indole-1-carboxylate trifluoroacetate | 9.7 | 16.3 |
| 59 | 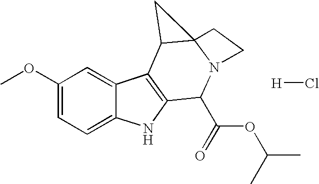 | isopropyl 7-methoxy-3,4,5,10-tetrahydro-1H-2,5-meth- anoazepino[3,4-b]indole-1-car- boxylate hydrochloride | 9.85 | >100 |
| 60 | 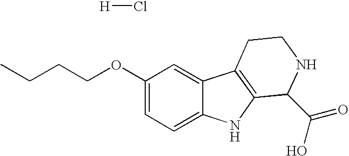 | 6-butoxy-2,3,4,9-tetrahydro-1H-beta-car- boline-1-carboxylic acid | 10.1 | >100 |
| 61 | 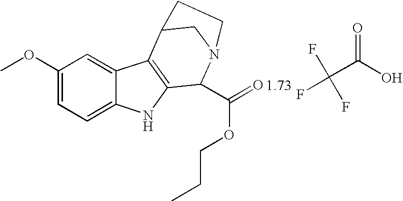 | propyl 7-methoxy-3,4,5,10-tetrahydro-1H-2,5-meth- anoazepino[3,4-b]indole-1-carboxylate trifluoroacetate | 10.3 | 0.879 |
| 62 | 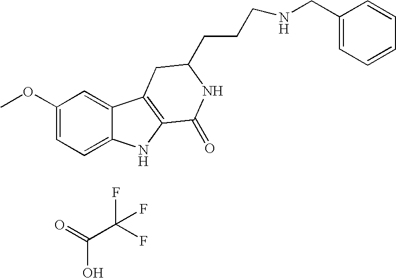 | 3-[3-(benzylamino)propyl]-6-methoxy-2,3,4,9-tetra- hydro-1H-beta-carbolin-1-one trifluoroacetate | 10.5 | 24.9 |
| 63 | 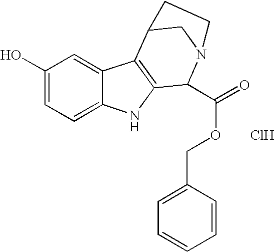 | benzyl 7-hydroxy-3,4,5,10-tetrahydro-1H-2,5-meth- anoazepino[3,4-b]indole-1-carboxylate hydrochloride | 11.4 | 35.9 |
| 64 | 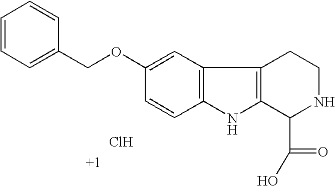 | 6-(benzyloxy)-2,3,4,9-tetrahydro-1H-beta-car- boline-1-carboxylic acid hydrochloride | 12.4 | 85.1 |
| 65 | 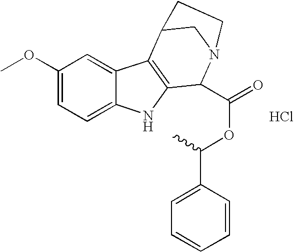 | 1-phenylethyl 7-meth- oxy-3,4,5,10-tetrahydro-1H-2,5-meth- anoazepino[3,4-b]indole-1-car- boxylate hydrochloride | 13 | 13.4 |
| 66 | 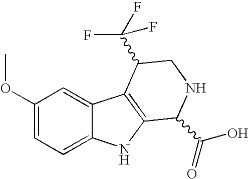 | 6-methoxy-4-(trifluoromethyl)-2,3,4,9-tetra- hydro-1H-beta-car- boline-1-carboxylic acid | 13.8 | 41.3 |
| 67 | 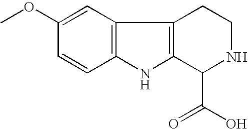 | (1S)-6-methoxy-2,3,4,9-tetrahydro-1H-beta-car- boline-1-carboxylic acid | 14.1 | >100 |
| 68 | 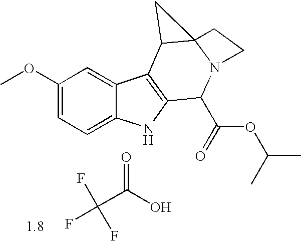 | isopropyl 7-methoxy-3,4,5,10-tetrahydro-1H-2,5-meth- anoazepino[3,4-b]indole-1-car- boxylate trifluoroacetate | 14.8 | |
| 69 | 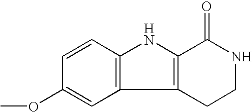 | 6-methoxy-2,3,4,9-tetrahydro-1H-beta-car- bolin-1-one | 15 | 45.7 |
| 70 | 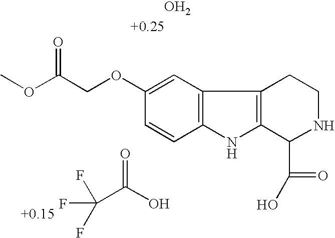 | 6-(2-methoxy-2-oxoeth- oxy)-2,3,4,9-tetrahydro-1H-beta-car- boline-1-carboxylic acid trifluoroacetate hydrate | 15.3 | >100 |
| 71 | 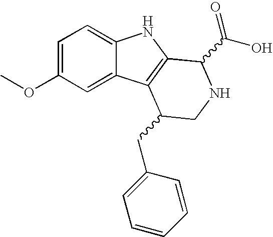 | 4-benzyl-6-methoxy-2,3,4,9-tetrahydro-1H-beta-car- boline-1-carboxylic acid | 15.5 | 54 |
| 72 | 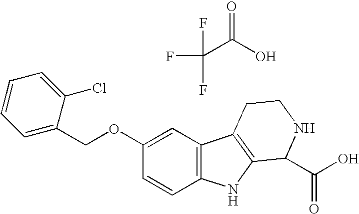 | 6-[(2-chlorobenzyl)oxy]-2,3,4,9-tetra- hydro-1H-beta-car- boline-1-carboxylic acid trifluoroacetate | 15.5 | 7.68 |
| 73 | 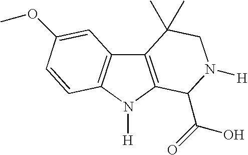 | 6-methoxy-4,4-dimeth- yl-2,3,4,9-tetrahydro-1H-beta-car- boline-1-carboxylic acid | 15.8 | >100 |
| 74 | 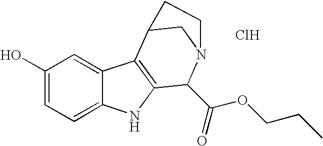 | propyl 7-hydroxy-3,4,5,10-tetrahydro-1H-2,5-meth- anoazepino[3,4-b]indole-1-carboxylate hydrochloride | 15.8 | 10.6 |
| 75 | 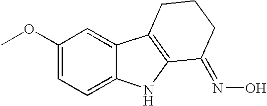 | 6-methoxy-2,3,4,9-tetrahydro-1H-carbazol-1-one oxime | 16.5 | 13.9 |
| 76 | 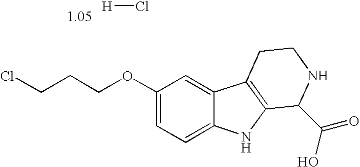 | 6-(3-chloropropoxy)-2,3,4,9-tetrahydro-1H-beta-car- boline-1-carboxylic acid hydrochloride | 16.7 | >100 |
| 77 | 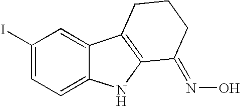 | 6-iodo-2,3,4,9-tetrahydro-1H-carbazol-1-one oxime | 17 | 23.2 |
| 78 | 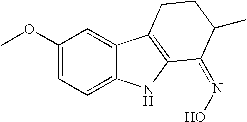 | 6-methoxy-2-methyl-2,3,4,9-tetrahydro-1H-car- bazol-1-one oxime | 17 | 1.59 |
| 79 | 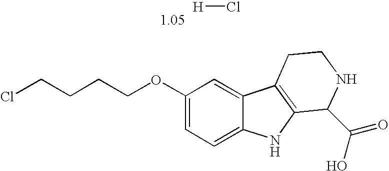 | 6-(4-chlorobutoxy)-2,3,4,9-tetrahydro-1H-beta-car- boline-1-carboxylic acid hydrochloride | 17.4 | >100 |
| 80 | 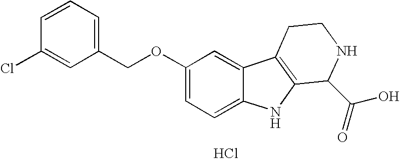 | 6-[(3-chlorobenzyl)oxy]-2,3,4,9-tetra- hydro-1H-beta-car- boline-1-carboxylic acid | 17.9 | 17.8 |
| 81 | 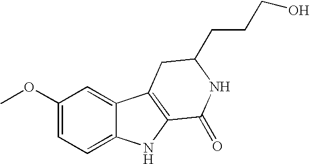 | 3-(3-hydroxypropyl)-6-methoxy-2,3,4,9-tetra- hydro-1H-beta-carbolin-1-one | 20.4 | |
| 82 | 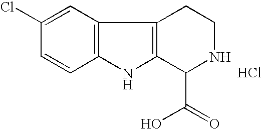 | 6-chloro-2,3,4,9-tetrahydro-1H-beta-carboline-1-car- boxylic acid hydrochloride | 20.4 | |
| 83 | 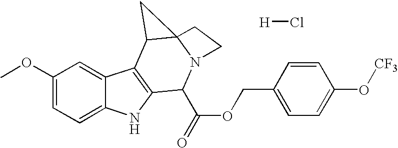 | 4-(trifluoromethoxy)benzyl 7-methoxy-3,4,5,10-tetra- hydro-1H-2,5-methano- azepino[3,4-b]indole-1-carboxylate hydrochloride | 20.4 | 10.3 |
| 84 | 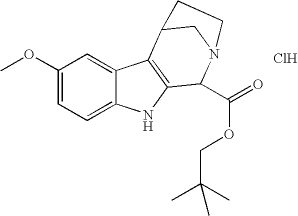 | neopentyl 7-meth- oxy-3,4,5,10-tetrahydro-1H-2,5-meth- anoazepino[3,4-b]indole-1-car- boxylate hydrochloride | 20.6 | 41 |
| 85 | 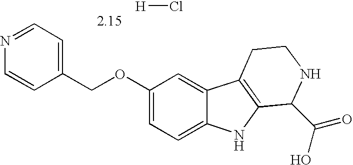 | 6-(pyridin-4-ylmeth- oxy)-2,3,4,9-tetrahydro-1H-beta-car- boline-1-carboxylic acid hydrochloride | 25.8 | 48.4 |
| 86 | 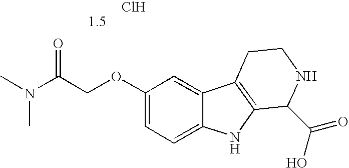 | 6-[2-(dimethylamino)-2-oxoethoxy]-2,3,4,9-tetra- hydro-1H-beta-carboline-1-carboxylic acid hydrochloride | 27.7 | >100 |
| 87 | 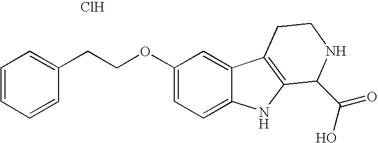 | 6-(2-phenylethoxy)-2,3,4,9-tetrahydro-1H-beta-car- boline-1-carboxylic acid hydrochloride | 27.9 | |
| 88 | 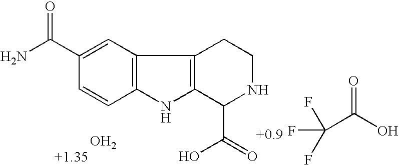 | 6-(aminocarbonyl)-2,3,4,9-tetrahydro-1H-beta-car- boline-1-carboxylic acid trifluoroacetate hydrate | 28.1 | >100 |
| 89 | 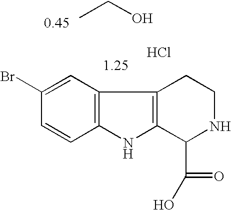 | 6-bromo-2,3,4,9-tetrahydro-1H-beta-carboline-1-car- boxylic acid compound with ethanol(1:1) hydrochloride | 28.2 | >100 |
| 90 | 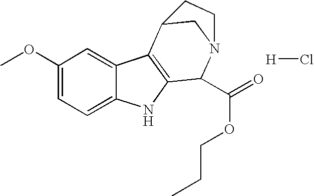 | propyl 7-methoxy-3,4,5,10-tetrahydro-1H-2,5-meth- anoazepino[3,4-b]indole-1-carboxylate hydrochloride | 28.6 | 47.9 |
| 91 | 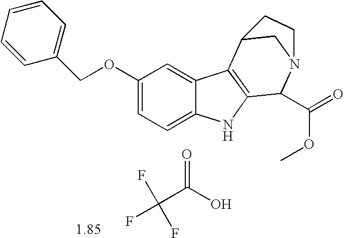 | methyl 7-(benzyloxy)-3,4,5,10-tetra- hydro-1H-2,5-meth- anoazepino[3,4-b]indole-1-car- boxylate compound with trifluoroacetic acid (1:1) | 28.7 | 24 |
| 92 | 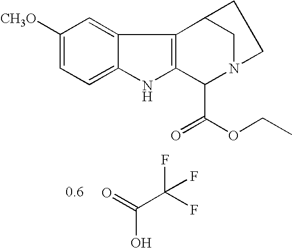 | ethyl 7-methoxy-3,4,5,10-tetrahydro-1H-2,5-meth- anoazepino[3,4-b]indole-1-carboxylate trifluoroacetate | 29.3 | 5.61 |
| 93 | | hexyl 7-methoxy-3,4,5,10-tetrahydro-1H-2,5-meth- | 29.7 | 9.65 |
| | | anoazepino[3,4-b]indole-1-carboxylate | | |
| | | hydrochloride | | |
| 94 | 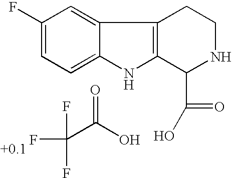 | 6-fluoro-2,3,4,9-tetrahydro-1H-beta-carboline-1-car- boxylic acid trifluoroacetate | 29.9 | >100 |
| 95 | 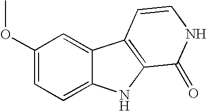 | 6-methoxy-2,9-dihydro-1H-beta-carbolin-1-one | 31.8 | |
| 96 | 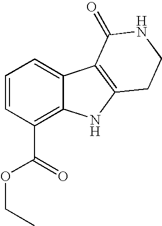 | ethyl 1-oxo-2,3,4,5-tetrahydro-1H-pyrido[4,3-b]in- dole-6-carboxylate | 34.5 | |
| 97 | 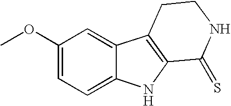 | 6-methoxy-2,3,4,9-tetrahydro-1H-beta-car- boline-1-thione | 34.8 | |

| | | | | TNF |
|---|---|---|---|---|
| | | | | Release |
| | | | MK-2 | Assay |
| | | | Avg. | Avg. |
| | | | IC50 | IC50 |
| No. | Structure^{a} | Compound Name(s)^{b} | (μM)^{c} | (μM)^{c} |
| 98 | 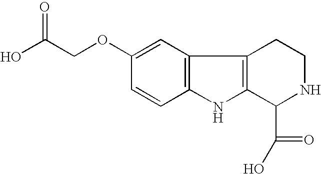 | 6-(carboxymethoxy)-2,3,4,9-tetrahydro-1H-beta-car- bolin-1-carboxylic acid | 35.5 | |
| 99 | 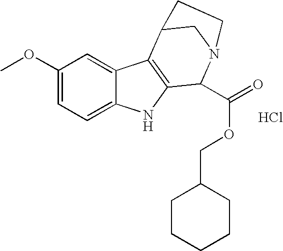 | cyclohexylmethyl 7-methoxy-3,4,5,10-tetra- hydro-1H-2,5-methanoazepino[3,4-b]in- dinole-1-carboxylate hydrochloride | 38.5 | 9.83 |
| 100 | 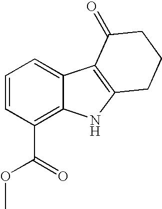 | methyl 4-oxo-2,3,4,9-tetrahydro-1H-carbazole-8-car- boxylate | 39.8 | |
| 101 | 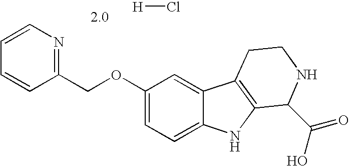 | 6-(pyridin-2-ylmeth- oxy)-2,3,4,9-tetrahydro-1H-beta-car- boline-1-carboxylic acid hydrochloride | 39.9 | |
| 102 | 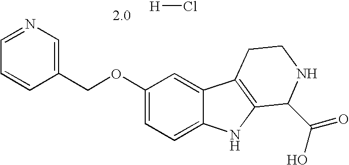 | 6-(pyridin-3-ylmeth- oxy)-2,3,4,9-tetrahydro-1H-beta-car- boline-1-carboxylic acid hydrochloride | 42.6 | |
| 103 | 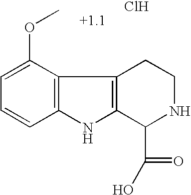 | 5-methoxy-2,3,4,9-tetrahydro-1H-beta-car- boline-1-carboxylic acid hydrochloride | 44.8 | 92.6 |
| 104 | 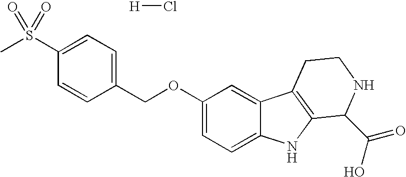 | 6-{[4-(methylsulfonyl)benzyl]oxy}-2,3,4,9-tetra- hydro-1H-beta-carboline-1-carboxylic acid hydrochloride | 44.9 | |
| 105 | 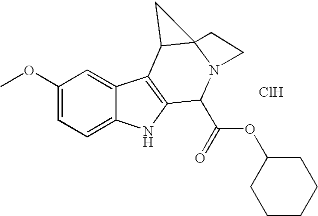 | cyclohexyl 7-meth- oxy-3,4,5,10-tetrahydro-1H-2,5-meth- anoazepino[3,4-b]indole-1-car- boxylate hydrochloride | 45.2 | >100 |
| 106 | 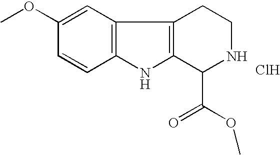 | methyl 6-methoxy-2,3,4,9-tetrahydro-1H-beta-car- boline-1-carboxylate hydrochloride | 47.4 | 26.1 |
| 107 | 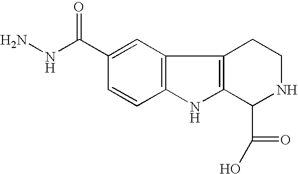 | 6-(hydrazinocarbonyl)-2,3,4,9-tetrahydro-1H-beta-car- boline-1-carboxylic acid | 48.6 | |
| 108 | 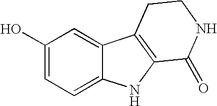 | 6-hydroxy-2,3,4,9-tetrahydro-1H-beta-car- bolin-1-one | 48.9 | |
| 109 | 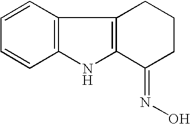 | 2,3,4,9-tetrahydro-1H-carbazol-1-one oxime | 49.9 | |
| 110 | 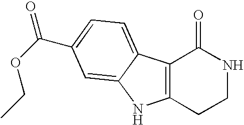 | ethyl 1-oxo-2,3,4,5-tetrahydro-1H-pyrido[4,3-b]in- dole-7-carboxylate | 50.1 | |
| 111 | 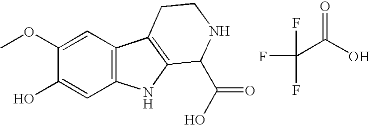 | 7-hydroxy-6-methoxy-2,3,4,9-tetrahydro-1H-beta-car- boline-1-carboxylic acid trifluoroacetate | 52.9 | |
| 112 | 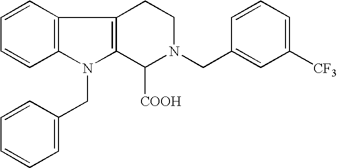 | 1H-Pyrido[3,4-b]indole-1-carboxylic acid, 2,3,4,9-tetrahydro-9-(phenylmethyl)-2-[[3-(tri- fluoromethyl)phenyl]methyl]- or 9-benzyl-2-[3-(tri- fluoromethyl)benzyl]-2,3,4,9-tetrahydro-1H-beta-car- boline-1-carboxylic acid | 53.7 | |
| 113 | 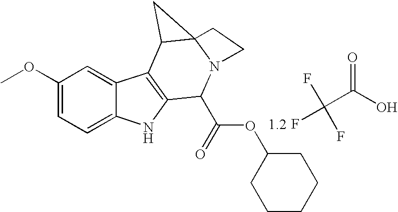 | cyclohexyl 7-meth- oxy-3,4,5,10-tetrahydro-1H-2,5-meth- anoazepino[3,4-b]indole-1-car- boxylate trifluoroacetate | 57.3 | 59.1 |
| 114 | 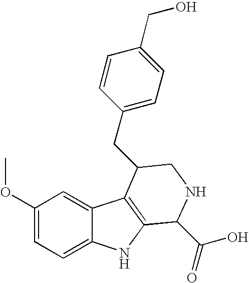 | 4-[4-(hydroxymethyl)benzyl]-6-methoxy-2,3,4,9-tetra- hydro-1H-beta-carboline-1-car- boxylic acid | 61.4 | |
| 115 | 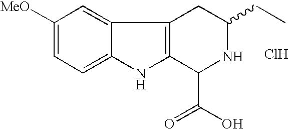 | 3-ethyl-6-methoxy-2,3,4,9-tetrahydro-1H-beta-car- boline-1-carboxylic acid | 65.7 | |
| 116 | 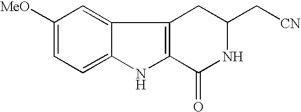 | (6-methoxy-1-oxo-2,3,4,9-tetrahydro-1H-beta-car- bolin-3-yl)acetontrile | 68.7 | |
| 117 | 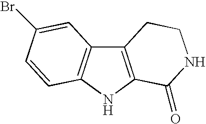 | 6-bromo-2,3,4,9-tetrahydro-1H-beta-carbolin-1-one | 69.6 | |
| 118 | 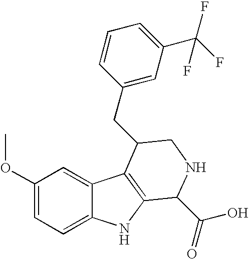 | 6-methoxy-4-[3-(trifluoromethyl)benzyl]-2,3,4,9-tetra- hydro-1H-beta-carboline-1-car- boxylic acid | 70.9 | |
| 119 | 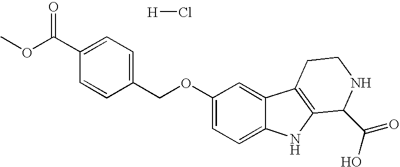 | 6-{[4-(methoxycarbonyl)benzyl]oxy}-2,3,4,9-tetra- hydro-1H-beta-carboline-1-carboxylic acid hydrochloride | 71.9 | |
| 120 | 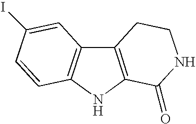 | 6-iodo-2,3,4,9-tetrahydro-1H-beta-carbolin-1-one | 75.2 | |
| 121 | 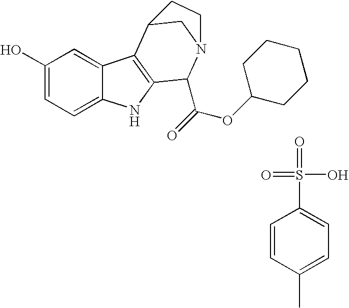 | cyclohexyl 7-hy- droxy-3,4,5,10-tetrahydro-1H-2,5-meth- anoazepino[3,4-b]indole-1-car- boxylate 4-methylbenzenesulfonate | 78.3 | 62.7 |
| 122 | 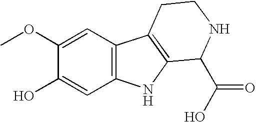 | 7-hydroxy-6-methoxy-2,3,4,9-tetrahydro-1H-beta-car- boline-1-carboxylic acid | 78.7 | |
| 123 | 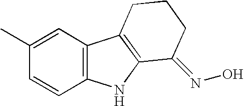 | 6-methyl-2,3,4,9-tetrahydro-1H-carbazol-1-one oxime | 82.9 | |
| | | | 88.1 | |
| 124 | 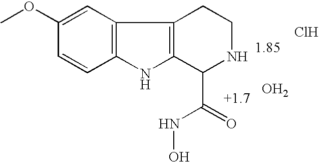 | N-hydroxy-6-methoxy-2,3,4,9-tetrahydro-1H-beta-car- boline-1-carboxamide hydrochloride hydrate | 88.5 | |
| 125 | 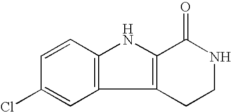 | 1H-Pyrido[3,4-b]indol-1-one, 6-chloro-2,3,4,9-tetra- hydro- or 6-chloro-2,3,4,9-tetrahydro-1H-beta-car- bolin-1-one | 89.6 | |
| 126 | 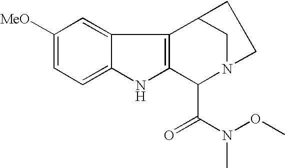 | N,7-dimethoxy-N-meth- yl-3,4,5,10-tetrahydro-1H-2,5-meth- anoazepino[3,4-b]indole-1-car- boxamide | 91 | |
| 127 | 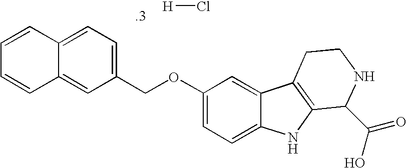 | 6-(2-naphthylmethoxy)-2,3,4,9-tetrahydro-1H-beta-car- boline-1-carboxylic acid hydrochloride | 91.3 | |
| 128 | 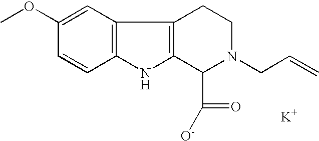 | potassium 2-allyl-6-methoxy-2,3,4,9-tetra- hydro-1H-beta-carboline-1-carboxylate | 95.1 | |
| | | | 98.2 | |
| 129 | 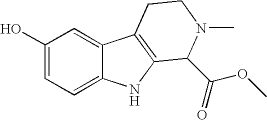 | methyl 6-hydroxy-2-meth- yl-2,3,4,9-tetrahydro-1H-beta-car- boline-1-carboxylate | 98.7 | 0.523 |
| 130 | 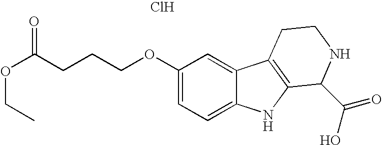 | 6-(4-ethoxy-4-oxobutoxy)-2,3,4,9-tetra- hydro-1H-beta-car- boline-1-carboxylic acid hydrochloride | 100 | |
| 131 | 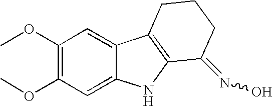 | 6,7-dimethoxy-2,3,4,9-tetrahydro-1H-carbazol-1-one oxime | 100 | |
| 132 | 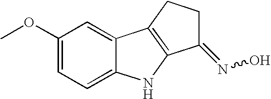 | 7-methoxy-1,4-dihydrocyclopenta[b]indol-3(2H)-one oxime | 104 | |
| 133 | 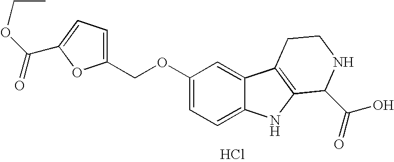 | 6-{[5-(ethoxycarbonyl)-2-furyl]meth- oxy}-2,3,4,9-tetra- hydro-1H-beta-carboline-1-car- boxylic acid | 105 | |
| 134 | 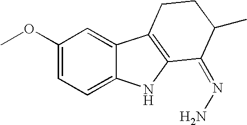 | 6-methoxy-2-methyl-2,3,4,9-tetrahydro-1H-car- bazol-1-one hydrazone | 108 | 7.33 |
| 135 | 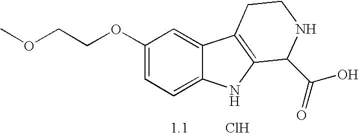 | 6-(2-methoxyethoxy)-2,3,4,9-tetrahydro-1H-beta-car- boline-1-carboxylic acid | 110 | |
| 136 | 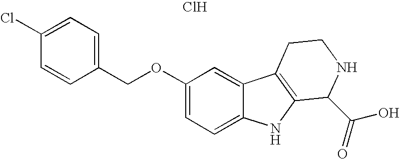 | 6-[(4-chlorobenzyl)oxy]-2,3,4,9-tetra- hydro-1H-beta-car- boline-1-carboxylic acid | 112 | |
| 137 | 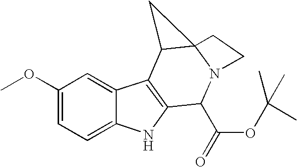 | tert-butyl 7-methoxy-3,4,5,10-tetrahydro-1H-2,5-meth- anoazepino[3,4-b]indole-1-car- boxylate | 112 | >100 |
| 138 | 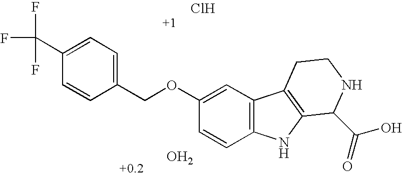 | 6-{[4-(trifluoromethyl)benzyl]oxy}-2,3,4,9-tetra- hydro-1H-beta-carboline-1-carboxylic acid hydrochloride hydrate | 115 | |
| 139 | 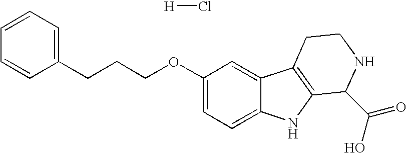 | 6-(3-phenylpropoxy)-2,3,49-tetrahydro-1H-beta-car- boline-1-carboxylic acid | 117 | |
| 140 | 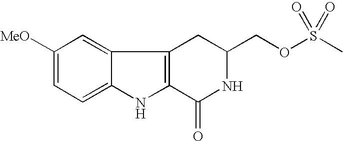 | (6-methoxy-1-oxo-2,3,4,9-tetrahydro-1H-beta-car- bolin-3-yl)methyl methanesulfonate | 117 | |
| 141 | 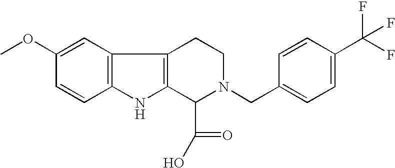 | 6-methoxy-2-[4-(trifluoromethyl)benzyl]-2,3,4,9-tetra- hydro-1H-beta-carboline-1-car- boxylic acid | 119 | |
| 142 | 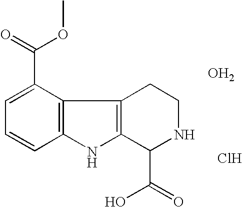 | 5-(methoxycarbonyl)-2,3,4,9-tetrahydro-1H-beta-car- boline-1-carboxylic acid hydrochloride hydrate | 121 | |
| 143 | 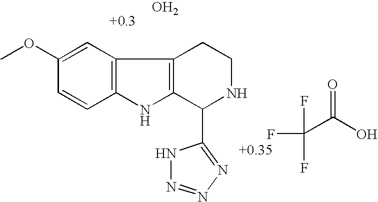 | 6-methoxy-1-(1H-tetraazol-5-yl)-2,3,4,9-tetra- hydro-1H-beta-carboline trifluoroacetate hydrate | 127 | 100 |
| 144 | 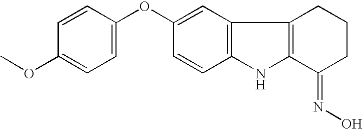 | 6-(4-methoxyphenoxy)-2,3,4,9-tetrahydro-1H-car- bazol-1-one oxime | 130 | |
| 145 | 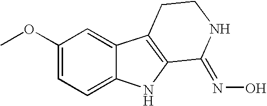 | 6-methoxy-2,3,49-tetrahydro-1H-beta-car- bolin-1-one oxime | 130 | |

| | | | | TNF |
|---|---|---|---|---|
| | | | | Release |
| | | | MK-2 | Assay |
| | | | Avg. | Avg. |
| | | | IC50 | IC50 |
| No. | Structure^{a} | Compound Name(s)^{b} | (μM)^{c} | (μM)^{c} |
| 146 | 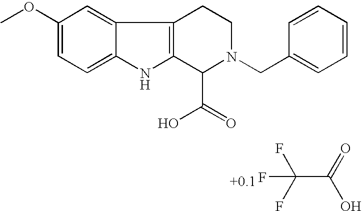 | 2-benzyl-6-meth- oxy-2,3,4,9-tetrahydro-1H-beta-car- boline-1-carboxylic acid trifluoroacetate | 132 | |
| 147 | 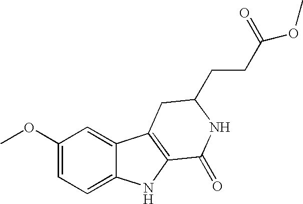 | methyl 3-(6-meth- oxy-1-oxo-2,3,4,9-tetrahydro-1H-beta-car- bolin-3-yl)propanoate | 136 | |
| 148 | 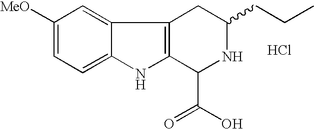 | 6-methoxy-3-propyl-2,3,4,9-tetra- hydro-1H-beta-car- boline-1-carboxylic acid | 137 | |
| 149 | 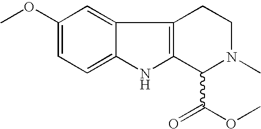 | methyl 6-methoxy-2-meth- yl-2,3,4,9-tetrahydro-1H-beta-car- boline-1-carboxylate | 149 | 8.25 |
| 150 | 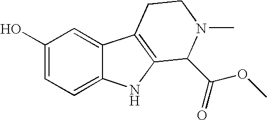 | methyl 6-hydroxy-2-methyl-2,3,4,9-tetra- hydro-1H-beta-car- boline-1-carboxylate | 149 | 0.523 |
| 151 | 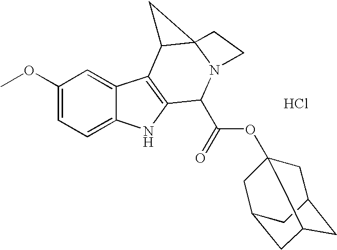 | 1-adamantyl 7-methoxy-3,4,5,10-tetra- hydro-1H-2,5-meth- anoazepino[3,4-b]indole-1-car- boxylate hydrochloride | 149 | 42.4 |
| | | | 151 | 0.338 |
| 152 | 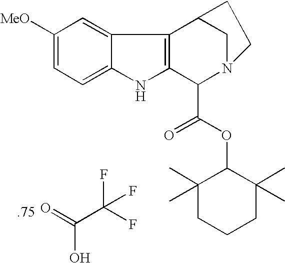 | 2,2,6,6-tetramethylcyclohexyl 7-methoxy-3,4,5,10-tetra- hydro-1H-2,5-meth- anoazepino[3,4-b]indole-1-carboxylate trifluoroacetate | 151 | 21.8 |
| 153 | 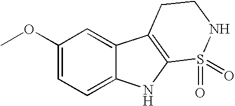 | 6-methoxy-2,3,4,9-tetra- hydro[1,2]thiazino[6,5-b]in- dole 1,1-dioxide | 155 | |
| 154 | 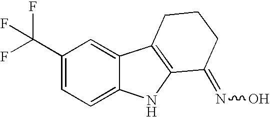 | 6-(trifluoromethyl)-2,3,4,9-tetrahydro-1H-car- bazol-1-one oxime | 156 | |
| 155 | 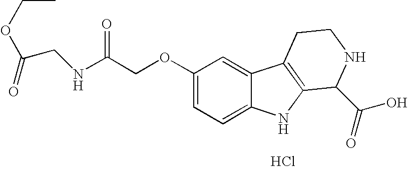 | 6-[2-(2-ethoxy-2-oxoethyl- amino)-2-oxoethoxy]-2,3,4,9-tetra- hydro-1H-beta-carboline-1-car- boxylic acid hydrochloride | 164 | |
| 156 | 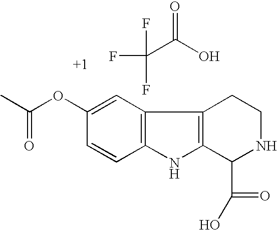 | 6-(acetyloxy)-2,3,4,9-tetrahydro-1H-beta-car- boline-1-carboxylic acid trifluoroacetate | 167 | 100 |
| | | | | 100 |
| 157 | 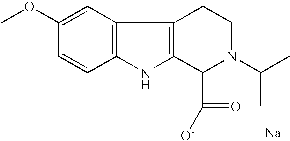 | sodium 2-isopropyl-6-methoxy-2,3,4,9-tetra- hydro-1H-beta-carboline-1-carboxylate | 170 | |
| 158 | 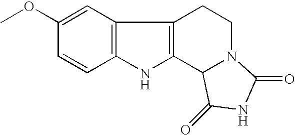 | 8-methoxy-5,6,11,11b-tetrahydro-1H-imi- dazo[1′,5′:1,2]pyrido[3,4-b]indole-1,3(2H)-dione | 175 | |
| 159 | 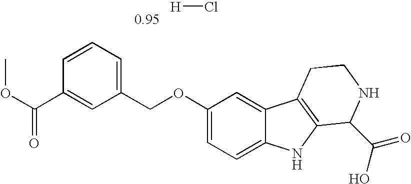 | 6-{[3-(methoxy- carbonyl)benzyl]oxy}-2,3,4,9-tetra- hydro-1H-beta-carboline-1-carboxylic acid hydrochloride | 182 | |
| 160 | 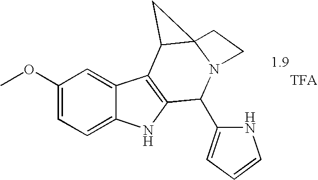 | 7-methoxy-1-(1H-pyrrol-2-yl)-3,4,5,10-tetra- hydro-1H-2,5-methanoazepino[3,4-b]in- dole trifluoroacetate | >200 | |
| 161 | 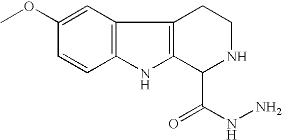 | 6-methoxy-2,3,4,9-tetrahydro-1H-beta-car- boline-1-carbohydrazide | >200 | |
| 162 | 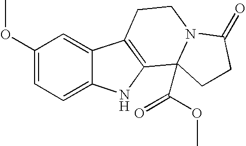 | methyl 8-meth- oxy-3-oxo-2,3,6,11-tetrahydro-1H-in- dolizino[8,7-b]indole-11b(5H)-car- boxylate | >200 | |
| 163 | 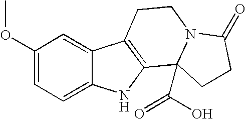 | 8-methoxy-3-oxo-2,3,6,11-tetrahydro-1H-in- dolizino[8,7-b]indole-11b(5H)-carboxylic acid | >200 | |
| 164 | 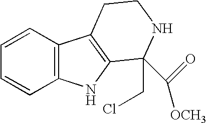 | methyl 1-(chlorometh- yl)-2,3,4,9-tetrahydro-1H-beta-car- boline-1-carboxylate | >200 | |
| 165 | 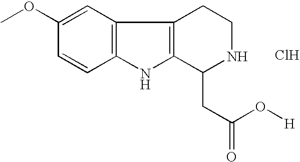 | (6-methoxy-2,3,4,9-tetrahydro-1H-beta-car- bolin-1-yl)acetic acid hydrochloride | >200 | |
| 166 | 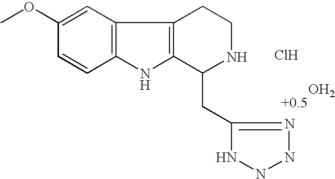 | 6-methoxy-1-(1H-tetra- azol-5-ylmethyl)-2,3,49-tetra- hydro-1H-beta-carboline hydrochloride hydrate | >200 | |
| 167 | 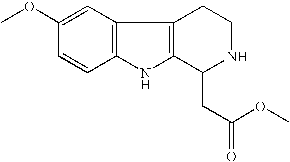 | methyl(6-meth- oxy-2,3,4,9-tetrahydro-1H-beta-car- bolin-1-yl)acetate | >200 | |
| 168 | 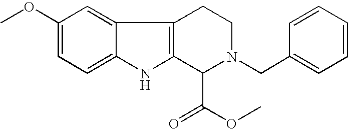 | methyl 2-benzyl-6-meth- oxy-2,3,4,9-tetrahydro-1H-beta-car- boline-1-carboxylate | >200 | |
| 169 | 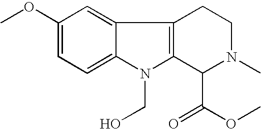 | methyl 9-(hydroxymethyl)-6-methoxy-2-meth- yl-2,3,4,9-tetrahydro-1H-beta-carboline-1-car- boxylate | >200 | |
| 170 | 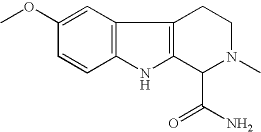 | 6-methoxy-2-meth- yl-2,3,4,9-tetrahydro-1H-beta-car- boline-1-carboxamide | >200 | >100 |
| 171 | 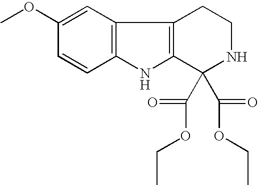 | diethyl 6-methoxy-2,3,4,9-tetra- hydro-1H-beta-car- boline-1,1-dicarboxylate | >200 | |
| 172 | 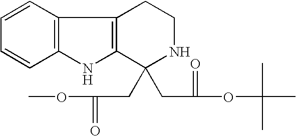 | 1h-Pyrido[3,4-B]indole-1-Acetic Acid,2,3,4,9-Tetra- hydro-1-(Methoxycarbonyl)-,1,1-Di- methylethyl Ester | >200 | |
| 173 | 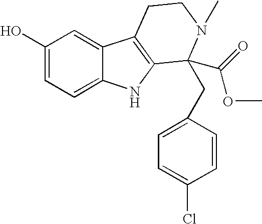 | methyl 1-[4-chlorobenzyl)-6-hy- droxy-2-methyl-2,3,4,9-tetra- hydro-1H-beta-carboline-1-car- boxylate | >200 | |
| 174 | 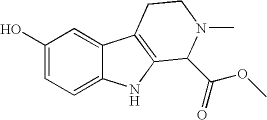 | methyl 6-hydroxy-2-meth- yl-2,3,4,9-tetrahydro-1H-beta-car- boline-1-carboxylate | >200 | 0.523 |
| | | | | 0.338 |
| | | | | 0.965 |
| | | | | 3 |
| 175 | 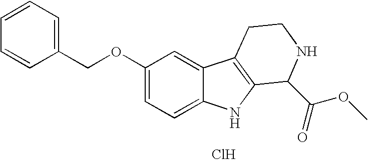 | methyl 6-(benzyl- oxy)-2,3,4,9-tetrahydro-1H-beta-car- boline-1-carboxylate hydrochloride | >200 | 26.5 |
| 176 | 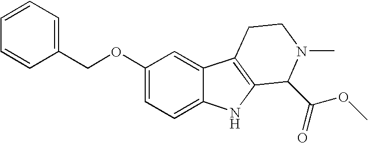 | methyl 6-(benzyloxy)-2-methyl-2,3,4,9-tetra- hydro-1H-beta-carboline-1-carboxylate | >200 | 36.4 |
| 177 | 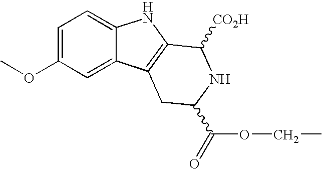 | 3-(ethoxycarbonyl)-6-methoxy-2,3,4,9-tetra- hydro-1H-beta-carboline-1-carboxylic acid | >200 | |
| 178 | 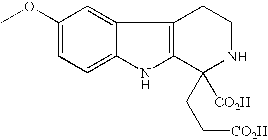 | 1-(2-carboxyethyl)-6-methoxy-2,3,4,9-tetra- hydro-1H-beta-carboline-1-carboxylic acid | >200 | |
| 179 | 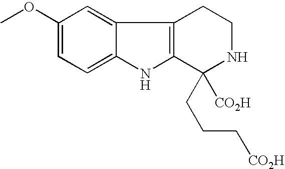 | 1-(3-carboxypropyl)-6-methoxy-2,3,4,9-tetra- hydro-1H-beta-carboline-1-carboxylic acid | >200 | |
| 180 | 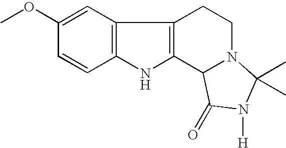 | 8-methoxy-3,3-dimethyl-2,3,5,6,11,11b-hexa- hydro-1H-imidazo[1′,5′:1,2]pyrido[3,4-b]in- dol-1-one | >200 | |
| 181 | 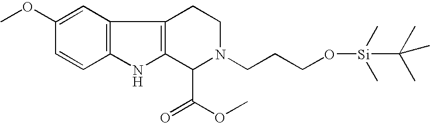 | methyl 2-(3-{[tert-bu- tyl(dimethyl)silyl]oxy}pro- pyl)-6-methoxy-2,3,4,9-tetra- hydro-1H-beta-carboline-1-car- boxylate | >200 | |
| 182 | 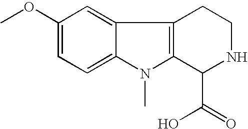 | 6-methoxy-9-meth- yl-2,3,4,9-tetrahydro-1H-beta-car- boline-1-carboxylic acid | >200 | |
| 183 | 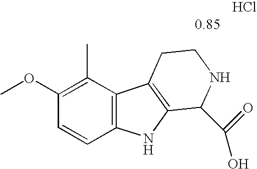 | 6-methoxy-5-meth- yl-2,3,4,9-tetrahydro-1H-beta-car- boline-1-carboxylic acid hydrochloride | >200 | |
| 184 | 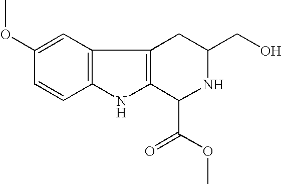 | methyl 3-(hydroxy- methyl)-6-methoxy-2,3,4,9-tetra- hydro-1H-beta-carboline-1-carboxylate | >200 | |
| 185 | 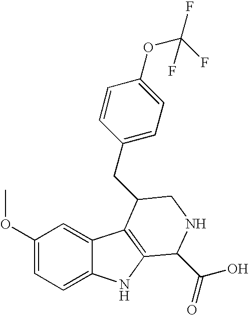 | 6-methoxy-4-[4-(trifluoro- methoxy)benzyl]-2,3,4,9-tetra- hydro-1H-beta-carboline-1-car- boxylic acid | >200 | |
| 186 | 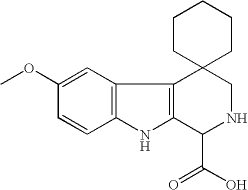 | 2′,3′,4′,9′-tetrahydro-6′-methoxy-spiro[cyclo- hexane-1,4′-[4H]pyrido[3,4-b]in- dole]-1-carboxylic acid | >200 | |
| 187 | 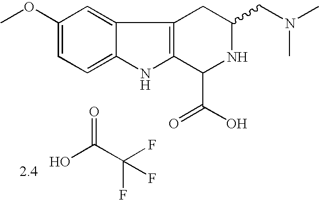 | 3-[(dimethyl- amino)methyl]-6-methoxy-2,3,4,9-tetra- hydro-1H-beta-carboline-1-carboxylic acid trifluoroacetate | >200 | |
| 188 | 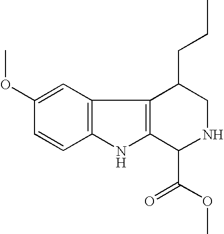 | methyl 6-methoxy-4-pro- pyl-2,3,4,9-tetrahydro-1H-beta-car- boline-1-carboxylate | >200 | 13.3 |
| 189 | 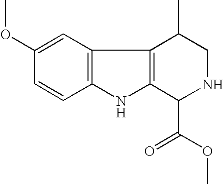 | methyl 6-methoxy-4-meth- yl-2,3,4,9-tetrahydro-1H-beta-car- boline-1-carboxylate | >200 | 53.2 |
| 190 | 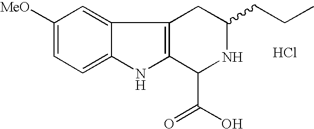 | 6-methoxy-3-pro- pyl-2,3,4,9-tetrahydro-1H-beta-car- boline-1-carboxylic acid | >200 | |
| 191 | 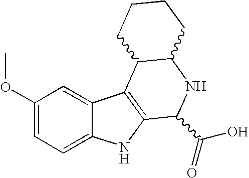 | 10-methoxy-2,3,4,4a,5,6,7,11c-octahydro-1H-in- dolo[2,3-c]quinoline-6-carboxylic acid | >200 | |
| 192 | 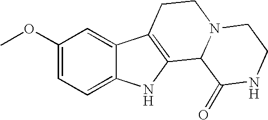 | 9-methoxy-3,4,6,7,12,12b-hexa- hydropyrazino[1′,2′:1,2]py- rido[3,4-b]indol-1(2H)-one | >200 | |
| 193 | 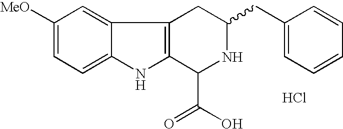 | 3-benzyl-6-meth- oxy-2,3,4,9-tetrahydro-1H-beta-car- boline-1-carboxylic acid | >200 | |

| | | | | TNF |
|---|---|---|---|---|
| | | | | Release |
| | | | MK-2 | Assay |
| | | | Avg. | Avg. |
| | | | 1C50 | 1C50 |
| No. | Structure^{a} | Compound Name(s)^{b} | (μM)^{c} | (μM)^{c} |
| 194 | 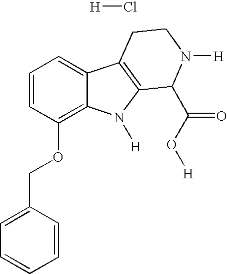 | 8-(benzyloxy)-2,3,4,9-tetrahydro-1H-beta- carboline-1-carboxylic acid | >200 | |
| 195 | | methyl 2-{2-[(tert-butoxycarbonyl)amino]ethyl}- | >200 | |
| | | 6-methoxy-2,3,4,9-tetrahydro-1H-beta- | | |
| | | carboline-1-carboxylate | | |
| 196 | 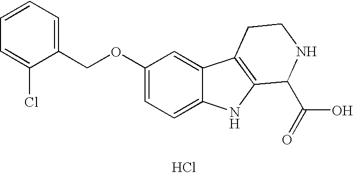 | 6-[(2-chlorobenzyl)oxy]-2,3,4,9-tetrahydro-1H- beta-carboline-1-carboxylic acid hydrochloride | >200 | |
| 197 | 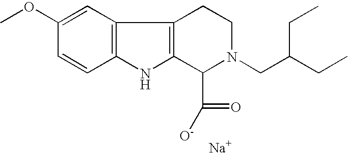 | sodium 2-(2-ethylbutyl)-6-methoxy-2,3,4,9- tetrahydro-1H-beta-carboline-1-carboxylate | >200 | |
| 198 | 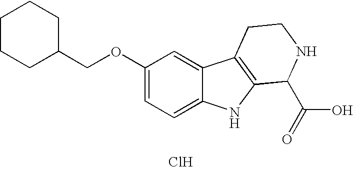 | 6-(cyclohexylmethoxy)-2,3,4,9-tetrahydro-1H- beta-carboline-1-carboxylic acid | >200 | |
| 199 | 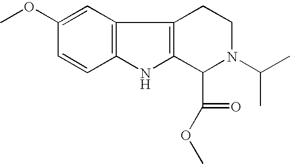 | methyl 2-isopropyl-6-methoxy-2,3,4,9- tetrahydro-1H-beta-carboline-1-carboxylate | >200 | |
| 200 | 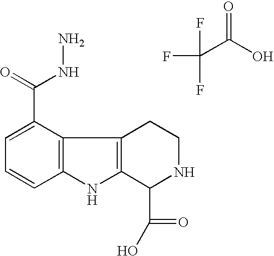 | 5-(hydrazinocarbonyl)-2,3,4,9-tetrahydro-1H- beta-carboline-1-carboxylic acid trifluoroacetate | >200 | |
| 201 | 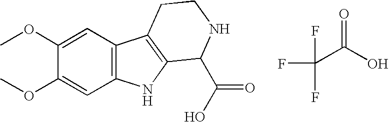 | 6,7-dimethoxy-2,3,4,9-tetrahydro-1H-beta- carboline-1-carboxylic acid trifluoroacetate | >200 | |
| 202 | 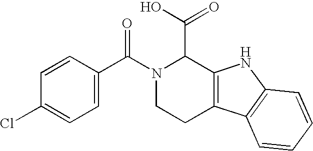 | 2-(4-chlorobenzeyl)-2,3,4,9-tetrahydro-1H- beta-carboline-1-carboxylic acid | >200 | >100 |
| 203 | 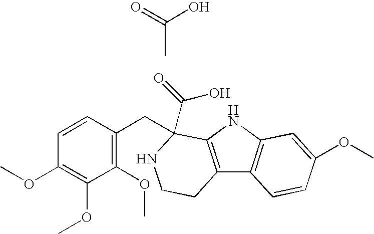 | 7-methoxy-1-[(2,3,4-trimethoxyphenyl)methyl]- 2,3,4,9-tetrahydro-1H-beta-carboline-1- carboxylic acid, acetate (1:1) | >200 | |
| 204 | 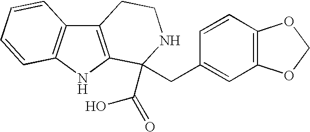 | 1-[(benzodioxolan-5-yl)methyl]-2,3,4,9- tetrahydro-1H-beta-carboline-1-carboxylic acid | >200 | |
| 205 | 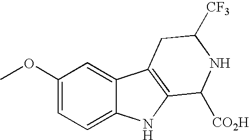 | 6-methoxy-3-(trifluoromethyl)-2,3,4,9- tetrahydro-1H-beta-carboline-1-carboxylic acid | >200 | |
| 206 | 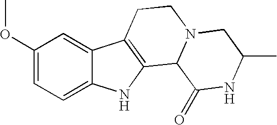 | 9-methoxy-3-methyl-3,4,6,7,12,12b- hexahydropyrazino[1′, 2′:1 , 2]pyrido[3,4-b]indol- 1(2H)-one | >200 | |
| 207 | 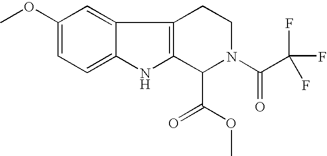 | methyl 6-methoxy-2-(trifluoroacetyl)-2,3,4,9- tetrahydro-1H-beta-carboline-1-carboxylate | >200 | |
| 208 | 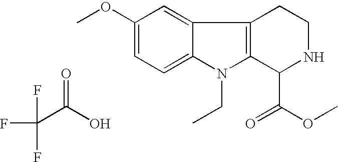 | methyl 9-ethyl-6-methoxy-2,3,4,9-tetrahydro- 1H-beta-carboline-1-carboxylate trifluoroacetate | >200 | 100 |
| 209 | 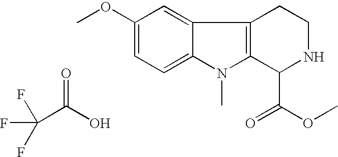 | methyl 6-methoxy-9-methyl-2,3,4,9-tetrahydro- 1H-beta-carboline-1-carboxylate trifluoroacetate | >200 | 88.9 |
| 210 | 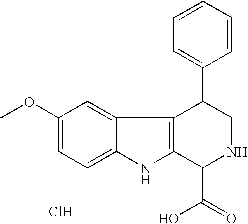 | 6-methoxy-4-phenyl-2,3,4,9-tetrahydro-1H- beta-carboline-1-carboxylic acid hydrochloride | >200 | |
| 211 | 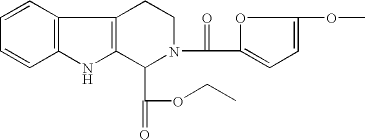 | 1h-Pyrido[3,4-B]Indole-1-Carboxylic Acid, 2,3,4,9-Tetrahydro-2-[(5-Methoxy-2- Furanyl)Carbonyl]-,Ethyl Ester | >200 | |
| 212 | 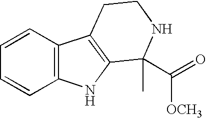 | 1h-Pyrido[3,4-B]Indole-1-Carboxylic Acid, 2,3,4,9-Tetrahydro-1-Methyl-,Methyl Ester | >200 | |
| 213 | 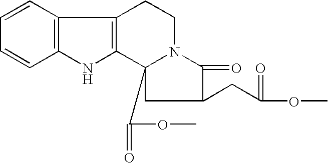 | 1h-Indolizino[8,7-B]Indole-2-Acetic Acid, 2,3,5,6,11,11b-(Methoxycarbonyl)-3-Oxo-, Methyl Ester | >200 | |
| 214 | 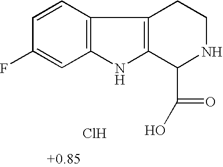 | 7-fluoro-2,3,4,9-tetrahydro-1H-beta-carboline- 1-carboxylic acid hydrochloride | >200 | |
| 215 | 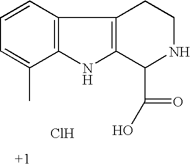 | 8-methyl-2,3,4,9-tetrahydro-1H-beta- carboline-1-carboxylic acid hydrochloride | >200 | |
| 216 | 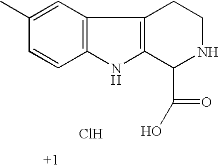 | 6-methyl-2,3,4,9-tetrahydro-1H-beta- carboline-1-carboxylic acid hydrochloride | >200 | |
| 217 | 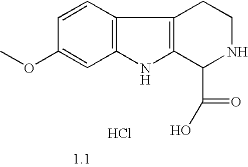 | 7-methoxy-2,3,4,9-tetrahydro-1H-beta- carboline-1-carboxylic acid hydrochloride | >200 | |
| 218 | 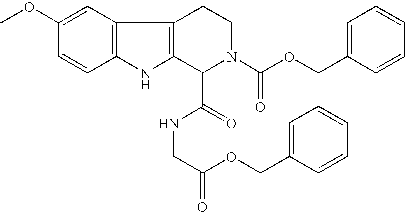 | 2-[(benzyloxy)carbonyl]-6-methoxy-N-[2- (benzyloxy)-2-oxo]ethyl-2,3,4,9-tetrahydro-1H- beta-carboline-1-carboxamide | >200 | |
| 219 | 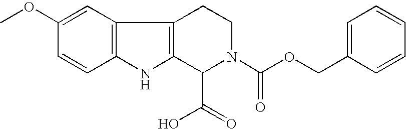 | 2-[(benzyloxy)carbonyl]-6-methoxy-2,3,4,9- tetrahydro-1H-beta-carboline-1-carboxylic acid | >200 | |
| 220 | 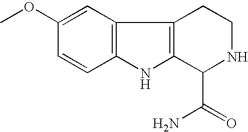 | 6-methoxy-2,3,4,9-tetrahydro-1H-beta- carboline-1-carboxamide | >200 | |
| 221 | 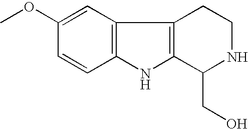 | (6-methoxy-2,3,4,9-tetrahydro-1H-beta- carbolin-1-yl)methanol | >200 | |
| 222 | 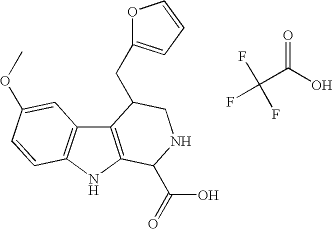 | 4-(2-furylmethyl)-6-methoxy-2,3,4,9- tetrahydro-1H-beta-carboline-1-carboxylic acid trifluoroacetate | >200 | |
| 223 | 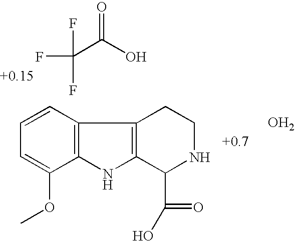 | 8-methoxy-2,3,4,9-tetrahydro-1H-beta- carboline-1-carboxylic acid trifluoroacetate hydrate | >200 | |
| 224 | 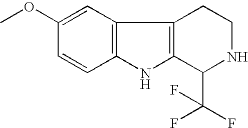 | 6-methoxy-1-(trifluoromethyl)-2,3,4,9- tetrahydro-1H-beta-carboline | >200 | |
| 225 | 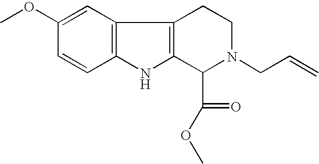 | methyl 2-allyl-6-methoxy-2,3,4,9-tetrahydro- 1H-beta-carboline-1-carboxylate | >200 | |
| 226 | 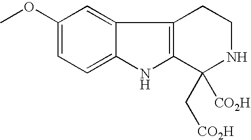 | 1-(carboxymethyl)-6-methoxy-2,3,4,9- tetrahydro-1H-beta-carboline-1-carboxylic acid | >200 | |
| 227 | 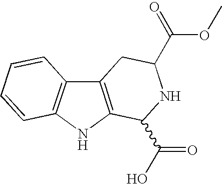 | 3-(methoxycarbonyl)-2,3,4,9-tetrahydro-1H- beta-carboline-1-carboxylic acid | >200 | |
| 228 | 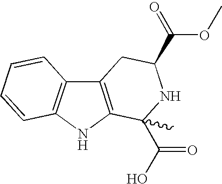 | 3-(methoxycarbonyl)-1-methyl-2,3,4,9- tetrahydro-1H-beta-carboline-1-carboxylic acid | >200 | |
| 229 | 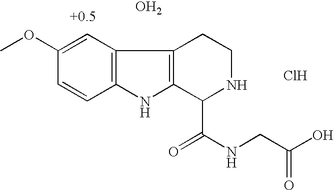 | N-[(6-methoxy-2,3,4,9-tetrahydro-1H-beta- carbolin-1-yl)carbonyl]glycine hydrochloride hydrate | >200 | |
| 230 | 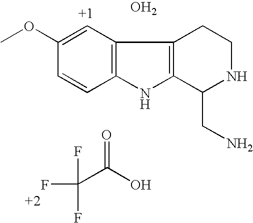 | 1-(6-methoxy-2,3,4,9-tetrahydro-1H-beta- carbolin-1-yl)methanamine trifluoroacetate hydrate | >200 | |
| 231 | 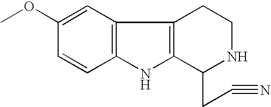 | (6-methoxy-2,3,4,9-tetrahydro-1H-beta- carbolin-1-yl)acetonitrile | >200 | |
| 232 | 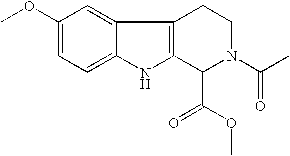 | methyl 2-acetyl-6-methoxy-2,3,4,9-tetrahydro- 1H-beta-carboline-1-carboxylate | >200 | |
| 233 | 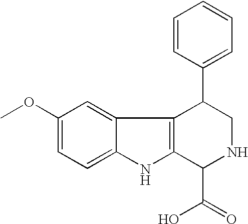 | 6-methoxy-4-phenyl-2,3,4,9-tetrahydro-1H- beta-carboline-1-carboxylic acid | >200 | |
| 234 | 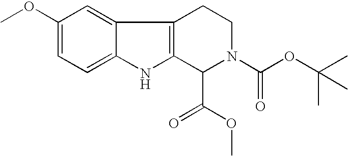 | 2-tert-butyl 1-methyl 6-methoxy-1,3,4,9- tetrahydro-2H-beta-carboline-1,2- dicarboxylate | >200 | |
| 235 | 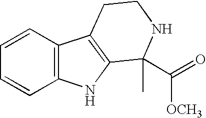 | methyl 1-methyl-2,3,4,9-tetrahydro-1H-beta- carboline-1-carboxylate | >200 | |
| 236 | 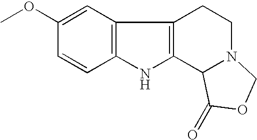 | 8-methoxy-5,6,11,11b-tetrahydro-1H- [1,3]oxazolo[3′,4′:1,2]pyrido[3,4-b]indol-1-one | >200 | |
| 237 | 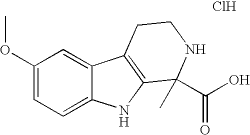 | 6-methoxy-1-methyl-2,3,4,9-tetrahydro-1H- beta-carboline-1-carboxylic acid hydrochloride | >200 | |
| 238 | 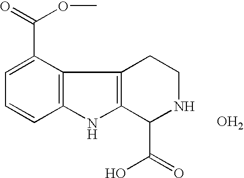 | 5-(methoxycarbonyl)-2,3,4,9-tetrahydro-1H- beta-carbolin-1-carboxylic acid hydrate | >200 | |
| 239 | 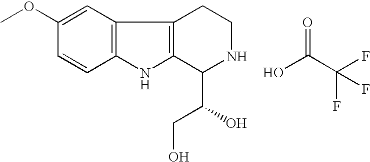 | (1S)-1-(6-methoxy-2,3,4,9-tetrahydro-1H-beta- carbolin-1-yl)ethane-1,2-diol trifluoroacetate | >200 | |
| 240 | 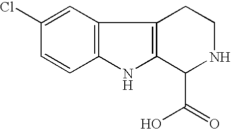 | 6-chloro-2,3,4,9-tetrahydro-1H-beta-carboline- 1-carboxylic acid | >200 | |
| 241 | 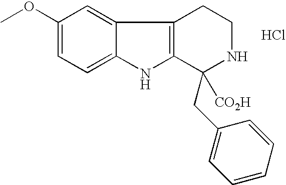 | 1-benzyl-6-methoxy-2,3,4,9-tetrahydro-1H- beta-carboline-1-carboxylic acid hydrochloride | >200 | |

| | | | | TNF |
|---|---|---|---|---|
| | | | | Release |
| | | | MK-2 | Assay |
| | | | Avg. | Avg. |
| | | | 1C50 | 1C50 |
| No. | Structure^{a} | Compound Name(s)^{b} | (μM)^{c} | (μM)^{c} |
| 242 | 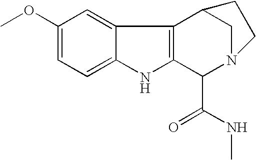 | 7-methoxy-N-methyl-3,4,5,10-tetrahydro-1H- 2,5-methanoazepino[3,4-b]indole-1- carboxamide | >200 | >100 |
| 243 | 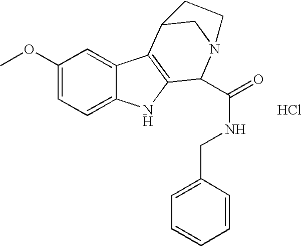 | N-benzyl-7-methoxy-3,4,5,10-tetrahydro-1H- 2,5-methanoazepino[3,4-b]indole-1- carboxamide hydrochloride | >200 | >100 |
| 244 | 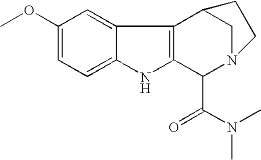 | 7-methoxy-N,N-dimethyl-3,4,5,10-tetrahydro 1H-2,5-methanoazepino[3,4-b]indole-1- carboxamide | >200 | |
| 245 | 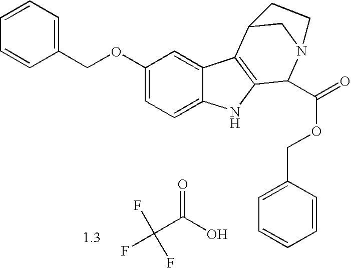 | benzyl 7-(benzyloxy)-3,4,5,10-tetrahydro-1H- 2,5-methanoazepino[3,4-b]indole-1- carboxylate trifluoroacetate | >200 | 76.7 |
| 246 | 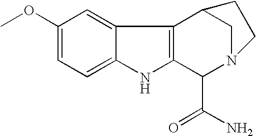 | 7-methoxy-3,4,5,10-tetrahydro-1H-2,5- methanoazepino[3,4-b]indole-1-carboxamide | >200 | >100 |
| 247 | 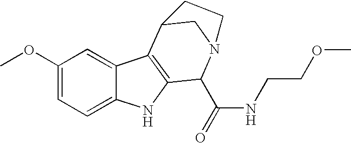 | 7-methoxy-N-(2-methoxyethyl)-3,4,5,10- tetrahydro-1H-2,5-methanoazepino[3,4- b]indole-1-carboxamide | >200 | |
| 248 | 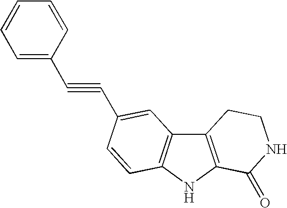 | 6-(phenylethynyl)-2,3,4,9-tetrahydro-1H-beta- carbolin-1-one | >200 | |
| 249 | 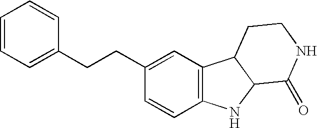 | 6-(2-phenylethyl)-2,3,4,9-tetrahydro-1H-beta- carbolin-1-one | >200 | |
| 250 | 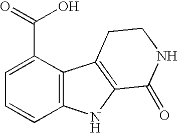 | 1-oxo-2,3,4,9-tetrahydro-1H-beta-carboline-5- carboxylic acid | >200 | |
| 251 | 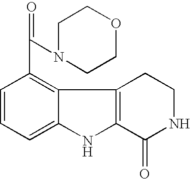 | 5-(morphoiln-4-ylcarbonyl)-2,3,4,9-tetrahydro- 1H-beta-carbolin-1-one | >200 | |
| 252 | 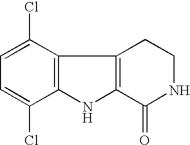 | 5,8-dichloro-2,3,4,9-tetrahydro-1H-beta- carbolin-1-one | >200 | |
| 253 | 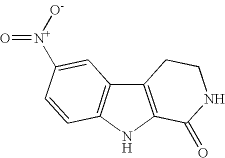 | 6-nitro-2,3,4,9-tetrahydro-1H-beta-carbolin-1-one | >200 | |
| 254 | 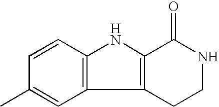 | 1h-Pyrido[3,4-B]Indol-1-One,2,3,4,9- Tetrahydro-6-Methyl- | >200 | |
| 255 | 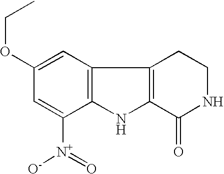 | 6-ethoxy-8-nitro-2,3,4,9-tetrahydro-1H-beta- carbolin-1-one | >200 | |
| 256 | 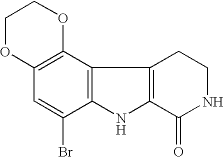 | 6-bromo-2,3,7,9,10,11-hexahydro-8H- [1,4]dioxino[2,3-e]pyrido[3,4-b]indol-8-one | >200 | |
| 257 | 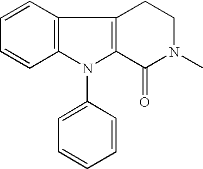 | 2-methyl-9-phenyl-2,3,4,9-tetrahydro-1H-beta- carbolin-1-one | >200 | |
| 258 | 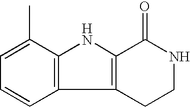 | 1H-Pyrido[3,4-b]indol-1-one,2,3,4,9- tetrahydro-8-methyl- or 8-methyl-2,3,4,9- tetrahydro-1H-beta-carbolin-1-one | >200 | |
| 259 | 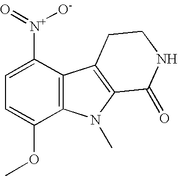 | 8-methoxy-9-methyl-5-nitro-2,3,4,9-tetrahydro- 1H-beta-carbolin-1-one | >200 | |
| 260 | 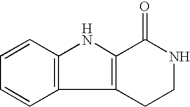 | 1H-Pyrido[3,4-b]indol-1-one,2,3,4,9- tetrahydro- or 2,3,4,9-tetrahydro-1H-beta- carbolin-1-one | >200 | |
| 261 | 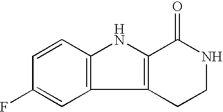 | 1H-Pyrido[3,4-b]indol-1-one,6-fluoro-2,3,4,9- tetrahydro- or 6-fluoro-2,3,4,9-tetrahydro-1H- beta-carbolin-1-one or 1h-Pyrido[3,4-B]Indol- 1-One,6-Fluoro-2,3,4,9-Tetrahydro- | >200 | |
| 262 | 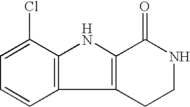 | 1H-Pyrido[3,4-b]indol-1-one,8-chloro-2,3,4,9- tetrahydro- or 8-chloro-2,3,4,9-tetrahydro-1H- beta-carbolin-1-one | >200 | |
| 263 | 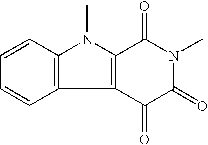 | 1H-Pyrido[3,4-b]indole-1,3,4(2H,9H)-trione, 2,9-dimethyl- or 2,9-dimethyl-1H-beta- carboline-1,3,4(2H,9H)-trione | >200 | |
| 264 | 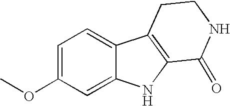 | 7-methoxy-2,3,4,9-tetrahydro-1H-beta- carbolin-1-one | >200 | |
| 265 | 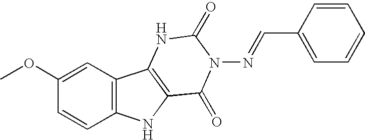 | 8-methoxy-3-{[(1E)- phenylmethylidene]amino}-1H-pyrimido[5,4- b]indole-2,4(3H,5H)-dione | >200 | >100 |
| 266 | 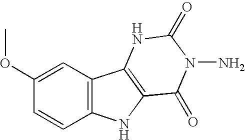 | 3-amino-8-methoxy-1H-pyrimido[5,4-b]indole- 2,4(3H,5H)-dione | >200 | >100 |
| 267 | 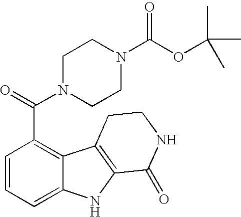 | tert-butyl 4-[(1-oxo-2,3,4,9-tetrahydro-1H-beta- carbolin-5-yl)carbonyl]piperazine-1- carboxylate | >200 | |
| 268 | 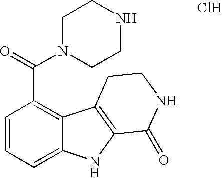 | 5-(piperazin-1-ylcarbonyl)-2,3,4,9-tetrahydro- 1H-beta-carbolin-1-one hydrochloride | >200 | |
| 269 | 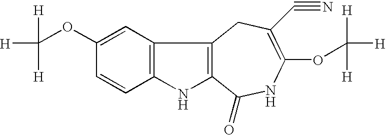 | 3,7-dimethoxy-1-oxo-1,2,5,10- tetrahydroazepino[3,4-b]indole-4-carbonitrile | >200 | |
| 270 | 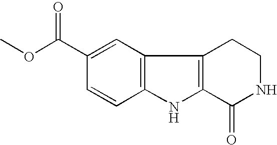 | methyl 1-oxo-2,3,4,9-tetrahydro-1H-beta- carboline-6-carboxylate | >200 | |
| 271 | 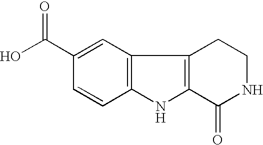 | 1-oxo-2,3,4,9-tetrahydro-1H-beta-carboline-6- carboxylic acid | >200 | |
| 272 | 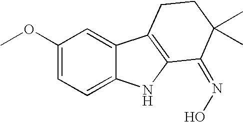 | 6-methoxy-2,2-dimethyl-2,3,4,9-tetrahydro-1H- carbazol-1-one oxime | >200 | |

Notes:
^{a}The beta-carboline compound may be shown with a solvent, such as, for example, trifluoroacetate, with which it can form a salt. Both the salt parent beta-carboline compound and the corresponding salt form are included in the present invention.
^{b}Compound names generated by ACD/Name software.
^{c}A mixture of stereoisomers is implied unless otherwise noted.

The present invention encompasses a method for treating or preventing a disease or disorder in a subject, which disease or disorder is one that can be treated or prevented by inhibiting the activity of MK-2, the method comprising administering to the subject a compound, or a pharmaceutically acceptable salt or prodrug thereof, wherein the compound has the structure according to formula I: 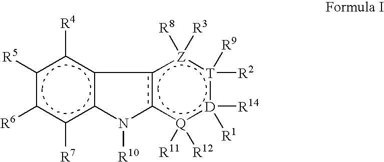
wherein:
Q, D, T and Z are selected from carbon, sulfur, or nitrogen;
when Q is sulfur, R¹² is oxo, and R¹¹ is optionally oxo or is absent;
when T is sulfur, R² is oxo, and R⁹ is optionally oxo or is absent;
when Z is nitrogen, D is nitrogen, and R² and R¹² are oxo;
R¹ is selected from H, alkyl, alkenyl, alkynyl, allyl, acetyl, amino, alkoxy, hydroxy, oxo, carboxy, allylalkyl, benzyl, 2-ethylalkyl, haloacetyl, t-butyldialkylsilyloxyalkyl, alkoxycarbonylaminoalkyl, haloalkylbenzyl, haloalkylcarbonyl, alkylcarbonyl, alkoxycarbonyl, arylcarbonyl, benzyloxycarbonyl, arylalkenylamino, alkoxyheterocyclylcarbonyl, haloarylcarbonyl, or substituted or unsubstituted alkyl, which, if substituted, has one or more substituent groups selected from H, halo, amino, aryl, or heteroaryl;
R² is selected from H, alkyl, alkoxy, hydroxy, carbonyl, oxo, benzyl, hydroxyalkyl, dialkylamino, carboxyl, amino, halo, alkoxycarbonyl, alkoxycarbonyl-R¹⁵, aminoalkyl, haloalkyl, hydroxyalkylbenzyl, haloalkylbenzyl, arylalkenylamino, haloalkoxybenzyl, cyanoalkyl, heterocyclylalkyl, arylalkyl-R¹⁵, alkyl-SO₂—R¹⁵, substituted or unsubstituted aryl, which, if substituted, has one or more substituent groups selected from H, alkyl, amino, nitro, halo, haloalkyl, or hydroxyalkyl, or substituted or unsubstituted alkyl, which, if substituted, has one or more substituent groups selected from H, halo, amino, aryl, or heteroaryl;
R³ is selected from H, oxo, alkyl, alkoxy, amino, halo, haloalkyl, hydroxy, carbonyl, alkoxycarbonyl, benzyl, aminoalkyl, hydroxyalkyl, dialkylamino, arylalkenylamino, carboxy, haloalkyl, hydroxyalkylbenzyl, haloalkylbenzyl, haloalkoxybenzyl, heterocyclylalkyl, substituted or unsubstituted aryl, which, if substituted, has one or more substituent groups selected from H, alkyl, amino, nitro, halo, haloalkyl, or hydroxyalkyl, or substituted or unsubstituted alkyl, which, if substituted, has one or more substituent groups selected from H, halo, amino, aryl, or heteroaryl, or R¹ and R³ optionally join to form an alkyl bridge between the ring atoms to which R¹ and R³ are attached, or R² and R³ optionally join to form a cycloalkyl ring;
R⁴, R⁶ and R⁷ are independently selected from H, alkyl, benzyl, halo, haloalkyl, alkoxy, haloalkoxy, carboxyl, CO₂—R¹⁵, carboxyl, nitro, amino, alkylamino, dialkylamino, benzyloxy, hydrazinocarbonyl, alkoxycarbonyl, alkoxycarbonylheterocyclylcarbonyl, hydroxy, or substituted or unsubstituted 6-membered heterocyclylcarbonyl, which, if substituted, has one or more substituent groups selected from H or alkoxycarbonyl, or R⁶ and R⁷ optionally join to form a heteroaryl ring;
R⁵ is selected from H, alkyl, alkenyl, alkylthio, hydroxy, carboxyl, halo, alkoxy, benzylalkyl, benzylalkoxy, arylalkyl, nitro, alkoxycarbonylalkoxy, alkylthio, benzyloxy, arylalkynyl, arylcarbonylalkoxy, haloalkoxy, haloalkyl, arylalkoxy, haloarylalkoxy, benzyloxoalkoxy, alkoxycarbonylbenzyloxy, alkoxycarbonyl, alkoxyoxoalkoxy, alkoxyoxoarylalkoxy, halobenzyloxy, heterocyclylalkoxy, dialkylaminooxoalkoxy, aminocarbonyl, arylalkynyl, hydrazinocarbonyl, carbonylalkoxy, alkylsulfonylbenzyloxy, CO₂—R¹⁵, carbamoyl, naphthylalkoxy, alkoxyaryloxy, alkoxycarbonylheterocyclylalkoxy, haloalkylbenzyloxy, alkoxyacetylaminoacetyloxy, acetoxy, cycloalkylalkoxy, acetyloxy, morpholinylcarbonyl, alkoxyalkoxy, or R⁴ and R⁵ optionally join to form a heterocyclic ring;
R⁸ and R⁹ are independently optionally present and if present, are independently selected from H, oxo, carbonitrile, alkyl, or is optionally absent, or R³ and R⁸ optionally join to form a cycloalkyl ring;
R¹⁰ is selected from H, alkyl, benzyl, aryl, or hydroxyalkyl;
R¹¹ is optionally present and if present, is selected from H, oxo, alkyl, halobenzyl, benzyl, haloalkyl, 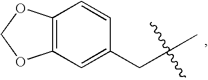
alkoxycarbonyl, carboxyalkyl, benzodioxinylalkyl, substituted or unsubstituted aryl or arylalkyl, which, if substituted, have one or more substituent groups selected from H, alkyl, or alkoxy;
R¹² is optionally present and if present, is selected from H, ═S, oxo, alkyl, carbonyl-R¹³, carboxyl-R¹³, ketone, cyano, cyanoalkyl, alkoxycarbonyl, aminoalkyl, alkoxycarbonylalkyl, carbamyl, hydroxycarbamyl, haloalkyl, heterocyclyl, alkylacetate, heterocyclylalkyl, alkoxycarbonylalkyl, hydroxyalkyl, dihydroxyalkyl, cyanoalkyl, ═N—OR¹⁵, ═N—N(R¹⁵)₂, or hydroxyamino, or R¹ and R¹² optionally join to form a substituted or unsubstituted 5 or 6-membered heteroaryl ring, which, if substituted, has one or more substituent groups selected from H, alkyl, oxo, or alkoxycarbonylalkyl, or R¹¹ and R¹² optionally join to form a ring system having the structure: 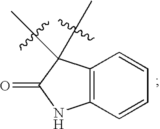
R¹³ is optionally present and if present, is selected from H, alkyl, alkoxy, amino, alkynyl, allyl, alkenyl, hydrazine, benzyl, glycine, allylalkyl, haloalkyl, hydroxyalkyl, hydroxyamino, dihydroxyalkyl, alkoxyalkyl, alkoxyamino, alkylthioalkyl, cycloalkyl, cyclohexylalkyl, amino, alkylamino, benzylalkyl, benzylamino, benzyloxycarbonylalkylamino, dialkylamino, adamantyl, haloalkoxybenzyl, alkoxyaminoalkyl, haloalkylarylalkyl, heterocyclylalkyl, alkoxyalkylamino, arylthioalkyl, substituted or unsubstituted aryl or arylalkyl, which, if substituted, have one or more substituent groups selected from H, alkyl, alkoxy, amino, nitro, halo, haloalkyl, or hydroxyalkyl;
R¹⁴ is optionally present and if present, is selected from H or alkyl; and
R¹⁵ is optionally present and if present, is selected from H, alkyl, alkoxy, or aminoalkyl.

The present invention also encompasses a method for treating or preventing a disease or disorder in a subject, which disease or disorder is one that can be treated or prevented by inhibiting the activity of MK-2, the method comprising administering to the subject a compound, or a pharmaceutically acceptable salt or prodrug thereof, wherein the compound has the structure: 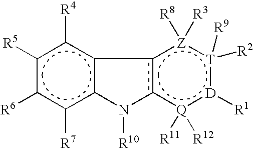
wherein:
Q, D, T and Z are selected from carbon, sulfur, or nitrogen;
when Q is sulfur, R¹² is oxo, and R¹¹ is optionally oxo or is absent;
when T is sulfur, R² is oxo, and R⁹ is optionally oxo or is absent;
when Z is nitrogen, D is nitrogen, and R² and R¹² are oxo;
R¹ is selected from H, alkyl, alkenyl, alkynyl, allyl, acetyl, amino, alkoxy, hydroxy, oxo, carboxy, allylalkyl, benzyl, 2-ethylalkyl, haloacetyl, alkoxycarbonylaminoalkyl, t-butyldialkylsilyloxyalkyl, alkoxycarbonylaminoalkyl, haloalkylbenzyl, haloalkylcarbonyl, alkylcarbonyl, alkoxycarbonyl, arylcarbonyl, benzyloxycarbonyl, alkoxycarbonyl, arylalkenylamino, alkoxyheterocyclylcarbonyl, haloarylcarbonyl, or substituted or unsubstituted alkyl, which, if substituted, has one or more substituent groups selected from H, halo, amino, aryl, or heteroaryl;
R² is selected from H, alkyl, alkoxy, hydroxy, carbonyl, oxo, benzyl, hydroxyalkyl, dialkylamino, carboxyl, amino, halo, alkoxycarbonyl, aminoalkyl, haloalkyl, hydroxyalkylbenzyl, haloalkylbenzyl, arylalkenylamino, haloalkoxybenzyl, heterocyclylalkyl, substituted or unsubstituted aryl, which, if substituted, has one or more substituent groups selected from H, alkyl, amino, nitro, halo, haloalkyl, or hydroxyalkyl, or substituted or unsubstituted alkyl, which, if substituted, has one or more substituent groups selected from H, halo, amino, aryl, or heteroaryl;
R³ is selected from H, alkyl, alkoxy, amino, halo, haloalkyl, hydroxy, carbonyl, alkoxycarbonyl, benzyl, aminoalkyl, hydroxyalkyl, dialkylamino, arylalkenylamino, carboxy, haloalkyl, hydroxyalkylbenzyl, haloalkylbenzyl, haloalkoxybenzyl, heterocyclylalkyl, substituted or unsubstituted aryl, which, if substituted, has one or more substituent groups selected from H, alkyl, amino, nitro, halo, haloalkyl, or hydroxyalkyl, or substituted or unsubstituted alkyl, which, if substituted, has one or more substituent groups selected from H, halo, amino, aryl, or heteroaryl, or R¹ and R³ optionally join to form an alkyl bridge between the ring atoms to which R¹ and R³ are attached, or R² and R³ optionally join to form a cycloalkyl ring;
R⁴, R⁶ and R⁷ are independently selected from H, alkyl, benzyl, halo, haloalkyl, alkoxy, haloalkoxy, carboxyl, nitro, amino, alkylamino, dialkylamino, benzyloxy, hydrazinocarbonyl, alkoxycarbonyl, alkoxycarbonylheterocyclylcarbonyl, hydroxy, or substituted or unsubstituted 6-membered heterocyclylcarbonyl, which, if substituted, has one or more substituent groups selected from H or alkoxycarbonyl;
R⁵ is selected from H, alkyl, alkenyl, alkylthio, hydroxy, carboxyl, halo, alkoxy, benzylalkyl, benzylalkoxy, arylalkyl, nitro, alkoxycarbonylalkoxy, alkylthio, benzyloxy, arylalkynyl, arylcarbonylalkoxy, haloalkoxy, arylalkoxy, arylcarbonylalkoxy, haloarylalkoxy, benzyloxoalkoxy, alkoxycarbonylbenzyloxy, alkoxycarbonyl, alkoxyoxoalkoxy, alkoxyoxoarylalkoxy, halobenzyloxy, heterocyclylalkoxy, dialkylaminooxoalkoxy, aminocarbonyl, arylalkynyl, hydrazinocarbonyl, carbonylalkoxy, alkylsulfonylbenzyloxy, carboxyalkoxy, carbamoyl, naphthylalkoxy, alkoxycarbonylheterocyclylalkoxy, haloalkylbenzyloxy, alkoxyacetylaminoacetyloxy, acetoxy, cycloalkylalkoxy, acetyloxy, morpholinylcarbonyl, alkoxyalkoxy, or R⁴ and R⁵ optionally join to form a heterocyclic ring;
R⁸ and R⁹ are independently selected from H, oxo, carbonitrile, alkyl, or is optionally absent, or R³ and R⁸ optionally join to form a cycloalkyl ring;
R¹⁰ is selected from H, alkyl, benzyl, aryl, or hydroxyalkyl;
R¹¹ is selected from H, oxo, alkyl, halobenzyl, benzyl, haloalkyl, 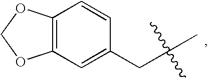
alkoxycarbonyl, carboxyalkyl, benzodioxinylalkyl, substituted or unsubstituted aryl or arylalkyl, which, if substituted, have one or more substituent groups selected from H, alkyl, or alkoxy;
R¹² is selected from H, oxo, alkyl, carbonyl-R¹³, carboxyl-R¹³, ketone, cyano, cyanoalkyl, alkoxycarbonyl, aminoalkyl, alkoxycarbonylalkyl, carbamyl, hydroxycarbamyl, haloalkyl, heterocyclyl, alkylacetate, heterocyclylalkyl, alkoxycarbonylalkyl, hydroxyalkyl, dihydroxyalkyl, cyanoalkyl, or hydroxyamino, or R¹ and R¹² optionally join to form a substituted or unsubstituted 5 or 6-membered heteroaryl ring, which, if substituted, has one or more substituent groups selected from H, alkyl, oxo, or alkoxycarbonylalkyl, or R¹¹ and R¹² optionally join to form a ring system having the structure: 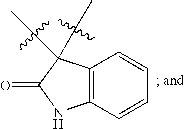
R¹³ is optionally present and if present, is selected from H, alkyl, alkoxy, amino, alkynyl, allyl, alkenyl, hydrazine, benzyl, glycine, allylalkyl, haloalkyl, hydroxyalkyl, hydroxyamino, dihydroxyalkyl, alkoxyalkyl, alkoxyamino, alkylthioalkyl, cycloalkyl, cyclohexylalkyl, amino, alkylamino, benzylalkyl, benzylamino, benzyloxycarbonylalkylamino, dialkylamino, adamantyl, haloalkoxybenzyl, alkoxyaminoalkyl, haloalkylarylalkyl, heterocyclylalkyl, alkoxyalkylamino, arylthioalkyl, substituted or unsubstituted aryl or arylalkyl, which, if substituted, have one or more substituent groups selected from H, alkyl, alkoxy, amino, nitro, halo, haloalkyl, or hydroxyalkyl.

The present invention also encompasses a method for treating or preventing a disease or disorder in a subject, which disease or disorder is one that can be treated or prevented by inhibiting the activity of MK-2, the method comprising administering to the subject a compound, or a pharmaceutically acceptable salt or prodrug thereof, wherein the compound has the structure according to formula I, wherein:
Q, D, T and Z are selected from carbon, sulfur, or nitrogen;
when Q is sulfur, R¹² is oxo, and R¹¹ is optionally oxo or is absent;
when T is sulfur, R² is oxo, and R⁹ is optionally oxo or is absent;
when Z is nitrogen, D is nitrogen, and R² and R¹² are oxo;
R¹ is selected from H, alkyl, alkenyl, alkynyl, allyl, acetyl, amino, alkoxy, hydroxy, oxo, carboxy, allylalkyl, benzyl, 2-ethylalkyl, haloacetyl, alkoxycarbonylaminoalkyl, t-butyldialkylsilyloxyalkyl, alkoxycarbonylaminoalkyl, haloalkylbenzyl, haloalkylcarbonyl, alkylcarbonyl, alkoxycarbonyl, arylcarbonyl, benzyloxycarbonyl, alkoxycarbonyl, arylalkenylamino, alkoxyheterocyclylcarbonyl, haloarylcarbonyl, or substituted or unsubstituted alkyl, which, if substituted, has one or more substituent groups selected from H, halo, amino, aryl, or heteroaryl;
R² is selected from H, alkyl, alkoxy, hydroxy, carbonyl, oxo, benzyl, hydroxyalkyl, dialkylamino, carboxyl, amino, halo, alkoxycarbonyl, aminoalkyl, haloalkyl, hydroxyalkylbenzyl, haloalkylbenzyl, alkoxycarbonyl-R¹⁵, cyanoalkyl, arylalkyl-R¹⁵, alkyl-SO₂—R¹⁵, arylalkenylamino, haloalkoxybenzyl, heterocyclylalkyl, substituted or unsubstituted aryl, which, if substituted, has one or more substituent groups selected from H, alkyl, amino, nitro, halo, haloalkyl, or hydroxyalkyl, or substituted or unsubstituted alkyl, which, if substituted, has one or more substituent groups selected from H, halo, amino, aryl, or heteroaryl;
R³ is selected from H, oxo, alkyl, alkoxy, amino, halo, haloalkyl, hydroxy, carbonyl, alkoxycarbonyl, benzyl, aminoalkyl, hydroxyalkyl, dialkylamino, arylalkenylamino, carboxy, haloalkyl, hydroxyalkylbenzyl, haloalkylbenzyl, haloalkoxybenzyl, furylalkyl, substituted or unsubstituted aryl, which, if substituted, has one or more substituent groups selected from H, alkyl, amino, nitro, halo, haloalkyl, or hydroxyalkyl, or substituted or unsubstituted alkyl, which, if substituted, has one or more substituent groups selected from H, halo, amino, aryl, or heteroaryl, or R¹ and R³ optionally join to form an alkyl bridge between the ring atoms to which R¹ and R³ are attached, or R² and R³ optionally join to form a cycloalkyl ring;
R⁴, R⁶ and R⁷ are independently selected from H, alkyl, benzyl, halo, haloalkyl, alkoxy, haloalkoxy, CO₂—R¹⁵, carboxyl, nitro, amino, alkylamino, dialkylamino, benzyloxy, hydrazinocarbonyl, alkoxycarbonyl, alkoxycarbonylpiperazinylcarbonyl, hydroxy, morpholinylcarbonyl, or piperazinylcarbonyl, or R⁶ and R⁷ optionally join to form a heteroaryl ring;
R⁵ is selected from H, alkyl, alkenyl, alkylthio, hydroxy, halo, alkoxy, benzylalkyl, benzylalkoxy, arylalkyl, nitro, haloalkyl, alkoxycarbonylalkoxy, alkylthio, benzyloxy, phenylalkynyl, phenylcarbonylalkoxy, benzyloxoalkoxy, CO₂—R¹⁵, haloalkoxy, phenylalkoxy, haloarylalkoxy, alkoxycarbonylbenzyloxy, alkoxycarbonyl, alkoxyoxoalkoxy, alkoxyoxophenylalkoxy, halobenzyloxy, pyridinylalkoxy, dialkylaminooxoalkoxy, aminocarbonyl, phenylalkynyl, hydrazinocarbonyl, carbonylalkoxy, alkylsulfonylbenzyloxy, carboxyalkoxy, carbamoyl, naphthylalkoxy, alkoxycarbonylfurylalkoxy, haloalkylbenzyloxy, alkoxyacetylaminoacetyloxy, acetoxy, cyclohexylalkoxy, acetyloxy, morpholinylcarbonyl, alkoxyalkoxy, or R⁴ and R⁵ optionally join to form a pyranyl or 1,4 dioxinyl ring;
R⁸ and R⁹ are independently selected from H, oxo, carbonitrile, alkyl, or R³ and R⁸ optionally join to form a cycloalkyl ring;
R¹⁰ is selected from H, alkyl, benzyl, aryl, or hydroxyalkyl;
R¹¹ is selected from H, oxo, alkyl, halobenzyl, benzyl, haloalkyl, 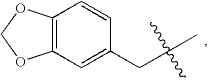
alkoxycarbonyl, carboxyalkyl, benzodioxinylalkyl, substituted or unsubstituted aryl or arylalkyl, which, if substituted, have one or more substituent groups selected from H, alkyl, or alkoxy;
R¹² is selected from H, oxo, alkyl, carbonyl-R¹³, carboxyl-R¹³, ═S, ═N—OR¹⁵, ═N—N(R¹⁵)₂, ketone, cyano, cyanoalkyl, alkoxycarbonyl, aminoalkyl, alkoxycarbonylalkyl, carbamyl, hydroxycarbamyl, haloalkyl, tetrazolyl, tetrazolylalkyl, alkylacetate, t-butoxycarbonylalkyl, hydroxyalkyl, dihydroxyalkyl, isoxazolyl, furanyl, pyrrolyl, pyrazolyl, imidazolyl, acetonitrile, or hydroxyamino, or R¹ and R¹² optionally join to form a ring system selected from: 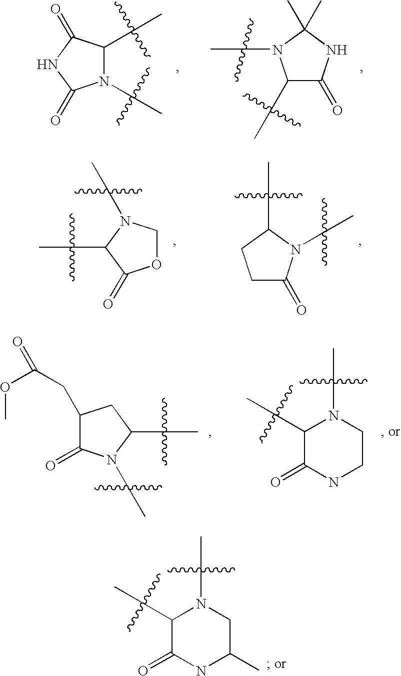
R¹¹ and R¹² optionally join to form a ring system having the structure: 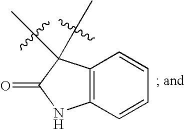
R¹³ is optionally present and if present, is selected from H, alkyl, alkoxy, amino, alkynyl, allyl, alkenyl, hydrazine, benzyl, glycine, allylalkyl, haloalkyl, hydroxyalkyl, hydroxyamino, dihydroxyalkyl, alkoxyalkyl, alkoxyamino, alkylthioalkyl, cyclohexyl, cyclohexylalkyl, amino, alkylamino, benzylalkyl, benzylamino, benzyloxycarbonylalkylamino, dialkylamino, adamantyl haloalkoxybenzyl, alkoxyaminoalkyl, haloalkylarylalkyl, pyridinylalkyl, alkoxyalkylamino, phenylthioalkyl, substituted or unsubstituted aryl or arylalkyl, which, if substituted, have one or more substituent groups selected from H, alkyl, alkoxy, amino, nitro, halo, haloalkyl, or hydroxyalkyl;
R¹⁴ is optionally present and if present, is selected from H or alkyl; and
R¹⁵ is optionally present and if present, is selected from H, alkyl, alkoxy, or aminoalkyl.

The present invention encompasses a method for treating or preventing a disease or disorder in a subject, which disease or disorder is one that can be treated or prevented by inhibiting the activity of MK-2, the method comprising administering to the subject a compound, or a pharmaceutically acceptable salt or prodrug thereof, wherein the compound has the structure according to formula I, wherein:
Q, D, T and Z are selected from carbon, sulfur, or nitrogen;
when Q is sulfur, R¹² is oxo, and R¹¹ is optionally oxo or is absent;
when T is sulfur, R² is oxo, and R⁹ is optionally oxo or is absent;
when Z is nitrogen, D is nitrogen, and R² and R¹² are oxo;
R¹ is selected from H, (C₁-C₆) alkyl, (C₁-C₄) alkenyl, (C₁-C₄) allyl, (C₁-C₄) alkynyl, acetyl, amino, (C₁-C₆) alkoxy, hydroxy, oxo, carboxy, allyl-(C₁-C₄) alkyl, benzyl, 2-ethyl-(C₁-C₄) alkyl, haloacetyl, (C₁-C₄) alkoxycarbonylamino-(C₁-C₄) alkyl, t-butyldimethylsilyloxy-(C₁-C₄) alkyl, t-butoxycarbonylamino-(C₁-C₄) alkyl, halo-(C₁-C₄) alkylbenzyl, halo-(C₁-C₄) alkylcarbonyl, (C₁-C₄) alkylcarbonyl, (C₁-C₄) alkoxycarbonyl, arylcarbonyl, benzyloxycarbonyl, t-butoxycarbonyl, phenylmethylideneamino, (C₁-C₄) alkoxyfuranylcarbonyl, or haloarylcarbonyl;
R² is selected from H, (C₁-C₆) alkyl, (C₁-C₆) alkoxy, hydroxy, carbonyl, oxo, benzyl, hydroxy-(C₁-C₄) alkyl, dimethylamino, carboxyl, amino, halo, (C₁-C₄) alkoxycarbonyl, amino-(C₁-C₄) alkyl, or halo-(C₁-C₄) alkyl, (C₁-C₆) alkoxycarbonyl-R¹⁵, cyano-(C₁-C₆) alkyl, aryl-(C₁-C₆) alkyl-R¹⁵, or (C₁-C₆) alkyl-SO₂—R¹⁵;
R³ is selected from H, (C₁-C₆) alkyl, oxo, halo-(C₁-C₄) alkyl, carboxy, oxo, hydroxy-(C₁-C₄) alkylbenzyl, halo-(C₁-C₄) alkylbenzyl, halo-(C₁-C₄) alkoxybenzyl, furyl-(C₁-C₄) alkyl, substituted or unsubstituted aryl, which, if substituted, has one or more substituent groups selected from H, (C₁-C₄) alkyl, amino, nitro, halo, halo-(C₁-C₄) alkyl, or hydroxy-(C₁-C₄) alkyl, or R¹ and R³ optionally join to form a (C₁-C₃) alkyl bridge between the ring atoms to which R¹ and R³ are attached, or R² and R³ optionally join to form a cyclohexyl ring;
R⁴, R⁶ and R⁷ are independently selected from H, (C₁-C₆) alkyl, benzyl, halo, halo-(C₁-C₄) alkyl, (C₁-C₆) alkoxy, CO₂—R¹⁵, halo-(C₁-C₄) alkoxy, carboxyl, nitro, benzyloxy, hydrazinocarbonyl, (C₁-C₄) alkoxycarbonyl, (C₁-C₄) alkoxycarbonylpiperazinylcarbonyl, hydroxy, morpholinylcarbonyl, or piperazinylcarbonyl, or R⁶ and R⁷ optionally join to form a pyridyl ring;
R⁵ is selected from H, (C₁-C₆) alkyl, (C₁-C₄) alkenyl, (C₁-C₄) alkylthio, hydroxy, halo, (C₁-C₆) alkoxy, benzyl-(C₁-C₄) alkyl, benzyl-(C₁-C₄) alkoxy, aryl-(C₁-C₄) alkyl, benzyloxo-(C₁-C₄) alkoxy, nitro, (C₁-C₄) alkoxycarbonylalkoxy, (C₁-C₄) alkylthio, benzyloxy, phenyl-(C₁-C₄) alkynyl, phenylcarbonyl-(C₁-C₄) alkoxy, halo-(C₁-C₆) alkyl, CO₂—R¹⁵, halo-(C₁-C₄) alkoxy, aryl-(C₁-C₄) alkoxy, arylcarbonyl-(C₁-C₄) alkoxy, haloaryl-(C₁-C₄) alkoxy, (C₁-C₄) alkoxycarbonylbenzyloxy, (C₁-C₄) alkoxycarbonyl, (C₁-C₄) alkoxyoxo-(C₁-C₄) alkoxy, -(C₁-C₄) alkoxyoxophenyl-(C₁-C₄) alkoxy, halobenzyloxy, pyridinyl-(C₁-C₄) alkoxy, dialkylaminooxo-(C₁-C₄) alkoxy, aminocarbonyl, phenyl-(C₁-C₄) alkynyl, hydrazinocarbonyl, carbonyl-(C₁-C₄) alkoxy, (C₁-C₄) alkylsulfonylbenzyloxy, carboxy-(C₁-C₄) alkoxy, carbamoyl, naphthyl-(C₁-C₄) alkoxy, (C₁-C₄) alkoxycarbonylfuryl-(C₁-C₄) alkoxy, halo-(C₁-C₄) alkylbenzyloxy, (C₁-C₄) alkoxyacetylaminoacetyloxy, acetoxy, cyclohexyl-(C₁-C₄) alkoxy, acetyloxy, morpholinylcarbonyl, (C₁-C₄) alkoxy-(C₁-C₄) alkoxy, or R⁴ and R⁵ optionally join to form a pyranyl or 1,4 dioxinyl ring;
R⁸ and R⁹ are independently selected from H, oxo, carbonitrile, (C₁-C₆) alkyl, or R³ and R⁸ optionally join to form a cycloalkyl ring;
R¹⁰ is selected from H, (C₁-C₆) alkyl, benzyl, phenyl, or hydroxy-(C₁-C₄) alkyl;
R¹¹ is selected from H, oxo, (C₁-C₆) alkyl, halobenzyl, benzyl, halo-(C₁-C₄) alkyl, C₁-C₄) alkoxycarbonyl, carboxy-(C₁-C₄) alkyl, 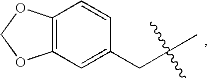
benzodioxinyl-(C₁-C₄) alkyl, substituted or unsubstituted aryl or aryl-(C₁-C₆) alkyl, which, if substituted, have one or more substituent groups selected from H, (C₁-C₆) alkyl, (C₁-C₆) alkoxy;
R¹² is selected from H, oxo, (C₁-C₆) alkyl, carbonyl-R¹³, carboxyl-R¹³, ═S, ═N—OR¹⁵, ═N—N(R¹⁵)₂, cyano, cyano-(C₁-C₄) alkyl, (C₁-C₄) alkoxycarbonyl, amino-(C₁-C₄) alkyl, (C₁-C₄) alkoxycarbonyl-(C₁-C₄) alkyl, carbamyl, hydroxycarbamyl, halo-(C₁-C₄) alkyl, tetrazolyl, tetrazolyl-(C₁-C₄) alkyl, (C₁-C₄) alkylacetate, t-butoxycarbonyl-(C₁-C₄) alkyl, hydroxy-(C₁-C₄) alkyl, dihydroxy-(C₁-C₄) alkyl, isoxazolyl, furanyl, pyrrolyl, pyrazolyl, imidazolyl, acetonitrile, or hydroxyamino, or R¹ and R¹² optionally join to form a ring system selected from: 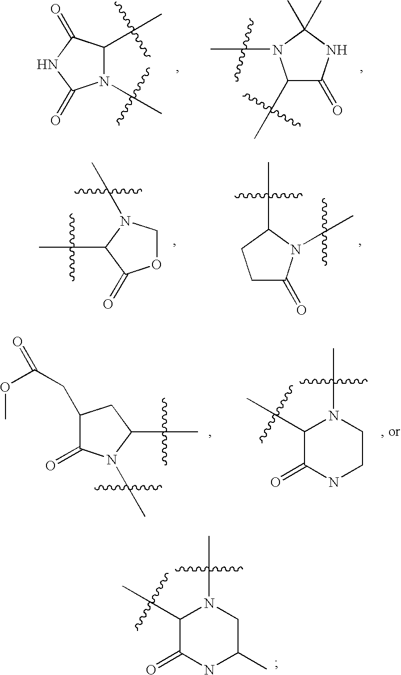
or R¹¹ and R¹² optionally join to form a ring system selected from: 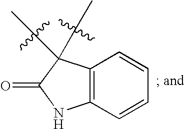
R¹³ is optionally present and if present, is selected from H, (C₁-C₆) alkyl, (C₁-C₆) alkoxy, amino, (C₁-C₆) allyl, (C₁-C₆) alkynyl, (C₁-C₆) alkenyl, hydrazine, benzyl, glycine, allyl-(C₁-C₄) alkyl, halo-(C₁-C₄) alkyl, hydroxy-(C₁-C₄) alkyl, hydroxyamino, dihydroxy-(C₁-C₄) alkyl, (C₁-C₄) alkoxy-(C₁-C₄) alkyl, (C₁-C₄) alkoxyamino, (C₁-C₄) alkylthio-(C₁-C₄) alkyl, cyclohexyl, cyclohexyl-(C₁-C₄) alkyl, amino, (C₁-C₄) alkylamino, benzyl-(C₁-C₄) alkyl, benzylamino, benzyloxycarbonyl-(C₁-C₄) alkylamino, dialkylamino, adamantyl halo-(C₁-C₄) alkoxybenzyl, (C₁-C₄) alkoxyamino-(C₁-C₄) alkyl, halo-(C₁-C₄) alkylaryl-(C₁-C₄) alkyl, pyridinyl-(C₁-C₄) alkyl, (C₁-C₄) alkoxy-(C₁-C₄) alkylamino, phenylthio-(C₁-C₄) alkyl, substituted or unsubstituted aryl or aryl-(C₁-C₄) alkyl, which, if substituted, have one or more substituent groups selected from H, (C₁-C₆) alkyl, (C₁-C₆) alkoxy, amino, nitro, halo, halo-(C₁-C₄) alkyl, or hydroxy-(C₁-C₄) alkyl;
R¹⁴ is optionally present and if present, is selected from H or (C₁-C₆) alkyl; and
R¹⁵ is optionally present and if present, is selected from H, (C₁-C₆) alkyl, (C₁-C₆) alkoxy, or amino-(C₁-C₆) alkyl.

The present invention encompasses a method for treating or preventing a disease or disorder in a subject, which disease or disorder is one that can be treated or prevented by inhibiting the activity of MK-2, the method comprising administering to the subject a compound, or a pharmaceutically acceptable salt or prodrug thereof, wherein the compound has the structure according to formula I, wherein:
Q, D, T and Z are selected from carbon, sulfur, or nitrogen;
when Q is sulfur, R¹² is oxo, and R¹¹ is optionally oxo or is absent;
when T is sulfur, R² is oxo, and R⁹ is optionally oxo or is absent;
when Z is nitrogen, D is nitrogen, and R² and R¹² are oxo;
R¹ is selected from H, methyl, ethyl, propyl, isopropyl, n-propyl, t-butyldimethylsilyloxypropyl, t-butoxycarbonylaminoethyl, 2-ethylbutyl, chlorophenylcarbonyl, trifluoroacetyl, methoxyfuranylcarbonyl, benzyloxycarbonyl, propene, acetyl, t-butoxycarbonyl, trifluoromethylbenzyl, benzyl, oxo, or phenylmethylideneamino.
R² is selected from H, methyl, ethyl, methoxy, propyl, n-propyl, amino-(C₁-C₄) alkyl, hydroxyl-(C₁-C₄) alkyl, dimethylaminomethyl, carboxyl, dimethylamino, benzyl, trifluoromethyl, methoxycarbonyl, oxo, methoxycarbonyl-R¹⁵, cyanomethyl, phenylalkyl-R¹⁵, or methyl-SO₂—R¹⁵;
R³ is selected from H, methyl, ethyl, propyl, n-propyl, carboxyl, trifluoromethyl, p-hydroxymethylbenzyl, m-trifluoromethylbenzyl, o-trifluoromethylbenzyl, trifluoromethoxybenzyl, phenyl, furylmethyl, or oxo, or R¹ and R³ optionally join to form a C₁-C₃ alkyl bridge between the ring atoms to which R¹ and R³ are attached, or R² and R³ optionally join to form a ring system consisting of: 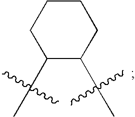
R⁴, R⁶ and R⁷ are independently selected from H, methyl, benzyl, methoxy, hydroxyl, methoxycarbonyl, benzyloxy, hydrazinocarbonyl, carbonxyl, fluoro, chloro, morpholinylcarbonyl, nitro, CO₂—R¹⁵, bromo, t-butoxycarbonylpiperazinylcarbonyl, piperazinylcarbonyl, or R⁶ and R⁷ optionally join to form a pyridyl ring;
R⁵ is selected from H, methyl, methoxy, acetoxy, carbamoyl, 2-oxo-2-phenylethoxy, hydroxyl, benzyloxy, benzyloxoethoxy, methoxycarbonyl, 2-ethoxy-2-oxoethoxy, 2-methoxy-2-oxo-1-phenylethoxy, butoxy, n-butoxy, 2-methoxy-2-oxoethoxy, p-chlorobenzyloxy, o-chlorobenzyloxy, m-chlorobenzyloxy, chloropropoxy, chlorobutoxy, chloro, 3-pyridylmethoxy, 4-pyridylmethoxy, N,N-dimethylcarbamylmethoxy, trifluoromethyl, CO₂—R¹⁵, 2-phenylethoxy, aminocarbonyl, bromo, fluoro, carboxymethoxy, p-(methylsulfonyl)benzyloxy, hydrazinocarbonyl, 2-naphthylmethoxy, 4-ethoxy-4-oxobutoxy, (5-ethoxycarbonyl-2-furyl)methoxy, 2-methoxyethoxy, p-(trifluoromethyl)benzyloxy, 3-phenylpropoxy, m-chlorobenzylmethoxy, 3-methoxycarbonylbenzyloxy, 2-pyridylmethoxy, cyclohexylmethoxy, ethoxycarbonylmethylaminocarbonylmethoxy, iodo, phenylethynyl, 2-phenylethyl, nitro, ethoxy, or methylthio, or R⁴ and R⁵ optionally join to form a pyranyl or 1,4 dioxinyl ring;
R⁸ and R⁹ are independently selected from H, oxo, carbonitrile, methyl, or R³ and R⁸ optionally join to form a cyclohexyl ring;
R¹⁰ is selected from H, benzyl, phenyl, methyl, ethyl, or hydroxymethyl;
R¹¹ is selected from H, oxo, methyl, benzyl, phenyl, chloromethyl, p-chlorobenzyl, methoxycarbonyl, ethoxycarbonyl, carboxyethyl, 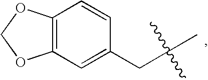
carboxypropyl, or trimethoxybenzyl;
R¹² is selected from H, oxo, methyl, ethyl, carbonyl-R¹³, carboxyl-R¹³, ═S, ═N—OR¹⁵, ═N—N(R¹⁵)₂, cyano, cyanomethyl, tetrazoyl, pyrrolyl, tetrazoylmethyl, methylacetate, t-butylacetate, hydroxymethyl, trifluoromethyl, aminomethyl, acetonitrile, ethane-1,2-diol, methoxycarbonyl, methoxycarbonylmethyl, carbamyl, hydroxycarbamyl, isoxazolyl, furanyl, pyrazolyl, imidazolyl, or hydroxyamino, or R¹ and R¹² optionally join to form a ring system selected from: 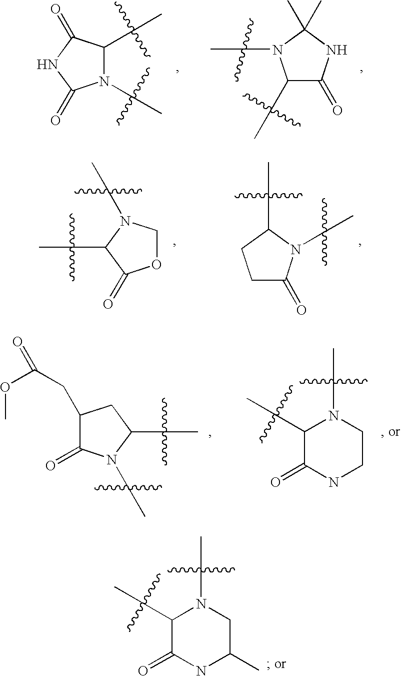
R¹¹ and R¹² optionally join to form a ring system having the structure: 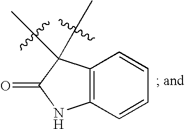
R¹³ is optionally present and if present, is selected from H, methyl, ethyl, i-propyl, n-propyl, propyl, benzyl, hydroxyamino, hydrazino, glycine, propene, benzyloxycarbonylmethylamino, 1-phenylethyl, 2,2,2-trifluoroethyl, 2-fluoroethyl, 2-hydroxyethyl, 2,3-dihydroxypropyl, 2-methoxyethyl, 2-(methylthio)ethyl, cyclohexyl, cyclohexylmethyl, t-butyl, neopentyl, 1-adamantyl, pentyl, amino, N,N-dimethylamino, benzylamino, 2-methoxyethylamino, n-hexyl, p-(trifluoromethylbenzyl), trifluoromethoxybenzyl, methylamino, pyridylmethyl, (N-methoxy-N-methyl)amino, 2,2,6,6-tetramethylcyclohexyl, or 2-(phenylthio)ethyl;
R¹⁴ is optionally present and if present, is selected from H or methyl; and
R¹⁵ is optionally present and if present, is selected from H, (C₁-C₄) alkyl, (C₁-C₄) alkoxy, or amino-(C₁-C₄) alkyl.

The present method can be practiced by the administration of a beta-carboline compound that provides an IC₅₀ of less than about 200 μM, in an in vitro assay of MK-2 inhibitory activity. Examples of such compounds comprise the compound having the structure shown in formula I, or a pharmaceutically acceptable salt or prodrug thereof, wherein the compound has the structure according to formula I, wherein:
Q, D, T and Z are selected from carbon, sulfur, or nitrogen;
when Q is sulfur, R¹² is oxo, and R¹¹ is optionally oxo or is absent;
when T is sulfur, R² is oxo, and R⁹ is optionally oxo or is absent;
when Z is nitrogen, D is nitrogen, and R² and R¹² are oxo;
R¹ is selected from H, methyl, ethyl, i-propyl, propenyl, or trifluoromethylbenzyl;
R² is selected from H, methyl, ethyl, propyl, hydroxyl-(C₁-C₄) alkyl, methoxycarbonyl-R¹⁵, cyanomethyl, phenylalkyl-R¹⁵, methyl-SO₂—R¹⁵, or amino-(C₁-C₄) alkyl;
R³ is selected from H, oxo, methyl, ethyl, carboxyl, propyl, benzyl, hydroxymethylbenzyl, trifluoromethyl, trifluoromethylbenzyl, or R¹ and R³ optionally join to form a C₁-C₃ alkyl bridge between the ring atoms to which R¹ and R³ are attached;
R⁴, R⁶ and R⁷ are independently selected from H, methoxy, CO₂—R¹⁵, methoxycarbonyl, hydroxyl, or R⁶ and R⁷ optionally join to form a pyridyl ring;
R⁵ is selected from H, methyl, hydroxy, methoxy, butyl, butoxy, methylcarboxymethoxy, ethylcarboxymethoxy, chloropropoxy, chlorobutoxy, methoxycarbonyl, 2-methoxy-2-oxoethoxy, trifluoromethyl, CO₂—R¹⁵, methoxycarbonylbenzyloxy, 2-oxo-2-phenylethoxy, phenylmethoxy, phenylethoxy, aminocarbonyl, 2-methoxyethoxy, chlorobenzyloxy, 3-phenylpropoxy, phenylcarbonylmethoxy, chlorobenzyloxy, hydrazinocarbonyl, benzyloxy, acetyloxy, 2-methoxy-2-oxo-1-phenylethoxy, trifluoromethylbenzyloxy, methylcarboxyphenylmethoxy, methylcarboxy, fluoro, chloro, iodo, bromo, ethoxycarbonylmethylaminocarbonylmethoxy, methylthio, furylmethoxy, N,N-dimethylcarbamylmethoxy, p-(methylsulfonyl)benzyloxy, 2-ethoxy-2-oxoethoxy, carboxymethoxy, pyridyloxomethoxy, pyridylmethoxy, carbazoyl, methylpropoxy, carbamyl, naphthlylmethoxy, or R⁴ and R⁵ optionally join to form a pyran ring;
R⁸, R⁹ and R¹⁰ are independently selected from H, benzyl or methyl;
R¹¹ is selected from H, or hydroxamate;
R¹² is selected from H, oxo, tetrazolyl, carbonyl-R¹³, or carboxyl-R¹³, ═S, ═N—OR¹⁵, ═N—N(R¹⁵)₂, or R¹ and R¹² optionally join to form a ring system having the structure: 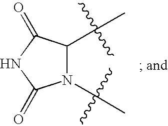
R¹³ is selected from H, methyl, ethyl, i-propyl, propyl, benzyl, 1-phenylethyl, 2-fluoroethyl, 2,2,2-trifluoroethyl, hydroxyethyl, 2,3-dihydroxypropyl, 3,5 hydroxypentyl, ethoxy, propoxy, methylthioethyl, cyclohexyl, cyclohexylmethyl, t-butyl, neopentyl, 1-adamantyl, 3-pentyl, 2-methoxyethylamino, methoxymethylamino, 2-methoxyethyl, hexyl, p-(trifluoromethylbenzyl), pyridylmethyl, 1-methoxy-1-methylamino, 2,2,6,6-tetramethylcyclohexyl, phenylthioethenyl, or allyl; and
R¹⁵ is optionally present and if present, is selected from H, alkyl, alkoxy, or aminoalkyl.

In another embodiment, the present method can be practiced by the administration of a beta-carboline compound that provides an IC₅₀ of less than about 100 μM, in an in vitro assay of MK-2 inhibitory activity. Examples of such compounds comprise the compound having the structure shown in formula I, or a pharmaceutically acceptable salt or prodrug thereof, wherein the compound has the structure according to formula I, wherein:
Q, D, T and Z are selected from carbon, sulfur, or nitrogen;
when Q is sulfur, R¹² is oxo, and R¹¹ is optionally oxo or is absent;
when T is sulfur, R² is oxo, and R⁹ is optionally oxo or is absent;
when Z is nitrogen, D is nitrogen, and R² and R¹² are oxo;
R¹ is selected from H, methyl, ethyl, propenyl, or trifluoromethylbenzyl;
R² is selected from H, methyl, ethyl, methoxycarbonyl-R¹⁵, cyanomethyl, phenylalkyl-R¹⁵, hydroxyl-(C₁-C₄) alkyl, or amino-(C₁-C₄) alkyl;
R³ is selected from H, oxo, methyl, ethyl, carboxyl, propyl, benzyl, hydroxymethylbenzyl, trifluoromethyl, trifluoromethylbenzyl, or R¹ and R³ optionally join to form a C₁-C₃ alkyl bridge between the ring atoms to which R¹ and R³ are attached;
R⁴, R⁶ and R⁷ are independently selected from H, CO₂—R¹⁵, methoxy, or hydroxyl, or R⁶ and R⁷ optionally join to form a pyridyl ring;
R⁵ is selected from H, CO₂—R¹⁵, methyl, hydroxy, methoxy, butyl, butoxy, methylcarboxymethoxy, ethylcarboxymethoxy, 2-oxo-2-phenylethoxy, 2-methoxy-2-oxo-1-phenylethoxy, 2-methoxy-2-oxoethoxy, chloropropoxy, chlorobutoxy, hydrazinocarbonyl, methoxycarbonyl, phenylmethoxy, phenylethoxy, phenylpropoxy, aminocarbonyl, phenylcarbonylmethoxy, chlorobenzyloxy, benzyloxy, methylcarboxyphenylmethoxy, methylcarboxy, fluoro, chloro, iodo, bromo, methylthio, furylmethoxy, N,N-dimethylcarbamylmethoxy, p-(methylsulfonyl)benzyloxy, 2-ethoxy-2-oxoethoxy, carboxymethoxy, pyridyloxomethoxy, pyridylmethoxy, carbazoyl, methylpropoxy, carbamyl, naphthlylmethoxy, or R⁴ and R⁵ optionally join to form a pyran ring;
R⁸, R⁹ and R¹⁰ are independently selected from H or methyl;
R¹¹ is selected from H, or hydroxamate;
R¹² is selected from H, oxo, carbonyl-R¹³, carboxyl-R¹³, ═S, or ═N—OR¹⁵;
R¹³ is selected from H, methyl, ethyl, i-propyl, propyl, benzyl, 1-phenylethyl, 2-fluoroethyl, 2,2,2-trifluoroethyl, hydroxyethyl, 2,3-dihydroxypropyl, 3,5 hydroxypentyl, ethoxy, propoxy, methylthioethyl, cyclohexyl, cyclohexylmethyl, t-butyl, neopentyl, 1-adamantyl, 3-pentyl, 2-methoxyethylamino, methoxymethylamino, 2-methoxyethyl, hexyl, p-(trifluoromethylbenzyl), pyridylmethyl, 1-methoxy-1-methylamino, 2,2,6,6-tetramethylcyclohexyl, phenylthioethenyl, or allyl; and
R¹⁵ is optionally present and if present, is selected from H, alkyl, alkoxy, or aminoalkyl.

The present method can be practiced by the administration of a beta-carboline compound that provides an IC₅₀ of less than about 50 μM, in an in vitro assay of MK-2 inhibitory activity. Examples of such compounds comprise the compound having the structure shown in formula I, or a pharmaceutically acceptable salt or prodrug thereof, wherein the compound has the structure according to formula I, wherein:
T, Q and Z are carbon;
D is selected from carbon or nitrogen;
R¹ is selected from H, methyl or ethyl;
R² is selected from H, methyl, hydroxyl-(C₁-C₄) alkyl, amino-(C₁-C₄) alkyl, or phenylalkyl-R¹⁵,
R³ is selected from H, oxo, methyl, ethyl, propyl, benzyl, trifluoromethyl, or R¹ and R³ optionally join to form a C₁-C₃ alkyl bridge between the ring atoms to which R¹ and R³ are attached;
R⁴, R⁶ and R⁷ are independently selected from H, CO₂—R¹⁵, methoxy, or R⁶ and R⁷ optionally join to form a pyridyl ring;
R⁵ is selected from H, CO₂—R¹⁵, hydroxyl, methoxy, butoxy, 2-oxo-2-phenylethoxy, 2-methoxy-2-oxo-1-phenylethoxy, chloropropoxy, chlorobutoxy, methoxycarbonyl, phenylethoxy, aminocarbonyl, hydrazinocarbonyl, chlorobenzyloxy, benzyloxy, chloro, bromo, iodo, methylthio, N,N-dimethylcarbamylmethoxy, p-(methylsulfonyl)benzyloxy, 2-ethoxy-2-oxoethoxy, 2-methoxy-2-oxoethoxy, carboxymethoxy, pyridylmethoxy, or R⁴ and R⁵ optionally join to form a pyran ring;
R⁸, R⁹ and R¹⁰ are independently selected from H or methyl;
R¹¹ is selected from H, or hydroxamate;
R¹² is selected from H, oxo, carboxyl-R¹³, ═S, or ═N—OR¹⁵;
R¹³ is selected from H, methyl, ethyl, i-propyl, propyl, benzyl, 1-phenylethyl, 2-fluoroethyl, 2,2,2-trifluoroethyl, hydroxyethyl, 2,3-dihydroxypropyl, methylthioethyl, cyclohexyl, cyclohexylmethyl, t-butyl, neopentyl, 2-methoxyethyl, trifluoromethoxybenzyl, hexyl, pyridylmethyl, or allyl; and
R¹⁵ is optionally present and if present, is selected from H, alkyl, alkoxy, or aminoalkyl.

The present method can be practiced by the administration of a beta-carboline compound that provides an IC₅₀ of less than about 20 μM, in an in vitro assay of MK-2 inhibitory activity. Examples of such compounds comprise the compound having the structure shown in formula I, or a pharmaceutically acceptable salt or prodrug thereof, wherein the compound has the structure according to formula I, wherein:
T, Q and Z are carbon;
D is selected from carbon or nitrogen;
R¹ is selected from H, methyl or ethyl;
R² is selected from H, methyl, hydroxymethyl, amino-(C₁-C₄) alkyl, methyl, or phenylalkyl-R¹⁵;
R³ is selected from H, oxo, methyl, ethyl, propyl, benzyl, trifluoromethyl, or R¹ and R³ optionally join to form a C₁-C₃ alkyl bridge between the ring atoms to which R¹ and R³ are attached;
R⁴, R⁶ and R⁷ are independently selected from H, CO₂—R¹⁵ methoxy, or R⁶ and R⁷ optionally join to form a pyridyl ring;
R⁵ is selected from H, CO₂—R¹⁵, hydroxyl, methoxy, butoxy, 2-oxo-2-phenylethoxy, 2-methoxy-2-oxo-1-phenylethoxy, chloropropoxy, chlorobutoxy, methoxycarbonyl, chlorobenzyloxy, benzyloxy, chloro, bromo, iodo, methylthio, 2-ethoxy-2-oxoethoxy, 2-methoxy-2-oxoethoxy, or R⁴ and R⁵ optionally join to form a pyran ring;
R⁸, R⁹ and R¹⁰ are independently selected from H or methyl;
R¹¹ is selected from H, or hydroxamate;
R¹² is selected from H, oxo, ═N—OR¹⁵, or carboxyl-R¹³;
R¹³ is selected from H, methyl, i-propyl, propyl, benzyl, 1-phenylethyl, 2-fluoroethyl, 2,2,2-trifluoroethyl, hydroxyethyl, 2,3-dihydroxypropyl, methylthioethyl, t-butyl, 2-methoxyethyl, trifluoromethoxybenzyl, pyridylmethyl, or allyl;
R¹⁵ is optionally present and if present, is selected from H, alkyl, alkoxy, or aminoalkyl.

The present method can be practiced by the administration of a beta-carboline compound that provides an IC₅₀ of less than about 5 μM, in an in vitro assay of MK-2 inhibitory activity. Examples of such compounds comprise the compound having the structure shown in formula I, or a pharmaceutically acceptable salt or prodrug thereof, wherein the compound has the structure according to formula I, wherein:
T, Q and Z are carbon;
D is selected from carbon or nitrogen;
R¹ is selected from H or methyl;
R² is selected from H, methyl, amino-(C₁-C₄) alkyl, hydroxymethyl, or phenylalkyl-R¹⁵;
R³ is selected from H, oxo, methyl, ethyl, benzyl, or R¹ and R³ optionally join to form a C₁-C₃ alkyl bridge between the ring atoms to which R¹ and R³ are attached;
R⁴, R⁶ and R⁷ are independently selected from H, methoxy, CO₂—R¹⁵, or R⁶ and R⁷ optionally join to form a pyridyl ring;
R⁵ is selected from H, hydroxyl, methoxy, 2-oxo-2-phenylethoxy, methoxycarbonyl, chlorobenzyloxy, benzyloxy, chloro, methylthio, 2-ethoxy-2-oxoethoxy, or R⁴ and R⁵ optionally join to form a pyran ring;
R⁸, R⁹ and R¹⁰ are independently selected from H or methyl;
R¹¹ is selected from H, or hydroxamate;
R¹² is selected from H, oxo, ═N—OR¹⁵, or carboxyl-R¹³;
R¹³ is selected from H, methyl, 2-fluoroethyl, 2,2,2-trifluoroethyl, 2,3-dihydroxypropyl, methylthioethyl, t-butyl, 2-methoxyethyl, pyridylmethyl, or allyl; and
R¹⁵ is optionally present and if present, is selected from H, alkyl, alkoxy, or aminoalkyl.

The present invention also encompasses a method for treating or preventing a disease or disorder in a subject, which disease or disorder is one that can be treated or prevented by inhibiting the activity of MK-2, the method comprising administering to the subject a compound, or a pharmaceutically acceptable salt or prodrug thereof, wherein the compound has the structure according to formula II: 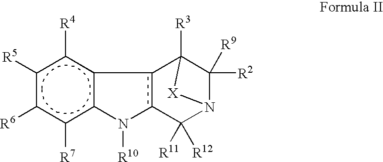
wherein:
X is alkyl;
R² is selected from H, alkyl, alkoxy, hydroxy, carbonyl, oxo, benzyl, hydroxyalkyl, dialkylamino, carboxyl, amino, halo, alkoxycarbonyl, aminoalkyl, haloalkyl, hydroxyalkylbenzyl, haloalkylbenzyl, haloalkoxybenzyl, heterocyclylalkyl, substituted or unsubstituted aryl, which, if substituted, has one or more substituent groups selected from H, alkyl, amino, nitro, halo, haloalkyl, or hydroxyalkyl;
R³ is selected from H, alkyl, alkoxy, hydroxy, carbonyl, benzyl, hydroxyalkyl, dialkylamino, carboxyl, amino, halo, alkoxycarbonyl, aminoalkyl, haloalkyl, hydroxyalkylbenzyl, haloalkylbenzyl, haloalkoxybenzyl, heterocyclylalkyl, substituted or unsubstituted aryl, which, if substituted, has one or more substituent groups selected from H, alkyl, amino, nitro, halo, haloalkyl, or hydroxyalkyl, or R² and R³ optionally join to form a cycloalkyl ring;
R⁴, R⁶ and R⁷ are independently selected from H, alkyl, benzyl, amino, alkylamino, dialkylamino, halo, haloalkyl, alkoxy, haloalkoxy, carboxyl, nitro, benzyloxy, hydrazinocarbonyl, alkoxycarbonyl, alkoxycarbonylheterocyclylcarbonyl, hydroxy, or heterocyclylcarbonyl;
R⁵ is selected from H, alkyl, hydroxy, alkenyl, alkylthio, hydroxy, halo, alkoxy, benzylalkyl, benzylalkoxy, arylalkyl, nitro, alkoxycarbonylalkoxy, alkylthio, benzyloxy, arylalkynyl, arylcarbonylalkoxy, haloalkoxy, arylalkoxy, arylcarbonylalkoxy, haloarylalkoxy, benzyloxoalkoxy, alkoxycarbonylbenzyloxy, alkoxycarbonyl, alkoxyoxoalkoxy, alkoxyoxoarylalkoxy, halobenzyloxy, heterocyclylalkoxy, dialkylaminooxoalkoxy, aminocarbonyl, arylalkynyl, hydrazinocarbonyl, carbonylalkoxy, alkylsulfonylbenzyloxy, carboxyalkoxy, carbamoyl, naphthylalkoxy, alkoxycarbonylheterocyclylalkoxy, haloalkylbenzyloxy, alkoxyacetylaminoacetyloxy, acetoxy, cycloalkylalkoxy, acetyloxy, morpholinylcarbonyl, alkoxyalkoxy, or R⁴ and R⁵ optionally join to form a heterocyclic ring;
R⁹ is selected from H, oxo, carbonitrile, or alkyl;
R¹⁰ is selected from H, alkyl, benzyl, aryl, or hydroxyalkyl;
R¹¹ is selected from H, oxo, alkyl, halobenzyl, benzyl, haloalkyl, 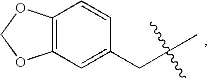
alkoxycarbonyl, carboxyalkyl, benzodioxinylalkyl, substituted or unsubstituted aryl or arylalkyl, which, if substituted, have one or more substituent groups selected from H, alkyl, or alkoxy;
R¹² is selected from H, oxo, alkyl, carbonyl-R¹³, carboxyl-R¹³, ketone, cyano, cyanoalkyl, alkoxycarbonyl, aminoalkyl, alkoxycarbonylalkyl, carbamyl, hydroxycarbamyl, haloalkyl, heterocyclyl, alkylacetate, heterocyclylalkyl, alkoxycarbonylalkyl, hydroxyalkyl, dihydroxyalkyl, cyanoalkyl, or hydroxyamino, substituted or unsubstituted 5 or 6-membered heterocyclic ring, or R¹¹ and R¹² optionally join to form a ring system having the structure: 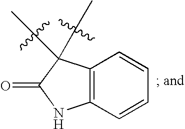
R¹³ is optionally present and if present, is selected from H, alkyl, alkoxy, amino, alkynyl, allyl, alkenyl, hydrazine, benzyl, glycine, allylalkyl, haloalkyl, hydroxyalkyl, hydroxyamino, dihydroxyalkyl, alkoxyalkyl, alkoxyamino, alkylthioalkyl, cycloalkyl, cyclohexylalkyl, amino, alkylamino, benzylalkyl, benzylamino, benzyloxycarbonylalkylamino, dialkylamino, adamantyl, haloalkoxybenzyl, alkoxyaminoalkyl, haloalkylarylalkyl, heterocyclylalkyl, alkoxyalkylamino, arylthioalkyl, substituted or unsubstituted aryl or arylalkyl, which, if substituted, have one or more substituent groups selected from H, alkyl, alkoxy, amino, nitro, halo, haloalkyl, or hydroxyalkyl.

The present invention encompasses a method for treating or preventing a disease or disorder in a subject, which disease or disorder is one that can be treated or prevented by inhibiting the activity of MK-2, the method comprising administering to the subject a compound, or a pharmaceutically acceptable salt or prodrug thereof, wherein the compound has the structure according to formula II, wherein:
X is C₁-C₃ alkyl;
R² is selected from H, (C₁-C₆) alkyl, carbonyl, (C₁-C₄) alkoxycarbonyl, amino-(C₁-C₄) alkyl, (C₁-C₆) alkoxy, amino-(C₁-C₄) alkyl, hydroxy, benzyl, hydroxyalkyl, dialkylamino, amino, halo, halo-(C₁-C₄) alkyl, carboxy, oxo, hydroxy-(C₁-C₄) alkylbenzyl, halo-(C₁-C₄) alkylbenzyl, halo-(C₁-C₄) alkoxybenzyl, furyl-(C₁-C₄) alkyl, substituted or unsubstituted aryl, which, if substituted, has one or more substituent groups selected from H, (C₁-C₄) alkyl, amino, nitro, halo, halo-(C₁-C₄) alkyl, or hydroxy-(C₁-C₄) alkyl;
R³ is selected from H, (C₁-C₆) alkyl, carbonyl, (C₁-C₄) alkoxycarbonyl, amino-(C₁-C₄) alkyl, (C₁-C₆) alkoxy, amino-(C₁-C₄) alkyl, hydroxy, benzyl, hydroxyalkyl, dialkylamino, amino, halo, halo-(C₁-C₄) alkyl, carboxy, hydroxy-(C₁-C₄) alkylbenzyl, halo-(C₁-C₄) alkylbenzyl, halo-(C₁-C₄) alkoxybenzyl, furyl-(C₁-C₄) alkyl, substituted or unsubstituted aryl, which, if substituted, has one or more substituent groups selected from H, (C₁-C₄) alkyl, amino, nitro, halo, halo-(C₁-C₄) alkyl, or hydroxy-(C₁-C₄) alkyl, or R² and R³ optionally join to form a cyclohexyl ring;
R⁴, R⁶ and R⁷ are independently selected from H, (C₁-C₆) alkyl, amino, (C₁-C₄) alkylamino, dialkylamino, benzyl, halo, halo-(C₁-C₄) alkyl, (C₁-C₆) alkoxy, halo-(C₁-C₄) alkoxy, carboxyl, nitro, benzyloxy, hydrazinocarbonyl, (C₁-C₄) alkoxycarbonyl, (C₁-C₄) alkoxycarbonylpiperazinylcarbonyl, hydroxy, morpholinylcarbonyl, or piperazinylcarbonyl;
R⁵ is selected from H, (C₁-C₆) alkyl, (C₁-C₄) alkenyl, (C₁-C₄) alkylthio, hydroxy, halo, (C₁-C₆) alkoxy, benzyl-(C₁-C₄) alkyl, benzyl-(C₁-C₄) alkoxy, aryl-(C₁-C₄) alkyl, benzyloxo-(C₁-C₄) alkoxy, nitro, (C₁-C₄) alkoxycarbonylalkoxy, (C₁-C₄) alkylthio, benzyloxy, phenyl-(C₁-C₄) alkynyl, phenylcarbonyl-(C₁-C₄) alkoxy, halo-(C₁-C₄) alkoxy, aryl-(C₁-C₄) alkoxy, arylcarbonyl-(C₁-C₄) alkoxy, haloaryl-(C₁-C₄) alkoxy, (C₁-C₄) alkoxycarbonylbenzyloxy, (C₁-C₄) alkoxycarbonyl, (C₁-C₄) alkoxyoxo-(C₁-C₄) alkoxy, (C₁-C₄) alkoxyoxophenyl-(C₁-C₄) alkoxy, halobenzyloxy, pyridinyl-(C₁-C₄) alkoxy, dialkylaminooxo-(C₁-C₄) alkoxy, aminocarbonyl, phenyl-(C₁-C₄) alkynyl, hydrazinocarbonyl, carbonyl-(C₁-C₄) alkoxy, (C₁-C₄) alkylsulfonylbenzyloxy, carboxy-(C₁-C₄) alkoxy, carbamoyl, naphthyl-(C₁-C₄) alkoxy, (C₁-C₄) alkoxycarbonylfuryl-(C₁-C₄) alkoxy, halo-(C₁-C₄) alkylbenzyloxy, (C₁-C₄) alkoxyacetylaminoacetyloxy, acetoxy, cyclohexyl-(C₁-C₄) alkoxy, acetyloxy, morpholinylcarbonyl, (C₁-C₄) alkoxy-(C₁-C₄) alkoxy, or R⁴ and R⁵ optionally join to form a pyranyl or 1,4 dioxinyl ring;
R⁹ is selected from H, oxo, carbonitrile, or (C₁-C₆) alkyl;
R¹⁰ is selected from H, (C₁-C₆) alkyl, benzyl, phenyl, or hydroxy-(C₁-C₄) alkyl;
R¹¹ is selected from H, oxo, (C₁-C₆) alkyl, halobenzyl, benzyl, halo-(C₁-C₄) alkyl, C₁-C₄) alkoxycarbonyl, carboxy-(C₁-C₄) alkyl, 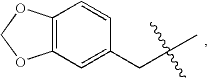
benzodioxinyl-(C₁-C₄) alkyl, substituted or unsubstituted aryl or aryl-(C₁-C₆) alkyl, which, if substituted, have one or more substituent groups selected from H, (C₁-C₆) alkyl, (C₁-C₆) alkoxy;
R¹² is selected from H, oxo, (C₁-C₆) alkyl, carbonyl-R¹³, carboxyl-R¹³, cyano, ketone, cyano-(C₁-C₄) alkyl, (C₁-C₄) alkoxycarbonyl, amino-(C₁-C₄) alkyl, (C₁-C₄) alkoxycarbonyl-(C₁-C₄) alkyl, carbamyl, hydroxycarbamyl, halo-(C₁-C₄) alkyl, tetrazolyl, tetrazolyl-(C₁-C₄) alkyl, (C₁-C₄) alkylacetate, t-butoxycarbonyl-(C₁-C₄) alkyl, hydroxy-(C₁-C₄) alkyl, dihydroxy-(C₁-C₄) alkyl, isoxazolyl, furanyl, pyrrolyl, pyrazolyl, imidazolyl, acetonitrile, or hydroxyamino, or R¹¹ and R¹² optionally join to form a ring system selected from: 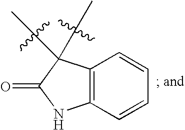
R¹³ is optionally present and if present, is selected from H, (C₁-C₆) alkyl, (C₁-C₆) alkoxy, amino, (C₁-C₆) allyl, (C₁-C₆) alkynyl, (C₁-C₆) alkenyl, hydrazine, benzyl, glycine, allyl-(C₁-C₄) alkyl, halo-(C₁-C₄) alkyl, hydroxy-(C₁-C₄) alkyl, hydroxyamino, dihydroxy-(C₁-C₄) alkyl, (C₁-C₄) alkoxy-(C₁-C₄) alkyl, (C₁-C₄) alkoxyamino, (C₁-C₄) alkylthio-(C₁-C₄) alkyl, cyclohexyl, cyclohexyl-(C₁-C₄) alkyl, amino, (C₁-C₄) alkylamino, benzyl-(C₁-C₄) alkyl, benzylamino, benzyloxycarbonyl-(C₁-C₄) alkylamino, dialkylamino, adamantyl halo-(C₁-C₄) alkoxybenzyl, (C₁-C₄) alkoxyamino-(C₁-C₄) alkyl, halo-(C₁-C₄) alkylaryl-(C₁-C₄) alkyl, pyridinyl-(C₁-C₄) alkyl, (C₁-C₄) alkoxy-(C₁-C₄) alkylamino, phenylthio-(C₁-C₄) alkyl, substituted or unsubstituted aryl or aryl-(C₁-C₄) alkyl, which, if substituted, have one or more substituent groups selected from H, (C₁-C₆) alkyl, (C₁-C₆) alkoxy, amino, nitro, halo, halo-(C₁-C₄) alkyl, or hydroxy-(C₁-C₄) alkyl.

The present invention encompasses a method for treating or preventing a disease or disorder in a subject, which disease or disorder is one that can be treated or prevented by inhibiting the activity of MK-2, the method comprising administering to the subject a compound, or a pharmaceutically acceptable salt or prodrug thereof, wherein the compound has the structure according to formula II, wherein:
X is C₁-C₃ alkyl;
R² is selected from H, (C₁-C₆) alkyl, carbonyl, (C₁-C₄) alkoxycarbonyl, amino-(C₁-C₄) alkyl, (C₁-C₆) alkoxy, amino-(C₁-C₄) alkyl, hydroxy, benzyl, hydroxyalkyl, dialkylamino, amino, halo, halo-(C₁-C₄) alkyl, carboxy, oxo, hydroxy-(C₁-C₄) alkylbenzyl, halo-(C₁-C₄) alkylbenzyl, halo-(C₁-C₄) alkoxybenzyl, furyl-(C₁-C₄) alkyl, substituted or unsubstituted aryl, which, if substituted, has one or more substituent groups selected from H, (C₁-C₄) alkyl, amino, nitro, halo, halo-(C₁-C₄) alkyl, or hydroxy-(C₁-C₄) alkyl;
R³ is selected from H, (C₁-C₆) alkyl, carbonyl, (C₁-C₄) alkoxycarbonyl, amino-(C₁-C₄) alkyl, (C₁-C₆) alkoxy, amino-(C₁-C₄) alkyl, hydroxy, benzyl, hydroxyalkyl, dialkylamino, amino, halo, halo-(C₁-C₄) alkyl, carboxy, hydroxy-(C₁-C₄) alkylbenzyl, halo-(C₁-C₄) alkylbenzyl, halo-(C₁-C₄) alkoxybenzyl, furyl-(C₁-C₄) alkyl, substituted or unsubstituted aryl, which, if substituted, has one or more substituent groups selected from H, (C₁-C₄) alkyl, amino, nitro, halo, halo-(C₁-C₄) alkyl, or hydroxy-(C₁-C₄) alkyl, or R² and R³ optionally join to form a cyclohexyl ring;
R⁴, R⁶ and R⁷ are independently selected from H, (C₁-C₆) alkyl, amino, (C₁-C₄) alkylamino, dialkylamino, benzyl, halo, halo-(C₁-C₄) alkyl, (C₁-C₆) alkoxy, halo-(C₁-C₄) alkoxy, carboxyl, nitro, benzyloxy, hydrazinocarbonyl, (C₁-C₄) alkoxycarbonyl, (C₁-C₄) alkoxycarbonylpiperazinylcarbonyl, hydroxy, morpholinylcarbonyl, or piperazinylcarbonyl;
R⁵ is selected from H, (C₁-C₆) alkyl, (C₁-C₄) alkenyl, (C₁-C₄) alkylthio, hydroxy, halo, (C₁-C₆) alkoxy, benzyl-(C₁-C₄) alkyl, benzyl-(C₁-C₄) alkoxy, aryl-(C₁-C₄) alkyl, benzyloxo-(C₁-C₄) alkoxy, nitro, (C₁-C₄) alkoxycarbonylalkoxy, (C₁-C₄) alkylthio, benzyloxy, phenyl-(C₁-C₄) alkynyl, phenylcarbonyl-(C₁-C₄) alkoxy, halo-(C₁-C₄) alkoxy, aryl-(C₁-C₄) alkoxy, arylcarbonyl-(C₁-C₄) alkoxy, haloaryl-(C₁-C₄) alkoxy, (C₁-C₄) alkoxycarbonylbenzyloxy, (C₁-C₄) alkoxycarbonyl, (C₁-C₄) alkoxyoxo-(C₁-C₄) alkoxy, -(C₁-C₄) alkoxyoxophenyl-(C₁-C₄) alkoxy, halobenzyloxy, pyridinyl-(C₁-C₄) alkoxy, dialkylaminooxo-(C₁-C₄) alkoxy, aminocarbonyl, phenyl-(C₁-C₄) alkynyl, hydrazinocarbonyl, carbonyl-(C₁-C₄) alkoxy, (C₁-C₄) alkylsulfonylbenzyloxy, carboxy-(C₁-C₄) alkoxy, carbamoyl, naphthyl-(C₁-C₄) alkoxy, (C₁-C₄) alkoxycarbonylfuryl-(C₁-C₄) alkoxy, halo-(C₁-C₄) alkylbenzyloxy, (C₁-C₄) alkoxyacetylaminoacetyloxy, acetoxy, cyclohexyl-(C₁-C₄) alkoxy, acetyloxy, morpholinylcarbonyl, (C₁-C₄) alkoxy-(C₁-C₄) alkoxy, or R⁴ and R⁵ optionally join to form a pyranyl or 1,4 dioxinyl ring;
R⁹ is selected from H, oxo, carbonitrile, or (C₁-C₆) alkyl;
R¹⁰ is selected from H, (C₁-C₆) alkyl, benzyl, phenyl, or hydroxy-(C₁-C₄) alkyl;
R¹¹ is selected from H, oxo, (C₁-C₆) alkyl, halobenzyl, benzyl, halo-(C₁-C₄) alkyl, C₁-C₄) alkoxycarbonyl, carboxy-(C₁-C₄) alkyl, 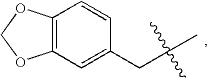
benzodioxinyl-(C₁-C₄) alkyl, substituted or unsubstituted aryl or aryl-(C₁-C₆) alkyl, which, if substituted, have one or more substituent groups selected from H, (C₁-C₆) alkyl, (C₁-C₆) alkoxy;
R¹² is selected from H, oxo, (C₁-C₆) alkyl, carbonyl-R¹³, carboxyl-R¹³, cyano, ketone, cyano-(C₁-C₄) alkyl, (C₁-C₄) alkoxycarbonyl, amino-(C₁-C₄) alkyl, (C₁-C₄) alkoxycarbonyl-(C₁-C₄) alkyl, carbamyl, hydroxycarbamyl, halo-(C₁-C₄) alkyl, tetrazolyl, tetrazolyl-(C₁-C₄) alkyl, (C₁-C₄) alkylacetate, t-butoxycarbonyl-(C₁-C₄) alkyl, hydroxy-(C₁-C₄) alkyl, dihydroxy-(C₁-C₄) alkyl, isoxazolyl, furanyl, pyrrolyl, pyrazolyl, imidazolyl, acetonitrile, or hydroxyamino, or R¹¹ and R¹² optionally join to form a ring system selected from: 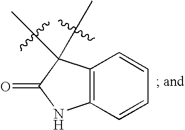
R¹³ is optionally present and if present, is selected from H, (C₁-C₆) alkyl, (C₁-C₆) alkoxy, amino, (C₁-C₆) allyl, (C₁-C₆) alkynyl, (C₁-C₆) alkenyl, hydrazine, benzyl, glycine, allyl-(C₁-C₄) alkyl, halo-(C₁-C₄) alkyl, hydroxy-(C₁-C₄) alkyl, hydroxyamino, dihydroxy-(C₁-C₄) alkyl, (C₁-C₄) alkoxy-(C₁-C₄) alkyl, (C₁-C₄) alkoxyamino, (C₁-C₄) alkylthio-(C₁-C₄) alkyl, cyclohexyl, cyclohexyl-(C₁-C₄) alkyl, amino, (C₁-C₄) alkylamino, benzyl-(C₁-C₄) alkyl, benzylamino, benzyloxycarbonyl-(C₁-C₄) alkylamino, dialkylamino, adamantyl halo-(C₁-C₄) alkoxybenzyl, (C₁-C₄) alkoxyamino-(C₁-C₄) alkyl, halo-(C₁-C₄) alkylaryl-(C₁-C₄) alkyl, pyridinyl-(C₁-C₄) alkyl, (C₁-C₄) alkoxy-(C₁-C₄) alkylamino, phenylthio-(C₁-C₄) alkyl, substituted or unsubstituted aryl or aryl-(C₁-C₄) alkyl, which, if substituted, have one or more substituent groups selected from H, (C₁-C₆) alkyl, (C₁-C₆) alkoxy, amino, nitro, halo, halo-(C₁-C₄) alkyl, or hydroxy-(C₁-C₄) alkyl.

The present invention encompasses a method for treating or preventing a disease or disorder in a subject, which disease or disorder is one that can be treated or prevented by inhibiting the activity of MK-2, the method comprising administering to the subject a compound, or a pharmaceutically acceptable salt or prodrug thereof, wherein the compound has the structure according to formula II, wherein:
X is C₁-C₃ alkyl;
R² is selected from H, methyl, ethyl, methoxy, propyl, n-propyl, aminomethyl, hydroxymethyl, dimethylaminomethyl, carboxy, dimethylamino, trifluoromethyl, methoxycarbonyl, oxo, carbonyl, alkoxycarbonyl, hydroxy, benzyl, amino, halo, trifluoromethyl, p-hydroxymethylbenzyl, m-trifluoromethylbenzyl, o-trifluoromethylbenzyl, trifluoromethoxybenzyl, phenyl, furylmethyl, or R² and R³ optionally join to form a ring system consisting of: 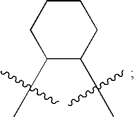
R³ is selected from H, methyl, ethyl, methoxy, propyl, n-propyl, aminomethyl, hydroxymethyl, dimethylaminomethyl, carboxy, dimethylamino, benzyl, trifluoromethyl, methoxycarbonyl, carbonyl, alkoxycarbonyl, hydroxy, amino, halo, trifluoromethyl, p-hydroxymethylbenzyl, m-trifluoromethylbenzyl, o-trifluoromethylbenzyl, trifluoromethoxybenzyl, phenyl, furylmethyl, or R² and R³ optionally join to form a ring system consisting of: 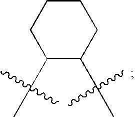
R⁴, R⁶ and R⁷ are independently selected from H, methyl, benzyl, methoxy, hydroxyl, methoxycarbonyl, benzyloxy, hydrazinocarbonyl, carbonxyl, fluoro, chloro, morpholinylcarbonyl, nitro, bromo, t-butoxycarbonylpiperazinylcarbonyl, or piperazinylcarbonyl;
R⁵ is selected from H, methyl, methoxy, acetoxy, carbamoyl, 2-oxo-2-phenylethoxy, hydroxyl, benzyloxy, benzyloxoethoxy, methoxycarbonyl, 2-ethoxy-2-oxoethoxy, 2-methoxy-2-oxo-1-phenylethoxy, butoxy, n-butoxy, 2-methoxy-2-oxoethoxy, p-chlorobenzyloxy, o-chlorobenzyloxy, m-chlorobenzyloxy, chloropropoxy, chlorobutoxy, chloro, 3-pyridylmethoxy, 4-pyridylmethoxy, N,N-dimethylcarbamylmethoxy, 2-phenylethoxy, aminocarbonyl, bromo, fluoro, carboxymethoxy, p-(methylsulfonyl)benzyloxy, hydrazinocarbonyl, 2-naphthylmethoxy, 4-ethoxy-4-oxobutoxy, (5-ethoxycarbonyl-2-furyl)methoxy, 2-methoxyethoxy, p-(trifluoromethyl)benzyloxy, 3-phenylpropoxy, m-chlorobenzylmethoxy, 3-methoxycarbonylbenzyloxy, 2-pyridylmethoxy, cyclohexylmethoxy, ethoxycarbonylmethylaminocarbonylmethoxy, iodo, phenylethynyl, 2-phenylethyl, nitro, ethoxy, or methylthio, or R⁴ and R⁵ optionally join to form a pyranyl or 1,4 dioxinyl ring;
R⁹ is selected from H, oxo, carbonitrile, or methyl
R¹⁰ is selected from H, benzyl, phenyl, methyl, ethyl, or hydroxymethyl;
R¹¹ is selected from H, oxo, methyl, benzyl, phenyl, chloromethyl, p-chlorobenzyl, methoxycarbonyl, ethoxycarbonyl, carboxyethyl, 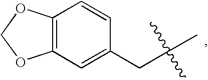
carboxypropyl, trimethoxybenzyl, or;
R¹² is selected from H, oxo, methyl, ethyl, carbonyl-R¹³, carboxyl-R¹³, ketone, cyano, cyanomethyl, tetrazoyl, pyrrolyl, tetrazoylmethyl, methylacetate, t-butylacetate, hydroxymethyl, trifluoromethyl, aminomethyl, acetonitrile, ethane-1,2-diol, methoxycarbonyl, methoxycarbonylmethyl, carbamyl, hydroxycarbamyl, isoxazolyl, furanyl, pyrazolyl, imidazolyl, or hydroxyamino, or R¹¹ and R¹² optionally join to form a ring system having the structure: 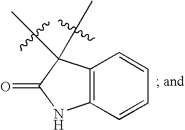
R¹³ is optionally present and if present, is selected from H, methyl, ethyl, i-propyl, n-propyl, propyl, benzyl, hydroxyamino, hydrazino, glycine, propene, benzyloxycarbonylmethylamino, 1-phenylethyl, 2,2,2-trifluoroethyl, 2-fluoroethyl, 2-hydroxyethyl, 2,3-dihydroxypropyl, 2-methoxyethyl, 2-(methylthio)ethyl, cyclohexyl, cyclohexylmethyl, t-butyl, neopentyl, 1-adamantyl, pentyl, amino, N,N-dimethylamino, benzylamino, 2-methoxyethylamino, n-hexyl, p-(trifluoromethylbenzyl), trifluoromethoxybenzyl, methylamino, pyridylmethyl, (N-methoxy-N-methyl)amino, 2,2,6,6-tetramethylcyclohexyl, or 2-(phenylthio)ethyl.

The present invention encompasses a method for treating or preventing a disease or disorder in a subject, which disease or disorder is one that can be treated or prevented by inhibiting the activity of MK-2, the method comprising administering to the subject a compound, or a pharmaceutically acceptable salt or prodrug thereof, wherein the compound has the structure according to formula II, wherein:
X is C₁-C₃ alkyl;
R² is selected from H, methyl, ethyl, methoxy, propyl, n-propyl, aminomethyl, hydroxymethyl, dimethylaminomethyl, carboxy, dimethylamino, trifluoromethyl, methoxycarbonyl, oxo, carbonyl, alkoxycarbonyl, hydroxy, benzyl, amino, halo, trifluoromethyl, p-hydroxymethylbenzyl, m-trifluoromethylbenzyl, o-trifluoromethylbenzyl, trifluoromethoxybenzyl, phenyl, furylmethyl, or R² and R³ optionally join to form a ring system consisting of: 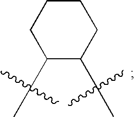
R³ is selected from H, methyl, ethyl, methoxy, propyl, n-propyl, aminomethyl, hydroxymethyl, dimethylaminomethyl, carboxy, dimethylamino, benzyl, trifluoromethyl, methoxycarbonyl, carbonyl, alkoxycarbonyl, hydroxy, amino, halo, trifluoromethyl, p-hydroxymethylbenzyl, m-trifluoromethylbenzyl, o-trifluoromethylbenzyl, trifluoromethoxybenzyl, phenyl, furylmethyl, or R² and R³ optionally join to form a ring system consisting of: 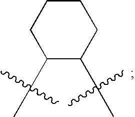
R⁴, R⁶ and R⁷ are independently selected from H, methyl, benzyl, methoxy, hydroxyl, methoxycarbonyl, benzyloxy, hydrazinocarbonyl, carbonxyl, fluoro, chloro, morpholinylcarbonyl, nitro, bromo, t-butoxycarbonylpiperazinylcarbonyl, or piperazinylcarbonyl;
R⁵ is selected from H, methyl, methoxy, acetoxy, carbamoyl, 2-oxo-2-phenylethoxy, hydroxyl, benzyloxy, benzyloxoethoxy, methoxycarbonyl, 2-ethoxy-2-oxoethoxy, 2-methoxy-2-oxo-1-phenylethoxy, butoxy, n-butoxy, 2-methoxy-2-oxoethoxy, p-chlorobenzyloxy, o-chlorobenzyloxy, m-chlorobenzyloxy, chloropropoxy, chlorobutoxy, chloro, 3-pyridylmethoxy, 4-pyridylmethoxy, N,N-dimethylcarbamylmethoxy, 2-phenylethoxy, aminocarbonyl, bromo, fluoro, carboxymethoxy, p-(methylsulfonyl)benzyloxy, hydrazinocarbonyl, 2-naphthylmethoxy, 4-ethoxy-4-oxobutoxy, (5-ethoxycarbonyl-2-furyl)methoxy, 2-methoxyethoxy, p-(trifluoromethyl)benzyloxy, 3-phenylpropoxy, m-chlorobenzylmethoxy, 3-methoxycarbonylbenzyloxy, 2-pyridylmethoxy, cyclohexylmethoxy, ethoxycarbonylmethylaminocarbonylmethoxy, iodo, phenylethynyl, 2-phenylethyl, nitro, ethoxy, or methylthio, or R⁴ and R⁵ optionally join to form a pyranyl or 1,4 dioxinyl ring;
R⁹ and R¹⁰ are H;
R¹¹ is selected from H, oxo, methyl, benzyl, phenyl, chloromethyl, p-chlorobenzyl, methoxycarbonyl, ethoxycarbonyl, carboxyethyl, 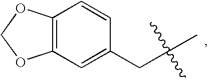
carboxypropyl, trimethoxybenzyl, or;
R¹² is selected from H, oxo, methyl, ethyl, carbonyl-R¹³, carboxyl-R¹³, ketone, cyano, cyanomethyl, tetrazoyl, pyrrolyl, tetrazoylmethyl, methylacetate, t-butylacetate, hydroxymethyl, trifluoromethyl, aminomethyl, acetonitrile, ethane-1,2-diol, methoxycarbonyl, methoxycarbonylmethyl, carbamyl, hydroxycarbamyl, isoxazolyl, furanyl, pyrazolyl, imidazolyl, or hydroxyamino, or R¹¹ and R¹² optionally join to form a ring system having the structure: 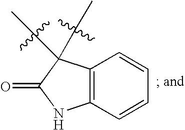
R¹³ is optionally present and if present, is selected from H, methyl, ethyl, i-propyl, n-propyl, propyl, benzyl, hydroxyamino, hydrazino, glycine, propene, benzyloxycarbonylmethylamino, 1-phenylethyl, 2,2,2-trifluoroethyl, 2-fluoroethyl, 2-hydroxyethyl, 2,3-dihydroxypropyl, 2-methoxyethyl, 2-(methylthio)ethyl, cyclohexyl, cyclohexylmethyl, t-butyl, neopentyl, 1-adamantyl, pentyl, amino, N,N-dimethylamino, benzylamino, 2-methoxyethylamino, n-hexyl, p-(trifluoromethylbenzyl), trifluoromethoxybenzyl, methylamino, pyridylmethyl, (N-methoxy-N-methyl)amino, 2,2,6,6-tetramethylcyclohexyl, or 2-(phenylthio)ethyl.

The present invention encompasses a method for treating or preventing a disease or disorder in a subject, which disease or disorder is one that can be treated or prevented by inhibiting the activity of MK-2, the method comprising administering to the subject a compound, or a pharmaceutically acceptable salt or prodrug thereof, wherein the compound has the structure according to formula II, wherein:
X is C₁-C₃ alkyl;
R², R³, R⁴, R⁶, R⁷, R⁹, R¹⁰ and R¹¹ are H;
R⁵ is selected from H, methoxy, hydroxy, benzyloxy, methylthio, or R⁴ and R⁵ optionally join to form a pyran ring;
R¹² is selected from carbonyl-R¹³, carboxyl-R¹³; and
R¹³ is optionally present and if present, is selected from H, methyl, ethyl, propyl, i-propyl, n-propyl, propenyl, trifluoromethyl, 4-(trifluoromethoxy)benzyl, trifluoroethyl, fluoroethyl, cyclohexylmethyl, 1-phenylethyl, cyclohexyl, 2-hydroxyethyl, benzyl, 2,3-dihydroxypropyl, 2-methoxyethyl, methylamino, benzylamino, 2-(methylthio)ethyl, hexyl, c-hexyl, n-hexyl, t-butyl, neopentyl, 1-adamantyl, 3-pentyl, amino, dimethylamino, benzylamino, methoxymethylamino, methoxyethylamino, pyridylmethyl, or 2,2,6,6-tetramethylcyclohexyl.

The present invention also encompasses a method for treating or preventing a disease or disorder in a subject, which disease or disorder is one that can be treated or prevented by inhibiting the activity of MK-2, the method comprising administering to the subject a compound, or a pharmaceutically acceptable salt or prodrug thereof, wherein the compound has the structure according to formula III: 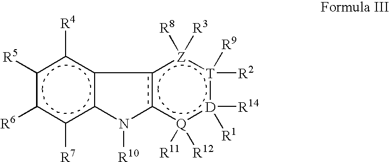
wherein:
Q, D, T and Z are selected from carbon, sulfur, or nitrogen;
when Q is sulfur, R¹² is oxo, and R¹¹ is optionally oxo or is absent;
when D is nitrogen, R¹² is selected from oxo, ═S, or ═N—OR¹⁵, and R¹¹ is optionally oxo or is absent;
when T is sulfur, R² is oxo, and R⁹ is optionally oxo or is absent;
when Z is nitrogen, D is nitrogen, and R² and R¹² are oxo;
at least one of R¹, R², R³, R¹¹ and R¹² is oxo, ═S, ═N—OR¹⁵, or ═N—N(R¹⁵)₂;
R¹, R² and R³ are independently selected from H, alkoxy, oxo, arylalkenylamino, alkoxycarbonyl-R¹⁵, arylalkyl-R¹⁵, alkyl-SO₂—R¹⁵, amino, or substituted or unsubstituted alkyl, which, if substituted, has one or more substituent groups selected from H, halo, amino, cyano, hydroxyl, aryl, or heteroaryl;
R⁴, R⁶ and R⁷ are independently selected from H, alkyl, alkoxy, halo, carboxyl, nitro, CO₂—R⁵, substituted or unsubstituted 6-membered heteroarylcarbonyl, which, if substituted, have one or more substituent groups selected from H or alkoxycarbonyl, or R⁶ and R⁷ optionally join to form a heteroaryl ring;
R⁵ is selected from H, alkoxy, hydroxyl, halo, benzylalkyl, arylalkynyl, CO₂—R¹⁵, nitro, haloalkyl, alkoxycarbonyl or carboxyl, or R⁴ and R⁵ optionally join to form a heteroaryl ring;
R⁹ is independently selected from H and oxo or is optionally absent;
R¹⁰ is selected from H, alkyl, or aryl;
R¹¹ and R¹² are optionally present and if present, are independently selected from H, oxo, ═S, ═N—OR¹⁵, or ═N—N(R¹⁵)₂;
R¹⁴ is optionally present and if present, is selected from H or alkyl; and
R¹⁵ is optionally present and if present, is selected from H, alkyl, alkoxy, or aminoalkyl.

The present invention also encompasses a method for treating or preventing a disease or disorder in a subject, which disease or disorder is one that can be treated or prevented by inhibiting the activity of MK-2, the method comprising administering to the subject a compound, or a pharmaceutically acceptable salt or prodrug thereof, wherein the compound has the structure according to formula III, wherein:
Q, D, T and Z are selected from carbon, sulfur, or nitrogen;
when Q is sulfur, R¹² is oxo, and R¹¹ is optionally oxo or is absent;
when D is nitrogen, R¹² is selected from oxo, ═S, or ═N—OR¹⁵, and R¹¹ is optionally oxo or is absent;
when T is sulfur, R² is oxo, and R⁹ is optionally oxo or is absent;
when Z is nitrogen, D is nitrogen, and R² and R¹² are oxo;
at least one of R¹, R², R³, R¹¹ and R¹² is oxo or oxime;
R¹, R² and R³ are independently selected from H, alkoxy, oxo, arylalkenylamino, alkoxycarbonyl-R¹⁵, arylalkyl-R¹⁵, alkyl-SO₂—R¹⁵, amino, or substituted or unsubstituted alkyl, which, if substituted, has one or more substituent groups selected from H, halo, amino, cyano, hydroxyl, aryl, or heteroaryl;
R⁴, R⁶ and R⁷ are independently selected from H, alkyl, alkoxy, halo, carboxyl, nitro, CO₂—R¹⁵, substituted or unsubstituted 6-membered heteroarylcarbonyl, which, if substituted, have one or more substituent groups selected from H or alkoxycarbonyl, or R⁶ and R⁷ optionally join to form a heteroaryl ring;
R⁵ is selected from H, alkoxy, hydroxyl, halo, benzylalkyl, arylalkynyl, CO₂—R¹⁵, nitro, haloalkyl, alkoxycarbonyl or carboxyl, or R⁴ and R⁵ optionally join to form a heteroaryl ring;
R⁹ is independently selected from H and oxo or is optionally absent;
R¹⁰ is selected from H, alkyl, or aryl;
R¹¹ and R¹² are optionally present and if present, are independently selected from H, oxo, or oxime;
R¹⁴ is optionally present and if present, is selected from H or alkyl; and
R¹⁵ is optionally present and if present, is selected from H, alkyl, alkoxy, or aminoalkyl.

The present invention also encompasses a method for treating or preventing a disease or disorder in a subject, which disease or disorder is one that can be treated or prevented by inhibiting the activity of MK-2, the method comprising administering to the subject a compound, or a pharmaceutically acceptable salt or prodrug thereof, wherein the compound has the structure according to formula II, wherein:
Q, D, T and Z are selected from carbon, sulfur, or nitrogen;
when Q is sulfur, R¹² is oxo, and R¹¹ is optionally oxo or is absent;
when D is nitrogen, R¹² is selected from oxo, ═S, or ═N—OR¹⁵, and R¹¹ is optionally oxo or is absent;
when T is sulfur, R² is oxo, and R⁹ is optionally oxo or is absent;
when Z is nitrogen, D is nitrogen, and R² and R¹² are oxo;
at least one of R¹, R², R³, R¹¹ and R¹² is oxo or oxime;
R¹, R² and R³ are independently selected from H, (C₁-C₆) alkoxy, oxo, phenyl-(C₁-C₄) alkenylamino, amino, (C₁-C₄) alkoxycarbonyl-R¹⁵, cyano-(C₁-C₄) alkyl, aryl-(C₁-C₄) alkyl-R¹⁵, alkyl-SO₂—R¹⁵, or substituted or unsubstituted (C₁-C₆) alkyl, which, if substituted, has one or more substituent groups selected from H, halo, cyano, hydroxyl, amino, aryl, or heteroaryl;
R⁴, R⁶ and R⁷ are independently selected from H, (C₁-C₆) alkyl, (C₁-C₆) alkoxy, CO₂—R¹⁵, halo, carboxyl, nitro, or substituted or unsubstituted 6-membered heterocycliccarbonyl, which, if substituted, have one or more substituent groups selected from H or (C₁-C₄) alkoxycarbonyl, or R⁶ and R⁷ optionally join to form a heteroaryl ring;
R⁵ is selected from H, (C₁-C₆) alkoxy, hydroxyl, halo, benzyl-(C₁-C₄) alkyl, phenyl-(C₁-C₄) alkynyl, CO₂—R¹⁵, nitro, halo-(C₁-C₄) alkyl, (C₁-C₄) alkoxycarbonyl or carboxyl, or R⁴ and R⁵ optionally join to form a 6-membered heterocyclic ring;
R⁹ is independently selected from H and oxo or is optionally absent;
R¹⁰ is selected from H, (C₁-C₆) alkyl, or phenyl;
R¹¹ and R¹² are optionally present and if present, are independently selected from H, oxo, and oxime;
R¹⁴ is optionally present and if present, is selected from H or alkyl; and
R¹⁵ is optionally present and if present, is selected from H, (C₁-C₆) alkyl, (C₁-C₆) alkoxy, or amino-(C₁-C₄) alkyl.

The present invention also encompasses a method for treating or preventing a disease or disorder in a subject, which disease or disorder is one that can be treated or prevented by inhibiting the activity of MK-2, the method comprising administering to the subject a compound, or a pharmaceutically acceptable salt or prodrug thereof, wherein the compound has the structure according to formula III, wherein:
Q, D, T and Z are selected from carbon, sulfur, or nitrogen;
when Q is sulfur, R¹² is oxo, and R¹¹ is optionally oxo or is absent;
when D is nitrogen, R¹² is selected from oxo, ═S, or ═N—OR¹⁵, and R¹¹ is optionally oxo or is absent;
when T is sulfur, R² is oxo, and R⁹ is optionally oxo or is absent;
when Z is nitrogen, D is nitrogen, and R² and R¹² are oxo;
at least one of R¹, R², R³, R¹¹ and R¹² is oxo or oxime;
R¹, R² and R³ are independently selected from H, methyl, oxo, phenylmethylideneamino, amino, hydroxy-(C₁-C₄) alkyl, cyano-(C₁-C₄) alkyl, amino-(C₁-C₄) alkyl, alkoxycarbonyl-R¹⁵, phenylalkyl-R¹⁵, or alkyl-SO₂—R¹⁵,
R⁴, R⁶ and R⁷ are independently selected from H, methyl, methoxy, chloro, bromo, fluoro, carboxyl, CO₂—R¹⁵, nitro, t-butoxycarbonylpiperazinylcarbonyl, or piperazinylcarbonyl, or R⁶ and R⁷ optionally join to form a pyridyl ring;
R⁵ is selected from H, methoxy, ethoxy, CO₂—R¹⁵, nitro, fluoro-(C₁-C₄) alkyl, hydroxyl, bromo, iodo, chloro, fluoro, 2-phenylethyl, phenylethynyl, nitro, methoxycarbonyl or carboxyl, or R⁴ and R⁵ optionally join to form a 1,4 dioxinyl ring;
R⁹ is independently selected from H and oxo or is optionally absent;
R¹⁰ is selected from H, methyl, or phenyl; and
R¹¹ and R¹² are optionally present and if present, are independently selected from H, oxo, and oxime;
R¹⁴ is optionally present and if present, is selected from H or methyl; and
R¹⁵ is optionally present and if present, is selected from H, (C₁-C₆) alkyl, (C₁-C₆) alkoxy, or amino-(C₁-C₄) alkyl.

The present invention also encompasses a method for treating or preventing a disease or disorder in a subject, which disease or disorder is one that can be treated or prevented by inhibiting the activity of MK-2, the method comprising administering to the subject a compound, or a pharmaceutically acceptable salt or prodrug thereof, wherein the compound has the structure according to formula II, wherein:
Q, D, T and Z are selected from carbon, sulfur, or nitrogen, and at least one of Q, D, T and Z is nitrogen;
when Q is sulfur, R¹² is oxo, and R¹¹ is optionally oxo or is absent;
when D is nitrogen, R¹² is selected from oxo, ═S, or ═N—OR¹⁵, and R¹¹ is optionally oxo or is absent;
when T is sulfur, R² is oxo, and R⁹ is optionally oxo or is absent;
when Z is nitrogen, D is nitrogen, and R² and R¹² are oxo;
at least one of R¹, R², R³, R¹¹ and R¹² is oxo;
R¹, R², R³ and R¹⁰ are H;
R⁵ is selected from H, methoxy, hydroxyl, bromo, iodo, or chloro;
R⁹ is independently selected from H and oxo or is optionally absent; R¹¹ and R¹² are independently selected from H and oxo, or are optionally absent; and
R¹⁴ is optionally present and if present, is selected from H or methyl.

The present invention also encompasses a method for treating or preventing a disease or disorder in a subject, which disease or disorder is one that can be treated or prevented by inhibiting the activity of MK-2, the method comprising administering to the subject a compound, or a pharmaceutically acceptable salt or prodrug thereof, wherein the compound has the structure according to formula III, wherein:
Q, D, T and Z are selected from carbon, sulfur, or nitrogen, and at least one of Q, D, T and Z is nitrogen;
when Q is sulfur, R¹² is oxo, and R¹¹ is optionally oxo or is absent;
when D is nitrogen, R¹² is selected from oxo, ═S, or ═N—OR¹⁵, and R¹¹ is optionally oxo or is absent;
when T is sulfur, R² is oxo, and R⁹ is optionally oxo or is absent;
when Z is nitrogen, D is nitrogen, and R² and R¹² are oxo;
at least one of R¹, R², R³, R¹¹ and R¹² is oxo;
R¹, R², R³ and R¹⁰ are H;
R⁵ is selected from H, methoxy, hydroxyl;
R⁹ is independently selected from H and oxo or is optionally absent;
R¹¹ and R¹² are independently selected from H and oxo or are optionally absent; and
R¹⁴ is optionally present and if present, is selected from H or methyl.

The present invention also encompasses a method for treating or preventing a disease or disorder in a subject, which disease or disorder is one that can be treated or prevented by inhibiting the activity of MK-2, the method comprising administering to the subject a compound, or a pharmaceutically acceptable salt or prodrug thereof, wherein the compound has the structure according to formula II, wherein:
Q, D, T and Z are selected from carbon, sulfur, or nitrogen, and at least one of Q, D, T and Z is nitrogen;
when Q is sulfur, R¹² is oxo, and R¹¹ is optionally oxo or is absent;
when D is nitrogen, R¹² is selected from oxo, ═S, or ═N—OR¹⁵, and R¹¹ is optionally oxo or is absent;
when T is sulfur, R² is oxo, and R⁹ is optionally oxo or is absent;
when Z is nitrogen, D is nitrogen, and R² and R¹² are oxo;
at least one of R¹, R², R³, R¹¹ and R¹² is oxo;
R¹, R², R³ and R¹⁰ are H;
R⁵ is selected from H or methoxy;
R⁹ is independently selected from H and oxo or is optionally absent;
R¹¹ and R¹² are independently selected from H and oxo or are optionally absent; and
R¹⁴ is optionally present and if present, is selected from H or methyl.

The present invention encompasses a method for treating or preventing a disease or disorder in a subject, which disease or disorder is one that can be treated or prevented by inhibiting the activity of MK-2, the method comprising administering to the subject a compound, or a pharmaceutically acceptable salt or prodrug thereof, wherein the compound has the structure according to formula IV: 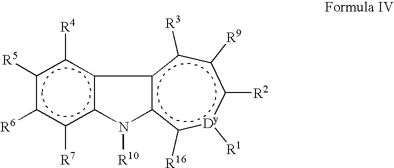
wherein:
D^{y} is selected from carbon or nitrogen;
R¹, R², R³ and R⁹ are independently selected from H, alkyl, alkoxy, carbonitrile, oxo, arylalkenylamino, or amino;
R⁴, R⁶ and R⁷ are independently selected from H, alkyl, alkoxy, halo, carboxyl, nitro, or substituted or unsubstituted 6-membered heteroarylcarbonyl, which, if substituted, have one or more substituent groups selected from H or alkoxycarbonyl;
R⁵ is selected from H or alkoxy;
R¹⁰ is selected from H, alkyl, or aryl; and
R¹⁶ is selected from H, oxo, CO₂H, or oxime.

In still other embodiments, the present invention encompasses a method for treating or preventing a disease or disorder in a subject, which disease or disorder is one that can be treated or prevented by inhibiting the activity of MK-2, the method comprising administering to the subject a compound, or a pharmaceutically acceptable salt or prodrug thereof, wherein the compound has the structure according to formula IV, wherein:
D^{y} is selected from carbon or nitrogen;
R¹, R², R³ and R⁹ are independently selected from H, alkyl, alkoxy, carbonitrile, oxo, arylalkenylamino, or amino;
R⁴, R⁶ and R⁷ are independently selected from H, alkyl, alkoxy, halo, carboxyl, nitro, or substituted or unsubstituted 6-membered heteroarylcarbonyl, which, if substituted, have one or more substituent groups selected from H or alkoxycarbonyl;
R⁵ is selected from H or alkoxy;
R¹⁰ is selected from H, alkyl, or aryl; and
R¹⁶ is selected from H, oxo, CO₂H, or oxime.

The present invention encompasses a method for treating or preventing a disease or disorder in a subject, which disease or disorder is one that can be treated or prevented by inhibiting the activity of MK-2, the method comprising administering to the subject a compound, or a pharmaceutically acceptable salt or prodrug thereof, wherein the compound has the structure according to formula IV, wherein:
D^{y} is selected from carbon or nitrogen;
when D^{y} is nitrogen, R¹⁶ is oxo;
when D^{y} is carbon, R¹⁶ oxime;
R¹, R², R³ and R⁹ are independently selected from H, (C₁-C₄) alkyl, methoxy, carbonitrile, oxo, phenylalkenylamino, or amino;
R⁴, R⁶ and R⁷ are independently selected from H, (C₁-C₄) alkyl, (C₁-C₄) alkoxy, halo, carboxyl, nitro, or substituted or unsubstituted 6-membered heteroarylcarbonyl, which, if substituted, have one or more substituent groups selected from H or alkoxycarbonyl;
R⁵ is selected from H or methoxy;
R¹⁰ is selected from H, (C₁-C₄) alkyl, or phenyl; and
R¹⁶ is selected from H, oxo, CO₂H, or oxime.

The present invention also encompasses a method for treating or preventing a disease or disorder in a subject, which disease or disorder is one that can be treated or prevented by inhibiting the activity of MK-2, the method comprising administering to the subject a compound, or a pharmaceutically acceptable salt or prodrug thereof, wherein the compound has the structure according to formula V: 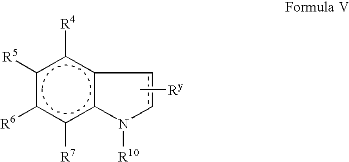
wherein:
R^{y} is optionally fused with the pyrrole ring and is selected from a substituted or unsubstituted 5-7 membered heterocyclic ring or a substituted or unsubstituted 5-7 membered cycloalkyl ring, which, if substituted, have one or more substituent groups selected from H, alkyl, alkoxy, carbonitrile, oxo, arylalkenylamino, halo, oxime, or amino;
R⁴, R⁶ and R⁷ are independently selected from H, alkyl, alkoxy, halo, carboxyl, nitro, or substituted or unsubstituted 6-membered heteroarylcarbonyl, which, if substituted, have one or more substituent groups selected from H or alkoxycarbonyl;
R⁵ is selected from H or alkoxy; and
R¹⁰ is selected from H, alkyl, or aryl.

The present invention also encompasses a method for treating or preventing a disease or disorder in a subject, which disease or disorder is one that can be treated or prevented by inhibiting the activity of MK-2, the method comprising administering to the subject a compound, or a pharmaceutically acceptable salt or prodrug thereof, wherein the compound has the structure according to formula V:
R^{y} is fused with the pyrrole ring and is selected from a substituted or unsubstituted 5 or 7 membered heterocyclic ring or a substituted or unsubstituted 5 or 7 membered cycloalkyl ring, which, if substituted, have one or more substituent groups selected from H, alkyl, alkoxy, carbonitrile, oxo, arylalkenylamino, halo, oxime, or amino;
R⁴, R⁶ and R⁷ are independently selected from H, alkyl, alkoxy, halo, carboxyl, nitro, or substituted or unsubstituted 6-membered heteroarylcarbonyl, which, if substituted, have one or more substituent groups selected from H or alkoxycarbonyl;
R⁵ is selected from H or alkoxy; and
R¹⁰ is selected from H, alkyl, or aryl.

The present invention also encompasses a method for treating or preventing a disease or disorder in a subject, which disease or disorder is one that can be treated or prevented by inhibiting the activity of MK-2, the method comprising administering to the subject a compound, or a pharmaceutically acceptable salt or prodrug thereof, wherein the compound has the structure according to formula VI:

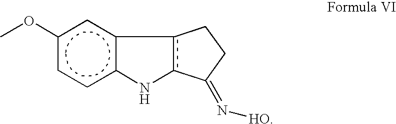

In yet other preferred embodiments, the present invention encompasses a method of modulating the activity of MK-2 in a subject in need of such modulation, the method comprising administering to the subject a MK-2 inhibiting compound, wherein the compound has the structure according to formula I, wherein:
Q, D, T and Z are selected from carbon, sulfur, or nitrogen;
when Q is sulfur, R¹² is oxo, and R¹¹ is optionally oxo or is absent;
when T is sulfur, R² is oxo, and R⁹ is optionally oxo or is absent;
when Z is nitrogen, D is nitrogen, and R² and R¹² are oxo;
R¹ is selected from H, alkyl, alkenyl, alkynyl, allyl, acetyl, amino, alkoxy, hydroxy, oxo, carboxy, allylalkyl, benzyl, 2-ethylalkyl, haloacetyl, t-butyldialkylsilyloxyalkyl, alkoxycarbonylaminoalkyl, haloalkylbenzyl, haloalkylcarbonyl, alkylcarbonyl, alkoxycarbonyl, arylcarbonyl, benzyloxycarbonyl, arylalkenylamino, alkoxyheterocyclylcarbonyl, haloarylcarbonyl, or substituted or unsubstituted alkyl, which, if substituted, has one or more substituent groups selected from H, halo, amino, aryl, or heteroaryl;
R² is selected from H, alkyl, alkoxy, hydroxy, carbonyl, oxo, benzyl, hydroxyalkyl, dialkylamino, carboxyl, amino, halo, alkoxycarbonyl, alkoxycarbonyl-R¹⁵, aminoalkyl, haloalkyl, hydroxyalkylbenzyl, haloalkylbenzyl, arylalkenylamino, haloalkoxybenzyl, cyanoalkyl, heterocyclylalkyl, arylalkyl-R¹⁵, alkyl-SO₂—R¹⁵, substituted or unsubstituted aryl, which, if substituted, has one or more substituent groups selected from H, alkyl, amino, nitro, halo, haloalkyl, or hydroxyalkyl, or substituted or unsubstituted alkyl, which, if substituted, has one or more substituent groups selected from H, halo, amino, aryl, or heteroaryl;
R³ is selected from H, oxo, alkyl, alkoxy, amino, halo, haloalkyl, hydroxy, carbonyl, alkoxycarbonyl, benzyl, aminoalkyl, hydroxyalkyl, dialkylamino, arylalkenylamino, carboxy, haloalkyl, hydroxyalkylbenzyl, haloalkylbenzyl, haloalkoxybenzyl, heterocyclylalkyl, substituted or unsubstituted aryl, which, if substituted, has one or more substituent groups selected from H, alkyl, amino, nitro, halo, haloalkyl, or hydroxyalkyl, or substituted or unsubstituted alkyl, which, if substituted, has one or more substituent groups selected from H, halo, amino, aryl, or heteroaryl, or R¹ and R³ optionally join to form an alkyl bridge between the ring atoms to which R¹ and R³ are attached, or R² and R³ optionally join to form a cycloalkyl ring;
R⁴, R⁶ and R⁷ are independently selected from H, alkyl, benzyl, halo, haloalkyl, alkoxy, haloalkoxy, carboxyl, CO₂—R¹⁵, carboxyl, nitro, amino, alkylamino, dialkylamino, benzyloxy, hydrazinocarbonyl, alkoxycarbonyl, alkoxycarbonylheterocyclylcarbonyl, hydroxy, or substituted or unsubstituted 6-membered heterocyclylcarbonyl, which, if substituted, has one or more substituent groups selected from H or alkoxycarbonyl, or R⁶ and R⁷ optionally join to form a heteroaryl ring;
R⁵ is selected from H, alkyl, alkenyl, alkylthio, hydroxy, carboxyl, halo, alkoxy, benzylalkyl, benzylalkoxy, arylalkyl, nitro, alkoxycarbonylalkoxy, alkylthio, benzyloxy, arylalkynyl, arylcarbonylalkoxy, haloalkoxy, haloalkyl, arylalkoxy, haloarylalkoxy, benzyloxoalkoxy, alkoxycarbonylbenzyloxy, alkoxycarbonyl, alkoxyoxoalkoxy, alkoxyoxoarylalkoxy, halobenzyloxy, heterocyclylalkoxy, dialkylaminooxoalkoxy, aminocarbonyl, arylalkynyl, hydrazinocarbonyl, carbonylalkoxy, alkylsulfonylbenzyloxy, CO₂—R¹⁵, carbamoyl, naphthylalkoxy, alkoxyaryloxy, alkoxycarbonylheterocyclylalkoxy, haloalkylbenzyloxy, alkoxyacetylaminoacetyloxy, acetoxy, cycloalkylalkoxy, acetyloxy, morpholinylcarbonyl, alkoxyalkoxy, or R⁴ and R⁵ optionally join to form a heterocyclic ring;
R⁸ and R⁹ are independently optionally present and if present, are independently selected from H, oxo, carbonitrile, alkyl, or is optionally absent, or R³ and R⁸ optionally join to form a cycloalkyl ring;
R¹⁰ is selected from H, alkyl, benzyl, aryl, or hydroxyalkyl;
R¹¹ is optionally present and if present, is selected from H, oxo, alkyl, halobenzyl, benzyl, haloalkyl, 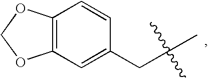
alkoxycarbonyl, carboxyalkyl, benzodioxinylalkyl, substituted or unsubstituted aryl or arylalkyl, which, if substituted, have one or more substituent groups selected from H, alkyl, or alkoxy;
R¹² is optionally present and if present, is selected from H, ═S, oxo, alkyl, carbonyl-R¹³, carboxyl-R¹³, ketone, cyano, cyanoalkyl, alkoxycarbonyl, aminoalkyl, alkoxycarbonylalkyl, carbamyl, hydroxycarbamyl, haloalkyl, heterocyclyl, alkylacetate, heterocyclylalkyl, alkoxycarbonylalkyl, hydroxyalkyl, dihydroxyalkyl, cyanoalkyl, ═N—OR¹⁵, ═N—N(R¹⁵)₂, or hydroxyamino, or R¹ and R¹² optionally join to form a substituted or unsubstituted 5 or 6-membered heteroaryl ring, which, if substituted, has one or more substituent groups selected from H, alkyl, oxo, or alkoxycarbonylalkyl, or R¹¹ and R¹² optionally join to form a ring system having the structure: 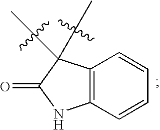
R¹³ is optionally present and if present, is selected from H, alkyl, alkoxy, amino, alkynyl, allyl, alkenyl, hydrazine, benzyl, glycine, allylalkyl, haloalkyl, hydroxyalkyl, hydroxyamino, dihydroxyalkyl, alkoxyalkyl, alkoxyamino, alkylthioalkyl, cycloalkyl, cyclohexylalkyl, amino, alkylamino, benzylalkyl, benzylamino, benzyloxycarbonylalkylamino, dialkylamino, adamantyl, haloalkoxybenzyl, alkoxyaminoalkyl, haloalkylarylalkyl, heterocyclylalkyl, alkoxyalkylamino, arylthioalkyl, substituted or unsubstituted aryl or arylalkyl, which, if substituted, have one or more substituent groups selected from H, alkyl, alkoxy, amino, nitro, halo, haloalkyl, or hydroxyalkyl;
R¹⁴ is optionally present and if present, is selected from H or alkyl; and
R¹⁵ is optionally present and if present, is selected from H, alkyl, alkoxy, or aminoalkyl.

In another preferred embodiment, the present invention encompasses a method of inhibiting MK-2 activity in a cell, the method comprising contacting the cell with a MK-2 inhibiting compound, wherein the compound has the structure according to formula I, wherein:
Q, D, T and Z are selected from carbon, sulfur, or nitrogen;
when Q is sulfur, R¹² is oxo, and R¹¹ is optionally oxo or is absent;
when T is sulfur, R² is oxo, and R⁹ is optionally oxo or is absent;
when Z is nitrogen, D is nitrogen, and R² and R¹² are oxo;
R¹ is selected from H, alkyl, alkenyl, alkynyl, allyl, acetyl, amino, alkoxy, hydroxy, oxo, carboxy, allylalkyl, benzyl, 2-ethylalkyl, haloacetyl, t-butyldialkylsilyloxyalkyl, alkoxycarbonylaminoalkyl, haloalkylbenzyl, haloalkylcarbonyl, alkylcarbonyl, alkoxycarbonyl, arylcarbonyl, benzyloxycarbonyl, arylalkenylamino, alkoxyheterocyclylcarbonyl, haloarylcarbonyl, or substituted or unsubstituted alkyl, which, if substituted, has one or more substituent groups selected from H, halo, amino, aryl, or heteroaryl;
R² is selected from H, alkyl, alkoxy, hydroxy, carbonyl, oxo, benzyl, hydroxyalkyl, dialkylamino, carboxyl, amino, halo, alkoxycarbonyl, alkoxycarbonyl-R¹⁵, aminoalkyl, haloalkyl, hydroxyalkylbenzyl, haloalkylbenzyl, arylalkenylamino, haloalkoxybenzyl, cyanoalkyl, heterocyclylalkyl, arylalkyl-R¹⁵, alkyl-SO₂—R¹⁵, substituted or unsubstituted aryl, which, if substituted, has one or more substituent groups selected from H, alkyl, amino, nitro, halo, haloalkyl, or hydroxyalkyl, or substituted or unsubstituted alkyl, which, if substituted, has one or more substituent groups selected from H, halo, amino, aryl, or heteroaryl;
R³ is selected from H, oxo, alkyl, alkoxy, amino, halo, haloalkyl, hydroxy, carbonyl, alkoxycarbonyl, benzyl, aminoalkyl, hydroxyalkyl, dialkylamino, arylalkenylamino, carboxy, haloalkyl, hydroxyalkylbenzyl, haloalkylbenzyl, haloalkoxybenzyl, heterocyclylalkyl, substituted or unsubstituted aryl, which, if substituted, has one or more substituent groups selected from H, alkyl, amino, nitro, halo, haloalkyl, or hydroxyalkyl, or substituted or unsubstituted alkyl, which, if substituted, has one or more substituent groups selected from H, halo, amino, aryl, or heteroaryl, or R¹ and R³ optionally join to form an alkyl bridge between the ring atoms to which R¹ and R³ are attached, or R² and R³ optionally join to form a cycloalkyl ring;
R⁴, R⁶ and R⁷ are independently selected from H, alkyl, benzyl, halo, haloalkyl, alkoxy, haloalkoxy, carboxyl, CO₂—R¹⁵, carboxyl, nitro, amino, alkylamino, dialkylamino, benzyloxy, hydrazinocarbonyl, alkoxycarbonyl, alkoxycarbonylheterocyclylcarbonyl, hydroxy, or substituted or unsubstituted 6-membered heterocyclylcarbonyl, which, if substituted, has one or more substituent groups selected from H or alkoxycarbonyl, or R⁶ and R⁷ optionally join to form a heteroaryl ring;
R⁵ is selected from H, alkyl, alkenyl, alkylthio, hydroxy, carboxyl, halo, alkoxy, benzylalkyl, benzylalkoxy, arylalkyl, nitro, alkoxycarbonylalkoxy, alkylthio, benzyloxy, arylalkynyl, arylcarbonylalkoxy, haloalkoxy, haloalkyl, arylalkoxy, haloarylalkoxy, benzyloxoalkoxy, alkoxycarbonylbenzyloxy, alkoxycarbonyl, alkoxyoxoalkoxy, alkoxyoxoarylalkoxy, halobenzyloxy, heterocyclylalkoxy, dialkylaminooxoalkoxy, aminocarbonyl, arylalkynyl, hydrazinocarbonyl, carbonylalkoxy, alkylsulfonylbenzyloxy, CO₂—R¹⁵, carbamoyl, naphthylalkoxy, alkoxyaryloxy, alkoxycarbonylheterocyclylalkoxy, haloalkylbenzyloxy, alkoxyacetylaminoacetyloxy, acetoxy, cycloalkylalkoxy, acetyloxy, morpholinylcarbonyl, alkoxyalkoxy, or R⁴ and R⁵ optionally join to form a heterocyclic ring;
R⁸ and R⁹ are independently optionally present and if present, are independently selected from H, oxo, carbonitrile, alkyl, or is optionally absent, or R³ and R⁸ optionally join to form a cycloalkyl ring;
R¹⁰ is selected from H, alkyl, benzyl, aryl, or hydroxyalkyl;
R¹¹ is optionally present and if present, is selected from H, oxo, alkyl, halobenzyl, benzyl, haloalkyl, 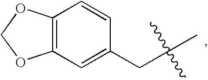
alkoxycarbonyl, carboxyalkyl, benzodioxinylalkyl, substituted or unsubstituted aryl or arylalkyl, which, if substituted, have one or more substituent groups selected from H, alkyl, or alkoxy;
R¹² is optionally present and if present, is selected from H, ═S, oxo, alkyl, carbonyl-R¹³, carboxyl-R¹³, ketone, cyano, cyanoalkyl, alkoxycarbonyl, aminoalkyl, alkoxycarbonylalkyl, carbamyl, hydroxycarbamyl, haloalkyl, heterocyclyl, alkylacetate, heterocyclylalkyl, alkoxycarbonylalkyl, hydroxyalkyl, dihydroxyalkyl, cyanoalkyl, ═N—OR¹⁵, ═N—N(R¹⁵)₂, or hydroxyamino, or R¹ and R¹² optionally join to form a substituted or unsubstituted 5 or 6-membered heteroaryl ring, which, if substituted, has one or more substituent groups selected from H, alkyl, oxo, or alkoxycarbonylalkyl, or R¹¹ and R¹² optionally join to form a ring system having the structure: 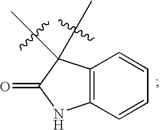
R¹³ is optionally present and if present, is selected from H, alkyl, alkoxy, amino, alkynyl, allyl, alkenyl, hydrazine, benzyl, glycine, allylalkyl, haloalkyl, hydroxyalkyl, hydroxyamino, dihydroxyalkyl, alkoxyalkyl, alkoxyamino, alkylthioalkyl, cycloalkyl, cyclohexylalkyl, amino, alkylamino, benzylalkyl, benzylamino, benzyloxycarbonylalkylamino, dialkylamino, adamantyl, haloalkoxybenzyl, alkoxyaminoalkyl, haloalkylarylalkyl, heterocyclylalkyl, alkoxyalkylamino, arylthioalkyl, substituted or unsubstituted aryl or arylalkyl, which, if substituted, have one or more substituent groups selected from H, alkyl, alkoxy, amino, nitro, halo, haloalkyl, or hydroxyalkyl;
R¹⁴ is optionally present and if present, is selected from H or alkyl; and
R¹⁵ is optionally present and if present, is selected from H, alkyl, alkoxy, or aminoalkyl.

The present invention encompasses a method for treating or preventing a disease or disorder in a subject, which disease or disorder is one that can be treated or prevented by inhibiting the activity of MK-2, the method comprising administering to the subject a compound, or a pharmaceutically acceptable salt, prodrug, stereoisomer, enantiomer, or isoform thereof, wherein the compound is chosen from:
7-hydroxy-3,4,5,10-tetrahydro-1H-2,5-methanoazepino[3,4-b]indole-1-carboxylic acid,
2,3,8,10,11,12-hexahydro-1H,7H-9,12-methanoazepino[3,4-b]pyrano[3,2-e]indole-8-carboxylic acid,
7-methoxy-3,4,5,10-tetrahydro-1H-2,5-methanoazepino[3,4-b]indole-1-carboxylic acid,
7-(methylthio)-3,4,5,10-tetrahydro-1H-2,5-methanoazepino[3,4-b]indole-1-carboxylic acid,
7-(benzyloxy)-3,4,5,10-tetrahydro-1H-2,5-methanoazepino[3,4-b]indole-1-carboxylic acid,
7-(methylthio)-3,4,5,10-tetrahydro-1H-2,5-methanoazepino[3,4-b]indole-1-carboxylic acid,
6-methoxy-2,3,4,9-tetrahydro-1H-beta-carboline-1-carboxylic acid,
6-(2-oxo-2-phenylethoxy)-2,3,4,9-tetrahydro-1H-beta-carboline-1-carboxylic acid,
6-methoxy-2-methyl-2,3,4,9-tetrahydro-1H-beta-carboline-1-carboxylic acid,
2,2,2-trifluoroethyl 7-methoxy-3,4,5,10-tetrahydro-1H-2,5-methanoazepino[3,4-b]indole-1-carboxylate,
6-methoxy-2,3,4,9-tetrahydro-1H-beta-carboline-1-carboxylic acid,
6-hydroxy-2-methyl-2,3,4,9-tetrahydro-1H-beta-carboline-1-carboxylic acid,
6-methoxy-2-methyl-2,3,4,9-tetrahydro-1H-beta-carboline-1-carboxylic acid,
2,3-dihydroxypropyl 7-methoxy-3,4,5,10-tetrahydro-1H-2,5-methanoazepino[3,4-b]indole-1-carboxylate,
4-ethyl-6-methoxy-2,3,4,9-tetrahydro-1H-beta-carboline-1-carboxylic acid,
6-methoxy-4-methyl-2,3,4,9-tetrahydro-1H-beta-carboline-1-carboxylic acid,
pyridin-4-ylmethyl 7-methoxy-3,4,5,10-tetrahydro-1H-2,5-methanoazepino[3,4-b]indole-1-carboxylate,
3-(hydroxymethyl)-6-methoxy-2,3,4,9-tetrahydro-1H-beta-carboline-1-carboxylic acid,
2-fluoroethyl 7-methoxy-3,4,5,10-tetrahydro-1H-2,5-methanoazepino[3,4-b]indole-1-carboxylate,
allyl 7-methoxy-3,4,5,10-tetrahydro-1H-2,5-methanoazepino[3,4-b]indole-1-carboxylate,
7-methoxy-3,4,5,10-tetrahydroazepino[3,4-b]indol-1 (2H)-one,
benzyl 7-methoxy-3,4,5,10-tetrahydro-1H-2,5-methanoazepino[3,4-b]indole-1-carboxylate,
2-(methylthio)ethyl 7-methoxy-3,4,5,10-tetrahydro-1H-2,5-methanoazepino[3,4-b]indole-1-carboxylate,
6-hydroxy-2,3,4,9-tetrahydro-1H-beta-carboline-1-carboxylic acid,
7-methoxy-2,3,4,9-tetrahydro-1H-beta-carboline-1-carboxylic acid,
2-methoxyethyl 7-methoxy-3,4,5,10-tetrahydro-1H-2,5-methanoazepino[3,4-b]indole-1-carboxylate,
sodium 6-(benzyloxy)-2-methyl-2,3,4,9-tetrahydro-1H-beta-carboline-1-carboxylate,
trans-8-methoxy-1,3,4,5,6,11-hexahydro-2,6-methanoazocino[3,4-b]indole-1-carboxylic acid,
methyl 7-hydroxy-3,4,5,10-tetrahydro-1H-2,5-methanoazepino[3,4-b]indole-1-carboxylate,
2-hydroxyethyl 7-methoxy-3,4,5,10-tetrahydro-1H-2,5-methanoazepino[3,4-b]indole-1-carboxylate,
2-ethyl-6-methoxy-2,3,4,9-tetrahydro-1H-beta-carboline-1-carboxylic acid,
3-(aminomethyl)-6-methoxy-2,3,4,9-tetrahydro-1H-beta-carboline-1-carboxylic acid,
2-methoxyethyl 7-methoxy-3,4,5,10-tetrahydro-1H-2,5-methanoazepino[3,4-b]indole-1-carboxylate,
2-methoxyethyl 7-hydroxy-3,4,5,10-tetrahydro-1H-2,5-methanoazepino[3,4-b]indole-1-carboxylate,
6-(methoxycarbonyl)-2,3,4,9-tetrahydro-1H-beta-carboline-1-carboxylic acid,
6-methoxy-3-methyl-2,3,4,9-tetrahydro-1H-beta-carboline-1-carboxylic acid,
benzyl 7-methoxy-3,4,5,10-tetrahydro-1H-2,5-methanoazepino[3,4-b]indole-1-carboxylate,
6-(2-ethoxy-2-oxoethoxy)-2,3,4,9-tetrahydro-1H-beta-carboline-1-carboxylic acid,
6-methoxy-3,3-dimethyl-2,3,4,9-tetrahydro-1H-beta-carboline-1-carboxylic acid,
6-methoxy-4-propyl-2,3,4,9-tetrahydro-1H-beta-carboline-1-carboxylic acid,
3,4,5,10-tetrahydro-1H-2,5-methanoazepino[3,4-b]indole-1-carboxylic acid,
6-(2-methoxy-2-oxo-1-phenylethoxy)-2,3,4,9-tetrahydro-1H-beta-carboline-1-carboxylic acid,
methyl 7-methoxy-3,4,5,10-tetrahydro-1H-2,5-methanoazepino[3,4-b]indole-1-carboxylate,
isopropyl 7-methoxy-3,4,5,10-tetrahydro-1H-2,5-methanoazepino[3,4-b]indole-1-carboxylate,
6-butoxy-2,3,4,9-tetrahydro-1H-beta-carboline-1-carboxylic acid,
propyl 7-methoxy-3,4,5,10-tetrahydro-1H-2,5-methanoazepino[3,4-b]indole-1-carboxylate,
benzyl 7-hydroxy-3,4,5,10-tetrahydro-1H-2,5-methanoazepino[3,4-b]indole-1-carboxylate,
6-(benzyloxy)-2,3,4,9-tetrahydro-1H-beta-carboline-1-carboxylic acid,
1-phenylethyl 7-methoxy-3,4,5,10-tetrahydro-1H-2,5-methanoazepino[3,4-b]indole-1-carboxylate,
6-methoxy-4-(trifluoromethyl)-2,3,4,9-tetrahydro-1H-beta-carboline-1-carboxylic acid,
6-methoxy-2,3,4,9-tetrahydro-1H-beta-carboline-1-carboxylic acid,
isopropyl 7-methoxy-3,4,5,10-tetrahydro-1H-2,5-methanoazepino[3,4-b]indole-1-carboxylate,
6-methoxy-2,3,4,9-tetrahydro-1H-beta-carbolin-1-one,
6-(2-methoxy-2-oxoethoxy)-2,3,4,9-tetrahydro-1H-beta-carboline-1-carboxylic acid,
4-benzyl-6-methoxy-2,3,4,9-tetrahydro-1H-beta-carboline-1-carboxylic acid,
6-[(2-chlorobenzyl)oxy]-2,3,4,9-tetrahydro-1H-beta-carboline-1-carboxylic acid,
6-methoxy-4,4-dimethyl-2,3,4,9-tetrahydro-1H-beta-carboline-1-carboxylic acid,
propyl 7-hydroxy-3,4,5,10-tetrahydro-1H-2,5-methanoazepino[3,4-b]indole-1-carboxylate,
6-(3-chloropropoxy)-2,3,4,9-tetrahydro-1H-beta-carboline-1-carboxylic acid,
6-(4-chlorobutoxy)-2,3,4,9-tetrahydro-1H-beta-carboline-1-carboxylic acid,
6-[(3-chlorobenzyl)oxy]-2,3,4,9-tetrahydro-1H-beta-carboline-1-carboxylic acid,
6-chloro-2,3,4,9-tetrahydro-1H-beta-carboline-1-carboxylic acid,
4-(trifluoromethoxy)benzyl 7-methoxy-3,4,5,10-tetrahydro-1H-2,5-methanoazepino[3,4-b]indole-1-carboxylate,
neopentyl 7-methoxy-3,4,5,10-tetrahydro-1H-2,5-methanoazepino[3,4-b]indole-1-carboxylate,
6-(pyridin-4-ylmethoxy)-2,3,4,9-tetrahydro-1H-beta-carboline-1-carboxylic acid,
6-[2-(dimethylamino)-2-oxoethoxy]-2,3,4,9-tetrahydro-1H-beta-carboline-1-carboxylic acid,
6-(2-phenylethoxy)-2,3,4,9-tetrahydro-1H-beta-carboline-1-carboxylic acid,
6-(aminocarbonyl)-2,3,4,9-tetrahydro-1H-beta-carboline-1-carboxylic acid,
6-bromo-2,3,4,9-tetrahydro-1H-beta-carboline-1-carboxylic acid,
propyl 7-methoxy-3,4,5,10-tetrahydro-1H-2,5-methanoazepino[3,4-b]indole-1-carboxylate,
methyl 7-(benzyloxy)-3,4,5,10-tetrahydro-1H-2,5-methanoazepino[3,4-b]indole-1-carboxylate,
ethyl 7-methoxy-3,4,5,10-tetrahydro-1H-2,5-methanoazepino[3,4-b]indole-1-carboxylate,
hexyl 7-methoxy-3,4,5,10-tetrahydro-1H-2,5-methanoazepino[3,4-b]indole-1-carboxylate,
6-fluoro-2,3,4,9-tetrahydro-1H-beta-carboline-1-carboxylic acid,
6-methoxy-2,9-dihydro-1H-beta-carbolin-1-one,
6-(carboxymethoxy)-2,3,4,9-tetrahydro-1H-beta-carboline-1-carboxylic acid,
cyclohexylmethyl 7-methoxy-3,4,5,10-tetrahydro-1H-2,5-methanoazepino[3,4-b]indole-1-carboxylate hydrochloride,
6-(pyridin-2-ylmethoxy)-2,3,4,9-tetrahydro-1H-beta-carboline-1-carboxylic acid,
6-(pyridin-3-ylmethoxy)-2,3,4,9-tetrahydro-1H-beta-carboline-1-carboxylic acid,
5-methoxy-2,3,4,9-tetrahydro-1H-beta-carboline-1-carboxylic acid,
6-{[4-(methylsulfonyl)benzyl]oxy}-2,3,4,9-tetrahydro-1H-beta-carboline-1-carboxylic acid,
cyclohexyl 7-methoxy-3,4,5,10-tetrahydro-1H-2,5-methanoazepino[3,4-b]indole-1-carboxylate,
methyl 6-methoxy-2,3,4,9-tetrahydro-1H-beta-carboline-1-carboxylate,
6-(hydrazinocarbonyl)-2,3,4,9-tetrahydro-1H-beta-carboline-1-carboxylic acid,
6-hydroxy-2,3,4,9-tetrahydro-1H-beta-carbolin-1-one,
7-hydroxy-6-methoxy-2,3,4,9-tetrahydro-1H-beta-carboline-1-carboxylic acid,
1H-pyrido[3,4-b]indole-1-carboxylic acid, 2,3,4,9-tetrahydro-9-(phenylmethyl)-2-[[3-(trifluoromethyl)phenyl]methyl]- or 9-benzyl-2-[3-(trifluoromethyl)benzyl]-2,3,4,9-tetrahydro-1H-beta-carboline-1-carboxylic acid,
cyclohexyl 7-methoxy-3,4,5,10-tetrahydro-1H-2,5-methanoazepino[3,4-b]indole-1-carboxylate,
4-[4-(hydroxymethyl)benzyl]-6-methoxy-2,3,4,9-tetrahydro-1H-beta-carboline-1-carboxylic acid,
3-ethyl-6-methoxy-2,3,4,9-tetrahydro-1H-beta-carboline-1-carboxylic acid
6-bromo-2,3,4,9-tetrahydro-1H-beta-carbolin-1-one,
6-methoxy-4-[3-(trifluoromethyl)benzyl]-2,3,4,9-tetrahydro-1H-beta-carboline-1-carboxylic acid,
6-{[4-(methoxycarbonyl)benzyl]oxy}-2,3,4,9-tetrahydro-1H-beta-carboline-1-carboxylic acid,
6-iodo-2,3,4,9-tetrahydro-1H-beta-carbolin-1-one,
cyclohexyl 7-hydroxy-3,4,5,10-tetrahydro-1H-2,5-methanoazepino[3,4-b]indole-1-carboxylate,
7-hydroxy-6-methoxy-2,3,4,9-tetrahydro-1H-beta-carboline-1-carboxylic acid,
N-hydroxy-6-methoxy-2,3,4,9-tetrahydro-1H-beta-carboline-1-carboxamide,
1H-Pyrido[3,4-b]indol-1-one, 6-chloro-2,3,4,9-tetrahdro- or 6-chloro-
2,3,4,9-tetrahydro-1H-beta-carbolin-1-one,
N,7-dimethoxy-N-methyl-3,4,5,10-tetrahydro-1H-2,5-methanoazepino[3,4-b]indole-1-carboxamide,
6-(2-naphthylmethoxy)-2,3,4,9-tetrahydro-1H-beta-carboline-1-carboxylic acid,
2-allyl-6-methoxy-2,3,4,9-tetrahydro-1H-beta-carboline-1-carboxylic acid,
methyl 6-hydroxy-2-methyl-2,3,4,9-tetrahydro-1H-beta-carboline-1-carboxylate,
6-(4-ethoxy-4-oxobutoxy)-2,3,4,9-tetrahydro-1H-beta-carboline-1-carboxylic acid,
6-{[5-(ethoxycarbonyl)-2-furyl]methoxy}-2,3,4,9-tetrahydro-1H-beta-carboline-1-carboxylic acid,
6-(2-methoxyethoxy)-2,3,4,9-tetrahydro-1H-beta-carboline-1-carboxylic acid,
6-[(4-chlorobenzyl)oxy]-2,3,4,9-tetrahydro-1H-beta-carboline-1-carboxylic acid,
tert-butyl 7-methoxy-3,4,5,10-tetrahydro-1H-2,5-methanoazepino[3,4-b]indole-1-carboxylate,
6-{[4-(trifluoromethyl)benzyl]oxy}-2,3,4,9-tetrahydro-1H-beta-carboline-1-carboxylic acid,
6-(3-phenylpropoxy)-2,3,4,9-tetrahydro-1H-beta-carboline-1-carboxylic acid,
6-methoxy-2-[4-(trifluoromethyl)benzyl]-2,3,4,9-tetrahydro-1H-beta-carboline-1-carboxylic acid,
5-(methoxycarbonyl)-2,3,4,9-tetrahydro-1H-beta-carboline-1-carboxylic acid,
6-methoxy-1-(1H-tetraazol-5-yl)-2,3,4,9-tetrahydro-1H-beta-carboline,
2-benzyl-6-methoxy-2,3,4,9-tetrahydro-1H-beta-carboline-1-carboxylic acid,
6-methoxy-3-propyl-2,3,4,9-tetrahydro-1H-beta-carboline-1-carboxylic acid,
methyl 6-methoxy-2-methyl-2,3,4,9-tetrahydro-1H-beta-carboline-1-carboxylate,
methyl 6-hydroxy-2-methyl-2,3,4,9-tetrahydro-1H-beta-carboline-1-carboxylate,
1-adamantyl 7-methoxy-3,4,5,10-tetrahydro-1H-2,5-methanoazepino[3,4-b]indole-1-carboxylate,
2,2,6,6-tetramethylcyclohexyl 7-methoxy-3,4,5,10-tetrahydro-1H-2,5-methanoazepino[3,4-b]indole-1-carboxylate,
6-[2-(2-ethoxy-2-oxoethylamino)-2-oxoethoxy]-2,3,4,9-tetrahydro-1H-beta-carboline-1-carboxylic acid,
6-(acetyloxy)-2,3,4,9-tetrahydro-1H-beta-carboline-1-carboxylic acid,
2-isopropyl-6-methoxy-2,3,4,9-tetrahydro-1H-beta-carboline-1-carboxylic acid,
8-methoxy-5,6,11,11b-tetrahydro-1H-imidazo[1′,5′:1,2]pyrido[3,4-b]indole-1,3(2H)-dione,
6-{[3-(methoxycarbonyl)benzyl]oxy}-2,3,4,9-tetrahydro-1H-beta-carboline-1-carboxylic acid,
7-methoxy-1-(1H-pyrrol-2-yl)-3,4,5,10-tetrahydro-1H-2,5-methanoazepino[3,4-b]indole,
6-methoxy-2,3,4,9-tetrahydro-1H-beta-carboline-1-carbohydrazide,
methyl 8-methoxy-3-oxo-2,3,6,11-tetrahydro-1H-indolizino[8,7-b]indole-11b(5H)-carboxylate,
8-methoxy-3-oxo-2,3,6,11-tetrahydro-1H-indolizino[8,7-b]indole-11b(5H)-carboxylic acid,
methyl 1-(chloromethyl)-2,3,4,9-tetrahydro-1H-beta-carboline-1-carboxylate,
(6-methoxy-2,3,4,9-tetrahydro-1H-beta-carbolin-1-yl)acetic acid,
6-methoxy-1-(1H-tetraazol-5-ylmethyl)-2,3,4,9-tetrahydro-1H-beta-carboline,
methyl(6-methoxy-2,3,4,9-tetrahydro-1H-beta-carbolin-1-yl)acetate,
methyl 2-benzyl-6-methoxy-2,3,4,9-tetrahydro-1H-beta-carboline-1-carboxylate,
methyl 9-(hydroxymethyl)-6-methoxy-2-methyl-2,3,4,9-tetrahydro-1H-beta-carboline-1-carboxylate,
6-methoxy-2-methyl-2,3,4,9-tetrahydro-1H-beta-carboline-1-carboxamide, diethyl 6-methoxy-2,3,4,9-tetrahydro-1H-beta-carboline-1,1-dicarboxylate,
1h-Pyrido[3,4-B]Indole-1-Acetic Acid, 2,3,4,9-Tetrahydro-1-(Methoxycarbonyl)-, 1,1-dimethylethyl ester,
methyl 1-(4-chlorobenzyl)-6-hydroxy-2-methyl-2,3,4,9-tetrahydro-1H-beta-carboline-1-carboxylate,
methyl 6-hydroxy-2-methyl-2,3,4,9-tetrahydro-1H-beta-carboline-1-carboxylate,
methyl 6-(benzyloxy)-2,3,4,9-tetrahydro-1H-beta-carboline-1-carboxylate,
methyl 6-(benzyloxy)-2-methyl-2,3,4,9-tetrahydro-1H-beta-carboline-1-carboxylate,
3-(ethoxycarbonyl)-6-methoxy-2,3,4,9-tetrahydro-1H-beta-carboline-1-carboxylic acid,
1-(2-carboxyethyl)-6-methoxy-2,3,4,9-tetrahydro-1H-beta-carboline-1-carboxylic acid,
1-(3-carboxypropyl)-6-methoxy-2,3,4,9-tetrahydro-1H-beta-carboline-1-carboxylic acid,
8-methoxy-3,3-dimethyl-2,3,5,6,11,11b-hexahydro-1H-imidazo[1′,5′:1,2]pyrido[3,4-b]indol-1-one,
methyl 2-(3-{[tert-butyl(dimethyl)silyl]oxy}propyl)-6-methoxy-2,3,4,9-tetrahydro-1H-beta-carboline-1-carboxylate,
6-methoxy-9-methyl-2,3,4,9-tetrahydro-1H-beta-carboline-1-carboxylic acid,
6-methoxy-5-methyl-2,3,4,9-tetrahydro-1H-beta-carboline-1-carboxylic acid,
methyl 3-(hydroxymethyl)-6-methoxy-2,3,4,9-tetrahydro-1H-beta-carboline-1-carboxylate,
6-methoxy-4-[4-(trifluoromethoxy)benzyl]-2,3,4,9-tetrahydro-1H-beta-carboline-1-carboxylic acid,
2′,3′,4′,9′-tetrahydro-6′-methoxy-spiro[cyclohexane-1,4′-[4H]pyrido[3,4-b]indole]-1-carboxylic acid,
3-[(dimethylamino)methyl]-6-methoxy-2,3,4,9-tetrahydro-1H-beta-carboline-1-carboxylic acid,
methyl 6-methoxy-4-propyl-2,3,4,9-tetrahydro-1H-beta-carboline-1-carboxylate,
methyl 6-methoxy-4-methyl-2,3,4,9-tetrahydro-1H-beta-carboline-1-carboxylate,
6-methoxy-3-propyl-2,3,4,9-tetrahydro-1H-beta-carboline-1-carboxylic acid,
10-methoxy-2,3,4,4a,5,6,7,11c-octahydro-1H-indolo[2,3-c]quinoline-6-carboxylic acid,
9-methoxy-3,4,6,7,12,12b-hexahydropyrazino[1′,2′:1,2]pyrido[3,4-b]indol-1(2H)-one,
3-benzyl-6-methoxy-2,3,4,9-tetrahydro-1H-beta-carboline-1-carboxylic acid,
8-(benzyloxy)-2,3,4,9-tetrahydro-1H-beta-carboline-1-carboxylic acid,
methyl 2-{2-[(tert-butoxycarbonyl)amino]ethyl}-6-methoxy-2,3,4,9-tetrahydro-1H-beta-carboline-1-carboxylate,
6-[(2-chlorobenzyl)oxy]-2,3,4,9-tetrahydro-1H-beta-carboline-1-carboxylic acid,
2-(2-ethylbutyl)-6-methoxy-2,3,4,9-tetrahydro-1H-beta-carboline-1-carboxylic acid,
6-(cyclohexylmethoxy)-2,3,4,9-tetrahydro-1H-beta-carboline-1-carboxylic acid,
methyl 2-isopropyl-6-methoxy-2,3,4,9-tetrahydro-1H-beta-carboline-1-carboxylate,
5-(hydrazinocarbonyl)-2,3,4,9-tetrahydro-1H-beta-carboline-1-carboxylic acid,
6,7-dimethoxy-2,3,4,9-tetrahydro-1H-beta-carboline-1-carboxylic acid,
2-(4-chlorobenzeyl)-2,3,4,9-tetrahydro-1H-beta-carboline-1-carboxylic acid,
7-methoxy-1-[(2,3,4-trimethoxyphenyl)methyl]-2,3,4,9-tetrahydro-1H-beta-carboline-1-carboxylic acid,
1-[(benzodioxolan-5-yl)methyl]-2,3,4,9-tetrahydro-1H-beta-carboline-1-carboxylic acid,
6-methoxy-3-(trifluoromethyl)-2,3,4,9-tetrahydro-1H-beta-carboline-1-carboxylic acid,
9-methoxy-3-methyl-3,4,6,7,12,12b-hexahydropyrazino[1′,2′:1,2]pyrido[3,4-b]indol-1 (2H)-one,
methyl 6-methoxy-2-(trifluoroacetyl)-2,3,4,9-tetrahydro-1H-beta-carboline-1-carboxylate,
methyl 9-ethyl-6-methoxy-2,3,4,9-tetrahydro-1H-beta-carboline-1-carboxylate,
methyl 6-methoxy-9-methyl-2,3,4,9-tetrahydro-1H-beta-carboline-1-carboxylate,
6-methoxy-4-phenyl-2,3,4,9-tetrahydro-1H-beta-carboline-1-carboxylic acid,
1h-Pyrido[3,4-B]Indole-1-Carboxylic Acid, 2,3,4,9-Tetrahydro-2-[(5-Methoxy-2-Furanyl)Carbonyl]-, ethyl ester,
1h-Pyrido[3,4-B]Indole-1-Carboxylic Acid, 2,3,4,9-Tetrahydro-1-methyl-, methyl ester,
1h-Indolizino[8,7-B]Indole-2-Acetic Acid, 2,3,5,6,11,11b-(methoxycarbonyl)-3-oxo-, methyl ester,
7-fluoro-2,3,4,9-tetrahydro-1H-beta-carboline-1-carboxylic acid,
8-methyl-2,3,4,9-tetrahydro-1H-beta-carboline-1-carboxylic acid,
6-methyl-2,3,4,9-tetrahydro-1H-beta-carboline-1-carboxylic acid,
7-methoxy-2,3,4,9-tetrahydro-1H-beta-carboline-1-carboxylic acid,
2-[(benzyloxy)carbonyl]-6-methoxy-N-[2-(benzyloxy)-2-oxo]ethyl-2,3,4,9-tetrahydro-1H-beta-carboline-1-carboxamide,
2-[(benzyloxy)carbonyl]-6-methoxy-2,3,4,9-tetrahydro-1H-beta-carboline-1-carboxylic acid,
6-methoxy-2,3,4,9-tetrahydro-1H-beta-carboline-1-carboxamide,
(6-methoxy-2,3,4,9-tetrahydro-1H-beta-carbolin-1-yl)methanol,
4-(2-furylmethyl)-6-methoxy-2,3,4,9-tetrahydro-1H-beta-carboline-1-carboxylic acid,
8-methoxy-2,3,4,9-tetrahydro-1H-beta-carboline-1-carboxylic acid,
6-methoxy-1-(trifluoromethyl)-2,3,4,9-tetrahydro-1H-beta-carboline,
methyl 2-allyl-6-methoxy-2,3,4,9-tetrahydro-1H-beta-carboline-1-carboxylate,
1-(carboxymethyl)-6-methoxy-2,3,4,9-tetrahydro-1H-beta-carboline-1-carboxylic acid,
3-(methoxycarbonyl)-2,3,4,9-tetrahydro-1H-beta-carboline-1-carboxylic acid,
3-(methoxycarbonyl)-1-methyl-2,3,4,9-tetrahydro-1H-beta-carboline-1-carboxylic acid,
N-[(6-methoxy-2,3,4,9-tetrahydro-1H-beta-carbolin-1-yl)carbonyl]glycine,
1-(6-methoxy-2,3,4,9-tetrahydro-1H-beta-carbolin-1-yl)methanamine,
(6-methoxy-2,3,4,9-tetrahydro-1H-beta-carbolin-1-yl)acetonitrile,
methyl 2-acetyl-6-methoxy-2,3,4,9-tetrahydro-1H-beta-carboline-1-carboxylate,
6-methoxy-4-phenyl-2,3,4,9-tetrahydro-1H-beta-carboline-1-carboxylic acid,
2-tert-butyl 1-methyl 6-methoxy-1,3,4,9-tetrahydro-2H-beta-carboline-1,2-dicarboxylate,
methyl 1-methyl-2,3,4,9-tetrahydro-1H-beta-carboline-1-carboxylate,
8-methoxy-5,6,11,11b-tetrahydro-1H-[1,3]oxazolo[3′,4′:1,2]pyrido[3,4-b]indol-1-one,
6-methoxy-1-methyl-2,3,4,9-tetrahydro-1H-beta-carboline-1-carboxylic acid,
5-(methoxycarbonyl)-2,3,4,9-tetrahydro-1H-beta-carboline-1-carboxylic acid,
(1S)-1-(6-methoxy-2,3,4,9-tetrahydro-1H-beta-carbolin-1-yl)ethane-1,2-diol,
6-chloro-2,3,4,9-tetrahydro-1H-beta-carboline-1-carboxylic acid,
1-benzyl-6-methoxy-2,3,4,9-tetrahydro-1H-beta-carboline-1-carboxylic acid,
7-methoxy-N-methyl-3,4,5,10-tetrahydro-1H-2,5-methanoazepino[3,4-b]indole-1-carboxamide,
N-benzyl-7-methoxy-3,4,5,10-tetrahydro-1H-2,5-methanoazepino[3,4-b]indole-1-carboxamide,
7-methoxy-N,N-dimethyl-3,4,5,10-tetrahydro-1H-2,5-methanoazepino[3,4-b]indole-1-carboxamide,
benzyl 7-(benzyloxy)-3,4,5,10-tetrahydro-1H-2,5-methanoazepino[3,4-b]indole-1-carboxylate,
7-methoxy-3,4,5,10-tetrahydro-1H-2,5-methanoazepino[3,4-b]indole-1-carboxamide,
7-methoxy-N-(2-methoxyethyl)-3,4,5,10-tetrahydro-1H-2,5-methanoazepino[3,4-b]indole-1-carboxamide,
6-(phenylethynyl)-2,3,4,9-tetrahydro-1H-beta-carbolin-1-one,
6-(2-phenylethyl)-2,3,4,9-tetrahydro-1H-beta-carbolin-1-one,
1-oxo-2,3,4,9-tetrahydro-1H-beta-carboline-5-carboxylic acid,
5-(morpholin-4-ylcarbonyl)-2,3,4,9-tetrahydro-1H-beta-carbolin-1-one,
5,8-dichloro-2,3,4,9-tetrahydro-1H-beta-carbolin-1-one,
6-nitro-2,3,4,9-tetrahydro-1H-beta-carbolin-1-one,
1h-Pyrido[3,4-B]Indol-1-One, 2,3,4,9-Tetrahydro-6-Methyl-
6-ethoxy-8-nitro-2,3,4,9-tetrahydro-1H-beta-carbolin-1-one, 6-bromo-2,3,7,9,10,11-hexahydro-8H-[1,4]dioxino[2,3-e]pyrido[3,4-b]indol-8-one,
2-methyl-9-phenyl-2,3,4,9-tetrahydro-1H-beta-carbolin-1-one,
1H-Pyrido[3,4-b]indol-1-one, 2,3,4,9-tetrahydro-6-methyl- or 6-methyl-2,3,4,9-tetrahydro-1H-beta-carbolin-1-one,
8-methoxy-9-methyl-5-nitro-2,3,4,9-tetrahydro-1H-beta-carbolin-1-one,
1H-Pyrido[3,4-b]indol-1-one, 2,3,4,9-tetrahydro- or 2,3,4,9-tetrahydro-1H-beta-carbolin-1-one,
1H-Pyrido[3,4-b]indol-1-one, 6-fluoro-2,3,4,9-tetrahydro- or 6-fluoro-2,3,4,9-tetrahydro-1H-beta-carbolin-1-one or 1h-Pyrido[3,4-B]Indol-1-One,
6-Fluoro-2,3,4,9-tetrahydro-1H-Pyrido[3,4-b]indol-1-one,
8-chloro-2,3,4,9-tetrahydro- or 8-chloro-2,3,4,9-tetrahydro-1H-beta-carbolin-1-one,
1H-Pyrido[3,4-b]indole-1,3,4(2H,9H)-trione, 2,9-dimethyl- or 2,9-dimethyl-1H-beta-carboline-1,3,4(2H,9H)-trione,
7-methoxy-2,3,4,9-tetrahydro-1H-beta-carbolin-1-one,
8-methoxy-3-{[(1E)-phenylmethylidene]amino}-1H-pyrimido[5,4-b]indole-2,4(3H,5H)-dione,
3-amino-8-methoxy-1H-pyrimido[5,4-b]indole-2,4(3H,5H)-dione,
tert-butyl 4-[(1-oxo-2,3,4,9-tetrahydro-1H-beta-carbolin-5-yl)carbonyl]piperazine-1-carboxylate,
5-(piperazin-1-ylcarbonyl)-2,3,4,9-tetrahydro-1H-beta-carbolin-1-one,
3,7-dimethoxy-1-oxo-1,2,5,10-tetrahydroazepino[3,4-b]indole-4-carbonitrile,
methyl 1-oxo-2,3,4,9-tetrahydro-1H-beta-carboline-6-carboxylate,
1-oxo-2,3,4,9-tetrahydro-1H-beta-carboline-6-carboxylic acid,
8,9,10,11-tetrahydro-7H-pyrido[3′,4′:4,5]pyrrolo[2,3-f]isoquinolin-7-one trifluoroacetate,
3-(aminomethyl)-6-methoxy-2,3,4,9-tetrahydro-1H-beta-carbolin-1-one trifluoroacetate,
3-(aminomethyl)-6-methoxy-2,3,4,9-tetrahydro-1H-beta-carbolin-1-one hydrochloride,
6-methoxy-3-{3-[(2-phenylethyl)amino]propyl}-2,3,4,9-tetrahydro-1H-beta-carbolin-1-one trifluoroacetate,
(1E)-6-methoxy-2,3,4,9-tetrahydro-1H-carbazol-1-one oxime,
(1Z)-6-methoxy-2,3,4,9-tetrahydro-1H-carbazol-1-one oxime,
6-methoxy-3-{3-[(3-phenylpropyl)amino]propyl}-2,3,4,9-tetrahydro-1H-beta-carbolin-1-one trifluoroacetate,
methyl 1-oxo-2,3,4,5-tetrahydro-1H-pyrido[4,3-b]indole-6-carboxylate,
3-(hydroxymethyl)-6-methoxy-2,3,4,9-tetrahydro-1H-beta-carbolin-1-one,
3-(3-aminopropyl)-6-methoxy-2,3,4,9-tetrahydro-1H-beta-carbolin-1-one trifluoroacetate,
3-(2-aminoethyl)-6-methoxy-2,3,4,9-tetrahydro-1H-beta-carbolin-1-one trifluoroacetate,
ethyl 1-(hydroxyimino)-2,3,4,9-tetrahydro-1H-carbazole-6-carboxylate,
2-methoxy-7,8,9,10-tetrahydrocyclohepta[b]indol-6(5H)-one oxime,
3-[3-(benzylamino)propyl]-6-methoxy-2,3,4,9-tetrahydro-1H-beta-carbolin-1-one trifluoroacetate,
6-methoxy-2,3,4,9-tetrahydro-1H-carbazol-1-one oxime,
6-iodo-2,3,4,9-tetrahydro-1H-carbazol-1-one oxime,
6-methoxy-2-methyl-2,3,4,9-tetrahydro-1H-carbazol-1-one oxime,
3-(3-hydroxypropyl)-6-methoxy-2,3,4,9-tetrahydro-1H-beta-carbolin-1-one,
ethyl 1-oxo-2,3,4,5-tetrahydro-1H-pyrido[4,3-b]indole-6-carboxylate,
6-methoxy-2,3,4,9-tetrahydro-1H-beta-carboline-1-thione,
methyl 4-oxo-2,3,4,9-tetrahydro-1H-carbazole-8-carboxylate,
2,3,4,9-tetrahydro-1H-carbazol-1-one oxime,
ethyl 1-oxo-2,3,4,5-tetrahydro-1H-pyrido[4,3-b]indole-7-carboxylate,
(6-methoxy-1-oxo-2,3,4,9-tetrahydro-1H-beta-carbolin-3-yl)acetonitrile,
6-methyl-2,3,4,9-tetrahydro-1H-carbazol-1-one oxime,
6,7-dimethoxy-2,3,4,9-tetrahydro-1H-carbazol-1-one oxime,
7-methoxy-1,4-dihydrocyclopenta[b]indol-3(2H)-one oxime,
6-methoxy-2-methyl-2,3,4,9-tetrahydro-1H-carbazol-1-one hydrazone,
(6-methoxy-1-oxo-2,3,4,9-tetrahydro-1H-beta-carbolin-3-yl)methyl methanesulfonate,
6-(4-methoxyphenoxy)-2,3,4,9-tetrahydro-1H-carbazol-1-one oxime,
6-methoxy-2,3,4,9-tetrahydro-1H-beta-carbolin-1-one oxime,
methyl 3-(6-methoxy-1-oxo-2,3,4,9-tetrahydro-1H-beta-carbolin-3-yl)propanoate,
6-methoxy-2,3,4,9-tetrahydro[1,2]thiazino[6,5-b]indole 1,1-dioxide,
6-(trifluoromethyl)-2,3,4,9-tetrahydro-1H-carbazol-1-one oxime, and
6-methoxy-2,2-dimethyl-2,3,4,9-tetrahydro-1H-carbazol-1-one oxime, mixtures thereof, and pharmaceutically acceptable salts thereof.

Compounds that are encompassed by the present invention include, but are not limited to, those compounds that have an MK-2 inhibition IC₅₀ value of below 200 μM that are chosen from:
7-methoxy-3,4,5,10-tetrahydro-1H-2,5-methanoazepino[3,4-b]indole-1-carboxylic acid,
7-hydroxy-3,4,5,10-tetrahydro-1H-2,5-methanoazepino[3,4-b]indole-1-carboxylic acid,
2,3,8,10,11,12-hexahydro-1H,7H-9,12-methanoazepino[3,4-b]pyrano[3,2-e]indole-8-carboxylic acid,
7-methoxy-3,4,5,10-tetrahydro-1H-2,5-methanoazepino[3,4-b]indole-1-carboxylic acid,
7-(methylthio)-3,4,5,10-tetrahydro-1H-2,5-methanoazepino[3,4-b]indole-1-carboxylic acid,
7-methoxy-3,4,5,10-tetrahydro-1H-2,5-methanoazepino[3,4-b]indole-1-carboxylic acid,
7-(benzyloxy)-3,4,5,10-tetrahydro-1H-2,5-methanoazepino[3,4-b]indole-1-carboxylic acid,
7-(methylthio)-3,4,5,10-tetrahydro-1H-2,5-methanoazepino[3,4-b]indole-1-carboxylic acid,
6-methoxy-2,3,4,9-tetrahydro-1H-beta-carboline-1-carboxylic acid,
6-(2-oxo-2-phenylethoxy)-2,3,4,9-tetrahydro-1H-beta-carboline-1-carboxylic acid,
6-methoxy-2-methyl-2,3,4,9-tetrahydro-1H-beta-carboline-1-carboxylic acid,
2,2,2-trifluoroethyl 7-methoxy-3,4,5,10-tetrahydro-1H-2,5-methanoazepino[3,4-b]indole-1-carboxylate,
6-methoxy-2,3,4,9-tetrahydro-1H-beta-carboline-1-carboxylic acid,
7-hydroxy-3,4,5,10-tetrahydro-1H-2,5-methanoazepino[3,4-b]indole-1-carboxylic acid,
6-hydroxy-2-methyl-2,3,4,9-tetrahydro-1H-beta-carboline-1-carboxylic acid,
7-methoxy-3,4,5,10-tetrahydro-1H-2,5-methanoazepino[3,4-b]indole-1-carboxylic acid,
6-methoxy-2-methyl-2,3,4,9-tetrahydro-1H-beta-carboline-1-carboxylic acid,
2,3-dihydroxypropyl 7-methoxy-3,4,5,10-tetrahydro-1H-2,5-methanoazepino[3,4-b]indole-1-carboxylate,
4-ethyl-6-methoxy-2,3,4,9-tetrahydro-1H-beta-carboline-1-carboxylic acid,
6-methoxy-4-methyl-2,3,4,9-tetrahydro-1H-beta-carboline-1-carboxylic acid,
pyridin-4-ylmethyl 7-methoxy-3,4,5,10-tetrahydro-1H-2,5-methanoazepino[3,4-b]indole-1-carboxylate,
3-(hydroxymethyl)-6-methoxy-2,3,4,9-tetrahydro-1H-beta-carboline-1-carboxylic acid,
2-fluoroethyl 7-methoxy-3,4,5,10-tetrahydro-1H-2,5-methanoazepino[3,4-b]indole-1-carboxylate,
allyl 7-methoxy-3,4,5,10-tetrahydro-1H-2,5-methanoazepino[3,4-b]indole-1-carboxylate,
7-methoxy-3,4,5,10-tetrahydroazepino[3,4-b]indol-1 (2H)-one,
benzyl 7-methoxy-3,4,5,10-tetrahydro-1H-2,5-methanoazepino[3,4-b]indole-1-carboxylate,
7-methoxy-3,4,5,10-tetrahydro-1H-2,5-methanoazepino[3,4-b]indole-1-carboxylic acid,
2-(methylthio)ethyl 7-methoxy-3,4,5,10-tetrahydro-1H-2,5-methanoazepino[3,4-b]indole-1-carboxylate,
6-hydroxy-2,3,4,9-tetrahydro-1H-beta-carboline-1-carboxylic acid,
7-methoxy-2,3,4,9-tetrahydro-1H-beta-carboline-1-carboxylic acid,
2-methoxyethyl 7-methoxy-3,4,5,10-tetrahydro-1H-2,5-methanoazepino[3,4-b]indole-1-carboxylate,
sodium 6-(benzyloxy)-2-methyl-2,3,4,9-tetrahydro-1H-beta-carboline-1-carboxylate,
8-methoxy-1,3,4,5,6,11-hexahydro-2,6-methanoazocino[3,4-b]indole-1-carboxylic acid,
methyl 7-hydroxy-3,4,5,10-tetrahydro-1H-2,5-methanoazepino[3,4-b]indole-1-carboxylate,
2-hydroxyethyl 7-methoxy-3,4,5,10-tetrahydro-1H-2,5-methanoazepino[3,4-b]indole-1-carboxylate,
2-ethyl-6-methoxy-2,3,4,9-tetrahydro-1H-beta-carboline-1-carboxylic acid,
3-(aminomethyl)-6-methoxy-2,3,4,9-tetrahydro-1H-beta-carboline-1-carboxylic acid,
2-methoxyethyl 7-methoxy-3,4,5,10-tetrahydro-1H-2,5-methanoazepino[3,4-b]indole-1-carboxylate acid,
2-methoxyethyl 7-hydroxy-3,4,5,10-tetrahydro-1H-2,5-methanoazepino[3,4-b]indole-1-carboxylate,
6-(methoxycarbonyl)-2,3,4,9-tetrahydro-1H-beta-carboline-1-carboxylic acid,
6-methoxy-3-methyl-2,3,4,9-tetrahydro-1H-beta-carboline-1-carboxylic acid,
benzyl 7-methoxy-3,4,5,10-tetrahydro-1H-2,5-methanoazepino[3,4-b]indole-1-carboxylate,
benzyl 7-methoxy-3,4,5,10-tetrahydro-1H-2,5-methanoazepino[3,4-b]indole-1-carboxylate,
6-(2-ethoxy-2-oxoethoxy)-2,3,4,9-tetrahydro-1H-beta-carboline-1-carboxylic acid,
6-methoxy-3,3-dimethyl-2,3,4,9-tetrahydro-1H-beta-carboline-1-carboxylic acid,
6-methoxy-4-propyl-2,3,4,9-tetrahydro-1H-beta-carboline-1-carboxylic acid,
3,4,5,10-tetrahydro-1H-2,5-methanoazepino[3,4-b]indole-1-carboxylic acid,
6-(2-methoxy-2-oxo-1-phenylethoxy)-2,3,4,9-tetrahydro-1H-beta-carboline-1-carboxylic acid,
methyl 7-methoxy-3,4,5,10-tetrahydro-1H-2,5-methanoazepino[3,4-b]indole-1-carboxylate,
isopropyl 7-methoxy-3,4,5,10-tetrahydro-1H-2,5-methanoazepino[3,4-b]indole-1-carboxylate,
6-butoxy-2,3,4,9-tetrahydro-1H-beta-carboline-1-carboxylic acid,
propyl 7-methoxy-3,4,5,10-tetrahydro-1H-2,5-methanoazepino[3,4-b]indole-1-carboxylate,
benzyl 7-hydroxy-3,4,5,10-tetrahydro-1H-2,5-methanoazepino[3,4-b]indole-1-carboxylate,
6-(benzyloxy)-2,3,4,9-tetrahydro-1H-beta-carboline-1-carboxylic acid,
1-phenylethyl 7-methoxy-3,4,5,10-tetrahydro-1H-2,5-methanoazepino[3,4-b]indole-1-carboxylate,
6-methoxy-4-(trifluoromethyl)-2,3,4,9-tetrahydro-1H-beta-carboline-1-carboxylic acid,
6-methoxy-2,3,4,9-tetrahydro-1H-beta-carboline-1-carboxylic acid,
isopropyl 7-methoxy-3,4,5,10-tetrahydro-1H-2,5-methanoazepino[3,4-b]indole-1-carboxylate,
6-methoxy-2,3,4,9-tetrahydro-1H-beta-carbolin-1-one,
6-(2-methoxy-2-oxoethoxy)-2,3,4,9-tetrahydro-1H-beta-carboline-1-carboxylic acid,
4-benzyl-6-methoxy-2,3,4,9-tetrahydro-1H-beta-carboline-1-carboxylic acid,
6-[(2-chlorobenzyl)oxy]-2,3,4,9-tetrahydro-1H-beta-carboline-1-carboxylic acid,
6-methoxy-4,4-dimethyl-2,3,4,9-tetrahydro-1H-beta-carboline-1-carboxylic acid,
propyl 7-hydroxy-3,4,5,10-tetrahydro-1H-2,5-methanoazepino[3,4-b]indole-1-carboxylate,
6-(3-chloropropoxy)-2,3,4,9-tetrahydro-1H-beta-carboline-1-carboxylic acid,
6-(4-chlorobutoxy)-2,3,4,9-tetrahydro-1H-beta-carboline-1-carboxylic acid,
6-[(3-chlorobenzyl)oxy]-2,3,4,9-tetrahydro-1H-beta-carboline-1-carboxylic acid,
6-chloro-2,3,4,9-tetrahydro-1H-beta-carboline-1-carboxylic acid,
4-(trifluoromethoxy)benzyl 7-methoxy-3,4,5,10-tetrahydro-1H-2,5-methanoazepino[3,4-b]indole-1-carboxylate,
neopentyl 7-methoxy-3,4,5,10-tetrahydro-1H-2,5-methanoazepino[3,4-b]indole-1-carboxylate,
6-(pyridin-4-ylmethoxy)-2,3,4,9-tetrahydro-1H-beta-carboline-1-carboxylic acid,
6-[2-(dimethylamino)-2-oxoethoxy]-2,3,4,9-tetrahydro-1H-beta-carboline-1-carboxylic acid,
6-(2-phenylethoxy)-2,3,4,9-tetrahydro-1H-beta-carboline-1-carboxylic acid,
6-(aminocarbonyl)-2,3,4,9-tetrahydro-1H-beta-carboline-1-carboxylic acid,
6-bromo-2,3,4,9-tetrahydro-1H-beta-carboline-1-carboxylic acid,
propyl 7-methoxy-3,4,5,10-tetrahydro-1H-2,5-methanoazepino[3,4-b]indole-1-carboxylate,
methyl 7-(benzyloxy)-3,4,5,10-tetrahydro-1H-2,5-methanoazepino[3,4-b]indole-1-carboxylate compound with trifluoroacetic acid (1:1),
ethyl 7-methoxy-3,4,5,10-tetrahydro-1H-2,5-methanoazepino[3,4-b]indole-1-carboxylate,
hexyl 7-methoxy-3,4,5,10-tetrahydro-1H-2,5-methanoazepino[3,4-b]indole-1-carboxylate,
6-fluoro-2,3,4,9-tetrahydro-1H-beta-carboline-1-carboxylic acid,
6-methoxy-2,9-dihydro-1H-beta-carbolin-1-one,
6-(carboxymethoxy)-2,3,4,9-tetrahydro-1H-beta-carboline-1-carboxylic acid,
cyclohexylmethyl 7-methoxy-3,4,5,10-tetrahydro-1H-2,5-methanoazepino[3,4-b]indole-1-carboxylate,
6-(pyridin-2-ylmethoxy)-2,3,4,9-tetrahydro-1H-beta-carboline-1-carboxylic acid,
6-(pyridin-3-ylmethoxy)-2,3,4,9-tetrahydro-1H-beta-carboline-1-carboxylic acid,
5-methoxy-2,3,4,9-tetrahydro-1H-beta-carboline-1-carboxylic acid,
6-{[4-(methylsulfonyl)benzyl]oxy}-2,3,4,9-tetrahydro-1H-beta-carboline-1-carboxylic acid,
cyclohexyl 7-methoxy-3,4,5,10-tetrahydro-1H-2,5-methanoazepino[3,4-b]indole-1-carboxylate,
methyl 6-methoxy-2,3,4,9-tetrahydro-1H-beta-carboline-1-carboxylate,
6-(hydrazinocarbonyl)-2,3,4,9-tetrahydro-1H-beta-carboline-1-carboxylic acid,
6-hydroxy-2,3,4,9-tetrahydro-1H-beta-carbolin-1-one,
7-hydroxy-6-methoxy-2,3,4,9-tetrahydro-1H-beta-carboline-1-carboxylic acid,
1H-Pyrido[3,4-b]indole-1-carboxylic acid, 2,3,4,9-tetrahydro-9-(phenylmethyl)-2-[[3-(trifluoromethyl)phenyl]methyl]- or 9-benzyl-2-[3-(trifluoromethyl)benzyl]-2,3,4,9-tetrahydro-1H-beta-carboline-1-carboxylic acid,
cyclohexyl 7-methoxy-3,4,5,10-tetrahydro-1H-2,5-methanoazepino[3,4-b]indole-1-carboxylate,
cyclohexyl 7-methoxy-3,4,5,10-tetrahydro-1H-2,5-methanoazepino[3,4-b]indole-1-carboxylate,
4-[4-(hydroxymethyl)benzyl]-6-methoxy-2,3,4,9-tetrahydro-1H-beta-carboline-1-carboxylic acid,
3-ethyl-6-methoxy-2,3,4,9-tetrahydro-1H-beta-carboline-1-carboxylic acid
6-bromo-2,3,4,9-tetrahydro-1H-beta-carbolin-1-one,
6-methoxy-4-[3-(trifluoromethyl)benzyl]-2,3,4,9-tetrahydro-1H-beta-carboline-1-carboxylic acid,
6-{[4-(methoxycarbonyl)benzyl]oxy}-2,3,4,9-tetrahydro-1H-beta-carboline-1-carboxylic acid,
6-iodo-2,3,4,9-tetrahydro-1H-beta-carbolin-1-one,
cyclohexyl 7-hydroxy-3,4,5,10-tetrahydro-1H-2,5-methanoazepino[3,4-b]indole-1-carboxylate 4-methylbenzenesulfonate,
7-hydroxy-6-methoxy-2,3,4,9-tetrahydro-1H-beta-carboline-1-carboxylic acid,
N-hydroxy-6-methoxy-2,3,4,9-tetrahydro-1H-beta-carboline-1-carboxamide,
1H-Pyrido[3,4-b]indol-1-one, 6-chloro-2,3,4,9-tetrahdro- or 6-chloro-2,3,4,9-tetrahydro-1H-beta-carbolin-1-one,
N,7-dimethoxy-N-methyl-3,4,5,10-tetrahydro-1H-2,5-methanoazepino[3,4-b]indole-1-carboxamide,
6-(2-naphthylmethoxy)-2,3,4,9-tetrahydro-1H-beta-carboline-1-carboxylic acid,
potassium 2-allyl-6-methoxy-2,3,4,9-tetrahydro-1H-beta-carboline-1-carboxylate,
methyl 6-hydroxy-2-methyl-2,3,4,9-tetrahydro-1H-beta-carboline-1-carboxylate,
6-(4-ethoxy-4-oxobutoxy)-2,3,4,9-tetrahydro-1H-beta-carboline-1-carboxylic acid,
6-{[5-(ethoxycarbonyl)-2-furyl]methoxy}-2,3,4,9-tetrahydro-1H-beta-carboline-1-carboxylic acid,
cyclohexyl 7-methoxy-3,4,5,10-tetrahydro-1H-2,5-methanoazepino[3,4-b]indole-1-carboxylate,
6-(2-methoxyethoxy)-2,3,4,9-tetrahydro-1H-beta-carboline-1-carboxylic acid,
6-[(4-chlorobenzyl)oxy]-2,3,4,9-tetrahydro-1H-beta-carboline-1-carboxylic acid,
tert-butyl 7-methoxy-3,4,5,10-tetrahydro-1H-2,5-methanoazepino[3,4-b]indole-1-carboxylate,
6-{[4-(trifluoromethyl)benzyl]oxy}-2,3,4,9-tetrahydro-1H-beta-carboline-1-carboxylic acid,
6-(3-phenylpropoxy)-2,3,4,9-tetrahydro-1H-beta-carboline-1-carboxylic acid,
6-methoxy-2-[4-(trifluoromethyl)benzyl]-2,3,4,9-tetrahydro-1H-beta-carboline-1-carboxylic acid,
5-(methoxycarbonyl)-2,3,4,9-tetrahydro-1H-beta-carboline-1-carboxylic acid,
6-methoxy-1-(1H-tetraazol-5-yl)-2,3,4,9-tetrahydro-1H-beta-carboline,
2-benzyl-6-methoxy-2,3,4,9-tetrahydro-1H-beta-carboline-1-carboxylic acid,
6-iodo-2,3,4,9-tetrahydro-1H-beta-carbolin-1-one,
6-methoxy-3-propyl-2,3,4,9-tetrahydro-1H-beta-carboline-1-carboxylic acid,
methyl 6-methoxy-2-methyl-2,3,4,9-tetrahydro-1H-beta-carboline-1-carboxylate,
methyl 6-hydroxy-2-methyl-2,3,4,9-tetrahydro-1H-beta-carboline-1-carboxylate,
1-adamantyl 7-methoxy-3,4,5,10-tetrahydro-1H-2,5-methanoazepino[3,4-b]indole-1-carboxylate,
2,2,6,6-tetramethylcyclohexyl 7-methoxy-3,4,5,10-tetrahydro-1H-2,5-methanoazepino[3,4-b]indole-1-carboxylate,
6-(acetyloxy)-2,3,4,9-tetrahydro-1H-beta-carboline-1-carboxylic acid,
8,9,10,11-tetrahydro-7H-pyrido[3′,4′:4,5]pyrrolo[2,3-f]isoquinolin-7-one trifluoroacetate,
3-(aminomethyl)-6-methoxy-2,3,4,9-tetrahydro-1H-beta-carbolin-1-one trifluoroacetate,
3-(aminomethyl)-6-methoxy-2,3,4,9-tetrahydro-1H-beta-carbolin-1-one hydrochloride,
6-methoxy-3-{3-[(2-phenylethyl)amino]propyl}-2,3,4,9-tetrahydro-1H-beta-carbolin-1-one trifluoroacetate,
(1E)-6-methoxy-2,3,4,9-tetrahydro-1H-carbazol-1-one oxime,
(1Z)-6-methoxy-2,3,4,9-tetrahydro-1H-carbazol-1-one oxime,
6-methoxy-3-{3-[(3-phenylpropyl)amino]propyl}-2,3,4,9-tetrahydro-1H-beta-carbolin-1-one trifluoroacetate,
methyl 1-oxo-2,3,4,5-tetrahydro-1H-pyrido[4,3-b]indole-6-carboxylate,
3-(hydroxymethyl)-6-methoxy-2,3,4,9-tetrahydro-1H-beta-carbolin-1-one,
3-(3-aminopropyl)-6-methoxy-2,3,4,9-tetrahydro-1H-beta-carbolin-1-one trifluoroacetate,
3-(2-aminoethyl)-6-methoxy-2,3,4,9-tetrahydro-1H-beta-carbolin-1-one trifluoroacetate,
ethyl 1-(hydroxyimino)-2,3,4,9-tetrahydro-1H-carbazole-6-carboxylate,
2-methoxy-7,8,9,10-tetrahydrocyclohepta[b]indol-6(5H)-one oxime,
3-[3-(benzylamino)propyl]-6-methoxy-2,3,4,9-tetrahydro-1H-beta-carbolin-1-one trifluoroacetate,
6-methoxy-2,3,4,9-tetrahydro-1H-carbazol-1-one oxime,
6-iodo-2,3,4,9-tetrahydro-1H-carbazol-1-one oxime,
6-methoxy-2-methyl-2,3,4,9-tetrahydro-1H-carbazol-1-one oxime,
3-(3-hydroxypropyl)-6-methoxy-2,3,4,9-tetrahydro-1H-beta-carbolin-1-one,
ethyl 1-oxo-2,3,4,5-tetrahydro-1H-pyrido[4,3-b]indole-6-carboxylate,
6-methoxy-2,3,4,9-tetrahydro-1H-beta-carboline-1-thione,
methyl 4-oxo-2,3,4,9-tetrahydro-1H-carbazole-8-carboxylate,
2,3,4,9-tetrahydro-1H-carbazol-1-one oxime,
ethyl 1-oxo-2,3,4,5-tetrahydro-1H-pyrido[4,3-b]indole-7-carboxylate,
(6-methoxy-1-oxo-2,3,4,9-tetrahydro-1H-beta-carbolin-3-yl)acetonitrile,
6-methyl-2,3,4,9-tetrahydro-1H-carbazol-1-one oxime,
6,7-dimethoxy-2,3,4,9-tetrahydro-1H-carbazol-1-one oxime,
7-methoxy-1,4-dihydrocyclopenta[b]indol-3(2H)-one oxime,
6-methoxy-2-methyl-2,3,4,9-tetrahydro-1H-carbazol-1-one hydrazone,
(6-methoxy-1-oxo-2,3,4,9-tetrahydro-1H-beta-carbolin-3-yl)methyl methanesulfonate,
6-(4-methoxyphenoxy)-2,3,4,9-tetrahydro-1H-carbazol-1-one oxime,
6-methoxy-2,3,4,9-tetrahydro-1H-beta-carbolin-1-one oxime,
methyl 3-(6-methoxy-1-oxo-2,3,4,9-tetrahydro-1H-beta-carbolin-3-yl)propanoate,
6-methoxy-2,3,4,9-tetrahydro[1,2]thiazino[6,5-b]indole 1,1-dioxide, and
6-(trifluoromethyl)-2,3,4,9-tetrahydro-1H-carbazol-1-one oxime, mixtures thereof, and pharmaceutically acceptable salts thereof.

Compounds that are encompassed by the present invention include, but are not limited to, those compounds that have an MK-2 inhibition IC₅₀ value of below 100 μM that are chosen from:
7-methoxy-3,4,5,10-tetrahydro-1H-2,5-methanoazepino[3,4-b]indole-1-carboxylic acid,
7-hydroxy-3,4,5,10-tetrahydro-1H-2,5-methanoazepino[3,4-b]indole-1-carboxylic acid,
2,3,8,10,11,12-hexahydro-1H,7H-9,12-methanoazepino[3,4-b]pyrano[3,2-e]indole-8-carboxylic acid,
7-methoxy-3,4,5,10-tetrahydro-1H-2,5-methanoazepino[3,4-b]indole-1-carboxylic acid,
7-(methylthio)-3,4,5,10-tetrahydro-1H-2,5-methanoazepino[3,4-b]indole-1-carboxylic acid,
7-methoxy-3,4,5,10-tetrahydro-1H-2,5-methanoazepino[3,4-b]indole-1-carboxylic acid,
7-(benzyloxy)-3,4,5,10-tetrahydro-1H-2,5-methanoazepino[3,4-b]indole-1-carboxylic acid,
7-(methylthio)-3,4,5,10-tetrahydro-1H-2,5-methanoazepino[3,4-b]indole-1-carboxylic acid,
6-methoxy-2,3,4,9-tetrahydro-1H-beta-carboline-1-carboxylic acid,
6-(2-oxo-2-phenylethoxy)-2,3,4,9-tetrahydro-1H-beta-carboline-1-carboxylic acid,
6-methoxy-2-methyl-2,3,4,9-tetrahydro-1H-beta-carboline-1-carboxylic acid,
2,2,2-trifluoroethyl 7-methoxy-3,4,5,10-tetrahydro-1H-2,5-methanoazepino[3,4-b]indole-1-carboxylate,
6-methoxy-2,3,4,9-tetrahydro-1H-beta-carboline-1-carboxylic acid,
7-hydroxy-3,4,5,10-tetrahydro-1H-2,5-methanoazepino[3,4-b]indole-1-carboxylic acid,
6-hydroxy-2-methyl-2,3,4,9-tetrahydro-1H-beta-carboline-1-carboxylic acid,
7-methoxy-3,4,5,10-tetrahydro-1H-2,5-methanoazepino[3,4-b]indole-1-carboxylic acid,
6-methoxy-2-methyl-2,3,4,9-tetrahydro-1H-beta-carboline-1-carboxylic acid,
2,3-dihydroxypropyl 7-methoxy-3,4,5,10-tetrahydro-1H-2,5-methanoazepino[3,4-b]indole-1-carboxylate,
4-ethyl-6-methoxy-2,3,4,9-tetrahydro-1H-beta-carboline-1-carboxylic acid,
6-methoxy-4-methyl-2,3,4,9-tetrahydro-1H-beta-carboline-1-carboxylic acid,
pyridin-4-ylmethyl 7-methoxy-3,4,5,10-tetrahydro-1H-2,5-methanoazepino[3,4-b]indole-1-carboxylate,
3-(hydroxymethyl)-6-methoxy-2,3,4,9-tetrahydro-1H-beta-carboline-1-carboxylic acid,
2-fluoroethyl 7-methoxy-3,4,5,10-tetrahydro-1H-2,5-methanoazepino[3,4-b]indole-1-carboxylate,
allyl 7-methoxy-3,4,5,10-tetrahydro-1H-2,5-methanoazepino[3,4-b]indole-1-carboxylate,
7-methoxy-3,4,5,10-tetrahydroazepino[3,4-b]indol-1 (2H)-one,
benzyl 7-methoxy-3,4,5,10-tetrahydro-1H-2,5-methanoazepino[3,4-b]indole-1-carboxylate,
7-methoxy-3,4,5,10-tetrahydro-1H-2,5-methanoazepino[3,4-b]indole-1-carboxylic acid,
2-(methylthio)ethyl 7-methoxy-3,4,5,10-tetrahydro-1H-2,5-methanoazepino[3,4-b]indole-1-carboxylate,
6-hydroxy-2,3,4,9-tetrahydro-1H-beta-carboline-1-carboxylic acid,
7-methoxy-2,3,4,9-tetrahydro-1H-beta-carboline-1-carboxylic acid,
2-methoxyethyl 7-methoxy-3,4,5,10-tetrahydro-1H-2,5-methanoazepino[3,4-b]indole-1-carboxylate,
sodium 6-(benzyloxy)-2-methyl-2,3,4,9-tetrahydro-1H-beta-carboline-1-carboxylate,
8-methoxy-1,3,4,5,6,11-hexahydro-2,6-methanoazocino[3,4-b]indole-1-carboxylic acid,
methyl 7-hydroxy-3,4,5,10-tetrahydro-1H-2,5-methanoazepino[3,4-b]indole-1-carboxylate,
2-hydroxyethyl 7-methoxy-3,4,5,10-tetrahydro-1H-2,5-methanoazepino[3,4-b]indole-1-carboxylate,
2-ethyl-6-methoxy-2,3,4,9-tetrahydro-1H-beta-carboline-1-carboxylic acid,
3-(aminomethyl)-6-methoxy-2,3,4,9-tetrahydro-1H-beta-carboline-1-carboxylic acid,
2-methoxyethyl 7-methoxy-3,4,5,10-tetrahydro-1H-2,5-methanoazepino[3,4-b]indole-1-carboxylate compound with trifluoroacetic acid (1:1),
2-methoxyethyl 7-hydroxy-3,4,5,10-tetrahydro-1H-2,5-methanoazepino[3,4-b]indole-1-carboxylate,
6-(methoxycarbonyl)-2,3,4,9-tetrahydro-1H-beta-carboline-1-carboxylic acid,
6-methoxy-3-methyl-2,3,4,9-tetrahydro-1H-beta-carboline-1-carboxylic acid,
benzyl 7-methoxy-3,4,5,10-tetrahydro-1H-2,5-methanoazepino[3,4-b]indole-1-carboxylate,
benzyl 7-methoxy-3,4,5,10-tetrahydro-1H-2,5-methanoazepino[3,4-b]indole-1-carboxylate,
6-(2-ethoxy-2-oxoethoxy)-2,3,4,9-tetrahydro-1H-beta-carboline-1-carboxylic acid,
6-methoxy-3,3-dimethyl-2,3,4,9-tetrahydro-1H-beta-carboline-1-carboxylic acid,
6-methoxy-4-propyl-2,3,4,9-tetrahydro-1H-beta-carboline-1-carboxylic acid,
3,4,5,10-tetrahydro-1H-2,5-methanoazepino[3,4-b]indole-1-carboxylic acid,
6-(2-methoxy-2-oxo-1-phenylethoxy)-2,3,4,9-tetrahydro-1H-beta-carboline-1-carboxylic acid,
methyl 7-methoxy-3,4,5,10-tetrahydro-1H-2,5-methanoazepino[3,4-b]indole-1-carboxylate,
isopropyl 7-methoxy-3,4,5,10-tetrahydro-1H-2,5-methanoazepino[3,4-b]indole-1-carboxylate,
6-butoxy-2,3,4,9-tetrahydro-1H-beta-carboline-1-carboxylic acid,
propyl 7-methoxy-3,4,5,10-tetrahydro-1H-2,5-methanoazepino[3,4-b]indole-1-carboxylate,
benzyl 7-hydroxy-3,4,5,10-tetrahydro-1H-2,5-methanoazepino[3,4-b]indole-1-carboxylate,
6-(benzyloxy)-2,3,4,9-tetrahydro-1H-beta-carboline-1-carboxylic acid,
1-phenylethyl 7-methoxy-3,4,5,10-tetrahydro-1H-2,5-methanoazepino[3,4-b]indole-1-carboxylate,
6-methoxy-4-(trifluoromethyl)-2,3,4,9-tetrahydro-1H-beta-carboline-1-carboxylic acid,
6-methoxy-2,3,4,9-tetrahydro-1H-beta-carboline-1-carboxylic acid,
isopropyl 7-methoxy-3,4,5,10-tetrahydro-1H-2,5-methanoazepino[3,4-b]indole-1-carboxylate,
6-methoxy-2,3,4,9-tetrahydro-1H-beta-carbolin-1-one,
6-(2-methoxy-2-oxoethoxy)-2,3,4,9-tetrahydro-1H-beta-carboline-1-carboxylic acid,
4-benzyl-6-methoxy-2,3,4,9-tetrahydro-1H-beta-carboline-1-carboxylic acid,
6-[(2-chlorobenzyl)oxy]-2,3,4,9-tetrahydro-1H-beta-carboline-1-carboxylic acid,
6-methoxy-4,4-dimethyl-2,3,4,9-tetrahydro-1H-beta-carboline-1-carboxylic acid,
propyl 7-hydroxy-3,4,5,10-tetrahydro-1H-2,5-methanoazepino[3,4-b]indole-1-carboxylate,
6-(3-chloropropoxy)-2,3,4,9-tetrahydro-1H-beta-carboline-1-carboxylic acid,
6-(4-chlorobutoxy)-2,3,4,9-tetrahydro-1H-beta-carboline-1-carboxylic acid,
6-[(3-chlorobenzyl)oxy]-2,3,4,9-tetrahydro-1H-beta-carboline-1-carboxylic acid,
6-chloro-2,3,4,9-tetrahydro-1H-beta-carboline-1-carboxylic acid,
4-(trifluoromethoxy)benzyl 7-methoxy-3,4,5,10-tetrahydro-1H-2,5-methanoazepino[3,4-b]indole-1-carboxylate,
neopentyl 7-methoxy-3,4,5,10-tetrahydro-1H-2,5-methanoazepino[3,4-b]indole-1-carboxylate,
6-(pyridin-4-ylmethoxy)-2,3,4,9-tetrahydro-1H-beta-carboline-1-carboxylic acid,
6-[2-(dimethylamino)-2-oxoethoxy]-2,3,4,9-tetrahydro-1H-beta-carboline-1-carboxylic acid,
6-(2-phenylethoxy)-2,3,4,9-tetrahydro-1H-beta-carboline-1-carboxylic acid,
6-(aminocarbonyl)-2,3,4,9-tetrahydro-1H-beta-carboline-1-carboxylic acid,
6-bromo-2,3,4,9-tetrahydro-1H-beta-carboline-1-carboxylic acid,
propyl 7-methoxy-3,4,5,10-tetrahydro-1H-2,5-methanoazepino[3,4-b]indole-1-carboxylate,
methyl 7-(benzyloxy)-3,4,5,10-tetrahydro-1H-2,5-methanoazepino[3,4-b]indole-1-carboxylate,
ethyl 7-methoxy-3,4,5,10-tetrahydro-1H-2,5-methanoazepino[3,4-b]indole-1-carboxylate,
hexyl 7-methoxy-3,4,5,10-tetrahydro-1H-2,5-methanoazepino[3,4-b]indole-1-carboxylate,
6-fluoro-2,3,4,9-tetrahydro-1H-beta-carboline-1-carboxylic acid,
6-methoxy-2,9-dihydro-1H-beta-carbolin-1-one,
6-(carboxymethoxy)-2,3,4,9-tetrahydro-1H-beta-carboline-1-carboxylic acid,
cyclohexylmethyl 7-methoxy-3,4,5,10-tetrahydro-1H-2,5-methanoazepino[3,4-b]indole-1-carboxylate,
6-(pyridin-2-ylmethoxy)-2,3,4,9-tetrahydro-1H-beta-carboline-1-carboxylic acid,
6-(pyridin-3-ylmethoxy)-2,3,4,9-tetrahydro-1H-beta-carboline-1-carboxylic acid,
5-methoxy-2,3,4,9-tetrahydro-1H-beta-carboline-1-carboxylic acid,
6-{[4-(methylsulfonyl)benzyl]oxy}-2,3,4,9-tetrahydro-1H-beta-carboline-1-carboxylic acid,
cyclohexyl 7-methoxy-3,4,5,10-tetrahydro-1H-2,5-methanoazepino[3,4-b]indole-1-carboxylate,
methyl 6-methoxy-2,3,4,9-tetrahydro-1H-beta-carboline-1-carboxylate,
6-(hydrazinocarbonyl)-2,3,4,9-tetrahydro-1H-beta-carboline-1-carboxylic acid,
6-hydroxy-2,3,4,9-tetrahydro-1H-beta-carbolin-1-one,
7-hydroxy-6-methoxy-2,3,4,9-tetrahydro-1H-beta-carboline-1-carboxylic acid,
1H-Pyrido[3,4-b]indole-1-carboxylic acid, 2,3,4,9-tetrahydro-9-(phenylmethyl)-2-[[3-(trifluoromethyl)phenyl]methyl]- or 9-benzyl-2-[3-(trifluoromethyl)benzyl]-2,3,4,9-tetrahydro-1H-beta-carboline-1-carboxylic acid,
cyclohexyl 7-methoxy-3,4,5,10-tetrahydro-1H-2,5-methanoazepino[3,4-b]indole-1-carboxylate,
cyclohexyl 7-methoxy-3,4,5,10-tetrahydro-1H-2,5-methanoazepino[3,4-b]indole-1-carboxylate,
4-[4-(hydroxymethyl)benzyl]-6-methoxy-2,3,4,9-tetrahydro-1H-beta-carboline-1-carboxylic acid,
3-ethyl-6-methoxy-2,3,4,9-tetrahydro-1H-beta-carboline-1-carboxylic acid 6-bromo-2,3,4,9-tetrahydro-1H-beta-carbolin-1-one,
6-methoxy-4-[3-(trifluoromethyl)benzyl]-2,3,4,9-tetrahydro-1H-beta-carboline-1-carboxylic acid,
6-{[4-(methoxycarbonyl)benzyl]oxy}-2,3,4,9-tetrahydro-1H-beta-carboline-1-carboxylic acid,
6-iodo-2,3,4,9-tetrahydro-1H-beta-carbolin-1-one,
cyclohexyl 7-hydroxy-3,4,5,10-tetrahydro-1H-2,5-methanoazepino[3,4-b]indole-1-carboxylate 4-methylbenzenesulfonate,
7-hydroxy-6-methoxy-2,3,4,9-tetrahydro-1H-beta-carboline-1-carboxylic acid,
N-hydroxy-6-methoxy-2,3,4,9-tetrahydro-1H-beta-carboline-1-carboxamide,
1H-Pyrido[3,4-b]indol-1-one, 6-chloro-2,3,4,9-tetrahdro- or 6-chloro-2,3,4,9-tetrahydro-1H-beta-carbolin-1-one,
N,7-dimethoxy-N-methyl-3,4,5,10-tetrahydro-1H-2,5-methanoazepino[3,4-b]indole-1-carboxamide,
6-(2-naphthylmethoxy)-2,3,4,9-tetrahydro-1H-beta-carboline-1-carboxylic acid,
potassium 2-allyl-6-methoxy-2,3,4,9-tetrahydro-1H-beta-carboline-1-carboxylate,
methyl 6-hydroxy-2-methyl-2,3,4,9-tetrahydro-1H-beta-carboline-1-carboxylate,
8,9,10,11-tetrahydro-7H-pyrido[3′,4′:4,5]pyrrolo[2,3-f]isoquinolin-7-one trifluoroacetate,
3-(aminomethyl)-6-methoxy-2,3,4,9-tetrahydro-1H-beta-carbolin-1-one trifluoroacetate,
3-(aminomethyl)-6-methoxy-2,3,4,9-tetrahydro-1H-beta-carbolin-1-one hydrochloride,
6-methoxy-3-{3-[(2-phenylethyl)amino]propyl}-2,3,4,9-tetrahydro-1H-beta-carbolin-1-one trifluoroacetate,
(1E)-6-methoxy-2,3,4,9-tetrahydro-1H-carbazol-1-one oxime,
(1Z)-6-methoxy-2,3,4,9-tetrahydro-1H-carbazol-1-one oxime,
6-methoxy-3-{3-[(3-phenylpropyl)amino]propyl}-2,3,4,9-tetrahydro-1H-beta-carbolin-1-one trifluoroacetate,
methyl 1-oxo-2,3,4,5-tetrahydro-1H-pyrido[4,3-b]indole-6-carboxylate,
3-(hydroxymethyl)-6-methoxy-2,3,4,9-tetrahydro-1H-beta-carbolin-1-one,
3-(3-aminopropyl)-6-methoxy-2,3,4,9-tetrahydro-1H-beta-carbolin-1-one trifluoroacetate,
3-(2-aminoethyl)-6-methoxy-2,3,4,9-tetrahydro-1H-beta-carbolin-1-one trifluoroacetate,
ethyl 1-(hydroxyimino)-2,3,4,9-tetrahydro-1H-carbazole-6-carboxylate,
2-methoxy-7,8,9,10-tetrahydrocyclohepta[b]indol-6(5H)-one oxime,
3-[3-(benzylamino)propyl]-6-methoxy-2,3,4,9-tetrahydro-1H-beta-carbolin-1-one trifluoroacetate,
6-methoxy-2,3,4,9-tetrahydro-1H-carbazol-1-one oxime,
6-iodo-2,3,4,9-tetrahydro-1H-carbazol-1-one oxime,
6-methoxy-2-methyl-2,3,4,9-tetrahydro-1H-carbazol-1-one oxime,
3-(3-hydroxypropyl)-6-methoxy-2,3,4,9-tetrahydro-1H-beta-carbolin-1-one,
ethyl 1-oxo-2,3,4,5-tetrahydro-1H-pyrido[4,3-b]indole-6-carboxylate,
6-methoxy-2,3,4,9-tetrahydro-1H-beta-carboline-1-thione,
methyl 4-oxo-2,3,4,9-tetrahydro-1H-carbazole-8-carboxylate,
2,3,4,9-tetrahydro-1H-carbazol-1-one oxime,
ethyl 1-oxo-2,3,4,5-tetrahydro-1H-pyrido[4,3-b]indole-7-carboxylate,
(6-methoxy-1-oxo-2,3,4,9-tetrahydro-1H-beta-carbolin-3-yl)acetonitrile, and
6-methyl-2,3,4,9-tetrahydro-1H-carbazol-1-one oxime, mixtures thereof, and pharmaceutically acceptable salts thereof.

Compounds that are encompassed by the present invention include, but are not limited to, those compounds that have an MK-2 inhibition IC₅₀ value of below 50 μM that are chosen from:
7-methoxy-3,4,5,10-tetrahydro-1H-2,5-methanoazepino[3,4-b]indole-1-carboxylic acid,
7-hydroxy-3,4,5,10-tetrahydro-1H-2,5-methanoazepino[3,4-b]indole-1-carboxylic acid,
2,3,8,10,11,12-hexahydro-1H,7H-9,12-methanoazepino[3,4-b]pyrano[3,2-e]indole-8-carboxylic acid,
7-methoxy-3,4,5,10-tetrahydro-1H-2,5-methanoazepino[3,4-b]indole-1-carboxylic acid,
7-(methylthio)-3,4,5,10-tetrahydro-1H-2,5-methanoazepino[3,4-b]indole-1-carboxylic acid,
7-methoxy-3,4,5,10-tetrahydro-1H-2,5-methanoazepino[3,4-b]indole-1-carboxylic acid,
7-(benzyloxy)-3,4,5,10-tetrahydro-1H-2,5-methanoazepino[3,4-b]indole-1-carboxylic acid,
7-(methylthio)-3,4,5,10-tetrahydro-1H-2,5-methanoazepino[3,4-b]indole-1-carboxylic acid,
6-methoxy-2,3,4,9-tetrahydro-1H-beta-carboline-1-carboxylic acid,
6-(2-oxo-2-phenylethoxy)-2,3,4,9-tetrahydro-1H-beta-carboline-1-carboxylic acid,
6-methoxy-2-methyl-2,3,4,9-tetrahydro-1H-beta-carboline-1-carboxylic acid,
2,2,2-trifluoroethyl 7-methoxy-3,4,5,10-tetrahydro-1H-2,5-methanoazepino[3,4-b]indole-1-carboxylate,
6-methoxy-2,3,4,9-tetrahydro-1H-beta-carboline-1-carboxylic acid,
7-hydroxy-3,4,5,10-tetrahydro-1H-2,5-methanoazepino[3,4-b]indole-1-carboxylic acid,
6-hydroxy-2-methyl-2,3,4,9-tetrahydro-1H-beta-carboline-1-carboxylic acid,
7-methoxy-3,4,5,10-tetrahydro-1H-2,5-methanoazepino[3,4-b]indole-1-carboxylic acid,
6-methoxy-2-methyl-2,3,4,9-tetrahydro-1H-beta-carboline-1-carboxylic acid,
2,3-dihydroxypropyl 7-methoxy-3,4,5,10-tetrahydro-1H-2,5-methanoazepino[3,4-b]indole-1-carboxylate,
4-ethyl-6-methoxy-2,3,4,9-tetrahydro-1H-beta-carboline-1-carboxylic acid,
6-methoxy-4-methyl-2,3,4,9-tetrahydro-1H-beta-carboline-1-carboxylic acid,
pyridin-4-ylmethyl 7-methoxy-3,4,5,10-tetrahydro-1H-2,5-methanoazepino[3,4-b]indole-1-carboxylate,
3-(hydroxymethyl)-6-methoxy-2,3,4,9-tetrahydro-1H-beta-carboline-1-carboxylic acid,
2-fluoroethyl 7-methoxy-3,4,5,10-tetrahydro-1H-2,5-methanoazepino[3,4-b]indole-1-carboxylate,
allyl 7-methoxy-3,4,5,10-tetrahydro-1H-2,5-methanoazepino[3,4-b]indole-1-carboxylate,
7-methoxy-3,4,5,10-tetrahydroazepino[3,4-b]indol-1 (2H)-one,
benzyl 7-methoxy-3,4,5,10-tetrahydro-1H-2,5-methanoazepino[3,4-b]indole-1-carboxylate,
7-methoxy-3,4,5,10-tetrahydro-1H-2,5-methanoazepino[3,4-b]indole-1-carboxylic acid,
2-(methylthio)ethyl 7-methoxy-3,4,5,10-tetrahydro-1H-2,5-methanoazepino[3,4-b]indole-1-carboxylate,
6-hydroxy-2,3,4,9-tetrahydro-1H-beta-carboline-1-carboxylic acid,
7-methoxy-2,3,4,9-tetrahydro-1H-beta-carboline-1-carboxylic acid,
2-methoxyethyl 7-methoxy-3,4,5,10-tetrahydro-1H-2,5-methanoazepino[3,4-b]indole-1-carboxylate,
sodium 6-(benzyloxy)-2-methyl-2,3,4,9-tetrahydro-1H-beta-carboline-1-carboxylate,
8-methoxy-1,3,4,5,6,11-hexahydro-2,6-methanoazocino[3,4-b]indole-1-carboxylic acid,
methyl 7-hydroxy-3,4,5,10-tetrahydro-1H-2,5-methanoazepino[3,4-b]indole-1-carboxylate,
2-hydroxyethyl 7-methoxy-3,4,5,10-tetrahydro-1H-2,5-methanoazepino[3,4-b]indole-1-carboxylate,
2-ethyl-6-methoxy-2,3,4,9-tetrahydro-1H-beta-carboline-1-carboxylic acid,
3-(aminomethyl)-6-methoxy-2,3,4,9-tetrahydro-1H-beta-carboline-1-carboxylic acid,
2-methoxyethyl 7-methoxy-3,4,5,10-tetrahydro-1H-2,5-methanoazepino[3,4-b]indole-1-carboxylate acid,
2-methoxyethyl 7-hydroxy-3,4,5,10-tetrahydro-1H-2,5-methanoazepino[3,4-b]indole-1-carboxylate,
6-(methoxycarbonyl)-2,3,4,9-tetrahydro-1H-beta-carboline-1-carboxylic acid,
6-methoxy-3-methyl-2,3,4,9-tetrahydro-1H-beta-carboline-1-carboxylic acid,
benzyl 7-methoxy-3,4,5,10-tetrahydro-1H-2,5-methanoazepino[3,4-b]indole-1-carboxylate,
benzyl 7-methoxy-3,4,5,10-tetrahydro-1H-2,5-methanoazepino[3,4-b]indole-1-carboxylate,
6-(2-ethoxy-2-oxoethoxy)-2,3,4,9-tetrahydro-1H-beta-carboline-1-carboxylic acid,
6-methoxy-3,3-dimethyl-2,3,4,9-tetrahydro-1H-beta-carboline-1-carboxylic acid,
6-methoxy-4-propyl-2,3,4,9-tetrahydro-1H-beta-carboline-1-carboxylic acid,
3,4,5,10-tetrahydro-1H-2,5-methanoazepino[3,4-b]indole-1-carboxylic acid,
6-(2-methoxy-2-oxo-1-phenylethoxy)-2,3,4,9-tetrahydro-1H-beta-carboline-1-carboxylic acid,
methyl 7-methoxy-3,4,5,10-tetrahydro-1H-2,5-methanoazepino[3,4-b]indole-1-carboxylate,
isopropyl 7-methoxy-3,4,5,10-tetrahydro-1H-2,5-methanoazepino[3,4-b]indole-1-carboxylate,
6-butoxy-2,3,4,9-tetrahydro-1H-beta-carboline-1-carboxylic acid,
propyl 7-methoxy-3,4,5,10-tetrahydro-1H-2,5-methanoazepino[3,4-b]indole-1-carboxylate,
benzyl 7-hydroxy-3,4,5,10-tetrahydro-1H-2,5-methanoazepino[3,4-b]indole-1-carboxylate,
6-(benzyloxy)-2,3,4,9-tetrahydro-1H-beta-carboline-1-carboxylic acid,
1-phenylethyl 7-methoxy-3,4,5,10-tetrahydro-1H-2,5-methanoazepino[3,4-b]indole-1-carboxylate,
6-methoxy-4-(trifluoromethyl)-2,3,4,9-tetrahydro-1H-beta-carboline-1-carboxylic acid,
6-methoxy-2,3,4,9-tetrahydro-1H-beta-carboline-1-carboxylic acid,
isopropyl 7-methoxy-3,4,5,10-tetrahydro-1H-2,5-methanoazepino[3,4-b]indole-1-carboxylate,
6-methoxy-2,3,4,9-tetrahydro-1H-beta-carbolin-1-one,
6-(2-methoxy-2-oxoethoxy)-2,3,4,9-tetrahydro-1H-beta-carboline-1-carboxylic acid,
4-benzyl-6-methoxy-2,3,4,9-tetrahydro-1H-beta-carboline-1-carboxylic acid,
6-[(2-chlorobenzyl)oxy]-2,3,4,9-tetrahydro-1H-beta-carboline-1-carboxylic acid,
6-methoxy-4,4-dimethyl-2,3,4,9-tetrahydro-1H-beta-carboline-1-carboxylic acid,
propyl 7-hydroxy-3,4,5,10-tetrahydro-1H-2,5-methanoazepino[3,4-b]indole-1-carboxylate,
6-(3-chloropropoxy)-2,3,4,9-tetrahydro-1H-beta-carboline-1-carboxylic acid,
6-(4-chlorobutoxy)-2,3,4,9-tetrahydro-1H-beta-carboline-1-carboxylic acid,
6-[(3-chlorobenzyl)oxy]-2,3,4,9-tetrahydro-1H-beta-carboline-1-carboxylic acid,
6-chloro-2,3,4,9-tetrahydro-1H-beta-carboline-1-carboxylic acid,
4-(trifluoromethoxy)benzyl 7-methoxy-3,4,5,10-tetrahydro-1H-2,5-methanoazepino[3,4-b]indole-1-carboxylate,
neopentyl 7-methoxy-3,4,5,10-tetrahydro-1H-2,5-methanoazepino[3,4-b]indole-1-carboxylate,
6-(pyridin-4-ylmethoxy)-2,3,4,9-tetrahydro-1H-beta-carboline-1-carboxylic acid,
6-[2-(dimethylamino)-2-oxoethoxy]-2,3,4,9-tetrahydro-1H-beta-carboline-1-carboxylic acid,
6-(2-phenylethoxy)-2,3,4,9-tetrahydro-1H-beta-carboline-1-carboxylic acid,
6-(aminocarbonyl)-2,3,4,9-tetrahydro-1H-beta-carboline-1-carboxylic acid,
6-bromo-2,3,4,9-tetrahydro-1H-beta-carboline-1-carboxylic acid,
propyl 7-methoxy-3,4,5,10-tetrahydro-1H-2,5-methanoazepino[3,4-b]indole-1-carboxylate,
methyl 7-(benzyloxy)-3,4,5,10-tetrahydro-1H-2,5-methanoazepino[3,4-b]indole-1-carboxylate,
ethyl 7-methoxy-3,4,5,10-tetrahydro-1H-2,5-methanoazepino[3,4-b]indole-1-carboxylate,
hexyl 7-methoxy-3,4,5,10-tetrahydro-1H-2,5-methanoazepino[3,4-b]indole-1-carboxylate,
6-fluoro-2,3,4,9-tetrahydro-1H-beta-carboline-1-carboxylic acid,
6-methoxy-2,9-dihydro-1H-beta-carbolin-1-one,
6-(carboxymethoxy)-2,3,4,9-tetrahydro-1H-beta-carboline-1-carboxylic acid,
cyclohexylmethyl 7-methoxy-3,4,5,10-tetrahydro-1H-2,5-methanoazepino[3,4-b]indole-1-carboxylate,
6-(pyridin-2-ylmethoxy)-2,3,4,9-tetrahydro-1H-beta-carboline-1-carboxylic acid,
6-(pyridin-3-ylmethoxy)-2,3,4,9-tetrahydro-1H-beta-carboline-1-carboxylic acid,
5-methoxy-2,3,4,9-tetrahydro-1H-beta-carboline-1-carboxylic acid,
6-{[4-(methylsulfonyl)benzyl]oxy}-2,3,4,9-tetrahydro-1H-beta-carboline-1-carboxylic acid,
cyclohexyl 7-methoxy-3,4,5,10-tetrahydro-1H-2,5-methanoazepino[3,4-b]indole-1-carboxylate,
methyl 6-methoxy-2,3,4,9-tetrahydro-1H-beta-carboline-1-carboxylate,
6-(hydrazinocarbonyl)-2,3,4,9-tetrahydro-1H-beta-carboline-1-carboxylic acid,
6-hydroxy-2,3,4,9-tetrahydro-1H-beta-carbolin-1-one, 8,9,10,11-tetrahydro-7H-pyrido[3′,4′:4,5]pyrrolo[2,3-f]isoquinolin-7-one trifluoroacetate,
3-(aminomethyl)-6-methoxy-2,3,4,9-tetrahydro-1H-beta-carbolin-1-one trifluoroacetate,
3-(aminomethyl)-6-methoxy-2,3,4,9-tetrahydro-1H-beta-carbolin-1-one hydrochloride,
6-methoxy-3-{3-[(2-phenylethyl)amino]propyl}-2,3,4,9-tetrahydro-1H-beta-carbolin-1-one trifluoroacetate,
(1E)-6-methoxy-2,3,4,9-tetrahydro-1H-carbazol-1-one oxime,
(1Z)-6-methoxy-2,3,4,9-tetrahydro-1H-carbazol-1-one oxime,
6-methoxy-3-{3-[(3-phenylpropyl)amino]propyl}-2,3,4,9-tetrahydro-1H-beta-carbolin-1-one trifluoroacetate,
methyl 1-oxo-2,3,4,5-tetrahydro-1H-pyrido[4,3-b]indole-6-carboxylate,
3-(hydroxymethyl)-6-methoxy-2,3,4,9-tetrahydro-1H-beta-carbolin-1-one,
3-(3-aminopropyl)-6-methoxy-2,3,4,9-tetrahydro-1H-beta-carbolin-1-one trifluoroacetate,
3-(2-aminoethyl)-6-methoxy-2,3,4,9-tetrahydro-1H-beta-carbolin-1-one trifluoroacetate,
ethyl 1-(hydroxyimino)-2,3,4,9-tetrahydro-1H-carbazole-6-carboxylate,
2-methoxy-7,8,9,10-tetrahydrocyclohepta[b]indol-6(5H)-one oxime,
3-[3-(benzylamino)propyl]-6-methoxy-2,3,4,9-tetrahydro-1H-beta-carbolin-1-one trifluoroacetate,
6-methoxy-2,3,4,9-tetrahydro-1H-carbazol-1-one oxime,
6-iodo-2,3,4,9-tetrahydro-1H-carbazol-1-one oxime,
6-methoxy-2-methyl-2,3,4,9-tetrahydro-1H-carbazol-1-one oxime,
3-(3-hydroxypropyl)-6-methoxy-2,3,4,9-tetrahydro-1H-beta-carbolin-1-one,
ethyl 1-oxo-2,3,4,5-tetrahydro-1H-pyrido[4,3-b]indole-6-carboxylate,
6-methoxy-2,3,4,9-tetrahydro-1H-beta-carboline-1-thione,
methyl 4-oxo-2,3,4,9-tetrahydro-1H-carbazole-8-carboxylate,
2,3,4,9-tetrahydro-1H-carbazol-1-one oxime, mixtures thereof, and pharmaceutically acceptable salts thereof.

Compounds that are encompassed by the present invention include, but are not limited to, those compounds that have an MK-2 inhibition IC₅₀ value of below 20 μM that are chosen from:
7-methoxy-3,4,5,10-tetrahydro-1H-2,5-methanoazepino[3,4-b]indole-1-carboxylic acid,
7-hydroxy-3,4,5,10-tetrahydro-1H-2,5-methanoazepino[3,4-b]indole-1-carboxylic acid,
2,3,8,10,11,12-hexahydro-1H,7H-9,12-methanoazepino[3,4-b]pyrano[3,2-e]indole-8-carboxylic acid,
7-methoxy-3,4,5,10-tetrahydro-1H-2,5-methanoazepino[3,4-b]indole-1-carboxylic acid,
7-(methylthio)-3,4,5,10-tetrahydro-1H-2,5-methanoazepino[3,4-b]indole-1-carboxylic acid,
7-methoxy-3,4,5,10-tetrahydro-1H-2,5-methanoazepino[3,4-b]indole-1-carboxylic acid,
7-(benzyloxy)-3,4,5,10-tetrahydro-1H-2,5-methanoazepino[3,4-b]indole-1-carboxylic acid,
7-(methylthio)-3,4,5,10-tetrahydro-1H-2,5-methanoazepino[3,4-b]indole-1-carboxylic acid,
6-methoxy-2,3,4,9-tetrahydro-1H-beta-carboline-1-carboxylic acid,
6-(2-oxo-2-phenylethoxy)-2,3,4,9-tetrahydro-1H-beta-carboline-1-carboxylic acid,
6-methoxy-2-methyl-2,3,4,9-tetrahydro-1H-beta-carboline-1-carboxylic acid,
2,2,2-trifluoroethyl 7-methoxy-3,4,5,10-tetrahydro-1H-2,5-methanoazepino[3,4-b]indole-1-carboxylate,
6-methoxy-2,3,4,9-tetrahydro-1H-beta-carboline-1-carboxylic acid,
7-hydroxy-3,4,5,10-tetrahydro-1H-2,5-methanoazepino[3,4-b]indole-1-carboxylic acid,
6-hydroxy-2-methyl-2,3,4,9-tetrahydro-1H-beta-carboline-1-carboxylic acid,
7-methoxy-3,4,5,10-tetrahydro-1H-2,5-methanoazepino[3,4-b]indole-1-carboxylic acid,
6-methoxy-2-methyl-2,3,4,9-tetrahydro-1H-beta-carboline-1-carboxylic acid,
2,3-dihydroxypropyl 7-methoxy-3,4,5,10-tetrahydro-1H-2,5-methanoazepino[3,4-b]indole-1-carboxylate,
4-ethyl-6-methoxy-2,3,4,9-tetrahydro-1H-beta-carboline-1-carboxylic acid,
6-methoxy-4-methyl-2,3,4,9-tetrahydro-1H-beta-carboline-1-carboxylic acid,
pyridin-4-ylmethyl 7-methoxy-3,4,5,10-tetrahydro-1H-2,5-methanoazepino[3,4-b]indole-1-carboxylate,
3-(hydroxymethyl)-6-methoxy-2,3,4,9-tetrahydro-1H-beta-carboline-1-carboxylic acid,
2-fluoroethyl 7-methoxy-3,4,5,10-tetrahydro-1H-2,5-methanoazepino[3,4-b]indole-1-carboxylate,
allyl 7-methoxy-3,4,5,10-tetrahydro-1H-2,5-methanoazepino[3,4-b]indole-1-carboxylate,
7-methoxy-3,4,5,10-tetrahydroazepino[3,4-b]indol-1 (2H)-one,
benzyl 7-methoxy-3,4,5,10-tetrahydro-1H-2,5-methanoazepino[3,4-b]indole-1-carboxylate,
7-methoxy-3,4,5,10-tetrahydro-1H-2,5-methanoazepino[3,4-b]indole-1-carboxylic acid,
2-(methylthio)ethyl 7-methoxy-3,4,5,10-tetrahydro-1H-2,5-methanoazepino[3,4-b]indole-1-carboxylate,
6-hydroxy-2,3,4,9-tetrahydro-1H-beta-carboline-1-carboxylic acid,
7-methoxy-2,3,4,9-tetrahydro-1H-beta-carboline-1-carboxylic acid,
2-methoxyethyl 7-methoxy-3,4,5,10-tetrahydro-1H-2,5-methanoazepino[3,4-b]indole-1-carboxylate,
sodium 6-(benzyloxy)-2-methyl-2,3,4,9-tetrahydro-1H-beta-carboline-1-carboxylate,
8-methoxy-1,3,4,5,6,11-hexahydro-2,6-methanoazocino[3,4-b]indole-1-carboxylic acid,
methyl 7-hydroxy-3,4,5,10-tetrahydro-1H-2,5-methanoazepino[3,4-b]indole-1-carboxylate,
2-hydroxyethyl 7-methoxy-3,4,5,10-tetrahydro-1H-2,5-methanoazepino[3,4-b]indole-1-carboxylate,
2-ethyl-6-methoxy-2,3,4,9-tetrahydro-1H-beta-carboline-1-carboxylic acid,
3-(aminomethyl)-6-methoxy-2,3,4,9-tetrahydro-1H-beta-carboline-1-carboxylic acid,
2-methoxyethyl 7-methoxy-3,4,5,10-tetrahydro-1H-2,5-methanoazepino[3,4-b]indole-1-carboxylate,
2-methoxyethyl 7-hydroxy-3,4,5,10-tetrahydro-1H-2,5-methanoazepino[3,4-b]indole-1-carboxylate,
6-(methoxycarbonyl)-2,3,4,9-tetrahydro-1H-beta-carboline-1-carboxylic acid,
6-methoxy-3-methyl-2,3,4,9-tetrahydro-1H-beta-carboline-1-carboxylic acid,
benzyl 7-methoxy-3,4,5,10-tetrahydro-1H-2,5-methanoazepino[3,4-b]indole-1-carboxylate,
benzyl 7-methoxy-3,4,5,10-tetrahydro-1H-2,5-methanoazepino[3,4-b]indole-1-carboxylate,
6-(2-ethoxy-2-oxoethoxy)-2,3,4,9-tetrahydro-1H-beta-carboline-1-carboxylic acid,
6-methoxy-3,3-dimethyl-2,3,4,9-tetrahydro-1H-beta-carboline-1-carboxylic acid,
6-methoxy-4-propyl-2,3,4,9-tetrahydro-1H-beta-carboline-1-carboxylic acid,
3,4,5,10-tetrahydro-1H-2,5-methanoazepino[3,4-b]indole-1-carboxylic acid,
6-(2-methoxy-2-oxo-1-phenylethoxy)-2,3,4,9-tetrahydro-1H-beta-carboline-1-carboxylic acid,
methyl 7-methoxy-3,4,5,10-tetrahydro-1H-2,5-methanoazepino[3,4-b]indole-1-carboxylate,
isopropyl 7-methoxy-3,4,5,10-tetrahydro-1H-2,5-methanoazepino[3,4-b]indole-1-carboxylate,
6-butoxy-2,3,4,9-tetrahydro-1H-beta-carboline-1-carboxylic acid,
propyl 7-methoxy-3,4,5,10-tetrahydro-1H-2,5-methanoazepino[3,4-b]indole-1-carboxylate,
benzyl 7-hydroxy-3,4,5,10-tetrahydro-1H-2,5-methanoazepino[3,4-b]indole-1-carboxylate,
6-(benzyloxy)-2,3,4,9-tetrahydro-1H-beta-carboline-1-carboxylic acid,
1-phenylethyl 7-methoxy-3,4,5,10-tetrahydro-1H-2,5-methanoazepino[3,4-b]indole-1-carboxylate,
6-methoxy-4-(trifluoromethyl)-2,3,4,9-tetrahydro-1H-beta-carboline-1-carboxylic acid,
6-methoxy-2,3,4,9-tetrahydro-1H-beta-carboline-1-carboxylic acid,
isopropyl 7-methoxy-3,4,5,10-tetrahydro-1H-2,5-methanoazepino[3,4-b]indole-1-carboxylate,
6-methoxy-2,3,4,9-tetrahydro-1H-beta-carbolin-1-one,
6-(2-methoxy-2-oxoethoxy)-2,3,4,9-tetrahydro-1H-beta-carboline-1-carboxylic acid,
4-benzyl-6-methoxy-2,3,4,9-tetrahydro-1H-beta-carboline-1-carboxylic acid,
6-[(2-chlorobenzyl)oxy]-2,3,4,9-tetrahydro-1H-beta-carboline-1-carboxylic acid,
6-methoxy-4,4-dimethyl-2,3,4,9-tetrahydro-1H-beta-carboline-1-carboxylic acid,
propyl 7-hydroxy-3,4,5,10-tetrahydro-1H-2,5-methanoazepino[3,4-b]indole-1-carboxylate,
6-(3-chloropropoxy)-2,3,4,9-tetrahydro-1H-beta-carboline-1-carboxylic acid,
6-(4-chlorobutoxy)-2,3,4,9-tetrahydro-1H-beta-carboline-1-carboxylic acid,
6-[(3-chlorobenzyl)oxy]-2,3,4,9-tetrahydro-1H-beta-carboline-1-carboxylic acid,
8,9,10,11-tetrahydro-7H-pyrido[3′,4′:4,5]pyrrolo[2,3-f]isoquinolin-7-one trifluoroacetate,
3-(aminomethyl)-6-methoxy-2,3,4,9-tetrahydro-1H-beta-carbolin-1-one trifluoroacetate,
3-(aminomethyl)-6-methoxy-2,3,4,9-tetrahydro-1H-beta-carbolin-1-one hydrochloride,
6-methoxy-3-{3-[(2-phenylethyl)amino]propyl}-2,3,4,9-tetrahydro-1H-beta-carbolin-1-one trifluoroacetate,
(1E)-6-methoxy-2,3,4,9-tetrahydro-1H-carbazol-1-one oxime,
(1Z)-6-methoxy-2,3,4,9-tetrahydro-1H-carbazol-1-one oxime,
6-methoxy-3-{3-[(3-phenylpropyl)amino]propyl}-2,3,4,9-tetrahydro-1H-beta-carbolin-1-one trifluoroacetate,
methyl 1-oxo-2,3,4,5-tetrahydro-1H-pyrido[4,3-b]indole-6-carboxylate,
3-(hydroxymethyl)-6-methoxy-2,3,4,9-tetrahydro-1H-beta-carbolin-1-one,
3-(3-aminopropyl)-6-methoxy-2,3,4,9-tetrahydro-1H-beta-carbolin-1-one trifluoroacetate,
3-(2-aminoethyl)-6-methoxy-2,3,4,9-tetrahydro-1H-beta-carbolin-1-one trifluoroacetate,
ethyl 1-(hydroxyimino)-2,3,4,9-tetrahydro-1H-carbazole-6-carboxylate,
2-methoxy-7,8,9,10-tetrahydrocyclohepta[b]indol-6(5H)-one oxime,
3-[3-(benzylamino)propyl]-6-methoxy-2,3,4,9-tetrahydro-1H-beta-carbolin-1-one trifluoroacetate,
6-methoxy-2,3,4,9-tetrahydro-1H-carbazol-1-one oxime,
6-iodo-2,3,4,9-tetrahydro-1H-carbazol-1-one oxime, and
6-methoxy-2-methyl-2,3,4,9-tetrahydro-1H-carbazol-1-one oxime, mixtures thereof, and pharmaceutically acceptable salts thereof.

Compounds that are encompassed by the present invention include, but are not limited to, those compounds that have an MK-2 inhibition IC₅₀ value of below 5 μM that are chosen from:
7-methoxy-3,4,5,10-tetrahydro-1H-2,5-methanoazepino[3,4-b]indole-1-carboxylic acid,
7-hydroxy-3,4,5,10-tetrahydro-1H-2,5-methanoazepino[3,4-b]indole-1-carboxylic acid,
2,3,8,10,11,12-hexahydro-1H,7H-9,12-methanoazepino[3,4-b]pyrano[3,2-e]indole-8-carboxylic acid,
7-methoxy-3,4,5,10-tetrahydro-1H-2,5-methanoazepino[3,4-b]indole-1-carboxylic acid,
7-(methylthio)-3,4,5,10-tetrahydro-1H-2,5-methanoazepino[3,4-b]indole-1-carboxylic acid,
7-methoxy-3,4,5,10-tetrahydro-1H-2,5-methanoazepino[3,4-b]indole-1-carboxylic acid,
7-(benzyloxy)-3,4,5,10-tetrahydro-1H-2,5-methanoazepino[3,4-b]indole-1-carboxylic acid,
7-(methylthio)-3,4,5,10-tetrahydro-1H-2,5-methanoazepino[3,4-b]indole-1-carboxylic acid,
6-methoxy-2,3,4,9-tetrahydro-1H-beta-carboline-1-carboxylic acid,
6-(2-oxo-2-phenylethoxy)-2,3,4,9-tetrahydro-1H-beta-carboline-1-carboxylic acid,
6-methoxy-2-methyl-2,3,4,9-tetrahydro-1H-beta-carboline-1-carboxylic acid,
2,2,2-trifluoroethyl 7-methoxy-3,4,5,10-tetrahydro-1H-2,5-methanoazepino[3,4-b]indole-1-carboxylate,
6-methoxy-2,3,4,9-tetrahydro-1H-beta-carboline-1-carboxylic acid,
7-hydroxy-3,4,5,10-tetrahydro-1H-2,5-methanoazepino[3,4-b]indole-1-carboxylic acid,
6-hydroxy-2-methyl-2,3,4,9-tetrahydro-1H-beta-carboline-1-carboxylic acid,
7-methoxy-3,4,5,10-tetrahydro-1H-2,5-methanoazepino[3,4-b]indole-1-carboxylic acid,
6-methoxy-2-methyl-2,3,4,9-tetrahydro-1H-beta-carboline-1-carboxylic acid,
2,3-dihydroxypropyl 7-methoxy-3,4,5,10-tetrahydro-1H-2,5-methanoazepino[3,4-b]indole-1-carboxylate,
4-ethyl-6-methoxy-2,3,4,9-tetrahydro-1H-beta-carboline-1-carboxylic acid,
6-methoxy-4-methyl-2,3,4,9-tetrahydro-1H-beta-carboline-1-carboxylic acid,
pyridin-4-ylmethyl 7-methoxy-3,4,5,10-tetrahydro-1H-2,5-methanoazepino[3,4-b]indole-1-carboxylate,
3-(hydroxymethyl)-6-methoxy-2,3,4,9-tetrahydro-1H-beta-carboline-1-carboxylic acid,
2-fluoroethyl 7-methoxy-3,4,5,10-tetrahydro-1H-2,5-methanoazepino[3,4-b]indole-1-carboxylate,
allyl 7-methoxy-3,4,5,10-tetrahydro-1H-2,5-methanoazepino[3,4-b]indole-1-carboxylate,
7-methoxy-3,4,5,10-tetrahydroazepino[3,4-b]indol-1 (2H)-one,
benzyl 7-methoxy-3,4,5,10-tetrahydro-1H-2,5-methanoazepino[3,4-b]indole-1-carboxylate,
7-methoxy-3,4,5,10-tetrahydro-1H-2,5-methanoazepino[3,4-b]indole-1-carboxylic acid,
2-(methylthio)ethyl 7-methoxy-3,4,5,10-tetrahydro-1H-2,5-methanoazepino[3,4-b]indole-1-carboxylate,
6-hydroxy-2,3,4,9-tetrahydro-1H-beta-carboline-1-carboxylic acid,
7-methoxy-2,3,4,9-tetrahydro-1H-beta-carboline-1-carboxylic acid,
2-methoxyethyl 7-methoxy-3,4,5,10-tetrahydro-1H-2,5-methanoazepino[3,4-b]indole-1-carboxylate,
sodium 6-(benzyloxy)-2-methyl-2,3,4,9-tetrahydro-1H-beta-carboline-1-carboxylate,
8,9,10,11-tetrahydro-7H-pyrido[3′,4′:4,5]pyrrolo[2,3-f]isoquinolin-7-one,
3-(aminomethyl)-6-methoxy-2,3,4,9-tetrahydro-1H-beta-carbolin-1-one,
6-methoxy-3-{3-[(2-phenylethyl)amino]propyl}-2,3,4,9-tetrahydro-1H-beta-carbolin-1-one,
(1E)-6-methoxy-2,3,4,9-tetrahydro-1H-carbazol-1-one oxime,
(1Z)-6-methoxy-2,3,4,9-tetrahydro-1H-carbazol-1-one oxime,
6-methoxy-3-{3-[(3-phenylpropyl)amino]propyl}-2,3,4,9-tetrahydro-1H-beta-carbolin-1-one, and
methyl 1-oxo-2,3,4,5-tetrahydro-1H-pyrido[4,3-b]indole-6-carboxylate, mixtures thereof, and pharmaceutically acceptable salts thereof.

The present invention encompasses a method for treating or preventing a disease or disorder in a subject, which disease or disorder is one that can be treated or prevented by inhibiting the activity of MK-2, the method comprising administering to the subject a compound, or a pharmaceutically acceptable salt or prodrug thereof, wherein the compound is chosen from:
7-methoxy-3,4,5,10-tetrahydro-1H-2,5-methanoazepino[3,4-b]indole-1-carboxylic acid,
7-hydroxy-3,4,5,10-tetrahydro-1H-2,5-methanoazepino[3,4-b]indole-1-carboxylic acid,
2,3,8,10,11,12-hexahydro-1H,7H-9,12-methanoazepino[3,4-b]pyrano[3,2-e]indole-8-carboxylic acid,
7-methoxy-3,4,5,10-tetrahydro-1H-2,5-methanoazepino[3,4-b]indole-1-carboxylic acid,
7-(methylthio)-3,4,5,10-tetrahydro-1H-2,5-methanoazepino[3,4-b]indole-1-carboxylic acid,
7-methoxy-3,4,5,10-tetrahydro-1H-2,5-methanoazepino[3,4-b]indole-1-carboxylic acid,
7-(benzyloxy)-3,4,5,10-tetrahydro-1H-2,5-methanoazepino[3,4-b]indole-1-carboxylic acid,
7-(methylthio)-3,4,5,10-tetrahydro-1H-2,5-methanoazepino[3,4-b]indole-1-carboxylic acid,
6-methoxy-2,3,4,9-tetrahydro-1H-beta-carboline-1-carboxylic acid,
6-(2-oxo-2-phenylethoxy)-2,3,4,9-tetrahydro-1H-beta-carboline-1-carboxylic acid,
6-methoxy-2-methyl-2,3,4,9-tetrahydro-1H-beta-carboline-1-carboxylic acid,
2,2,2-trifluoroethyl 7-methoxy-3,4,5,10-tetrahydro-1H-2,5-methanoazepino[3,4-b]indole-1-carboxylate,
6-methoxy-2,3,4,9-tetrahydro-1H-beta-carboline-1-carboxylic acid,
7-hydroxy-3,4,5,10-tetrahydro-1H-2,5-methanoazepino[3,4-b]indole-1-carboxylic acid,
6-hydroxy-2-methyl-2,3,4,9-tetrahydro-1H-beta-carboline-1-carboxylic acid,
7-methoxy-3,4,5,10-tetrahydro-1H-2,5-methanoazepino[3,4-b]indole-1-carboxylic acid,
6-methoxy-2-methyl-2,3,4,9-tetrahydro-1H-beta-carboline-1-carboxylic acid,
2,3-dihydroxypropyl 7-methoxy-3,4,5,10-tetrahydro-1H-2,5-methanoazepino[3,4-b]indole-1-carboxylate,
4-ethyl-6-methoxy-2,3,4,9-tetrahydro-1H-beta-carboline-1-carboxylic acid, and
6-methoxy-4-methyl-2,3,4,9-tetrahydro-1H-beta-carboline-1-carboxylic acid,
8,9,10,11-tetrahydro-7H-pyrido[3′,4′:4,5]pyrrolo[2,3-f]isoquinolin-7-one trifluoroacetate,
3-(aminomethyl)-6-methoxy-2,3,4,9-tetrahydro-1H-beta-carbolin-1-one, mixtures thereof, and pharmaceutically acceptable salts thereof.

The present invention encompasses a method for treating or preventing a disease or disorder in a subject, which disease or disorder is one that can be treated or prevented by inhibiting the activity of MK-2, the method comprising administering to the subject a compound, or a pharmaceutically acceptable salt or prodrug thereof, wherein the compound is chosen from:
7-methoxy-3,4,5,10-tetrahydro-1H-2,5-methanoazepino[3,4-b]indole-1-carboxylic acid,
7-hydroxy-3,4,5,10-tetrahydro-1H-2,5-methanoazepino[3,4-b]indole-1-carboxylic acid, and
2,3,8,10,11,12-hexahydro-1H,7H-9,12-methanoazepino[3,4-b]pyrano[3,2-e]indole-8-carboxylic acid,
8,9,10,11-tetrahydro-7H-pyrido[3′,4′:4,5]pyrrolo[2,3-f]isoquinolin-7-one,
3-(aminomethyl)-6-methoxy-2,3,4,9-tetrahydro-1H-beta-carbolin-1-one,
3-(aminomethyl)-6-methoxy-2,3,4,9-tetrahydro-1H-beta-carbolin-1-one, mixtures thereof, and pharmaceutically acceptable salts thereof.

In still other embodiments, the present invention is directed to a novel compound, or a pharmaceutically acceptable salt or isomer thereof, the compound having the structure: 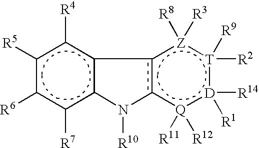
wherein:
Q, D, T and Z are selected from carbon, sulfur, or nitrogen;
when Q is sulfur, R¹² is oxo, and R¹¹ is optionally oxo or is absent;
when T is sulfur, R² is oxo, and R⁹ is optionally oxo or is absent;
when Z is nitrogen, D is nitrogen, and R² and R¹² are oxo;
R¹ is selected from H, alkyl, alkenyl, alkynyl, allyl, acetyl, amino, alkoxy, hydroxy, oxo, carboxy, allylalkyl, benzyl, 2-ethylalkyl, haloacetyl, t-butyldialkylsilyloxyalkyl, alkoxycarbonylaminoalkyl, haloalkylbenzyl, haloalkylcarbonyl, alkylcarbonyl, alkoxycarbonyl, arylcarbonyl, benzyloxycarbonyl, arylalkenylamino, alkoxyheterocyclylcarbonyl, haloarylcarbonyl, or substituted or unsubstituted alkyl, which, if substituted, has one or more substituent groups selected from H, halo, amino, aryl, or heteroaryl;
R² is selected from H, alkyl, alkoxy, hydroxy, carbonyl, oxo, benzyl, hydroxyalkyl, dialkylamino, carboxyl, amino, halo, alkoxycarbonyl, alkoxycarbonyl-R¹⁵, aminoalkyl, haloalkyl, hydroxyalkylbenzyl, haloalkylbenzyl, arylalkenylamino, haloalkoxybenzyl, cyanoalkyl, heterocyclylalkyl, arylalkyl-R¹⁵, alkyl-SO₂—R¹⁵, substituted or unsubstituted aryl, which, if substituted, has one or more substituent groups selected from H, alkyl, amino, nitro, halo, haloalkyl, or hydroxyalkyl, or substituted or unsubstituted alkyl, which, if substituted, has one or more substituent groups selected from H, halo, amino, aryl, or heteroaryl;
R³ is selected from H, oxo, alkyl, alkoxy, amino, halo, haloalkyl, hydroxy, carbonyl, alkoxycarbonyl, benzyl, aminoalkyl, hydroxyalkyl, dialkylamino, arylalkenylamino, carboxy, haloalkyl, hydroxyalkylbenzyl, haloalkylbenzyl, haloalkoxybenzyl, heterocyclylalkyl, substituted or unsubstituted aryl, which, if substituted, has one or more substituent groups selected from H, alkyl, amino, nitro, halo, haloalkyl, or hydroxyalkyl, or substituted or unsubstituted alkyl, which, if substituted, has one or more substituent groups selected from H, halo, amino, aryl, or heteroaryl, or R¹ and R³ optionally join to form an alkyl bridge between the ring atoms to which R¹ and R³ are attached, or R² and R³ optionally join to form a cycloalkyl ring;
R⁴, R⁶ and R⁷ are independently selected from H, alkyl, benzyl, halo, haloalkyl, alkoxy, haloalkoxy, carboxyl, CO₂—R⁵, carboxyl, nitro, amino, alkylamino, dialkylamino, benzyloxy, hydrazinocarbonyl, alkoxycarbonyl, alkoxycarbonylheterocyclylcarbonyl, hydroxy, or substituted or unsubstituted 6-membered heterocyclylcarbonyl, which, if substituted, has one or more substituent groups selected from H or alkoxycarbonyl, or R⁶ and R⁷ optionally join to form a heteroaryl ring;
R⁵ is selected from H, alkyl, alkenyl, alkylthio, hydroxy, carboxyl, halo, alkoxy, benzylalkyl, benzylalkoxy, arylalkyl, nitro, alkoxycarbonylalkoxy, alkylthio, benzyloxy, arylalkynyl, arylcarbonylalkoxy, haloalkoxy, haloalkyl, arylalkoxy, haloarylalkoxy, benzyloxoalkoxy, alkoxycarbonylbenzyloxy, alkoxycarbonyl, alkoxyoxoalkoxy, alkoxyoxoarylalkoxy, halobenzyloxy, heterocyclylalkoxy, dialkylaminooxoalkoxy, aminocarbonyl, arylalkynyl, hydrazinocarbonyl, carbonylalkoxy, alkylsulfonylbenzyloxy, CO₂—R¹⁵, carbamoyl, naphthylalkoxy, alkoxyaryloxy, alkoxycarbonylheterocyclylalkoxy, haloalkylbenzyloxy, alkoxyacetylaminoacetyloxy, acetoxy, cycloalkylalkoxy, acetyloxy, morpholinylcarbonyl, alkoxyalkoxy, or R⁴ and R⁵ optionally join to form a heterocyclic ring;
R⁸ and R⁹ are independently optionally present and if present, are independently selected from H, oxo, carbonitrile, alkyl, or is optionally absent, or R³ and R⁸ optionally join to form a cycloalkyl ring;
R¹⁰ is selected from H, alkyl, benzyl, aryl, or hydroxyalkyl;
R¹¹ is optionally present and if present, is selected from H, oxo, alkyl, halobenzyl, benzyl, haloalkyl, 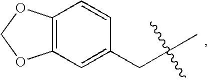
alkoxycarbonyl, carboxyalkyl, benzodioxinylalkyl, substituted or unsubstituted aryl or arylalkyl, which, if substituted, have one or more substituent groups selected from H, alkyl, or alkoxy;
R¹² is optionally present and if present, is selected from H, ═S, oxo, alkyl, carbonyl-R¹³, carboxyl-R¹³, ketone, cyano, cyanoalkyl, alkoxycarbonyl, aminoalkyl, alkoxycarbonylalkyl, carbamyl, hydroxycarbamyl, haloalkyl, heterocyclyl, alkylacetate, heterocyclylalkyl, alkoxycarbonylalkyl, hydroxyalkyl, dihydroxyalkyl, cyanoalkyl, ═N—OR¹⁵, ═N—N(R¹⁵)₂, or hydroxyamino, or R¹ and R¹² optionally join to form a substituted or unsubstituted 5 or 6-membered heteroaryl ring, which, if substituted, has one or more substituent groups selected from H, alkyl, oxo, or alkoxycarbonylalkyl, or R¹¹ and R¹² optionally join to form a ring system having the structure: 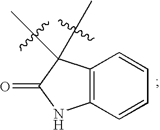
R¹³ is optionally present and if present, is selected from H, alkyl, alkoxy, amino, alkynyl, allyl, alkenyl, hydrazine, benzyl, glycine, allylalkyl, haloalkyl, hydroxyalkyl, hydroxyamino, dihydroxyalkyl, alkoxyalkyl, alkoxyamino, alkylthioalkyl, cycloalkyl, cyclohexylalkyl, amino, alkylamino, benzylalkyl, benzylamino, benzyloxycarbonylalkylamino, dialkylamino, adamantyl, haloalkoxybenzyl, alkoxyaminoalkyl, haloalkylarylalkyl, heterocyclylalkyl, alkoxyalkylamino, arylthioalkyl, substituted or unsubstituted aryl or arylalkyl, which, if substituted, have one or more substituent groups selected from H, alkyl, alkoxy, amino, nitro, halo, haloalkyl, or hydroxyalkyl;
R¹⁴ is optionally present and if present, is selected from H or alkyl; and
R¹⁵ is optionally present and if present, is selected from H, alkyl, alkoxy, or aminoalkyl.

It should be understood that salts and prodrugs of the beta-carboline compounds that are described herein, as well as isomeric forms, tautomers, racemic mixtures of the compounds, stereoisomers, and enantiomers, are to be considered to be included within the description of the compound.

The present invention encompasses a beta-carboline MK-2 inhibiting compound that provides a TNFα release IC₅₀ value of below 200 μM in an in vitro cell assay. More preferably, the MK-2 inhibiting compound provides a TNFα release IC₅₀ values of below 50 μM in an in vitro cell assay. Even more preferably, the MK-2 inhibiting compound provides a TNFα release IC₅₀ values of below 10 μM in an in vitro cell assay. Even more preferably still, the MK-2 inhibiting compound provides a TNFα release IC₅₀ values of below 1 μM in an in vitro cell assay.

The present invention encompasses a beta-carboline MK-2 inhibiting compound that provides a degree of inhibition of TNFα in a rat LPS assay of at least about 25%. More preferably, the MK-2 inhibiting compound provides a degree of inhibition of TNFα in a rat LPS assay of above 50%. Even more preferably, the MK-2 inhibiting compound provides a degree of inhibition of TNFα in a rat LPS assay of above 70%. Even more preferably still, the MK-2 inhibiting compound provides a degree of inhibition of TNFα in a rat LPS assay of above 80%.

The present invention encompasses a beta-carboline MK-2 inhibiting compound that provides an MK-2 inhibition IC₅₀ value of below 200 μM. More preferably, the MK-2 inhibiting compound provides an MK-2 inhibition IC₅₀ value of below 50 μM. Even more preferably, the MK-2 inhibiting compound provides an MK-2 inhibition IC₅₀ value of below 20 μM. Even more preferably still, the MK-2 inhibiting compound provides an MK-2 inhibition IC₅₀ value of below 1 μM.

The present invention encompasses a method for treating or preventing a disease or disorder in a subject, which disease or disorder is one that can be treated or prevented by inhibiting the activity of MK-2, the method comprising administering to the subject any of the compounds described herein, or a pharmaceutically acceptable salt or prodrug thereof, wherein the disease or disorder that can be treated or prevented by inhibiting the activity of MK-2 is a disease or disorder that is mediated by TNFα.

Thus, one embodiment of the present invention includes a method for the treatment, prevention, or amelioration of an inflammatory disease or disorder which is mediated by TNFα in a subject in need of such treatment, prevention, or amelioration.

The method comprises administering to the subject an MK-2 inhibitor. The MK-2 inhibitor can be one or more of the beta-carboline compounds that are described above. The present method also includes the administration to the subject of pharmaceutically acceptable prodrugs or salts of a beta-carboline MK-1 inhibitor compound. In a preferred embodiment, the beta-carboline MK-2 inhibitor is administered to the subject in a MK-2 kinase-inhibitory amount. An MK-2 kinase-inhibitory amount of the beta-carboline MK-2 inhibitor is preferably a therapeutically effective amount.

The method of the present invention is useful for, but not limited to, the prevention and/or treatment of diseases and disorders that are mediated by TNFα and/or mediated by MK-2, including pain, inflammation and/or arthritis. For example, the compounds described herein would be useful for the treatment of any inflammation-related disorder described below, such as an analgesic in the treatment of pain and headaches, or as an antipyretic for the treatment of fever. The compounds described herein would also be useful for the treatment of an inflammation-related disorder in a subject suffering from such an inflammation-associated disorder.

As used herein, the terms “treating”, “treatment”, “treated”, or “to treat,” mean to alleviate symptoms, eliminate the causation either on a temporary or permanent basis. The term “treatment” includes alleviation, elimination of causation of pain and/or inflammation associated with, but not limited to, any of the diseases or disorders described herein. The terms “prevent”, “prevention”, “prevented”, or “to prevent,” mean to prevent or to slow the appearance of symptoms associated with, but not limited to, any of the diseases or disorders described herein. For methods of prevention, the subject is any subject, and preferably is a subject that is in need of prevention of a TNFα-mediated inflammatory disease or disorder.

The term “subject” for purposes of treatment includes any human or animal subject who is in need of the prevention of, or who has a TNFα-mediated inflammatory disease or disorder. The subject is typically a mammal. “Mammal”, as that term is used herein, refers to any animal classified as a mammal, including humans, domestic and farm animals, and zoo, sports, or pet animals, such as dogs, horses, cats, cattle, etc., Preferably, the mammal is a human.

The subject may be a human subject who is at risk for a TNFα-mediated inflammatory disease or disorder, such as those described above. The subject may be at risk due to genetic predisposition, sedentary lifestyle, diet, exposure to disorder-causing agents, exposure to pathogenic agents and the like.

In preferred embodiments, the methods and compositions of the present invention encompass the prevention and/or treatment of pain, inflammation and inflammation-related disorders.

In other preferred embodiments, the methods and compositions of the present invention encompass the treatment of any one or more of the disorders selected from the group consisting of connective tissue and joint disorders, neoplasia disorders, cardiovascular disorders, otic disorders, ophthalmic disorders, respiratory disorders, gastrointestinal disorders, angiogenesis-related disorders, immunological disorders, allergic disorders, nutritional disorders, infectious diseases and disorders, endocrine disorders, metabolic disorders, neurological and neurodegenerative disorders, psychiatric disorders, hepatic and biliary disorders, musculoskeletal disorders, genitourinary disorders, gynecologic and obstetric disorders, injury and trauma disorders, surgical disorders, dental and oral disorders, sexual dysfunction disorders, dermatologic disorders, hematological disorders, and poisoning disorders.

As used herein, the terms “neoplasia” and “neoplasia disorder”, used interchangeably herein, refer to new cell growth that results from a loss of responsiveness to normal growth controls, e.g. to “neoplastic” cell growth. Neoplasia is also used interchangeably herein with the term “cancer” and for purposes of the present invention; cancer is one subtype of neoplasia. As used herein, the term “neoplasia disorder” also encompasses other cellular abnormalities, such as hyperplasia, metaplasia and dysplasia. The terms neoplasia, metaplasia, dysplasia and hyperplasia can be used interchangeably herein and refer generally to cells experiencing abnormal cell growth.

Both of the terms, “neoplasia” and “neoplasia disorder”, refer to a “neoplasm” or tumor, which may be benign, premalignant, metastatic, or malignant. Also encompassed by the present invention are benign, premalignant, metastatic, or malignant neoplasias. Also encompassed by the present invention are benign, premalignant, metastatic, or malignant tumors. Thus, all of benign, premalignant, metastatic, or malignant neoplasia or tumors are encompassed by the present invention and may be referred to interchangeably, as neoplasia, neoplasms or neoplasia-related disorders. Tumors are generally known in the art to be a mass of neoplasia or “neoplastic” cells. Although, it is to be understood that even one neoplastic cell is considered, for purposes of the present invention to be a neoplasm or alternatively, neoplasia.

In still other preferred embodiments, the methods and compositions of the present invention encompass the prevention and treatment of the connective tissue and joint disorders selected from the group consisting of arthritis, rheumatoid arthritis, spondyloarthopathies, gouty arthritis, lumbar spondylarthrosis, carpal tunnel syndrome, canine hip dysplasia, systemic lupus erythematosus, juvenile arthritis, osteoarthritis, tendonitis and bursitis.

In other preferred embodiments, the methods and compositions of the present invention encompass the prevention and treatment of the neoplasia disorders selected from the group consisting of acral lentiginous melanoma, actinic keratoses, adenocarcinoma, adenoid cycstic carcinoma, adenomas, familial adenomatous polyposis, familial polyps, colon polyps, polyps, adenosarcoma, adenosquamous carcinoma, adrenocortical carcinoma, AIDS-related lymphoma, anal cancer, astrocytic tumors, bartholin gland carcinoma, basal cell carcinoma, bile duct cancer, bladder cancer, brain stem glioma, brain tumors, breast cancer, bronchial gland carcinomas, capillary carcinoma, carcinoids, carcinoma, carcinosarcoma, cavernous, central nervous system lymphoma, cerebral astrocytoma, cholangiocarcinoma, chondosarcoma, choriod plexus papilloma/carcinoma, clear cell carcinoma, skin cancer, brain cancer, colon cancer, colorectal cancer, cutaneous T-cell lymphoma, cystadenoma, endodermal sinus tumor, endometrial hyperplasia, endometrial stromal sarcoma, endometrioid adenocarcinoma, ependymal, epitheloid, esophageal cancer, Ewing's sarcoma, extragonadal germ cell tumor, fibrolamellar, focal nodular hyperplasia, gallbladder cancer, gastrinoma, germ cell tumors, gestational trophoblastic tumor, glioblastoma, glioma, glucagonoma, hemangiblastomas, hemangioendothelioma, hemangiomas, hepatic adenoma, hepatic adenomatosis, hepatocellular carcinoma, Hodgkin's lymphoma, hypopharyngeal cancer, hypothalamic and visual pathway glioma, insulinoma, intaepithelial neoplasia, interepithelial squamous cell neoplasia, intraocular melanoma, invasive squamous cell carcinoma, large cell carcinoma, islet cell carcinoma, Kaposi's sarcoma, kidney cancer, laryngeal cancer, leiomyosarcoma, lentigo maligna melanomas, leukemia-related disorders, lip and oral cavity cancer, liver cancer, lung cancer, lymphoma, malignant mesothelial tumors, malignant thymoma, medulloblastoma, medulloepithelioma, melanoma, meningeal, merkel cell carcinoma, mesothelial, metastatic carcinoma, mucoepidermoid carcinoma, multiple myeloma/plasma cell neoplasm, mycosis fungoides, myelodysplastic syndrome, myeloproliferative disorders, nasal cavity and paranasal sinus cancer, nasopharyngeal cancer, neuroblastoma, neuroepithelial adenocarcinoma nodular melanoma, non-Hodgkin's lymphoma, oat cell carcinoma, oligodendroglial, oral cancer, oropharyngeal cancer, osteosarcoma, pancreatic polypeptide, ovarian cancer, ovarian germ cell tumor, pancreatic cancer, papillary serous adenocarcinoma, pineal cell, pituitary tumors, plasmacytoma, pseudosarcoma, pulmonary blastoma, parathyroid cancer, penile cancer, pheochromocytoma, pineal and supratentorial primitive neuroectodermal tumors, pituitary tumor, plasma cell neoplasm, pleuropulmonary blastoma, prostate cancer, rectal cancer, renal cell carcinoma, retinoblastoma, rhabdomyosarcoma, sarcoma, serous carcinoma, small cell carcinoma, small intestine cancer, soft tissue carcinomas, somatostatin-secreting tumor, squamous carcinoma, squamous cell carcinoma, submesothelial, superficial spreading melanoma, supratentorial primitive neuroectodermal tumors, thyroid cancer, undifferentiatied carcinoma, urethral cancer, uterine sarcoma, uveal melanoma, verrucous carcinoma, vaginal cancer, vipoma, vulvar cancer, Waldenstrom's macroglobulinemia, well differentiated carcinoma, and Wilm's tumor.

In other preferred embodiments, the methods and compositions of the present invention encompass the prevention and treatment of the cardiovascular disorders selected from the group consisting of myocardial ischemia, hypertension, hypotension, heart arrhythmias, pulmonary hypertension, hypokalemia, cardiac ischemia, myocardial infarction, cardiac remodeling, cardiac fibrosis, myocardial necrosis, aneurysm, arterial fibrosis, embolism, vascular plaque inflammation, vascular plaque rupture, bacterial-induced inflammation and viral induced inflammation, edema, swelling, fluid accumulation, cirrhosis of the liver, Bartter's syndrome, myocarditis, arteriosclerosis, atherosclerosis, calcification (such as vascular calcification and valvar calcification), coronary artery disease, heart failure, congestive heart failure, shock, arrhythmia, left ventricular hypertrophy, angina, diabetic nephropathy, kidney failure, eye damage, vascular diseases, migraine headaches, aplastic anemia, cardiac damage, diabetic cardiac myopathy, renal insufficiency, renal injury, renal arteriopathy, peripheral vascular disease, left ventricular hypertrophy, cognitive dysfunction, stroke, and headache.

In other preferred embodiments, the methods and compositions of the present invention encompass the prevention and treatment of the metabolic disorders selected from the group consisting of obesity, overweight, type I and type II diabetes, hypothyroidism, and hyperthyroidism.

In other preferred embodiments, the methods and compositions of the present invention encompass the prevention and treatment of the respiratory disorders selected from the group consisting of asthma, bronchitis, chronic obstructive pulmonary disease (COPD), cystic fibrosis, pulmonary edema, pulmonary embolism, pneumonia, pulmonary sarcoisosis, silicosis, pulmonary fibrosis, respiratory failure, acute respiratory distress syndrome and emphysema.

In other preferred embodiments, the methods and compositions of the present invention encompass the prevention and treatment of the angiogenesis-related disorders selected from the group consisting of angiofibroma, neovascular glaucoma, arteriovenous malformations, arthritis, osler-weber syndrome, atherosclerotic plaques, psoriasis, corneal graft neovascularization, pyogenic granuloma, delayed wound healing, retrolental fibroplasias, diabetic retinopathy, scleroderma, granulations, solid tumors, hemangioma, trachoma, hemophilic joints, vascular adhesions, hypertrophic scars, age-related macular degeneration, coronary artery disease, stroke, cancer, AIDS complications, ulcers and infertility.

In other preferred embodiments, the methods and compositions of the present invention encompass the prevention and treatment of the infectious diseases and disorders selected from the group consisting of viral infections, bacterial infections, prion infections, spirochetes infections, mycobacterial infections, rickettsial infections, chlamydial infections, parasitic infections and fungal infections.

In still further embodiments, the methods and compositions of the present invention encompass the prevention and treatment of the infectious diseases and disorders selected from the group consisting of hepatitis, HIV (AIDS), small pox, chicken pox, common cold, bacterial influenza, viral influenza, warts, oral herpes, genital herpes, herpes simplex infections, herpes zoster, bovine spongiform encephalopathy, septicemia, streptococcus infections, staphylococcus infections, anthrax, severe acquired respiratory syndrome (SARS), malaria, African sleeping sickness, yellow fever, chlamydia, botulism, canine heartworm, rocky mountain spotted fever, lyme disease, cholera, syphilis, gonorrhea, encephalitis, pneumonia, conjunctivitis, yeast infections, rabies, dengue fever, Ebola, measles, mumps, rubella, West Nile virus, meningitis, gastroenteritis, tuberculosis, hepatitis, and scarlet fever.

In other preferred embodiments, the methods and compositions of the present invention encompass the prevention and treatment of the neurological and neurodegenerative disorders selected from the group consisting of headaches, migraine headaches, Alzheimer's disease, Parkinson's disease, dementia, memory loss, senility, amyotrophy, ALS, amnesia, seizures, multiple sclerosis, muscular dystrophies, epilepsy, schizophrenia, depression, anxiety, attention deficit disorder, hyperactivity, bulimia, anorexia nervosa, anxiety, autism, phobias, spongiform encephalopathies, Creutzfeldt-Jakob disease, Huntington's Chorea, ischemia, obsessive-compulsive disorder, manic depression, bipolar disorders, drug addiction, alcoholism and smoking addiction.

In other preferred embodiments, the methods and compositions of the present invention encompass the prevention and treatment of the dermatological disorders selected from the group consisting of acne, psoriasis, eczema, burns, poison ivy, poison oak and dermatitis.

In other preferred embodiments, the methods and compositions of the present invention encompass the prevention and treatment of the surgical disorders selected from the group consisting of pain and swelling following surgery, infection following surgery and inflammation following surgery.

In other preferred embodiments, the methods and compositions of the present invention encompass the prevention and treatment of the gastrointestinal disorders selected from the group consisting of inflammatory bowel disease, irritable bowel syndrome, Crohn's disease, gastritis, irritable bowel syndrome, diarrhea, constipation, dysentery, ulcerative colitis, gastric esophageal reflux, gastric ulcers, gastric varices, ulcers, and heartburn.

In other preferred embodiments, the methods and compositions of the present invention encompass the prevention and treatment of the otic disorders selected from the group consisting of otic pain, inflammation, otorrhea, otalgia, fever, otic bleeding, Lermoyez's syndrome, Meniere's disease, vestibular neuronitis, benign paroxysmal positional vertigo, herpes zoster oticus, Ramsay Hunt's syndrome, viral neuronitis, ganglionitis, geniculate herpes, labyrinthitis, purulent labyrinthitis, viral endolymphatic labyrinthitis, perilymph fistulas, noise-induced hearing loss, presbycusis, drug-induced ototoxicity, acoustic neuromas, aerotitis media, infectious myringitis, bullous myringitis, otitis media, otitis media with effusion, acute otitis media, secretory otitis media, serous otitis media, acute mastoiditis, chronic otitis media, otitis extema, otosclerosis, squamous cell carcinoma, basal cell carcinoma, nonchromaffin paragangliomas, chemodectomas, globus jugulare tumors, globus tympanicum tumors, external otitis, perichondritis, aural eczematoid dermatitis, malignant external otitis, subperichondrial hematoma, ceruminomas, impacted cerumen, sebaceous cysts, osteomas, keloids, otalgia, tinnitus, vertigo, tympanic membrane infection, typanitis, otic furuncles, otorrhea, acute mastoiditis, petrositis, conductive and sensorineural hearing loss, epidural abscess, lateral sinus thrombosis, subdural empyema, otitic hydrocephalus, Dandy's syndrome, bullous myringitis, cerumen-impacted, diffuse external otitis, foreign bodies, keratosis obturans, otic neoplasm, otomycosis, trauma, acute barotitis media, acute eustachian tube obstruction, post-otic surgery, postsurgical otalgia, cholesteatoma, conductive and sensorineural hearing loss, epidural abscess, lateral sinus thrombosis, subdural empyema and otitic hydrocephalus.

In other preferred embodiments, the methods and compositions of the present invention encompass the prevention and treatment of the ophthalmic disorders selected from the group consisting of retinopathies, uveitis, ocular photophobia, acute injury to the eye tissue, conjunctivitis, age-related macular degeneration diabetic retinopathy, detached retina, glaucoma, vitelliform macular dystrophy type 2, gyrate atrophy of the choroid and retina, conjunctivitis, corneal infection, fuchs' dystrophy, iridocorneal endothelial syndrome, keratoconus, lattice dystrophy, map-dot-fingerprint dystrophy, ocular herpes, pterygium, myopia, hyperopia, and cataracts.

In other preferred embodiments, the methods and compositions of the present invention encompass the prevention and treatment of menstrual cramps, kidney stones, minor injuries, wound healing, vaginitis, candidiasis, sinus headaches, tension headaches, dental pain, periarteritis nodosa, thyroiditis, myasthenia gravis, multiple sclerosis, sarcoidosis, nephrotic syndrome, Bahcet's syndrome, polymyositis, gingivitis, hypersensitivity, swelling occurring after injury, closed head injury, liver disease, and endometriosis.

As used herein, the terms “TNFα mediated disease or disorder” are meant to include, without limitation, each of the symptoms or diseases that are mentioned below.

For example, the compounds of the present invention are useful for, but not limited to, the prevention and treatment of diseases and disorders which are mediated by TNFα. As used herein, the terms “TNFα mediated disease or disorder” or “diseases or disorders which are mediated by TNFα” are meant to include, without limitation, each of the symptoms or diseases that are described herein. For example, the beta-carboline MK-2 inhibitors of the invention would be useful to treat such TNFα mediated symptoms, diseases and disorders as pain and inflammation and/or arthritis-type disorders, including, but not limited to, rheumatoid arthritis, spondyloarthopathies, gouty arthritis, osteoarthritis, systemic lupus erythematosus and juvenile arthritis. Such beta-carboline MK-2 inhibitor compounds of the invention would be useful in the treatment of asthma, bronchitis, menstrual cramps, tendinitis, bursitis, connective tissue injuries or disorders, and skin related conditions such as psoriasis, eczema, burns and dermatitis.

The beta-carboline MK-2 inhibitor compounds that are useful in the method of the invention also would be useful to treat gastrointestinal conditions such as inflammatory bowel disease, gastric ulcer, gastric varices, Crohn's disease, gastritis, irritable bowel syndrome and ulcerative colitis and for the prevention or treatment of cancer, such as colorectal cancer. Such beta-carboline MK-2 inhibiting compounds would be useful in treating inflammation in diseases and conditions such as herpes simplex infections, HIV, pulmonary edema, kidney stones, minor injuries, wound healing, vaginitis, candidiasis, lumbar spondylanhrosis, lumbar spondylarthrosis, vascular diseases, migraine headaches, sinus headaches, tension headaches, dental pain, periarteritis nodosa, thyroiditis, aplastic anemia, Hodgkin's disease, sclerodoma, rheumatic fever, type I diabetes, myasthenia gravis, multiple sclerosis, sarcoidosis, nephrotic syndrome, Behcet's syndrome, polymyositis, gingivitis, hypersensitivity, swelling occurring after injury, myocardial ischemia, and the like.

The beta-carboline MK-2 inhibitors would also be useful in the treatment of ophthalmic diseases, such as retinitis, retinopathies, conjunctivitis, uveitis, ocular photophobia, and of acute injury to the eye tissue. These compounds would also be useful in the treatment of pulmonary inflammation, such as that associated with viral infections and cystic fibrosis. The compounds would also be useful for the treatment of certain central nervous system disorders such as cortical dementias including Alzheimer's disease.

As used herein, an “effective amount” means the dose or effective amount to be administered to a patient and the frequency of administration to the subject which is readily determined by one or ordinary skill in the art, by the use of known techniques and by observing results obtained under analogous circumstances. The dose or effective amount to be administered to a patient and the frequency of administration to the subject can be readily determined by one of ordinary skill in the art by the use of known techniques and by observing results obtained under analogous circumstances. In determining the effective amount or dose, a number of factors are considered by the attending diagnostician, including but not limited to, the potency and duration of action of the compounds used; the nature and severity of the illness to be treated as well as on the sex, age, weight, general health and individual responsiveness of the patient to be treated, and other relevant circumstances.

The phrase “therapeutically-effective” indicates the capability of an agent to prevent, or improve the severity of, the disorder, while avoiding adverse side effects typically associated with alternative therapies. The phrase “therapeutically-effective” is to be understood to be equivalent to the phrase “effective for the treatment, prevention, or amelioration”, and both are intended to qualify the amount of each agent for use in the combination therapy which will achieve the goal of improvement in the severity of cancer, cardiovascular disease, or pain and inflammation and the frequency of incidence over treatment of each agent by itself, while avoiding adverse side effects typically associated with alternative therapies.

The term “pharmacologically effective amount” shall mean that amount or dosage of a drug or pharmaceutical agent that will elicit the biological or medical response of a tissue, system, animal or human that is being sought by a researcher or clinician. This amount can be a therapeutically effective amount.

The MK-2 inhibiting activity of a beta-carboline compound can be determined by any one of several methods that are well known to those having skill in the art of enzyme activity testing. One such method is described in detail in the general methods section of the examples. In addition, the efficacy of a beta-carboline MK-2 inhibiting compound in therapeutic applications can be determined by testing for inhibition of TNFα production in cell culture and in animal model assays. In general, it is preferred that the beta-carboline MK-2 inhibiting compounds of the present invention be capable of inhibiting the production and/or the release of TNFα in cell cultures and in animal models.

In the present method, the beta-carboline MK-2 inhibitor compounds that are described herein can be used as inhibitors of MAPKAP kinase-2. When this inhibition is for a therapeutic purpose, one or more of the present compounds can be administered to a subject that is in need of MK-2 inhibition. As used herein, a “subject in need of MK-2 inhibition” is a subject who has, or who is at risk of contracting a TNFα mediated disease or disorder.

In an embodiment of the present method, a subject in need of prevention or treatment of a TNFα mediated disease or disorder is treated with one or more of the present beta-carboline compounds. In one embodiment, the subject is treated with an effective amount of the beta-carboline MK-2 inhibitor compound. The effective amount can be an amount that is sufficient for preventing or treating the TNFα mediated disease or disorder.

The beta-carboline compound that is used in the subject method can be any compound that is described above.

In the subject method, the beta-carboline MK-2 inhibitor compound can be used in any amount that is an effective amount. It is preferred, however, that the amount of the beta-carboline compound that is administered is within a range of about 0.1 mg/day per kilogram of the subject to about 150 mg/day/kg. It is more preferred that the amount of the beta-carboline compound is within a range of about 0.1 mg/day/kg to about 20 mg/day/kg. An amount that is within a range of about 0.1 mg/day/kg to about 10 mg/day/kg, is even more preferred.

When the term “about” is used herein in relation to a dosage amount of the beta-carboline compound, it is to be understood to mean an amount that is within ±0.05 mg. By way of example, “about 0.1-10 mg/day” includes all dosages within 0.05 to 10.05 mg/day.

For the purposes of calculating and expressing a dosage rate, all dosages that are expressed herein are calculated on an average amount-per-day basis irrespective of the dosage rate. For example, one 100 mg dosage of a beta-carboline MK-2 inhibitor taken once every two days would be expressed as a dosage rate of 50 mg/day. Similarly, the dosage rate of an ingredient where 50 mg is taken twice per day would be expressed as a dosage rate of 100 mg/day.

For purposes of calculation of dosage amounts, the weight of a normal adult human will be assumed to be 70 kg.

The amount or dosage of the beta-carboline MK-2 inhibitor will necessarily vary depending upon the host treated and the particular mode of administration.

Daily dosages can vary within wide limits and will be adjusted to the individual requirements in each particular case. In general, for administration to adults, an appropriate daily dosage has been described above, although the limits that were identified as being preferred may be exceeded if expedient. The daily dosage can be administered as a single dosage or in divided dosages.

Those skilled in the art will appreciate that dosages may also be determined with guidance from Goodman & Gilman's The Pharmacological Basis of Therapeutics, Ninth Edition (1996), Appendix II, pp. 1707-1711.

The frequency of dose will depend upon the half-life of the beta-carboline MK-2 inhibitor molecule. If the beta-carboline MK-2 inhibitor has a short half life (e.g., from about 2 to 10 hours) it may be necessary to give one or more doses per day. Alternatively, if the half-life is longer (e.g., from about 2 to about 15 days) it may only be necessary to give a dosage once per day, per week, or even once every 1 or 2 months.

In one embodiment of the present invention, any of the compounds described herein may be considered as novel compositions, novel therapeutic compositions and/or novel pharmaceutical compositions.

While it is possible to administer the compounds of the invention to a subject directly without any formulation, the compounds are preferably employed in the form of a pharmaceutical composition comprising a pharmaceutically acceptable carrier and at least one beta-carboline compound of the present invention.

The beta-carboline MK-2 inhibitors that are useful in the present invention can be of any purity or grade, as long as the preparation is of a quality suitable for pharmaceutical use. The beta-carboline MK-2 inhibitor can be provided in pure form, or it can be accompanied with impurities or commonly associated compounds that do not affect its physiological activity or safety.

The beta-carboline MK-2 inhibitors can be supplied in the form of a pharmaceutically active salt, a prodrug, an isomer, a tautomer, a racemic mixture, or in any other chemical form or combination that, under physiological conditions, still provides for any physiological function that the beta-carboline MK-2 inhibitor may perform. The present invention includes all possible diastereomers as well as their racemic and resolved, enantiomerically pure forms.

The compounds useful in the present invention can have no asymmetric carbon atoms, or, alternatively, the useful compounds can have one or more asymmetric carbon atoms. When the useful compounds have one or more asymmetric carbon atoms, they, therefore, include racemates and stereoisomers, such as diastereomers and enantiomers, in both pure form and in admixture. Such stereoisomers can be prepared using conventional techniques, either by reacting enantiomeric starting materials, or by separating isomers of compounds of the present invention.

Isomers may include geometric isomers, for example cis-isomers or trans-isomers across a double bond. All such isomers are contemplated among the compounds useful in the present invention. Also included in the methods, combinations and compositions of the present invention are the tautomeric forms of the described compounds.

Also included in the methods and compositions of the present invention are the prodrugs of the described compounds and the pharmaceutically-acceptable salts thereof.

The term “prodrug” refers to drug precursor compounds which, following administration to a subject and subsequent absorption, are converted to an active species in vivo via some process, such as a metabolic process. Other products from the conversion process are easily disposed of by the body. More preferred prodrugs produce products from the conversion process that are generally accepted as safe.

The compounds of the present invention can also be supplied in the form of a pharmaceutically acceptable salt. The term “pharmaceutically acceptable” is used adjectivally herein to mean that the modified noun is appropriate for use in a pharmaceutical product. The terms “pharmaceutically acceptable salt” refer to salts prepared from pharmaceutically acceptable inorganic and organic acids and bases.

Pharmaceutically acceptable inorganic bases include metallic ions. More preferred metallic ions include, but are not limited to, appropriate alkali metal salts, alkaline earth metal salts and other physiological acceptable metal ions. Salts derived from inorganic bases include aluminum, ammonium, calcium, copper, ferric, ferrous, lithium, magnesium, manganic salts, manganous, potassium, sodium, zinc, and the like and in their usual valences. Exemplary salts include aluminum, calcium, lithium, magnesium, potassium, sodium and zinc. Particularly preferred are the ammonium, calcium, magnesium, potassium, and sodium salts.

Salts derived from pharmaceutically acceptable organic non-toxic bases include salts of primary, secondary, and tertiary amines, including in part, trimethylamine, diethylamine, N,N′-dibenzylethylenediamine, chloroprocaine, choline, diethanolamine, ethylenediamine, meglumine (N-methylglucamine) and procaine; substituted amines including naturally occurring substituted amines; cyclic amines; quaternary ammonium cations; and basic ion exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

Illustrative pharmaceutically acceptable acid addition salts of the compounds of the present invention can be prepared from the following acids, including, without limitation formic, acetic, propionic, benzoic, succinic, glycolic, gluconic, lactic, maleic, malic, tartaric, citric, nitic, ascorbic, glucuronic, maleic, fumaric, pyruvic, aspartic, glutamic, benzoic, hydrochloric, hydrobromic, hydroiodic, isocitric, trifluoroacetic, pamoic, propionic, anthranilic, mesylic, oxalacetic, oleic, stearic, salicylic, p-hydroxybenzoic, nicotinic, phenylacetic, mandelic, embonic (pamoic), methanesulfonic, phosphoric, phosphonic, ethanesulfonic, benzenesulfonic, pantothenic, toluenesulfonic, 2-hydroxyethanesulfonic, sulfanilic, sulfuric, salicylic, cyclohexylaminosulfonic, algenic, β-hydroxybutyric, galactaric and galacturonic acids. Exemplary pharmaceutically acceptable salts include the salts of hydrochloric acid and trifluoroacetic acid.

All of the above salts can be prepared by those skilled in the art by conventional means from the corresponding compound of the present invention. For example, the pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts can also be found in Remington's Pharmaceutical Sciences, 17^{th} ed., Mack Publishing Company, Easton, Pa., p. 1418 (1985), the disclosure of which is hereby incorporated by reference only with regards to the lists of suitable salts.

In another embodiment of the present invention, the beta-carboline MK-2 inhibitor can be provided in a “pharmaceutically acceptable carrier” or “pharmaceutically acceptable excipient”, both of which are used interchangeably herein, to form a pharmaceutical composition.

When the beta-carboline MK-2 inhibitor is supplied along with a pharmaceutically acceptable carrier, a pharmaceutical composition can be formed. A pharmaceutical composition of the present invention may in one embodiment, be directed to a composition suitable for the prevention, treatment, or amelioration of a TNFα-mediated inflammatory disease or disorder. The pharmaceutical composition comprises a pharmaceutically acceptable carrier and a beta-carboline MK-2 inhibitor.

Pharmaceutically acceptable carriers and excipients include, but are not limited to, physiological saline, Ringer's solution, phosphate solution or buffer, buffered saline and other carriers known in the art. Pharmaceutical compositions may also include stabilizers, anti-oxidants, colorants, and diluents. Pharmaceutically acceptable carriers and additives are chosen such that side effects from the pharmaceutical compound are minimized and the performance of the compound is not canceled or inhibited to such an extent that treatment is ineffective. The pharmaceutically acceptable carrier can also be selected on the basis of the desired route of administration of the compound.

For example, in a preferred embodiment the carrier is suitable for oral administration. In some embodiments, the composition includes a carrier or additional agent that is suitable for promoting delivery of the compound to the brain. Carriers that can promote delivery of the compound to the brain can include any carrier that promotes translocation across the blood-brain barrier and any carrier that promotes uptake of the compound by neural cells. Examples of such carriers include those disclosed in U.S. Pat. No. 5,604,198 (issued to Poduslo, et al.), U.S. Pat. No. 5,827,819 (issued to Yatvin, et al.), 5,919,815 (issued to Bradley, et al.), U.S. Pat. No. 5,955,459 (issued to Bradley, et al.), and U.S. Pat. No. 5,977,174 (issued to Bradley, et al.).

The carrier should be acceptable in the sense of being compatible with the other ingredients of the composition and not be deleterious to the recipient. The carrier can be a solid or a liquid, or both, and is preferably formulated with the compound as a unit-dose composition, for example, a tablet, which can contain from 0.05% to 95% by weight of the active compound.

Other pharmacologically active substances can also be present, including other compounds of the present invention. The pharmaceutical compositions of the invention can be prepared by any of the well-known techniques of pharmacy, consisting essentially of admixing the components.

The MK-2 inhibitors can be administered by any conventional means available for use in conjunction with pharmaceuticals, either as individual therapeutic compounds or as a combination of therapeutic compounds or as a single pharmaceutical composition or as independent multiple pharmaceutical compositions.

Pharmaceutical compositions according to the present invention include those suitable for oral, inhalation spray, rectal, topical, buccal (e.g., sublingual), or parenteral (e.g., subcutaneous, intramuscular, intravenous, intrathecal, intramedullary and intradermal injections, or infusion techniques) administration, although the most suitable route in any given case will depend on the nature and severity of the condition being treated and on the nature of the particular compound which is being used. In most cases, the preferred route of administration is oral or parenteral.

The compositions of the present invention can be administered enterally, by inhalation spray, rectally, topically, buccally or parenterally in dosage unit formulations containing conventional nontoxic pharmaceutically acceptable carriers, adjuvants, and vehicles as desired. Parenteral administration includes subcutaneous, intramuscular, intradermal, intramammary, intravenous, and other administrative methods known in the art. Enteral administration includes solution, tablets, sustained release capsules, enteric-coated capsules, and syrups. When administered, the pharmaceutical composition may be at or near body temperature.

The compounds of the present invention can be delivered orally either in a solid, in a semi-solid, or in a liquid form. Oral (intra-gastric) is a preferred route of administration. Pharmaceutically acceptable carriers can be in solid dosage forms for the methods of the present invention, which include tablets, capsules, pills, and granules, which can be prepared with coatings and shells, such as enteric coatings and others well known in the art. Liquid dosage forms for oral administration include pharmaceutically acceptable emulsions, solutions, suspensions, syrups, and elixirs.

Compositions intended for oral use may be prepared according to any method known in the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients, which are suitable for the manufacture of tablets. These excipients may be, for example, inert diluents, such as calcium carbonate, sodium carbonate, lactose, calcium phosphate or sodium phosphate, granulating and disintegrating agents, for example, maize starch, or alginic acid, binding agents, for example starch, gelatin or acacia, and lubricating agents, for example magnesium stearate, stearic acid, or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredients are mixed with an inert solid diluent, for example, calcium carbonate, calcium phosphate or kaolin, or as soft gelatin capsules wherein the active ingredients are present as such, or mixed with water or an oil medium, for example, peanut oil, liquid paraffin, or olive oil.

Aqueous suspensions can be produced that contain the active materials in a mixture with excipients suitable for the manufacture of aqueous suspensions. Such excipients are suspending agents, for example, sodium carboxymethylcellulose, methylcellulose, hydroxypropylmethyl-cellulose, sodium alginate, polyvinylpyrrolidone gum tragacanth and gum acacia; dispersing or wetting agents may be naturally-occurring phosphatides, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyoxyethylene sorbitan monooleate.

The aqueous suspensions may also contain one or more preservatives, for example, ethyl or n-propyl p-hydroxybenzoate, one or more coloring agents, one or more flavoring agents, or one or more sweetening agents, such as sucrose or saccharin. Solutions and suspensions may be prepared from sterile powders or granules having one or more pharmaceutically acceptable carriers or diluents, or a binder such as gelatin or hydroxypropylmethyl cellulose, together with one or more of a lubricant, preservative, surface active or dispersing agent.

Oily suspensions may be formulated by suspending the active ingredients in an omega-3 fatty acid, a vegetable oil, for example, arachis oil, olive oil, sesame oil or coconut oil, or in a mineral oil such as liquid paraffin. The oily suspensions may contain a thickening agent, for example beeswax, hard paraffin or cetyl alcohol.

Sweetening agents, such as those set forth above, and flavoring agents may be added to provide a palatable oral preparation. These compositions may be preserved by the addition of an antioxidant such as ascorbic acid.

Dispersible powders and granules suitable for preparation of an aqueous suspension by the addition of water provide the active ingredient in admixture with a dispersing or wetting agent, a suspending agent and one or more preservatives. Suitable dispersing or wetting agents and suspending agents are exemplified by those already mentioned above. Additional excipients, for example sweetening, flavoring and coloring agents, may also be present.

Dosing for oral administration may be with a regimen calling for single daily dose, or for a single dose every other day, or for multiple, spaced doses throughout the day. For oral administration, the pharmaceutical composition may be in the form of, for example, a tablet, capsule, suspension, or liquid. Capsules, tablets, etc., can be prepared by conventional methods well known in the art. The pharmaceutical composition is preferably made in the form of a dosage unit containing a particular amount of the active ingredient or ingredients. Examples of dosage units are tablets or capsules, and may contain one or more therapeutic compounds in an amount described herein. For example, in the case of an MK-2 inhibitor, the dose range may be from about 0.01 mg to about 5,000 mg or any other dose, dependent upon the specific modulator, as is known in the art. When in a liquid or in a semi-solid form, the combinations of the present invention can, for example, be in the form of a liquid, syrup, or contained in a gel capsule (e.g., a gel cap). In one embodiment, when an MK-2 inhibitor is used in a combination of the present invention, the MK-2 inhibitor can be provided in the form of a liquid, syrup, or contained in a gel capsule.

Oral delivery of the combinations of the present invention can include formulations, as are well known in the art, to provide prolonged or sustained delivery of the drug to the gastrointestinal tract by any number of mechanisms. These include, but are not limited to, pH sensitive release from the dosage form based on the changing pH of the small intestine, slow erosion of a tablet or capsule, retention in the stomach based on the physical properties of the formulation, bioadhesion of the dosage form to the mucosal lining of the intestinal tract, or enzymatic release of the active drug from the dosage form. For some of the therapeutic compounds useful in the methods, combinations and compositions of the present invention the intended effect is to extend the time period over which the active drug molecule is delivered to the site of action by manipulation of the dosage form. Thus, enteric-coated and enteric-coated controlled release formulations are within the scope of the present invention. Suitable enteric coatings include cellulose acetate phthalate, polyvinylacetate phthalate, hydroxypropylmethylcellulose phthalate and anionic polymers of methacrylic acid and methacrylic acid methyl ester.

Pharmaceutical compositions suitable for oral administration can be presented in discrete units, such as capsules, cachets, lozenges, or tablets, each containing a predetermined amount of at least one therapeutic compound useful in the present invention; as a powder or granules; as a solution or a suspension in an aqueous or non-aqueous liquid; or as an oil-in-water or water-in-oil emulsion. As indicated, such compositions can be prepared by any suitable method of pharmacy, which includes the step of bringing into association the active compound(s) and the carrier (which can constitute one or more accessory ingredients). In general, the compositions are prepared by uniformly and intimately admixing the active compound with a liquid or finely divided solid carrier, or both, and then, if necessary, shaping the product. For example, a tablet can be prepared by compressing or molding a powder or granules of the compound, optionally with one or more accessory ingredients. Compressed tablets can be prepared by compressing, in a suitable machine, the compound in a free-flowing form, such as a powder or granules optionally mixed with a binder, lubricant, inert diluent and/or surface active/dispersing agent(s). Molded tablets can be made by molding, in a suitable machine, the powdered compound moistened with an inert liquid diluent.

Syrups and elixirs containing the MK-2 inhibitor may be formulated with sweetening agents, for example glycerol, sorbitol, or sucrose. Such formulations may also contain a demulcent, a preservative and flavoring and coloring agents. Liquid dosage forms for oral administration can include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, and sweetening, flavoring, and perfuming agents.

Also encompassed by the present invention is buccal or “sub-lingual” administration, which includes lozenges or a chewable gum comprising the compounds, set forth herein. The compounds can be deposited in a flavored base, usually sucrose, and acacia or tragacanth, and pastilles comprising the compounds in an inert base such as gelatin and glycerin or sucrose and acacia.

The subject method of prescribing an MK-2 inhibitor can also be administered parenterally, either subcutaneously, or intravenously, or intramuscularly, or intrasternally, or by infusion techniques, in the form of sterile injectable aqueous or olagenous suspensions. Such suspensions may be formulated according to the known art using those suitable dispersing of wetting agents and suspending agents, which have been mentioned above, or other acceptable agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed, including synthetic mono- or diglycerides. In addition, n-3 polyunsaturated fatty acids may find use in the preparation of injectables.

Pharmaceutical compositions suitable for parenteral administration can conveniently comprise sterile aqueous preparations of a compound of the present invention. These preparations are preferably administered intravenously, although administration can also be effected by means of subcutaneous, intramuscular, or intradermal injection or by infusion. Such preparations can conveniently be prepared by admixing the compound with water and rendering the resulting solution sterile and isotonic with the blood. Injectable compositions according to the invention will generally contain from 0.1 to 10% w/w of a compound disclosed herein.

Injectable preparations, for example, sterile injectable aqueous or oleaginous suspensions may be formulated according to the known art using suitable dispersing or setting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid find use in the preparation of injectables.

The active ingredients may also be administered by injection as a composition wherein, for example, saline, dextrose, or water may be used as a suitable carrier. A suitable daily dose of each active therapeutic compound is one that achieves the same blood serum level as produced by oral administration as described above.

The dose of any of these therapeutic compounds can be conveniently administered as an infusion of from about 10 ng/kg body weight to about 10,000 ng/kg body weight per minute. Infusion fluids suitable for this purpose can contain, for example, from about 0.1 ng to about 10 mg, preferably from about 1 ng to about 10 mg per milliliter. Unit doses can contain, for example, from about 1 mg to about 10 g of the compound of the present invention. Thus, ampoules for injection can contain, for example, from about 1 mg to about 100 mg.

Administration of the beta-carboline MK-2 inhibitor can also be by inhalation, in the form of aerosols or solutions for nebulizers. Therefore, in one embodiment, the beta-carboline MK-2 inhibitor is administered by direct inhalation into the respiratory system of a subject for delivery as a mist or other aerosol or dry powder. Delivery of drugs or other active ingredients directly to the subject's lungs provides numerous advantages including, providing an extensive surface area for drug absorption, direct delivery of therapeutic agents to the disease site in the case of regional drug therapy, eliminating the possibility of drug degradation in the subject's intestinal tract (a risk associated with oral administration), and eliminating the need for repeated subcutaneous injections.

Aerosols of liquid particles comprising the active materials may be produced by any suitable means, such as inhalatory delivery systems. Nebulizers are commercially available devices which transform solutions or suspensions of the active ingredient into a therapeutic aerosol mist either by means of acceleration of compressed gas, typically air or oxygen, through a narrow venturi orifice or by means of ultrasonic agitation. Suitable formulations for use in nebulizers consist of the active ingredient in a liquid carrier. The carrier is typically water, and most preferably sterile, pyrogen-free water, or a dilute aqueous alcoholic solution, preferably made isotonic, but may be hypertonic with body fluids by the addition of, for example, sodium chloride. Optional additives include preservatives if the formulation is not made sterile, for example, methyl hydroxybenzoate, as well as antioxidants, flavoring agents, volatile oils, buffering agents and surfactants, which are normally used in the preparation of pharmaceutical compositions.

Aerosols of solid particles comprising the active materials may likewise be produced with any solid particulate medicament aerosol generator. Aerosol generators for administering solid particulate medicaments to a subject produce particles, which are respirable, as explained above, and generate a volume of aerosol containing a predetermined metered dose of a medicament at a rate suitable for human administration.

One type of solid particulate aerosol generator is an insufflator. Suitable formulations for administration by insufflation include finely comminuted powders, which may be delivered by means of an insufflator or taken into the nasal cavity in the manner of a snuff. In the insufflator, the powder is contained in capsules or cartridges, typically made of gelatin or plastic, which are either pierced or opened in situ and the powder delivered by means of air drawn through the device upon inhalation or by means of a manually-operated pump. The powder employed in the insufflator either consists solely of the active ingredient or of a powder blend comprising the active materials, a suitable powder diluent, such as lactose, and an optional surfactant.

A second type of aerosol generator is a metered dose inhaler. Metered dose inhalers are pressurized aerosol dispensers, typically containing a suspension or solution formulation of the MK-2 inhibitor in a liquefied propellant. During use, the metered dose inhaler discharges the formulation through a valve, adapted to deliver a metered volume, to produce a fine particle spray containing the active materials. Any propellant may be used for aerosol delivery, including both chlorofluorocarbon-containing propellants and non-chlorofluorocarbon-containing propellants.

A third type of aerosol generator is a electrohydrodynamic (EHD) aerosol generating device, which has the advantage of being adjustable to create substantially monomodal aerosols having particles more uniform in size than aerosols generated by other devices or methods. Typical EHD devices include a spray nozzle in fluid communication with a source of liquid to be aerosolized, at least one discharge electrode, a first voltage source for maintaining the spray nozzle at a negative (or positive) potential relative to the potential of the discharge electrode, and a second voltage source for maintaining the discharge electrode at a positive (or negative) potential relative to the potential of the spray nozzle. Most EHD devices create aerosols by causing a liquid to form droplets that enter a region of high electric field strength. The electric field then imparts a net electric charge to these droplets, and this net electric charge tends to remain on the surface of the droplet. The repelling force of the charge on the surface of the droplet balances against the surface tension of the liquid in the droplet, thereby causing the droplet to form a cone-like structure known as a Taylor Cone. In the tip of this cone-like structure, the electric force exerted on the surface of the droplet overcomes the surface tension of the liquid, thereby generating a stream of liquid that disperses into a many smaller droplets of roughly the same size. These smaller droplets form a mist, which constitutes the aerosol cloud that the user ultimately inhales.

Administration of the compositions of the present invention can also be rectally. Pharmaceutical compositions suitable for rectal administration are preferably presented as unit-dose suppositories. These can be prepared by admixing a compound or compounds of the present invention with one or more suitable non-irritating excipients, for example, cocoa butter, synthetic mono- di- or triglycerides, fatty acids and polyethylene glycols that are solid at ordinary temperatures, but liquid at the rectal temperature and will therefore melt in the rectum and release the drug; and then shaping the resulting mixture.

Administration may also be by transvaginal delivery through the use of an intravaginal device. Transvaginal delivery may be desirable for many certain subjects because 10 to 30 times more treatment agent can be delivered transvaginally as can be delivered orally due to the absorption from the vagina, which far exceeds the absorption of drugs from the gastrointestinal tract. Further, vaginal administration generally avoids major problems connected with oral administration, such as gastric and esophageal reflux and ulceration.

Pharmaceutical compositions suitable for topical application to the skin preferably take the form of an ointments, creams, lotions, pastes, gels, sprays, powders, jellies, collyriums, solutions or suspensions, aerosols, or oils. Carriers, which can be used, include petroleum jelly (e.g., Vaseline®), lanolin, polyethylene glycols, alcohols, and combinations of two or more thereof. The active compound or compounds are generally present at a concentration of from 0.1 to 50% w/w of the composition, for example, from 0.5 to 2%.

Transdermal administration is also possible. Pharmaceutical compositions suitable for transdermal administration can be presented as discrete patches adapted to remain in intimate contact with the epidermis of the recipient for a prolonged period of time. Such patches suitably contain a compound or compounds of the present invention in an optionally buffered, aqueous solution, dissolved and/or dispersed in an adhesive, or dispersed in a polymer. A suitable concentration of the active compound or compounds is about 1% to 35%, preferably about 3% to 15%. As one particular possibility, the compound or compounds can be delivered from the patch by electrotransport or iontophoresis, for example, as described in Pharmaceutical Research 3(6): 318 (1986).

The compositions of the present invention can optionally be supplemented with additional agents such as, for example, viscosity enhancers, preservatives, surfactants and penetration enhancers.

Viscosity is an important attribute of many medications. Drops that have a high viscosity tend to stay in the body for longer periods and thus, increase absorption of the active compounds by the target tissues or increase the retention time. Such viscosity-building agents include, for example, polyvinyl alcohol, polyvinyl pyrrolidone, methylcellulose, hydroxy propyl methylcellulose, hydroxyethyl cellulose, carboxymethyl cellulose, hydroxy propyl cellulose or other agents know to those skilled in the art. Such agents are typically employed at a level of from 0.01% to 2% by weight.

Preservatives are optionally employed to prevent microbial contamination during use. Suitable preservatives include polyquaternium-1, benzalkonium chloride, thimerosal, chlorobutanol, methyl paraben, propyl paraben, phenylethyl alcohol, edetate disodium, sorbic acid, or other agents known to those skilled in the art. The use of polyquaternium-1 as the antimicrobial preservative is preferred. Typically, such preservatives are employed at a level of from 0.001% to 1.0% by weight.

The solubility of the components of the present compositions may be enhanced by a surfactant or other appropriate co-solvent in the composition. Such co-solvents include polysorbate 20, 60, and 80, polyoxyethylene/polyoxypropylene surfactants (e.g., Pluronic F-68, F-84 and P-103), cyclodextrin, or other agents known to those skilled in the art. Typically, such co-solvents are employed at a level of from 0.01% to 2% by weight.

A penetration enhancer is an agent used to increase the permeability of the skin to an active agent to increase the rate at which the drug diffuses through the skin and enters the tissues and bloodstream. Thus, in one embodiment of the present invention, a penetration enhancer may be added to a beta-carboline MK-2 inhibitor topical composition.

Examples of penetration enhancers suitable for use with the compositions of the present invention include: alcohols, such as ethanol and isopropanol; polyols, such as n-alkanols, limonene, terpenes, dioxolane, propylene glycol, ethylene glycol, other glycols, and glycerol; sulfoxides, such as dimethylsulfoxide (DMSO), dimethylformamide, methyl dodecyl sulfoxide, dimethylacetamide; esters, such as isopropyl myristate/palmitate, ethyl acetate, butyl acetate, methyl proprionate, and capric/caprylic triglycerides; ketones; amides, such as acetamides; oleates, such as triolein; various surfactants, such as sodium lauryl sulfate; various alkanoic acids, such as caprylic acid; lactam compounds, such as azone; alkanols, such as oleyl alcohol; dialkylamino acetates, and admixtures thereof.

Pharmaceutically acceptable excipients and carriers encompass all the foregoing and the like. The above considerations concerning effective formulations and administration procedures are well known in the art and are described in standard textbooks. See e.g., Gennaro, A. R., Remington: The Science and Practice of Pharmacy, 20^{th} Edition, (Lippincott, Williams and Wilkins), 2000; Hoover, John E., Remington's Pharmaceutical Sciences, Mack Publishing Co., Easton, Pa., 1975; Liberman, et al., Eds., Pharmaceutical Dosage Forms, Marcel Decker, New York, N.Y., 1980; and Kibbe, et al., Eds., Handbook of Pharmaceutical Excipients (3^{rd} Ed.), American Pharmaceutical Association, Washington, 1999.

In another embodiment, a kit can be produced that is suitable for use in the prevention or treatment of a TNFα mediated disease or disorder. The kit comprises a dosage form comprising a beta-carboline MK-2 inhibitor in an amount which comprises a therapeutically effective amount.

The following examples describe preferred embodiments of the invention. Other embodiments within the scope of the claims herein will be apparent to one skilled in the art from consideration of the specification or practice of the invention as disclosed herein. It is intended that the specification, together with the examples, be considered to be exemplary only, with the scope and spirit of the invention being indicated by the claims which follow the examples. In the examples all percentages are given on a weight basis unless otherwise indicated.

General Information for Preparation Methods:

Unless otherwise noted, reagents and solvents were used as received from commercial suppliers.

NMR Analysis:

Proton nuclear magnetic resonance spectra were obtained on a Varian Unity Innova 400, a Varian Unity Innova 300, a Varian Unity 300, a Bruker AMX 500 or a Bruker AV-300 spectrometer. Chemical shifts are given in ppm (δ) and coupling constants, J, are reported in Hertz. The solvent peak was used as the reference peak for proton spectra. Mass spectra were obtained on a Perkin Elmer Sciex 100 atmospheric pressure ionization (APCI) mass spectrometer, a Finnigan LCQ Duo LCMS ion trap electrospray ionization (ESI) mass spectrometer, a PerSeptive Biosystems Mariner TOF HPLC-MS (ESI), or a Waters ZQ mass spectrometer (ESI).

Determination of MK-2 IC₅₀:

Recombinant MK-2 was phosphorylated at a concentration of 42-78 M by incubation with 0.23 M of active p38 in 50 mM HEPES, 0.1 mM EDTA, 10 mM magnesium acetate, and 0.25 mM ATP, pH 7.5 for one hour at 30 C.

The phosphorylation of HSP-peptide (KKKALSRQLSVAA) by MAPKAPK2 was measured using an anion exchange resin capture assay method. The reaction was carried out in 50 mM β-glycerolphosphate, 0.04% BSA, 10 mM magnesium acetate, 2% DMSO and 0.8 mM dithiotheritol, pH 7.5 in the presence of the HSP-peptide with 0.2 μCi [³³P]ATP and 0.03 mM ATP. The reaction was initiated by the addition of 15 nM MAPKAPK2 and was allowed to incubate at 30° C. for 30 min. The reaction was terminated and [³³P]ATP was removed from solution by the addition of 150 l of AG 1×8 ion exchange resin in 900 mM sodium formate pH 3.0. A 50 l aliquot of head volume was removed from the quenched reaction mixture and added to a 96-well plate, 150 l of Microscint-40 (Packard) was added and the amount of phosphorylated-peptide was determined. Allow the Microscint to sit in the plates for 60 minutes prior to counting.

Compounds are evaluated as potential inhibitors of MK-2 by measuring their effects on MK2 phosphorylation of the peptide substrate. Compounds may be screened initially at two concentrations prior to determination of IC₅₀ values. Screening results are expressed as percent inhibition at the concentrations of compound tested. For IC₅₀ value determinations, compounds are tested at six concentrations in ten-fold serial dilutions with each concentration tested in triplicate. Results are expressed as IC₅₀ values in micromolar. The assay is performed at a final concentration of 2% DMSO.

Preferred beta-carboline MK-2 inhibiting compounds of the present invention provide IC₅₀ values for MK-2 inhibition of below 200 μM. One method that can be used for determining the MK-2 inhibition IC₅₀ value is that described just above. More preferred beta-carboline MK-2 inhibiting compounds have the capability of providing MK-2 inhibition IC₅₀ values of below 100 μM, yet more preferred of below 50 μM, even more preferred of below 20 μM, yet more preferred of below 10 μM, and even more preferred of below 1 μM.

U937 Cell TNFα Release Assay

The human monocyte-like cell line, U937 (ATCC #CRL-1593.2), is cultured in RPMI1640 media with 10% heat-inactivated fetal calf serum (GIBCO), glutamine and pen/strep at 37° C. and 5% CO₂. Differentiation of U937 to monocytic/macrophage-like cells is induced by the addition of phorbol12-myristate 13-acetate (Sigma) at final concentration of 20 ng/ml to a culture of U937 cells at ˜0.5 million cells/ml and incubated for 24 hrs. The cells are centrifuged, washed with PBS and resuspended in fresh media without PMA and incubated for 24 hrs. Cells adherent to the culture flask are harvested by scraping, centrifugation, and resuspended in fresh media to 2 million cells/ml, and 0.2 ml is aliquoted to each of 96 wells in flat-bottom plate. Cells are then incubated for an additional 24 hours to allow for recovery. The media is removed from the cells, and 0.1 ml of fresh media is added per well. 0.05 ml of serially diluted compound or control vehicle (Media with DMSO) is added to the cells. The final DMSO concentration does not exceed 1%. After a 1 hour incubation, 0.05 ml of 400 ng/ml LPS (E Coli serotype 0111:B4, Sigma) in media is added for final concentration of 100 ng/ml. Cells are incubated at 37° C. for 4 hours. After 4 hours incubation, supernatants are harvest and assayed by ELISA for the presence of TNFα.

U937 Cell TNFα ELISA

ELISA plates (NUNC-Immuno™ Plate Maxisorb™ Surface) were coated with purified mouse monoclonal IgG1 anti-human TNFα antibody (R&D Systems #MAB610; 1.25 ug/ml in sodium bicarbonate pH 8.0, 0.1 ml/well) and incubated at 4° C. Coating solution was aspirated the following day and wells were blocked with 1 mg/ml gelatin in PBS (plus 1× thimerasol) for 2 days at 4° C. Prior to using, wells were washed 3× with wash buffer (PBS with 0.05% Tween). Cultured media samples were diluted in EIA buffer (5 mg/ml bovine γ-globulin, 1 mg/ml gelatin, 1 ml/l Tween-20, 1 mg/ml thimerasol in PBS), added to wells (0.1 ml/well) in triplicate and allowed to incubate for 1.5 hours at 37° C. in a humidified chamber. Plates were again washed and 0.1 ml/well of a mixture of rabbit anti-human TNFα polyclonal antibodies in EIA buffer (1:400 dilution of Sigma #T8300, and 1:400 dilution of Calbiochem #654250) was added for 1 hr at 37° C. Plates were washed as before and peroxidase-conjugated goat anti-rabbit IgG (H+L) antibody (Jackson ImmunoResearch #111-035-144,1 ug/ml in EIA buffer, 0.1 ml/well) was added for 45 min. After final washing, plates were developed with peroxidase-ABTS solution (Kirkegaard/Perry #50-66-01, 0.1 ml/well). Enzymatic conversion of ABTS to colored product was measured after 5-30 minutes using a SpectroMax 340 spectrophotometer (Molecular Devices) at 405 nm. TNF levels were quantitated from a recombinant human TNFα (R&D Systems #210-TA-010) standard curve using a quadratic parameter fit generated by SoftMaxPRO software. ELISA sensitivity was approximately 30 pg TNF/ml. IC₅₀ values for compounds were generated using BioAssay Solver.

Preferred beta-carboline MK-2 inhibiting compounds of the present invention provide TNFα release IC₅₀ values of below 200 μM in an in vitro cell assay. One method that can be used for determining TNFα release IC₅₀ in an in vitro cell assay is that described just above. More preferred beta-carboline MK-2 inhibiting compounds have the capability of providing TNFα release IC₅₀ values of below 50 μM, yet more preferred of below 10, and even more preferred of below 1.0 μM.

Lipopolysaccharide (LPS)-Induced TNFα Production.

Adult male 225-250 gram Lewis rats (Harlan Sprague-Dawley) were used. Rats were fasted 18 hours prior to oral dosing, and allowed free access to water throughout the experiment. Each treatment group consisted of 5 animals.

Compounds were prepared as a suspension in a vehicle consisting of 0.5% methylcellulose, 0.025% Tween-20 in PBS. Compounds or vehicle were orally administered in a volume of 1 ml using an 18 gauge gavage needle. LPS (E. coli serotype 0111:B4, Lot #39H4103, Cat. # L-2630, Sigma) was administered 1-4 hr later by injection into the penile vein at a dose of 1 mg/kg in 0.5 ml sterile saline. Blood was collected in serum separator tubes via cardiac puncture 1.5 hours after LPS injection, a time point corresponding to maximal TNFα production. After clotting, serum was withdrawn and stored at −20° C. until assay by ELISA (described below).

Rat LPS TNFα ELISA

ELISA plates (NUNC-Immuno™ Plate Maxisorb™ Surface) were coated with 0.1 ml per well of a Protein G purified fraction of a 2.5 ug/ml of hamster anti-mouse/rat TNFα monoclonal antibody TN19.12 (2.5 ug/ml in PBS, 0.1 ml/well). The hybridoma cell line was kindly provided by Dr. Robert Schreiber, Washington University. Wells were blocked the following day with 1 mg/ml gelatin in PBS. Serum samples were diluted in a buffer consisting of 5 mg/ml bovine γ-globulin, 1 mg/ml gelatin, 1 ml/l Tween-20, 1 mg/ml thimerasol in PBS, and 0.1 ml of diluted serum was added wells in duplicate and allowed to incubate for 2 hr at 37° C. Plates were washed with PBS-Tween, and 0.1 ml per well of a 1:300 dilution of rabbit anti-mouse/rat TNFα antibody (BioSource International, Cat. #AMC3012) was added for 1.5 hr at 37° C. Plates were washed, and a 1:1000 fold dilution of peroxidase-conjugated donkey anti-rabbit IgG antibody (Jackson ImmunoResearch, Cat. #711-035-152) was added for 45 min. After washing, plates were developed with 0.1 ml of ABTS-peroxide solution (Kirkegaard/Perry, Cat. #50-66-01). Enzymatic conversion of ABTS to colored product was measured after ˜30 minutes using a SpectroMax 340 spectrophotometer (Molecular Devices Corp.) at 405 nm. TNF levels in serum were quantitated from a recombinant rat TNFα (BioSource International, Cat. #PRC3014.) standard curve using a quadratic parameter fit generated by SoftMaxPRO software. ELISA sensitivity was approximately 30 pg TNF/ml. Results are expressed in percent inhibition of the production of TNFα as compared to blood collected from control animals dosed only with vehicle.

Preferred beta-carboline MK-2 inhibiting compounds of the present invention are capable of providing some degree of inhibition of TNFα in animals. That is, the degree of inhibition of TNFα in animals is over 0%. One method for determining the degree of inhibition of TNFα is the rat LPS assay that is described just above. More preferred beta-carboline MK-2 inhibiting compounds have the capability of providing rat LPS TNFα inhibition values of at least about 25%, even more preferred of above 50%, yet more preferred of above 70%, and even more preferred of above 80%.

Synthesis of Beta-Carboline Compounds:

One of ordinary skill in the art will appreciate how to synthesize the beta-carboline MK-2 inhibitors of the present invention from the following descriptions and examples.

A general method for the synthesis of beta-carbolines can be found in, for example, U.S. Pat. No. 4,705,856 to Biere, et al., (describing general methods for the synthesis of beta-carbolines).

The synthesis of several other beta-carbolines have also been described in, for example: Pari, K., et al., “Carbolines That Accumulate in Human Tissues May Serve a Protective Role against Oxidative Stress” J. Biol. Chem. 275(4): 2455-2462 (2000); Brossi, A., et al., “Alkaloids in mammalian tissues. 3. Condensations of L-tryptophan and L-5-hydroxytryptophan with formaldehyde and acetaldehyde” J. Med. Chem. 16(4): 418-20 (1973); Lippke et al., “beta-Carbolines as benzodiazepine receptor ligands. 1. Synthesis and benzodiazepine receptor interaction of esters of beta-carboline-3-carboxylic acid.” J. Med. Chem. 26:499 (1983); Cain, M., et al., “beta-Carbolines: synthesis and neurochemical and pharmacological actions on brain benzodiazepine receptors.” J. Med. Chem. 25: 1081 (1982); Saxena, A., et al., J. Med. Chem. 16(5): 560 (1973); Zhang, H., et al., “Synthesis of beta-and gamma-carbolines by the palladium-catalyzed iminoannulation of internal alkynes” Org. Lett. 3(20): 3083-3086 (2001); Kanekiyo, N., et al., “Total synthesis of beta-carboline alkaloids, (R-(−)-pyridindolol K1, (R(−)-pyridindolol K2, and (R(−)-pyrindolol” J. Org. Chem. 66(26): 8793-8798 (2001); The synthesis of various 1-aryl-1,2,3,4-tetrahydro-.beta.-carboline-3-carboxylic acids have also been described in the literature. See, J. Biol. Chem. 113, 759 (1936) and J. Amer. Chem. Soc. 70, 219 (1948); U.S. Pat. No. 5,550,118 to Jakubowski, et al., (describing polyhydronorharman synthesis); U.S. Pat. No. 4,612,317 to lijimi, et al., (describing tetrahydro-beta-carboline dithioic acid derivative synthesis); U.S. Pat. No. 4,600,715 to Huth, et al., (describing certain beta-carboline derivatives' synthesis); U.S. Pat. No. 4,894,457 to Kubo, et al., (describing 7-bromo-beta-carboline derivative synthesis); U.S. Pat. No. 4,778,800 to Huth et al., (describing the synthesis of 3-oxadiazole and 3-carboxylic acid beta-carbolines); U.S. Pat. No. 4,435,403 to Braestrup, et al., (describing the synthesis of certain 3-substituted beta-carbolines); U.S. Pat. No. 3,202,666 to Szmuszkovicz, et al., (describing the synthesis of substituted 9H-pyrido(3-4-b)indole-1 carboxylic acid and derivatives); U.S. Pat. No. 3,202,667 to Szzuszkovicz, et al., (describing the synthesis of 6-methoxy-1 methyl-9H-pyrido(3-4-b)indole-1 carboxylic acids); European Patent No. EP 781,281 B1 to Fourtillan, J., et al., (describing the synthesis of certain beta-carboline derivatives); PCT published application No. WO 00/59904 A3 to Mavunkel, B., et al., (describing certain beta-carboline derivatives); U.S. Pat. No. 4,291,039 to Van Dyke, et al., (describing the synthesis of tetrahydro beta-carbolines); U.S. Pat. No. 6,350,757 to Goldstein, et al., (describing the synthesis of certain beta-carboline derivatives); EP 0 620, 223 (describing certain tetrahydro-pyrido-indole compounds); EP 0 320, 079 and EP 0 300, 542 (describing beta-carbolines, dihydro beta-carbolines and tetrahydro beta-carbolines); PCT published application No. WO 95/24200 (describing certain tetrahydro beta-carbolines); PCT published application No. WO 01/87038 A2 to Sui, Z., et al., (describing the synthesis of certain beta-carboline derivatives); PCT published application No. WO 00/02878 to Hudson, A., et al., (describing beta-carboline derivatives); U.S. Pat. No. 5,162,336 to Molino, et al., (describing the synthesis of certain tetra-pyrido-indoles); PCT published application No. WO 97/37658 to Spinelli, S., et al., (describing certain tetrahydro-beta-carbolines); and U.S. Pat. No. 5,591,738 to Lotsof (describing the synthesis of beta-carbolines).

Further details of the synthesis of beta-carboline compounds encompassed by the present invention are provided below.

### EXAMPLE 1

This example illustrates the production of 7-methoxy-3,4,5,10-tetrahydro-1H-2,5-methanoazepino[3,4-b]indole-1-carboxylic acid hydrochloride.

### Step A: Production of 3-(5-methoxy-1H-indol-3-yl)pyrrolidine-2,5-dione

A solution of 5-methoxyindole (99.0 g, 672 mmol) and maleimide (130 g, 1344 mmol) in glacial acetic acid was heated to reflux with mechanical stirring under nitrogen for 8 hours. The reaction mixture was cooled to room temp. The reaction mixture was filtered and the yellow solid was washed with acetic acid, EtOAc, and diethyl ether to give the title compound as a pale yellow solid (123 g, 505 mmol, 75% yield): ¹HNMR (400 MHz, DMSO) δ 11.3 (s, 1H), 10.9 (s, 1H), 7.27 (s, 1H), 7.25 (d, 1H), 6.90 (d, 1H), 6.75 (dd, 1H), 4.29 (dd, 1H), 3.73 (s, 3H), 3.17 (dd, 1H), 2.74 (dd, 1H). m/z 245 (M+H).

### Step B: Production of 5-methoxy-3-pyrrolidin-3-yl-1H-indole

A 5 L round bottom flask was equipped with a mechanical stirrer and reflux condenser and was charged with THF (500 mL) and LAH solution (1000 mL of 1.0 M soln in THF, 1000 mmol) under nitrogen. The solution was cooled to ˜5° C. with an ice-water bath. The product of Step A (50.0 g, 204 mmol) was added portionwise over 30 min. The solution was heated to reflux for 2.5 h. After cooling to 15° C., water (38 mL), 15% NaOH (38 mL), and water (115 mL) were added dropwise and the mixture was allowed to warm to room temp. The mixture was filtered, and the cake was washed with 300 mL THF. The filtrate was concentrated to give the title compound as a thick brown oil (44.1 g, 204 mmol, quantitative yield): ¹HNMR (400 MHz, MeOD) δ 7.21 (d, 1H), 7.03 (s, 1H), 7.02 (d, 1H), 6.75 (dd, 1H), 3.81 (s, 3H), 3.46 (quint, 1H), 3.32 (dd, 1H), 3.12-2.99 (m, 2H), 2.84 (dd, 1H), 2.30-2.21 (m, 1H), 2.00-1.90 (m, 1H). m/z 217 (M+H).

### Step C: Production of 7-methoxy-3,4,5,10-tetrahydro-1H-2,5-methanoazepino[3,4-b]indole-1-carboxylic Acid

The product of Step B (5-methoxy-3-pyrrolidin-3-yl-1H-indole, 91.6 g, 424 mmol) was dissolved in MeOH (230 mL). A solution of glyoxylic acid monohydrate (40.9 g, 444 mmol) in water (1000 mL), followed by additional water (375 mL), was added to the reaction soln under vigorous mechanical stirring. The reaction mixture was heated to 60° C. for 10 h. The reaction mixture was cooled to room temp and diluted with water (200 mL). The mixture was filtered and the resulting solids were washed with water (400 mL) and ether (1000 mL) to give the title compound as a tan solid (105 g, 385 mmol, 91% yield, 9:1 mixture of diastereomers): ¹HNMR (400 MHz, DMSO+TFA; minor diastereomer indicated by asterisk) δ 11.42 (s, 1H), 10.85 (s, 1H), 7.35 (d, 1H), 7.28* (d, 1H), 7.09* (d, 1H), 7.06 (d, 1H), 6.74 (dd, 1H), 6.21 (s, 1H), 5.66* (s, 1H), 3.82-3.69 (m, 3H), 3.75 (s, 3H), 3.58-3.44 (m, 2H), 2.34-2.24 (m, 1H), 2.08-2.00 (m, 1H). m/z 273 (M+H). Anal. calcd for C₁₅H₁₆N₂O₃.1.1H₂O: C, 61.67; H, 6.28; N, 9.59. Found: C, 61.77; H, 6.21; N, 9.54.

### Step D: Production of 7-methoxy-3,4,5,10-tetrahydro-1H-2,5-methanoazepino[3,4-b]indole-1-carboxylic Acid Hydrochloride

The product of Step C (7-methoxy-3,4,5,10-tetrahydro-1H-2,5-methanoazepino[3,4-b]indole-1-carboxylic acid, 9.1 g, 33.4 mmol) was suspended in MeOH (200 mL) and treated with 2 N HCl in ether (18 mL, 36 mmol). The mixture was stirred until the solids had completely dissolved. The solution was concentrated, and the solid was suspended in MeOH and concentrated again. The solids were suspended in 50% MeOH/ether and filtered. The precipitate was washed with 30% MeOH/ether and ether to give the title compound as a pale gray solid (8.53 g, 27.6 mmol, 83% yield; 9:1 mixture of diastereomers): ¹HNMR (400 MHz, DMSO; minor diastereomer indicated by asterisk) δ 11.82 (s, 1H), 10.84 (s, 1H), 7.35 (d, 1H), 7.28* (d, 1H), 7.09* (d, 1H), 7.06 (d, 1H), 6.73 (dd, 1H), 6.21 (s, 1H), 5.66* (s, 1H), 3.80-3.66 (m, 3H), 3.75 (s, 3H), 3.58-3.44 (m, 2H), 2.32-2.22 (m, 1H), 2.06-1.98 (m, 1H). m/z 273 (M+H). Anal. calcd for C₁₅H₁₆N₂O₃.HCl: C, 58.35; H, 5.54; N, 9.07. Found: C, 58.37; H, 5.62; N, 9.12.

### EXAMPLE 2

This example illustrates the production of 7-hydroxy-3,4,5,10-tetrahydro-1H-2,5-methanoazepino[3,4-b]indole-1-carboxylic acid hydrochloride.

### Step A.: Production of 3-(5-benzyloxy-1H-indol-3-yl)pyrrolidine-2,5-dione

A mixture of 5-benzyloxyindole (11.1 g, 0.05 mol) and maleimide (9.7 g, 0.1 mol) in 100 ml of glacial acetic acid was heated at reflux for 6 h. After cooling to room temperature, the mixture was concentrated by evaporation to ˜50 ml and then gradually poured on to 200 ml of ice-water with stirring. The resultant precipitate was filtered, washed with water and air-dried. The solid was taken up in ethyl acetate, dried (MgSO4) and evaporated. The residue was stirred with 200 ml of 1:1 ether-hexane, filtered and air-dried to give 12.1 g (75.6%) of a yellow solid: ¹HNMR (400 MHZ, DMSO) δ 12.27 (s, 1H), 10.87 (s, 1H), 7.25-7.46 (m, 9H), 5.05 (s, 1H), 4.27 (dd, 1H), 3.15 (dd, 1H), 2.71 (dd, 1H); m/z 321 (M+H).

### Step B: Production of 7-(benzyloxy)-3,4,5,10-tetrahydro-1H-2,5-methanoazepino[3,4-b]indole-1-carboxylic Acid

To a slurry of lithium aluminum hydride, 1.0 M in THF (120 ml, 120 mmol) in 120 ml THF was added 3-(5-benzyloxy-1H-indol-3-yl)pyrrolidine-2,5-dione (8.3 g, 25.9 mmol) via dry addition funnel at 0° C. After addition was complete, the mixture was maintained at 0° C. for 30 minutes then warmed to ambient temperature for one hour. The resultant solution was heated to reflux overnight. The reaction mixture was cooled to 0° C. and quenched with water. Sodium hydroxide, 2.5 N (150 ml) was added followed by EtOAc. The slurry was filtered through a cake of Celite. The layers were separated and aqueous extracted with EtOAc (3×200 ml). The organic washes were combined, dried over Na₂SO₄ and concentrated to give 5-benzyloxy-3-pyrrolidin-3-yl-1H-indole as a greenish foam (5.47 g, 72.3%).

To a slurry of 5-benzyloxy-3-pyrrolidin-3-yl-1H-indole (5.47 g, 18.73 mmol) in 200 ml water was added glyoxylic acid (1.92 g, 20.62 mmol). The reaction mixture was heated to 60° C. 48 hours. The mixture was cooled to ambient temperature and filtered. The brown solids were washed with water and dried in the air overnight to give 5.12 g (78.5%) of brown solids: ¹HNMR (400 MHz, DMSO+TFA) δ 11.06 (s, 0.2H), 10.84 (s, 0.7H), 7.42 (d, 2H), 7.40-7.26 (m, 4H), 7.15 (d, 1H), 6.79 (dd, 1H), 6.19 (s, 1H), 5.05 (s, 2H), 3.70 (m, 3H), 3.46 (m, 2H), 2.25 (m, 1H), 2.00 (m, 1H); m/z 349 (M+H).

### Step C: Production of 7-hydroxy-3,4,5,10-tetrahydro-1H-2,5-methanoazepino[3,4-b]indole-1-carboxylic Acid Hydrochloride

To a mixture of 7-(benzyloxy)-3,4,5,10-tetrahydro-1H-2,5-methanoazepino[3,4-b]indole-1-carboxylic acid (789.3 mg, 2.26 mmol) in 7 ml anhydrous MeOH was added 4.0 N HCl in dioxane (0.4 ml). The resultant solution was added via cannula to a Fisher-Porter bottle charged with 10% Pd/C (318 mg). The resultant slurry was purged with hydrogen and pressurized to 55 psi at ambient temperature. Additional catalyst (300 mg) was added after several hours and bottle charged with hydrogen. The reaction mixture was purged with nitrogen and filtered through a cake of Celite washing with methanol. The solution was concentrated yielding 668.4 mg of dark brown foam: ¹HNMR (400 MHz, MeOH+TFA) δ 7.14 (d, 1H), 6.87 (m, 1H), 6.67 (dd, 1H), 4.91 (d, 1H), 4.42 (d, 1H), 3.88 (m, 1H), 3.71 (m, 1H), 3.62-3.54 (m, 2H), 3.41 (m, 1H), 2.42 (m, 1H), 2.26 (m, 1H); m/z 259 (M+H).

### EXAMPLE 3

This example illustrates the production of 7-hydroxy-3,4,5,10-tetrahydro-1H-2,5-methanoazepino[3,4-b]indole-1-carboxylic acid trifluoroacetate.

To a suspension of 7-benzyloxy-3,4,5,10-tetrahydro-1H-2,5-methanoazepino[3,4-b]indole-1-carboxylic acid (Compound of Example 2, step B, 1.4 mmol, 500 mg) in methanol (10 mL), was added conc. HCl (0.2 mL) followed by 10% Pd(C) (90 mg). The homogeneous solution was hydrogenated at 48 psi H₂ for 16 hours. The reaction mixture was filtered through Celite and evaporated to dryness. The residue was purified by preparative reverse-phase HPLC using 0.5% trifluoroacetic acid in acetonitrile-water eluent and lyopholized to give an off-white solid (110 mg, 30% yield; 5:1 mixture of diasteromers): ¹HNMR (400 MHz, DMSO; minor diasteromer indicated by asterisk) δ 10.74* (s, 1H), 10.50 (s, 1H), 7.19 (d, 1H), 7.11* (d, 1H), 6.75* (s, 1H), 6.72 (s, 1H), 6.52 (dd, 1H), 5.70 (s, 1H), 5.29* (s, 1H), 3.71-3.29 (m, 6H), 2.22-2.09 (m, 1H), 1.97-1.83 (m, 1H), m/z 259 (M+H).

### EXAMPLE 4

This example illustrates the production of 7-(benzyloxy)-3,4,5,10-tetrahydro-1H-2,5-methanoazepino[3,4-b]indole-1-carboxylic acid trifluoroacetate.

Treatment of 7-benzyloxy-3,4,5,10-tetrahydro-1H-2,5-methanoazepino[3,4-b]indole-1-carboxylic acid (Compound of Example 2, step B) with 0.5% trifluoroacetic acid in acetonitrile-water and evaporation of the solvent gave the title compound as solid (12:1 mix of diasteroisomers): ¹HNMR (400 MHz, DMSO; minor diasteromer indicated by asterisk) δ 10.82 (s, 1H), 7.47-7.25 (m, 6H), 7.14 (s, 1H), 6.79 (dd, 1H), 6.12 (s, 1H), 5.60* (s, 1H), 5.05 (s, 2H), 3.81-3.63 (m, 3H), 3.53-3.43 (m, 3H), 2.32-2.20 (m, 1H), 2.03-1.94 (m, 1H); m/z 349 (M+H).

### EXAMPLE 5

This example illustrates the production of 2,3,8,10,11,12-hexahydro-1H,7H-9,12-methanoazepino[3,4-b]pyrano[3,2-e]indole-8-carboxylic acid hydrochloride.

### Step A: Production of 5-(prop-2-ynyloxy)-1H-indole

To a solution of 5-hydroxyindole (3.25 g, 24.4 mmol) in acetone at room temperature was added cesium carbonate (9.54 g, 28.8 mmoll) and propargyl bromide (80% solution in toluene, 2.94 mL, 26.3 mmol). The reaction mixture was stirred at room temperature overnight and then poured into water and ethyl acetate. The layers were separated and the organic layer washed with brine and dried (Na₂SO₄). The solvent was removed in vacuo to give a brown oil, which after chromatography (silica, 20-30% ethyl acetate:hexane) gave the desired product as an oil (3.45 g, 83%): ¹HNMR (400 MHZ, CDCl₃) δ 8.11 (bs, 1H), 7.28 (d, 1H), 7.20-7.18 (m, 2H), 6.91 (dd, 1H), 6.49-6.48 (m, 1H), 4.72 (d, 2H), 2.50 (t, 1H); m/z 172 (M+H).

### Step B: Production of 1-benzyl-3-[5-(prop-2-ynyloxy)-1H-indol-3-yl]pyrrolidine-2,5-dione

To a solution of 5-(prop-2-ynyloxy)-1H-indole (1.20 g, 6.91 mmol) in acetic acid (40 mL) was added N-benzylmaleimide (3.87 g, 2.09 mmol) at room temperature. The reaction mixture was heated to reflux for 4 hrs and then cooled to room temperature. The solvent was removed in vacuo to give an oil, which was taken up in ethyl acetate and washed with saturated sodium bicarbonate and brine. The organic extract was dried (Na₂SO₄) and the solvent removed to give a brown oil, which after chromatography (silica, 15-50% ethyl acetate:hexanes) gave the product as a brown foam (0.46 g, 18%): m/z 359 (M+H).

### Step C: Production of 1-benzyl-3-(3,7-dihydropyrano[3,2-e]indol-1-yl)pyrrolidine-2,5-dione

A solution of 1-benzyl-3-[5-(prop-2-ynyloxy)-1H-indol-3-yl]pyrrolidine-2,5-dione (0.46 g, 1.30 mmol) in bromobenzene (20 mL) was heated to reflux for 24 hrs. The reaction mixture was allowed to cool to room temperature. The crude reaction mixture was purified by chromatography (silica, 30-55% ethyl acetate:hexane) to give the desired product (0.17 g, 37%): ¹HNMR (400 MHz, CDCl₃) δ 7.97 (bs, 1H), 7.45-7.42 (m, 2H), 7.34-7.28 (m, 3H), 7.11 (d, 1H), 6.89 (d, 1H), 6.79-6.74 (m, 2H), 5.75-5.72 (m, 1H), 4.75 (s, 2H), 4.69-4.66 (m, 2H), 4.46 (dd, 1H), 3.25 (dd, 1H), 2.78 (dd, 1H); m/z 359 (M+H).

### Step D: Production of 1-(1-benzylpyrrolidin-3-yl)-3,7-dihydropyrano[3,2-e]indole

To a solution of lithium aluminum hydride (1 M in THF, 2.11 mL) in THF (10 mL) was added 1-benzyl-3-(3,7-dihydropyrano-[3,2-e]indol-1-yl)pyrrolidine-2,5-dione slowly in a solution of THF (5 mL). The reaction mixture was heated to reflux for 3 hrs and then allowed to cool. The reaction mixture was diluted with 2.5N NaOH (5 mL). The resulting aqueous mixture was extracted with ethyl acetate (2×10 mL). The organic extracts were combined and washed with brine and dried (Na₂SO₄). The solvent was removed in vacuo to give a brown oil (0.14 g, 100%): ¹HNMR (400 MHz, CD₃OD) δ7.34-7.22 (m, 7H), 7.07-7.05 (m, 3H), 6.60 (d, 1H), 5.84-5.79 (m, 1H), 4.60-4.58 (m, 2H), 3.77-3.60 (m, 4H), 3.16-3.12 (m, 1H), 2.87-2.81 (m, 1H), 2.67-2.62 (m, 1H), 2.52 (t, 1H), 2.34-2.28 (m, 1H), 1.98-1.38 (m, 1H); m/z 331 (M+H).

### Step E: Production of 1-pyrrolidin-3-yl-3,7,8,9-tetrahydropyrano[3,2-e]indole

To a solution of 1-(1-benzylpyrrolidin-3-yl)-3,7-dihydropyrano[3,2-e]indole (0.14 g, 4.10 mmol) in methanol was added 10% Pd/C (0.1 g) and ammonium formate (0.12 g, 20.5 mmol) at room temperature. The reaction mixture was heated to reflux for 2 hrs and then allowed to cool to room temperature. The reaction was filtered through Celite and the solvent removed to give a brown oil, which was used in the next step without purification (0.098 g, 100%): m/z 243 (M+H).

### Step F: Production of 2,3,8,10,11,12-hexahydro-1H,7H-9,12-methanoazepino[3,4-b]pyrano[3,2-e]indole-8-carboxylic Acid Hydrochloride

To a solution of 1-pyrrolidin-3-yl-3,7,8,9-tetrahydropyrano[3,2-e]indole (0.098 g, 4.10 mmol) in H₂O:MeOH (8 mL:3 mL) was added glyoxylic acid monohydrate (0.037 g, 4.10 mmol). The reaction mixture was heated to 60° C. overnight and then allowed to cool to room temperature. The precipitate was filtered and washed with ether and dried on high vacuum to give a tan powder (0.045 g, 37%). To the tan powder in a vial was added 5 mL of 2.0 M HCl in ether. The mixture was stirred for 1 hr and then the solvent was removed to give the HCl salt (44 mg): ¹HNMR MHZ, DMSO) δ 11.28 (bs, 1H), 10.79 (bs, 1H), 7.13 (d, 1H), 6.52 (d, 1H), 6.18 (s, 1H), 4.09-3.97 (m, 2H), 3.86 (bs, 1H), 3.79-3.71 (bm, 1H), 3.66 (d, 1H), 3.47-3.44 (m, 2H), 3.13-3.05 (m, 1H), 2.94-2.87 (m, 1H), 2.28-2.25 (m, 1H), 2.14-2.09 (m, 1H), 1.99-1.89 (m, 2H); m/z 299 (M+H).

### EXAMPLE 6

This example illustrates the production of 7-(methylthio)-3,4,5,10-tetrahydro-1H-2,5-methanoazepino[3,4-b]indole-1-carboxylic acid.

The title compound was prepared according to the procedures described in Example 1 (steps A, B and C) starting from 5-(methylthio)indole: ¹HNMR (400 MHZ, DMSO+TFA) δ 11.28 (s, 0.4H), 11.05 (s, 0.7H), 7.54 (dd, 1H), 7.40 (d, 0.7H), 7.34 (d, 0.4H), 7.07 (dt, 1H), 6.22 (s, 0.7H), 5.67 (s, 0.4H), 3.85-3.67 (m, 2H), 3.58-3.32 (m, 3H), 2.44 (s, 3H), 2.40-2.23 (m, 1H), 2.03 (m, 1H): MS (EI)m/z: 289 (M+H).

### EXAMPLE 7

This example illustrates the production of 7-(methylthio)-3,4,5,10-tetrahydro-1H-2,5-methanoazepino[3,4-b]indole-1-carboxylic acid hydrochloride.

The title compound was prepared according to the procedure described in Example 1 (step D) starting from the compound of Example 6. ¹HNMR (400 MHZ, DMSO+TFA) δ 11.27 (s, 0.3H), 11.03 (s, 0.7H), 7.55 (d, 1H), 7.40 (d, 0.7H), 7.34 (0.3H), 7.07 (dd, 1H), 6.21 (s, 0.7H, 5.64 (s, 0.3H), 3.77 (m, 2H), 3.57-3.32 (m, 3H), 2.42 (s, 3H), 2.32-2.21 (m, 1H), 2.05 (m, 1H); MS (EI)m/z: 289 (M+H).

### EXAMPLE 8

This example illustrates the production of 3,4,5,10-Tetrahydro-1H-2,5-methanoazepino[3,4-b]indole-1-carboxylic acid hydrochloride.

A solution of 3-pyrrolidin-3-yl-1H-indole (1.3 g, 7.0 mmol) in 15% MeOH/water was treated with glyoxylic acid (700 mg, 7.7 mmol) and heated to 60° C. for 16 hours. The resultant solid was cooled to room temperature and water (50 mL) was added and the solid filtered (1.1 g). The solid (150 mg, 0.6 mmol) was suspended in MeOH and treated with 2N HCl in ether (0.34 mL, 0.68 mmol). After being stirred for 5 minutes the homogeneous solution was evaporated to dryness. The residue was then dissolved in methanol and evaporated to dryness to give 150 mg of a brown foam. (45:1 mixture of diasteromers): ¹HNMR (400 MHz, DMSO; minor diasteromer indicated by asterisk) δ 11.24* (s, 1H), 10.99 (s, 1H), 7.52 (d, 1H), 7.44 (d, 1H), 7.07 (t, 1H), 6.99 (t, 1H), 6.26 (s, 1H), 5.69* (s, 1H), 3.82-3.64 (m, 3H), 3.53-3.41 (m, 3H), 2.32-2.20 (m, 1H), 2.06-1.96 (m, 1H), m/z 243 (M+H).

### EXAMPLE 9

This example illustrates the production of trans-8-methoxy-1,3,4,5,6,11-hexahydro-2,6-methanoazocino[3,4-b]indole-1-carboxylic acid hydrochloride.

A mixture of 5-methoxy-3-(1-benzyl-1,2,5,6-tetrahydropyridine-3-yl)-1H-indole (1.04 g, 3.27 mmol; P. Gharagozloo et al., Tetrahedron, 1996, 52, 10185-10192) and Pd(OH)₂ (200 mg) in EtOAc (30 mL) and EtOH (30 mL) was placed on a Parr shaker under 40 psi H₂ for 16 h. The mixture was filtered through celite and concentrated to give a 1:1 mixture of 5-methoxy-3-piperidin-3-yl-1H-indole and 3-(1-benzylpiperidin-3-yl)-5-methoxy-1H-indole. The residue (˜1 g) was dissolved in MeOH (20 mL) and treated with ammonium formate (1.03 g, 16.3 mmol) and 10% Pd/C (200 mg). The mixture was refluxed for 1.25 h, cooled to room temp, and filtered. The filtrate was concentrated, and the resultant residue was suspended in half-saturated NaHCO₃ and extracted with CH₂Cl₂ repeatedly. The organic layers were dried (Na₂SO₄) and concentrated to furnish 5-methoxy-3-piperidin-3-yl-1H-indole as an off-white solid (630 mg, 2.74 mmol, 84% yield): ¹HNMR (400 MHz, acetone) δ 9.84 (s, 1H), 7.24 (d, 1H), 7.09 (d, 1H), 7.06 (s, 1H), 6.73 (dd, 1H), 3.79 (s, 3H), 3.32-3.24 (m, 1H), 3.08-2.99 (m, 1H), 2.96-2.87 (m, 1H), 2.68 (br s, 1H), 2.64-2.54 (m, 2H), 2.12-2.06 (m, 1H), 1.74-1.58 (m, 3H). HRMS calcd for C₁₄H₁₉N₂O (M+H) 231.1492, found 231.1477.

A suspension of 5-methoxy-3-piperidin-3-yl-1H-indole (200 mg, 0.868 mmol) in MeOH (0.50 mL) and water (3.0 mL) was treated with glyoxylic acid monohydrate (96 mg, 1.04 mmol). The mixture was warmed to 60° C. for 45 h. The reaction mixture was cooled to room temp and diluted with water. The mixture was filtered, and the tan solid was washed with water to furnish a tan solid (55.1 mg, 0.192 mmol, 22% yield). A suspension of the solid in 2 N HCl in ether (2 mL) was stirred for 4 h. The mixture was diluted with ether and filtered to give the title compound as an off-white solid: ¹HNMR (400 MHz, DMSO) δ 11.08 (s, 1H), 10.26 (br s, 1H), 7.32 (d, 1H), 7.00 (d, 1H), 6.76 (dd, 1H), 5.59 (s, 1H), 3.74 (s, 1H), 3.64-3.48 (m, 3H), 3.46 (s, 1H), 3.40-3.28 (m, 1H), 1.93-1.82 (m, 1H), 1.70-1.50 (m, 2H), 1.37-1.21 (m, 1H). HRMS calcd for C₁₆H₁₉N₂O₃ (M+H) 287.1396, found 287.1357. Anal. calcd for C₁₆H₁₈N₂O₃.1.2HCl.0.1H₂O: C, 57.93; H, 5.90; N, 8.39. Found: C, 57.93; H, 5.90; N, 8.39.

### EXAMPLE 10

This example illustrates the production of 6-(benzyloxy)-3,4,5,10-tetrahydro-1H-2,5-methanoazepino[3,4-b]indole-1-carboxylic acid trifluoroacetate.

The title compound was prepared according to the procedure described in Example 2 and 4 starting from 4-(benzyloxy)indole. ¹HNMR (400 MHZ, DMSO) δ 10.9 (s, 1H), 7.5 (d, 1H), 7.4 (t, 1H), 7.3 (m, 1H), 7.1 (s, 1H), 6.9 (t, 1H), 6.6 (d, 1H), 5.9 (s, 1H), 5.2 (s, 2H), 3.9 (s, 1H), 3.7 (m, 2H), 3.4 (m, 2H), 2.3 (m, 1H), 2.1 (m, 1H); MS (EI)m/z: 349.1563 (M+H) Theo: 349.1547.

### EXAMPLE 11

This example illustrates the production of cyclohexyl 7-hydroxy-3,4,5,10-tetrahydro-1H-2,5-methanoazepino[3,4-b]indole-1-carboxylate hydrochloride.

To a suspension of 7-(benzyloxy)-3,4,5,10-tetrahydro-1H-2,5-methanoazepino[3,4-b]indole-1-carboxylic acid (Compound of Example 2, Step B, 4.08 mmol, 1.57 g) in pyridine (26 ml) was added p-toluenesulfonylchloride (9.02 mmol, 1.72 g) at room temperature. This solution was cooled down to 0°, followed by adding cyclohexanol (22.5 mmol, 2.254 g, 2.38 ml), then gradually warmed to room temperature and stirred for 18 hours. The reaction mixture was then evaporated to dryness under vacuum, the residue was diluted with 50 ml of water and extracted with dichloromethane (3×60 ml). The combined organic layers were washed with water, dried (MgSO4) and evaporated to give an oily residue. Trituration of the residue with ether gave greenish brown solid.

To a suspension of the above solid (3.7 mmol, 1.6 g) in methanol (20 ml) was added cyclohexanol (18 ml), 10% Pd/C (552 mg) and 4M HCl/dioxane (1 ml), and the mixture was hydrogenated in a Parr apparatus (75 psi, 50° C.) for 72 hours. The reaction mixture was then filtered through a pad of celite and the filtrate was evaporated to give a brown oil. Purification of the residue by reverse phase HPLC (acetonitrile+0.05% HCl eluent) gave the title compound as a gray solid (260 mg, 21% yield): ¹HNMR (300 MHZ, CD₃OD) δ 7.696 (d, 2H), 7.270 (d, 1H), 7.200 (d, 2H), 6.933 (d, 1H), 6.771 (dd, 1H), 5.578 (s, 1H) 4.965 (m, 1H), 3.927 (m, 1H), 3.776 (m, 2H), 3.600 (dd, 1H), 3.442 (m, 1H), 2.470 (m, 1H), 2.353 (s, 3H), 2.208 (m, 1H), 2.005 (m, 1H), 1.854 (m, 2H), 1.621 (m, 4H), 1.348 (m, 3H); m/z 341 (M+H).

### EXAMPLE 12

This example illustrates the production of propyl 7-hydroxy-3,4,5,10-tetrahydro-1H-2,5-methanoazepino[3,4-b]indole-1-carboxylate hydrochloride.

7-(Benzyloxy)-3,4,5,10-tetrahydro-1H-2,5-methanoazepino[3,4-b]indole-1-carboxylic acid (Compound of Example 2, Step B, 1.44 mmol, 500 mg) was suspended in n-propanol (50 mL) at room temperature before HCl gas was purged in for 30 minutes. The reaction mixture was then stirred at room temperature for 24 hours. The mixture was concentrated to about 10 mL and purified by reverse phase HPLC, then lyophilized to gave the product as dark gray amorphous solid (156 mg, 46%): ¹HNMR (400 MHZ, MeOH-d4) δ 7.268 (d, 1H), 6.925 (s, 1H), 6.765 (d, 1H), 5.649 (s, 1H) 4.328 (t., 2H), 3.950 (m, 7H), 1.801 (m, 2H), 0.958 (t, 3H); MS (EI) m/z: 301 (M+H).

### EXAMPLE 13

This example illustrates the production of 2-Methoxyethyl 7-hydroxy-3,4,5,10-tetrahydro-1H-2,5-methanoazepino[3,4-b]indole-1-carboxylate hydrochloride.

The title compound was prepared according to the procedure described in Example 12 starting from 7-(benzyloxy)-3,4,5,10-tetrahydro-1H-2,5-methanoazepino[3,4-b]indole-1-carboxylic acid (Compound of Example 2, Step B) and 2-methoxyethanol.

¹HNMR (400 MHZ, MeOH-d4) 67.983 (s, 1H), 7.249 (d, 1H), 6.918 (s, 1H), 6.766 (d, 1H), 5.641 (s, 1H), 4.558 (t, 2H), 4.490 (t, 2H), 3.957 (m, 7H) 2.993 (s, 3H); MS (EI) m/z: 317 (M+H).

### EXAMPLE 14

This example illustrates the production of 7-methoxy-N-(2-methoxyethyl)-3,4,5,10-tetrahydro-1H-2,5-methanoazepino[3,4-b]indole-1-carboxamide.

A mixture of 7-(methoxy)-3,4,5,10-tetrahydro-1H-2,5-methanoazepino[3,4-b]indole-1-carboxylic acid hydrochloride (Compound of Example 1, Step D, 1 mmol, 308 mg), 1-[3-(dimethylamino)propyl]-3-ethylcarbodiimide hydrochloride (1.2 mmol, 230.1 mg), and 4-(dimethylamino)pyridine (0.5 mmol, 61.1 mg) in DMF (4 ml) was stirred at room temperature for 5 minutes. Methoxyethylamine (3 mmol, 225.3 mg), was added to the reaction mixture and the stirring was continued for 1 hour. The reaction was then quenched by the addition of saturated ammonium chloride solution and the mixture was evaporated to dryness under vacuum. Purification of the residue by flash chromatography over silica gel using 4% methanol in dichloromethane as eluent., followed by recrystallization from ether-hexane gave the title compound as a yellow solid (99 mg, 30% yield): ¹HNMR (400 MHZ, CD₃OD) δ 7.154 (d, 1H), 6.940 (d, 1H), 6.670 (dd, 1H), 4.150 (s, 1H), 3.757 (s, 3H), 3.419 (m, 4H), 3.298 (s, 3H), 3.237 (m, 2H), 3.062 (d, 1H), 2.751 (m, 1H), 2.677 (dd, 1H), 1.986 (m, 1H), 1.899 (m, 1H); m/z 330 (M+H).

### EXAMPLE 15

This example illustrates the production of N,7-dimethoxy-N-methyl-3,4,5,10-tetrahydro-1H-2,5-methanoazepino[3,4-b]indole-1-carboxamide.

A mixture of 7-methoxy-3,4,5,10-tetrahydro-1H-2,5-methanoazepino[3,4-b]indole-1-carboxylic acid (Compound of Example 1, step C, 1.00 g, 3.24 mmol), HOBt (481 mg, 3.56 mmol), and EDC-HCl (807 mg, 4.21 mmol) was dissolved in DMF (20 mL) and stirred for 30 min. N-(methoxy)methylamine hydrochloride (632 mg, 6.48 mmol) was added to the dark purple soln, which was stirred for 3.5 h. The reaction was concentrated to dryness. The residue was treated with 1 N NaOH and extracted repeatedly with EtOAc. The organic layers were concentrated to furnish a yellow oil which was purified by flash chromatography (2→10% MeOH/CH₂Cl₂). The title compound was obtained as a yellow foam (192 mg, 0.609 mmol, 19% yield): ¹HNMR (300 MHz, MeOD) δ 7.20 (d, 1H), 6.99 (d, 1H), 6.72 (dd, 1H), 5.03 (br s, 1H), 3.94 (s, 3H), 3.80 (s, 3H), 3.75-3.66 (m, 1H), 3.52-3.38 (m, 2H), 3.31 (s, 3H), 3.12-2.99 (m, 1H), 2.88-2.79 (m, 1H), 2.24-2.10 (m, 1H), 2.10-1.99 (m, 1H). HRMS calcd for C₁₇H₂₂N₃O₃ (M+H) 316.1667, found 316.1668.

### EXAMPLE 16

This example illustrates the production of 7-methoxy-3,4,5,10-tetrahydro-1H-2,5-methanoazepino[3,4-b]indole-1-carboxamide.

The title compound was prepared from 7-methoxy-3,4,5,10-tetrahydro-1H-2,5-methanoazepino[3,4-b]indole-1-carboxylic acid (Compound of Example 1, step C, 250 mg, 0.810 mmol) and NH₃ (g) as described for Example 15. The crude material was purified by recrystallization from MeOH/CH₂Cl₂ to furnish the title compound as a white solid (85.6 mg). The mother liquor was purified by flash chromatography (5→10% MeOH/CH₂Cl₂) to provide another 26.2 mg of the title compound (112 mg total, 0.413 mmol, 51% yield; 95:5 mixture of diastereomers): ¹HNMR (300 MHz, DMSO; minor diastereomer indicated by asterisk) δ 10.34 (s, 1H), 10.28* (s, 1H), 7.59 (s, 1H), 7.38 (s, 1H), 7.20 (d, 1H), 6.93 (d, 1H), 6.61 (dd, 1H), 4.96* (s, 1H), 4.16 (s, 1H), 3.73 (s, 3H), 3.34-3.14 (m, 2H), 2.90-2.78 (m, 2H), 2.70-2.62 (m, 1H), 2.04-1.90 (m, 1H), 1.90-1.77 (m, 1H). HRMS calcd for C₁₅H₁₈N₃O₂ (M+H) 272.1394, found 272.1355.

### EXAMPLE 17

This example illustrates the production of 7-methoxy-N-methyl-3,4,5,10-tetrahydro-1H-2,5-methanoazepino[3,4-b]indole-1-carboxamide.

The title compound was prepared from 7-methoxy-3,4,5,10-tetrahydro-1H-2,5-methanoazepino[3,4-b]indole-1-carboxylic acid (Compound of Example 1, step C, 250 mg, 0.810 mmol) and methylamine (1.6 mL of 2.0 M soln in THF, 3.24 mmol) as described for Example 15. The crude material was purified by flash chromatography (5→10% MeOH/CH₂Cl₂) to provide the title compound as an off-white solid (89.2 mg, 0.313 mmol, 39% yield): ¹HNMR (300 MHz, DMSO) δ 10.35 (s, 1H), 8.12 (d, 1H), 7.21 (d, 1H), 6.93 (d, 1H), 6.61 (dd, 1H), 4.20 (s, 1H), 3.73 (s, 3H), 3.30-3.14 (m, 2H), 2.92-2.78 (m, 2H), 2.70-2.62 (m, 4H), 2.05-1.92 (m, 1H), 1.89-1.78 (m, 1H). HRMS calcd for C₁₆H₂₀N₃O₂ (M+H) 286.1550, found 286.1497.

### EXAMPLE 18

This example illustrates the production of 7-methoxy-N,N-dimethyl-3,4,5,10-tetrahydro-1H-2,5-methanoazepino[3,4-b]indole-1-carboxamide.

The title compound was prepared 7-methoxy-3,4,5,10-tetrahydro-1H-2,5-methanoazepino[3,4-b]indole-1-carboxylic acid (Compound of Example 1, step C, 250 mg, 0.810 mmol) and dimethylamine (1.6 mL of 2.0 M soln in THF, 3.24 mmol) as described for Example 15. The crude material was purified by flash chromatography (5→10% MeOH/CH₂Cl₂) to provide the title compound as an off-white solid (118 mg, 0.394 mmol, 49% yield): ¹HNMR (300 MHz, DMSO) δ 10.33 (s, 1H), 7.09 (d, 1H), 6.94 (d, 1H), 6.61 (dd, 1H), 4.58 (s, 1H), 3.74 (s, 3H), 3.29 (d, 1H), 3.25 (s, 3H), 3.23-3.12 (m, 1H), 3.05 (d, 1H), 2.91-2.80 (m, 1H), 2.88 (s, 3H), 2.45 (dd, 1H), 1.98-1.75 (m, 2H). HRMS calcd for C₁₇H₂₂N₃O₂ (M+H) 300.1707, found 300.1664.

### EXAMPLES 19-40

These examples illustrate the production of several beta-carboline compounds of the present invention.

The following Examples 19-40 in Table 2 were prepared in accordance with the procedures described for Examples 11, 12, 13 and 14. NMR analysis was carried out for each compound and selected data is presented for each compound as shown in the tables.

TABLE 2

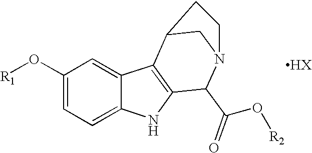

| | | | | (1H NMR, 400 MHZ, DMSO) | |
|---|---|---|---|---|---|
| Ex. No. | R¹ | R² | HX | (minor diastereomer denoted by sterisk) | HR MS |
| 19 | CH₃ | CH₂CH₂F | CF₃CO₂H | δ 10.34 (s, 1H), 10.13* (s, 1H), 7.36* (d, | m/z 319 |
| | | | | 1H), 7.34 (d, 1H), 7.16 (d, 1H), 7.15* (d, | (M + H) |
| | | | | 1H), 6.83 (dd, 1H), 6.81* (dd, 1H), 6.25* (s, | |
| | | | | 1H), 5.63 (s, 1H), 4.8-4.4 (m, 5H), 4.05 (td, | |
| | | | | 1H), 3.92 (t, 1H), 3.81 (s, 3H), 3.67 (dd, | |
| | | | | 1H), 3.52-3.44 (m, 1H), 2.54-2.46 (m, 1H), | |
| | | | | 2.29-2.23 (m, 1H) | |
| 20 | CH₃ | CH₂CH₂OCH₃ | CF₃CO₂H | δ 9.49* (s, 1H), 9.44 (s, 1H), 7.45* (d, 1H), | m/z 331 |
| | | | | 7.44 (d, 1H), 7.21 (d, 1H), 7.20* (d, 1H), | (M + H). |
| | | | | 6.95 (dd, 1H), 6.94* (dd, 1H), 6.09* (s, 1H), | |
| | | | | 5.45 (s, 1H), 4.48 (t, 2H), 4.06 (td, 1H), | |
| | | | | 3.91 (s, 3H), 3.91-3.87 (m, 1H), 3.76-3.73 | |
| | | | | (m, 4H), 3.65 (dd, 1H), 3.50* (s, 3H), 3.40 | |
| | | | | (s, 3H), 2.54-2.46 (m, 1H), 2.29-2.23 (m, 1H) | |
| 21 | H | CH₃ | HCl | δ 7.25 (d, 1H), 7.21* (d, 1H), 6.89* (d, 1H), | m/z 273 |
| | | | | 6.88 (d, 1H), 6.74-6.70 (m, 1H), 6.19 (s, 1H), | (M + H). |
| | | | | 5.61* (s, 1H), 5.58 (s, 3H), 4.01* (s, 3H), | |
| | | | | 3.91-3.48 (m, 5H), 2.51-2.37 (m, 1H), | |
| | | | | 2.14 (m, 1H) | |
| 22 | CH₃ | CH₂Ph | CF₃CO₂H | δ 10.36 (s, 1H), 10.20* (s, 1H), 7.52* (d, 1H), | m/z 363 |
| | | | | 7.45-7.425 (m, 2H), 7.38-7.29 (m, 4H), 7.14 | (M + H). |
| | | | | (d, 1H), 7.13* (d, 1H), 6.82 (dd, 1H), 6.81* | |
| | | | | (dd, 1H), 6.28* (s, 1H), 5.68 (s, 1H), 5.40 | |
| | | | | (AB, 2H), 5.22 (d, 1H), 4.07 (td, 1H), 3.91 | |
| | | | | (t, 1H), 3.80 (s, 3H), 3.68 (dd, 1H), 3.52-3.47 | |
| | | | | (m, 1H), 2.55-2.44 (m, 1H), 2.27-2.24 (m, 1H) | |
| 23 | H | CH₂Ph | HCl | δ 10.63* (s, 1H), 10.45 (s, 1H), 7.49 (d, 1H), | m/z 349 |
| | | | | 7.41-7.31 (m, 5H), 7.24* (d, 1H), 7.20 (d, | (M + H). |
| | | | | 1H), 6.98* (dd, 1H), 6.73 (dd, 1H), 6.22* (s, | |
| | | | | 1H), 5.63 (s, 1H), 5.50* (AB, 2H), 5.40 (AB, | |
| | | | | 2H), 3.9-3.4 (m, 5H), 2.50-2.41 (m, 1H), | |
| | | | | 2.41-2.32* (m, 1H), 2.27-2.24 (m, 1H). | |
| 24 | CH₃ | 1-adamantyl | HCl | δ 7.30 (d, 1H), 7.05 (d, 1H), 6.82 (dd, 1H), | m/z 409 |
| | | | | 6.03* (s, 1H), 5.43 (s, 1H), 3.88-3.82 (m, 1H), | (M + H). |
| | | | | 3.81 (s, 3H), 3.70-3.40, (m, 4H), 2.50-2.34 | |
| | | | | (m, 1H), 2.22 (m, 9H) 2.20-2.14 (m, 1H), | |
| | | | | 1.71 (m, 3H). | |
| 25 | CH₃ | CH(CH₂CH₃)₂ | HCl | δ 7.29 (d, 1H), 7.05 (d, 1H), 6.82 (dd, 1H), | m/z 343 |
| | | | | 6.27* (s, 1H), 5.66 (s, 1H), 4.97 (p, 1H), 3.90- | (M + H). |
| | | | | 3.86 (m, 2H), 3.81 (s, 3H), 3.74-3.44, (m, | |
| | | | | 3H), 2.63-2.44 (m, 1H), 2.26-2.21 (m, 1H), | |
| | | | | 1.77 (p, 2H), 1.64 (p, 2H), 0.99, (t, 3H), | |
| | | | | 0.67 (t, 3H) | |
| 26 | CH₂Ph | CH₂Ph | CF₃CO₂H | δ 10.37 (s, 1H), 10.20* (s, 1H), 7.66-7.22 | m/z 439 |
| | | | | (m 12H), 6.92 (dd, 1H), 6.91* (dd, 1H), | (M + H). |
| | | | | 6.28* (s, 1H), 5.67 (s, 1H), 5.41 (AB, 2H), | |
| | | | | 5.21 (d, 1H), 5.13 (s, 2H), 4.08 (td, 1H), | |
| | | | | 3.90 (t, 1H), 3.68 (dd, 1H), 3.52-3.47 (m, 1H), | |
| | | | | 2.55-2.44 (m, 1H), 2.27-2.24 (m, 1H) | |
| 27 | CH₃ | CH₂CF₃ | CF₃CO₂H | δ 11.1 (s, 1H), 7.35* (d, 1H), 7.3 (d, 1H), | 355.1248 |
| | | | | 7.1 (s, 1H), 6.8 (d, 1H), 6.55* (s, 1H), | |
| | | | | 5.9 (s, 1H), 5.1 (m, 1H), 4.9 (m, 1H), | |
| | | | | 3.8 (m, 2H), 3.7 (s, 3H), 3.6-3.4 (m, 3H), | |
| | | | | 2.3 (m, 1H), 2.1 (m, 1H) | |
| 28 | CH₃ | CH₂CH(OH)CH₂OH | HCl | δ 11.1 (s, 1H), 7.35* (d, 1H), 7.3 (d, 1H), | 347.1644 |
| | | | | 7.1 (s, 1H), 6.8 (d, 1H), 6.4* (s, 1H), | |
| | | | | 5.9 (s, 1H), 4.3 (m, 1H), | |
| | | | | 4.1 (m, 1H), 3.8 (m, 2H), 3.7 (s, 3H), | |
| | | | | 3.6-3.3 (m, 6H), 2.3 (m, 1H), 2.1 (m, 1H) | |
| 29 | CH₃ | CH₂-(4-Pyridyl) | — | δ 10.6 (s, 1H), 8.6 (d, 2H), 7.4 (d, 2H), | 364.1682 |
| | | | | 7.25* (d, 1H), 7.2 (d, 1H), 7.0 (s, 1H), | |
| | | | | 6.7 (d, 1H), 5.4* (m, 2H), 5.3 (m, 2H), | |
| | | | | 4.6 (s, 1H), 4.5* (s, 1H), 3.8 (s, 3H), | |
| | | | | 3.3-3.1 (m, 3H), 2.9 (m, 1H), 2.7 (m, 1H) | |
| | | | | 2.3 (m, 1H), 2.1 (m, 1H) | |
| 30 | CH₃ | CH₂—CH═CH₂ | HCl | δ 11.1 (s, 1H), 7.35* (d, 1H), 7.3 (d, 1H), | 313.12 |
| | | | | 7.1 (s, 1H), 6.8 (d, 1H), 6.45* (s, 1H), | |
| | | | | 6.0 (m, 1H), 5.9 (s, 1H), | |
| | | | | 5.3 (d, 1H), 5.1 (d, 1H), 4.8 (m, 2H), | |
| | | | | 3.8 (m, 2H), 3.7 (s, 3H), 3.6-3.4 (m, 3H), | |
| | | | | 2.3 (m, 1H), 2.1 (m, 1H) | |
| 31 | CH₃ | CH₂CH₂SCH₃ | HCl | δ 11.1 (s, 1H), 7.35* (d, 1H), 7.3 (d, 1H), | 347.1441 |
| | | | | 7.1 (s, 1H), 6.8 (d, 1H), 6.4* (s, 1H), | |
| | | | | 5.9 (s, 1H), 4.4 (m, 2H), | |
| | | | | 3.8 (m, 2H), 3.7 (s, 3H), 3.6-3.4 (m, 3H), | |
| | | | | 2.8 (m, 2H), 2.3 (m, 1H), 2.1 (m, 4H) | |
| 32 | CH₃ | CH₂CH₂OH | HCl | δ 11.1 (s, 1H), 7.35* (d, 1H), 7.3 (d, 1H), | 317.1515 |
| | | | | 7.1 (s, 1H), 6.8 (d, 1H), 6.4* (s, 1H), | |
| | | | | 5.9 (s, 1H), 4.4* (m, 2H), 4.3 (m, 1H), | |
| | | | | 4.1 (m, 1H), 3.8 (m, 4H), 3.7 (s, 3H), | |
| | | | | 3.6-3.4 (m, 3H), 2.3 (m, 1H), 2.1 (m, 1H) | |
| 33 | CH₃ | CH₂Ph | HCl | δ 11.1 (s, 1H), 7.55* (d, 2H), 7.5 (d, 2H), | 363.1709 |
| | | | | 7.4 (m, 3H), 7.3 (d, 1H), 7.1 (s, 1H), | |
| | | | | 6.8 (d, 1H), 6.45* (s, 1H), 5.9 (s, 1H), | |
| | | | | 5.4 (m, 1H), 5.2 (d, 1H), 3.8 (m, 2H), | |
| | | | | 3.7 (s, 3H), 3.6-3.4 (m, 3H), | |
| | | | | 2.3 (m, 1H), 2.1 (m, 1H) | |
| 34 | CH₃ | CH₂-(4-OCF₃-phenyl) | HCl | δ 11.1 (s, 1H), 7.7 (d, 2H), 7.5 (d, 2H), | 447.1520 |
| | | | | 7.3 (d, 1H), 7.1 (s, 1H), 6.8 (d, 1H), | |
| | | | | 6.6* (s, 1H), 5.9 (s, 1H), 5.5 (d, 1H), | |
| | | | | 5.45* (d, 1H), 5.3 (d, 1H), 3.8 (m, 2H), | |
| | | | | 3.7 (s, 3H), 3.6-3.4 (m, 3H), 2.3 | |
| | | | | (m, 1H), 2.1 (m, 1H) | |
| 35 | CH₃ | CH(CH₃)₂ | CF₃CO₂H | δ 11.1 (s, 1H), 7.35* (d, 1H), 7.3 (d, 1H), | 315.1681 |
| | | | | 7.1 (s, 1H), 6.8 (d, 1H), 6.3* (s, 1H), | |
| | | | | 5.7 (s, 1H), 5.3* (q, 1H), 5.1 (q, 1H), | |
| | | | | 3.8 (m, 2H), 3.7 (s, 3H), 3.6-3.4 (m, 3H), | |
| | | | | 2.3 (m, 1H), 2.1 (m, 1H), 1.3 (2, 3H), | |
| | | | | 1.1 (d, 3H) | |
| 36 | CH₃ | CH₂CH₂OCH₃ | HCl | δ 7.29 (d, 1H), 7.07 (d, 1H), 6.83 (dd, 1H), 6.25* (s, | m/z 331 |
| | | | | 1H), 5.66 (s, 1H), 4.59* (AB of ABX₂, 2H), 4.50 (AB | (M + H) |
| | | | | of ABX₂, 2H), 3.98-3.86 (m, 2H), 3.81 (s, 3H), 3.79- | |
| | | | | 3.74 (m, 1H), 3.70 (X₂ of ABX₂, 2H), 3.61 (dd, 1H), | |
| | | | | 3.54-3.46 (m, 1H), 3.41* (s, 3H), 3.33 (s, 3H), 2.50 | |
| | | | | (tdd, 1H), 2.29-2.20 (m, 1H) | |
| 37 | CH₃ | CH₃ | CF₃CO₂H | δ 11.12 (s, 1H), 10.90* (s, 1H), 7.34* (d, 1H), 7.28 | 287.1389 |
| | | | | (d, 1H), 7.11 (d, 1H), 7.09* (d, 1H), 6.78 (dd, 1H), | |
| | | | | 6.36* (s, 1H), 5.80 (s, 1H), 3.92* (s, 3H), 3.85 (s, | |
| | | | | 3H), 3.83-3.76 (m, 2H), 3.75 (s, 3H), 3.64-3.59 | |
| | | | | (m, 1H), 3.54-3.41 (m, 2H), 2.40-2.30 (m, 1H), | |
| | | | | 2.08-2.00 (m, 1H) | |
| 38 | CH₃ | CH(CH₃)₂ | HCl | δ 11.33 (bs, 1H), 11.03 (s, 1H), 10.79* (s, 1H), 7.36* | m/z 315 |
| | | | | (d, 1H), 7.31 (d, 1H), 7.10 (d, 1H), 7.08* (d, 1H), | (M + H) |
| | | | | 6.78 (dd, 1H), 6.34* (s, 1H), 5.74 (s, 1H), 5.18* (sept, | |
| | | | | 1H), 5.08 (sept, 1H), 3.83-3.77 (m, 2H), 3.75 (s, 3H), | |
| | | | | 3.66-3.60 (m, 1H), 3.54-3.41 (m, 2H), 2.40-2.30 | |
| | | | | (m, 1H), 2.07-1.99 (m, 1H), 1.35 (d, 3H), 1.25 | |
| | | | | (d, 3H) | |
| 39 | CH₃ | CH₂CH₃ | CF₃CO₂H | δ 11.07 (s, 1H), 10.84* (s, 1H), 7.35* (d, 1H), 7.29 | m/z 301 |
| | | | | (d, 1H), 7.10 (d, 1H), 7.09* (d, 1H), 6.78 (dd, 1H), | (M + H) |
| | | | | 6.35* (s, 1H), 5.77 (s, 1H), 4.40-4.23 (m, 2H), 3.83- | |
| | | | | 3.75 (m, 2H), 3.75 (s, 3H), 3.64-3.60 (m, 1H), 3.56- | |
| | | | | 3.41 (m, 2H), 2.40-2.30 (m, 1H), 2.08-2.00 (m, 1H), | |
| | | | | 1.35* (t, 3H), 1.30 (t, 3H) | |
| 40 | CH₃ | 2,2,6,6-tetramethyl | CF₃CO₂H | δ 10.49 (s, 1H), 10.13* (s, 1H), 7.33 (d, 1H), 7.08 (d, | 411.2639 |
| | | | | 1H), 6.86 (dd, 1H), 6.41* (s, 1H), 5.77 (s, 1H), 4.98* | |
| | | | | (s, 1H), 4.81 (s, 1H), 3.95-3.87 (m, 2H), 3.83 (s, 3H), | |
| | | | | 3.74-3.63 (m, 2H), 3.52-3.43 (m, 1H), 2.55-2.45 (m, | |
| | | | | 1H), 2.28-2.20 (m, 1H), 1.72-1.56 (m, 2H), 1.48-1.22 | |
| | | | | (m, 4H), 1.18 (s, 3H), 1.17* (s, 3H), 1.14* (s, 3H), | |
| | | | | 0.98 (s, 3H), 0.94* (s, 3H), 0.90 (s, 3H), 0.54 (s, 3H) | |

### EXAMPLE 41

This example illustrates the production of 6-(benzyloxy)-2,3,4,9-tetrahydro-1H-beta-carboline-1-carboxylic acid hydrochloride.

The title compound was prepared by adding to a suspension of 5-benzyloxytryptamine (250 mg, 0.83 mmol) in 10 mL of water and 2 mL of methanol, glyoxylic acid (80 mg, 0.87 mmol) and 0.5 mL of a 1 N potassium hydroxide solution. The reaction was heated to 65° C. for 18 hours. The reaction was cooled to room temperature and the solids were filtered off and washed with water and dried. The solid was suspended in 5 mL of methanol and treated with 2 mL of 2 M HCl ether solution. The homogeneous mixture was condensed to dryness and the residue taken up in 5 mL of methanol and triturated with diethyl ether. The resulting solid was collected by filtration and dried (165 mg, 55% yield) ¹HNMR (300 MHz, CD₃OD) δ 7.50-7.28 (m, 6H), 7.09 (s, 1H), 6.94 (d, 1H), 5.52 (s, 1H), 5.11 (s, 2H), 3.79-3.58 (m, 2H), 3.07 (m, 2H); m/z 323 (M+H).

### EXAMPLE 42

This example illustrates the production of 6-hydroxy-2,3,4,9-tetrahydro-1H-beta-carboline-1-carboxylic acid hydrochloride.

The title compound was prepared by adding to a solution of 6-(benzyloxy)-2,3,4,9-tetrahydro-1H-beta-carboline-1-carboxylic acid hydrochloride (147 mg, 0.41 mmol) in 15 mL of methanol, 10% Pd(C) (36 mg) and 7 drops of conc. hydrochloric acid. The homogeneous solution was hydrogenated at 42 psi hydrogen for 45 minutes. The reaction mixture was then filtered through Celite and evaporated to dryness. 5 mL of water was added to the residue and the resultant solid was collected by filtration, washed with water and dried (60 mg, 60%). ¹HNMR (400 MHz, DMSO+KOH) δ 6.98 (d, 1H), 6.51 (s, 1H), 6.37 (d, 1H), 4.07 (s, 1H), 3.13-3.03 (m, 2H), 2.73-2.60 (m, 2H).

### EXAMPLE 43

This example illustrates the production of sodium 6-(benzyloxy)-2-methyl-2,3,4,9-tetrahydro-1H-beta-carboline-1-carboxylate.

### Step A: Production of Methyl 6-(benzyloxy)-2,3,4,9-tetrahydro-1H-beta-carboline-1-carboxylate Hydrochloride

Hydorchloric acid (g) was bubbled into 400 mL of methanol for 3 minutes then 6-(benzyloxy)-2,3,4,9-tetrahydro-1H-beta-carboline-1-carboxylic acid (8.2 g, 25.4 mmol) was added. Hydrochloric acid (g) was then bubbled into the reaction for 4 minuets stirred 3 hours. Then bubbled hydrochloric acid (g) into reaction for 5 minutes and the reaction stirred for 16 hours.

Then bubbled hydrochloric acid (g) into reaction for 5 minutes and stirred 64 hours. Nitrogen (g) was then bubbled into the solution for 1 hour and the reaction condensed to dryness. Diethyl ether was added and the yellow solid collected by filtration and dried (8.5 g, 89%) ¹HNMR (300 MHz, DMSO) δ 10.96 (s, 1H), 7.46-7.27 (m, 6H), 7.07 (s, 1H), 6.85 (d, 1H), 5.64 (s, 1H), 5.07 (s, 2H), 3.84 (s, 3H), 3.49 (br s, 2H), 2.90 (br s, 2H).

### Step B: Production of Methyl 6-(benzyloxy)-2-methyl-2,3,4,9-tetrahydro-1H-beta-carboline-1-carboxylate

To a suspension of methyl 6-(benzyloxy)-2,3,4,9-tetrahydro-1H-beta-carboline-1-carboxylate hydrochloride (product of Step A, 4.8 g, 12.9 mmol) in 150 mL methylene chloride was added a 37% solution of formaldehyde (1.3 mL, 15.5 mmol) followed by sodium triacetoxyborohydride (6.8 g, 32.2 mmol) the reaction was stirred at room temperature for 3 hours. The reaction was quenched with water and extracted 3× with methylene chloride, washed 1× with brine, dried over magnesium sulfate and evaporated to give an off-whit foam (4.3 g, 95%). ¹HNMR (300 MHz, DMSO) δ 10.59 (s, 1H), 7.46-7.26 (m, 6H), 7.19 (d, 1H), 7.00 (s, 1H), 6.76 (d, 1H), 5.06 (s, 2H), 4.39 (br s, 1H), 3.67 (s, 3H), 3.23-3.13 (m, 1H), 2.76-2.62 (m, 3H), 2.48 (d, 3H). m/z 351 (M+H).

### Step C: Production of Sodium 6-(benzyloxy)-2-methyl-2,3,4,9-tetrahydro-1H-beta-carboline-1-carboxylate

To a solution of methyl 6-(benzyloxy)-2-methyl-2,3,4,9-tetrahydro-1H-beta-carboline-1-carboxylate (product of Step B, 200 mg, 0.57 mmol) in a mixture of water (2 mL) and tetrahydrofuran (3 mL) was added 0.5017 N sodium hydroxide (1.2 mL, 0.60 mmol). The reaction was allowed to stir at room temperature for 18 hours then condensed to a yellow solid. Diethyl ether was added and decanted off, and then the solid was dried. The solid was then dissolved in water and lyophilized to give a yellow solid (220 mg, 99%). ¹HNMR (300 MHz, DMSO) δ 10.00 (s, 1H), 7.42 (d, 2H), 7.34 (t, 2H), 7.27 (d, 1H), 7.20 (d. 1H), 6.87 (s, 1H), 6.62 (d, 1H), 5.02 (s, 2H), 3.63 (s, 1H), 3.14-3.07 (m, 1H), 2.70-2.60 (m, 1H), 2.53-2.35 (m, 5H).

### EXAMPLE 44

This example illustrates the production of 3-(aminomethyl)-6-methoxy-2,3,4,9-tetrahydro-1H-beta-carbolin-1-one hydrochloride.

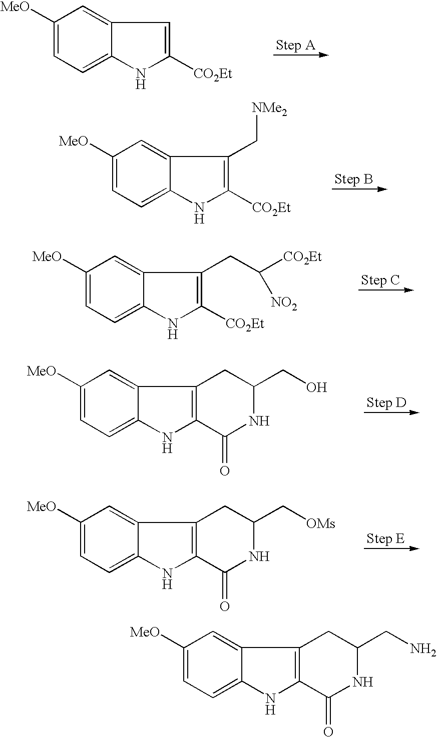

### Step A: Production of Ethyl 3-[(dimethylamino)methyl]-5-methoxy-1H-indole-2-carboxylate

A mixture of ethyl 5-methoxy-1H-indole-2-carboxylate (29.9 grams (g), 136 millimoles (mmol)) in methylene chloride (600 milliliters (mL)) was treated with N,N-dimethylmethylene-ammonium chloride (19.2 g, 205 mmol) at room temperature with mechanical stirring. The reaction mixture was stirred at 40° C. for 20 hours. The mixture was diluted with diethyl ether and then filtered. The white solid was washed with diethyl ether and was then partitioned between 1 N NaOH and methylene chloride. The aqueous layer was extracted with methylene chloride. The organic layer was washed with brine, dried (Na₂SO₄), and concentrated to give the title compound as a white solid (37.5 g, quantitative): ¹HNMR (300 MHz, CDCl₃) δ 8.71 (s, 1H), 7.21 (s, 1H), 7.17 (d, 1H), 6.94 (dd, 1H), 4.37 (q, 2H), 3.88 (s, 2H), 3.82 (s, 3H), 2.28 (s, 6H), 1.38 (t, 3H). m/z 277 (M+H).

### Step B: Production of Ethyl 3-(3-ethoxy-2-nitro-3-oxopropyl)-5-methoxy-1H-indole-2-carboxylate

A mixture of ethyl 3-[(dimethylamino)methyl]-5-methoxy-1H-indole-2-carboxylate (9.0 g, 32.6 mmol) and ethyl nitroacetate (7.7 mL, 65.1 mmol) in o-xylene (90 mL) was stirred at 120° C. for 12 hours. A precipitate formed as the reaction was cooled to room temperature. The mixture was diluted with hexanes (45 mL) and filtered. The solid was washed with toluene/hexanes and dried under vacuum to afford the title compound as an off-white solid (9.0 g, 76%): ¹HNMR (300 MHz, DMSO-d₆) δ 11.75 (s, 1H), 7.34 (d, 1H), 7.20 (s, 1H), 6.96 (dd, 1H), 5.84 (t, 1H), 4.36 (q 2H), 4.18 (q, 2H), 4.05-3.84 (m, 2H), 3.81 (s, 3H), 1.37 (t, 3H), 1.11 (t, 3H).

### Step C: Production of 3-(Hydroxymethyl)-6-methoxy-2,3,4,9-tetrahydro-1H-beta-carbolin-1-one

A slurry of ethyl 3-(3-ethoxy-2-nitro-3-oxopropyl)-5-methoxy-1H-indole-2-carboxylate (3.0 g, 8.23 mmol), Raney nickel (50% aq. slurry, 3 mL), tetrahydrofuran (50 mL), ethanol (50 mL), and 6 N HCl (2 mL) were mixed on a Parr shaker under hydrogen (40 psi). After 26 hours, additional amounts of Raney nickel (3 mL of 50% aq. slurry) and 6 N HCl (2 mL) were added and the reaction was resumed for 5 hours. The reaction mixture was purged with nitrogen and filtered through a pad of Celite. The cake was washed with methanol, ethanol, and tetrahydrofuran. The filtrate was concentrated and partitioned between half-saturated sodium bicarbonate and ethyl acetate. The aqueous layer was extracted with ethyl acetate. The combined organic extracts were washed with 1 N NaOH and brine, dried (Na₂SO₄), and concentrated to give ethyl 2-(ethoxycarbonyl)-5-methoxytryptophanate as an off-white solid (2.52 g, 92%): ¹HNMR (300 MHz, DMSO-d₆) δ 11.45 (s, 1H), 7.28 (d, 1H), 7.06 (s, 1H), 6.89 (dd, 1H), 4.31 (q 2H), 3.89 (q, 2H), 3.76 (s, 3H), 3.57 (t, 1H), 3.32-3.14 (m, 2H), 1.34 (t, 3H), 0.95 (t, 3H). m/z 335 (M+H).

A suspension of ethyl 2-(ethoxycarbonyl)-5-methoxytryptophanate in ethanol (50 mL) was treated with sodium borohydride at room temperature. The mixture was warmed to 60° C. and stirred for 4.5 hours. The reaction was cooled to 0° C. and treated with a few mL of water and 1 N HCl until gas evolution had ceased. The clumpy mixture was stirred for 1 hour, and the pH was adjusted to pH 6. The mixture was diluted with water (100 mL) and filtered. The white precipitate was washed with water and ether to give the title compound as a white solid (1.05 g, 66%): ¹HNMR (300 MHz, DMSO-d₆) δ 11.40 (s, 1H), 7.28 (s, 1H), 7.25 (d, 1H), 7.02 (d, 1H), 6.84 (dd, 1H), 4.93 (t, 1H), 3.75 (s, 3H), 3.74-3.66 (m, 1H), 3.55-3.39 (m, 2H), 3.05-2.79 (m, 2H). m/z 247 (M+H).

### Step D. Production of 6-methoxy-1-oxo-2,3,4,9-tetrahydro-1H-beta-carbolin-3-yl)-methyl Methanesulfonate

Methanesulfonyl chloride (0.440 mL, 5.63 mmol) was added to a solution of 3-(hydroxymethyl)-6-methoxy-2,3,4,9-tetrahydro-1H-beta-carbolin-1-one (925 mg, 3.75 mmol) and triethylamine (1.6 mL, 11.3 mmol) in dimethylformamide (20 mL) at 0° C. After 30 minutes, the reaction was quenched with saturated ammonium chloride and extracted with ethyl acetate. The organic extracts were washed with saturated lithium chloride, water, and brine, dried (Na₂SO₄), and concentrated to furnish the title compound as an off-white solid (1.18 g, 97%): ¹HNMR (300 MHz, DMSO-d₆) δ 11.49 (s, 1H), 7.67 (s, 1H), 7.27 (d, 1H), 7.06 (d, 1H), 6.87 (dd, 1H), 4.26 (d, 1H), 4.10-4.00 (m, 1H), 3.76 (s, 3H), 3.21 (s, 3H), 3.17-2.89 (m, 2H). m/z 325 (M+H).

### Step E. Production of 3-(aminomethyl)-6-methoxy-2,3,4,9-tetrahydro-1H-beta-carbolin-1-one Trifluoroacetate

A solution of (6-methoxy-1-oxo-2,3,4,9-tetrahydro-1H-beta-carbolin-3-yl)methyl methanesulfonate (400 mg, 1.23 mmol) and sodium azide (120 mg, 1.85 mmol) in dimethylformamide (8 mL) was stirred at 80° C. for 10 hours. The reaction mixture was partitioned between saturated ammonium chloride and ethyl acetate. The organic extracts were washed with saturated lithium chloride, water, and brine, dried (Na₂SO₄), and concentrated to give 3-(azidomethyl)-6-methoxy-2,3,4,9-tetrahydro-1H-beta-carbolin-1-one as an off-white foam (343 mg). m/z 272 (M+H).

A mixture of 3-(azidomethyl)-6-methoxy-2,3,4,9-tetrahydro-1-H-beta-carbolin-1-one (343 mg) and 5% Pd/C (200 mg) in dimethylformamide (4 mL) and methanol (2 mL) was stirred under an atmosphere of hydrogen (balloon). After 5 hours, the reaction mixture was filtered. The filtrate was purified by reverse-phase HPLC (acetonitrile/water/0.05% trifluoroacetic acid) to give the title compound as a white solid (247 mg, 56%): ¹HNMR (300 MHz, DMSO-d₆) δ 11.54 (s, 1H), 7.95 (br s, 2H), 7.55 (s, 1H), 7.29 (d, 1H), 6.99 (d, 1H), 6.89 (dd, 1H), 4.00 (m, 1H), 3.76 (s, 3H), 3.14-2.84 (m, 4H). HRMS calculated for C₁₃H₁₆N₃O₂ (M+H) 246.1237, found 246.1271.

### EXAMPLE 45

This example illustrates the production of 3-(2-aminoethyl)-6-methoxy-2,3,4,9-tetrahydro-1H-beta-carbolin-1-one trifluoroacetate.

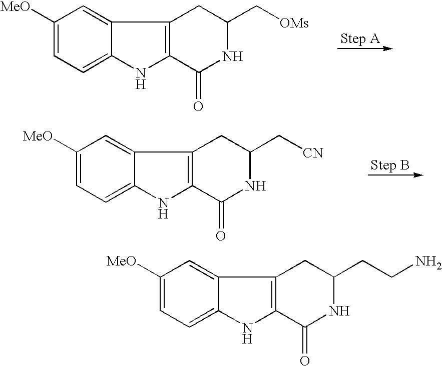

### Step A. Production of (6-methoxy-1-oxo-2,3,4,9-tetrahydro-1H-beta-carbolin-3-yl)acetonitrile

A solution of (6-methoxy-1-oxo-2,3,4,9-tetrahydro-1H-beta-carbolin-3-yl)methyl methanesulfonate (product of Example 44, step D; 500 mg, 1.54 mmol) and potassium cyanide (150 mg, 2.31 mmol) in dimethylformamide (8 mL) was stirred at 80° C. for 16 hours. The reaction mixture was partitioned between water and ethyl acetate. The aqueous layer was extracted with ethyl acetate. The combined organic extracts were washed with water, saturated LiCl and brine, dried (Na₂SO₄), and concentrated. The residue was triturated with hot ethyl acetate to give the title compound as an off-white solid (218 mg). A second crop was obtained from the mother liquor (39 mg). The mother liquor was purified by flash chromatography (25→75% ethyl acetate/hexanes) to yield another 95 mg. Total yield of the title compound was 352 mg (90%): ¹HNMR (300 MHz, DMSO-d₆) δ 11.50 (s, 1H), 7.72 (s, 1H), 7.28 (d, 1H), 7.07 (d, 1H), 6.87 (dd, 1H), 4.12-4.00 (m, 1H), 3.76 (s, 3H), 3.31-3.12 (m, 1H), 2.92-2.71 (m, 3H). HRMS calculated for C₁₄H₁₄N₃O₂ (M+H) 256.1081, found 256.1085.

### Step B: Production of 3-(2-aminoethyl)-6-methoxy-2,3,4,9-tetrahydro-1H-beta-carbolin-1-one Trifluoroacetate

A solution of (6-methoxy-1-oxo-2,3,4,9-tetrahydro-1H-beta-carbolin-3-yl)acetonitrile (250 mg, 0.979 mmol) in tetrahydrofuran (10 mL) was added to a 0° C. 1 M solution of lithium aluminum hydride (4 mL of 1 M solution in tetrahydrofuran, 4 mmol) in 6 mL THF. The reaction was allowed to warm to room temperature. After 90 minutes, the reaction was quenched by the dropwise addition of water (0.152 mL), 15% NaOH (0.152 mL), and water (0.456 mL). The mixture was diluted with tetrahydrofuran and filtered. The cake was washed with tetrahydrofuran. The filtrate was concentrated and purified by reverse-phase HPLC (acetonitrile/water/0.05% trifluoroacetic acid) to give the title compound as a yellow solid (80 mg, 22%): ¹HNMR (300 MHz, DMSO-d₆) δ 11.48 (s, 1H), 7.80 (br s, 2H), 7.68 (s, 1H), 7.30 (d, 1H), 7.09 (d, 1H), 6.89 (dd, 1H), 3.91-3.81 (m, 1H), 3.79 (s, 3H), 3.12 (dd, 1H), 3.05-2.91 (m, 2H), 2.74 (dd, 1H), 2.05-1.77 (m, 2H). HRMS calculated for C₁₄H₁₈N₃O₂ (M+H) 260.1394, found 260.1398.

### EXAMPLE 46

This example illustrates the production of 3-(3-aminopropyl)-6-methoxy-2,3,4,9-tetrahydro-1H-beta-carbolin-1-one trifluoroacetate.

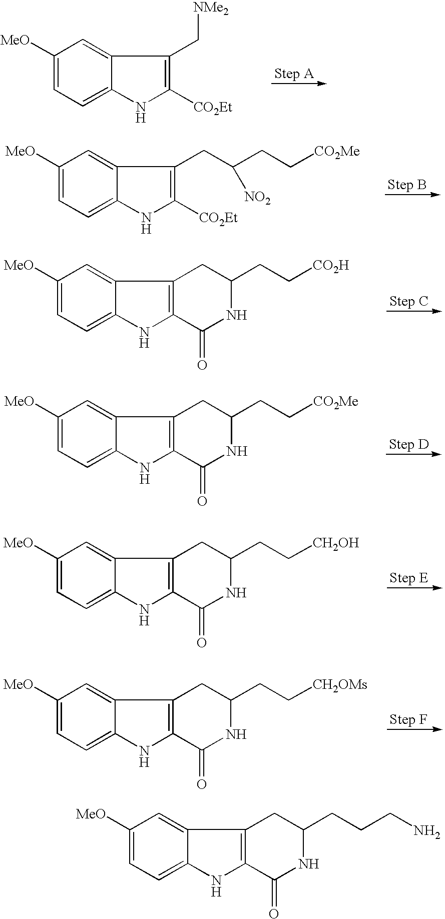

### Step A: Production of Ethyl 5-methoxy-3-(5-methoxy-2-nitro-5-oxopentyl)-1H-indole-2-carboxylate

A solution of ethyl 3-[(dimethylamino)methyl]-5-methoxy-1H-indole-2-carboxylate (product of Example 44, step A; 2.0 g, 7.2 mmol) in methanol (50 mL) was treated with dimethylsulfate (1.4 mL, 14.4 mmol), methyl 4-nitro-butyrate (4.0 mL, 31.2 mmol), and sodium methoxide (16.0 mL, 8.0 mmol) and allowed to stir at room temperature for 2 hours. The reaction contents were poured into water (200 mL) and the resulting precipitate was filtered and dried to give ethyl 5-methoxy-3-(5-methoxy-2-nitro-5-oxopentyl)-1H-indole-2-carboxylate as a white solid (2.0 g, 73%): ¹HNMR (400 MHz, DMSO-d₆) δ 11.64 (s, 1H), 7.29 (d, 1H), 7.06 (d, 1H), 6.90 (dd, 1H), 4.92 (bs, 1H), 4.30 (q, 2H), 3.76 (s, 3H), 3.62 (m, 1H), 3.52 (s, 3H), 3.49 (m, 1H), 2.36 (t, 1H), 2.20 (m, 1H), 2.07 (m, 1H), 1.33 (t, 3H).

### Step B: Production of 3-(6-methoxy-1-oxo-2,3,4,9-tetrahydro-1H-beta-carbolin-3-yl)propanoic Acid

A solution of ethyl 5-methoxy-3-(5-methoxy-2-nitro-5-oxopentyl)-1H-indole-2-carboxylate (1.92 g, 5.06 mmol) in tetrahydrofuran (30 mL) and ethanol (30 mL) was treated with 1 N HCl (10 mL) and cooled to 0° C. The mixture was treated with Raney nickel (50% aqueous slurry, 1.5 mL) and mixed under hydrogen (35 psi) with a Parr shaker for 80 hours. The slurry was filtered through Celite. The filtrate was concentrated to give ethyl 3-(2-amino-5-methoxy-5-oxopentyl)-5-methoxy-1H-indole-2-carboxylate as an off-white solid (2.67 g).

A suspension of crude ethyl 3-(2-amino-5-methoxy-5-oxopentyl)-5-methoxy-1H-indole-2-carboxylate (2.61 g) in acetic acid (30 mL) and 6 N HCl (60 mL) was stirred at 70° C. for 45 min. The solution was concentrated on the rotavap until a white solid precipitate formed. The solid was filtered and washed with water to give 4-amino-5-[2-(ethoxycarbonyl)-5-methoxy-1H-indol-3-yl]pentanoic acid (800 mg). The filtrate was extracted with ethyl acetate to give an oil (˜1.4 g). The solid and oil were combined and dissolved in MeOH (30 mL) and ammonium hydroxide (30 mL). The solution was stirred at room temperature overnight. The solution was concentrated until neutral, diluted with water, and treated with 1 N HCl until a precipitate formed. Ethyl acetate was added to the mixture. The mixture was filtered, and the solid was washed with ethyl acetate to give the title compound as a white solid (494 mg, 34% overall yield): ¹HNMR (300 MHz, DMSO-d₆) δ 11.40 (s, 1H), 7.54 (s, 1H), 7.25 (d, 1H), 7.04 (d, 1H), 6.84 (dd, 1H), 3.75 (s, 3H), 3.71 (m, 1H), 3.03 (dd, 1H), 2.66 (dd, 1H), 2.39 (t, 2H), 1.97-1.71 (m, 2H). m/z 289 (M+H).

### Step C: Production of Methyl 3-(6-methoxy-1-oxo-2,3,4,9-tetrahydro-1H-beta-carbolin-3-yl)propanoate

HCl (g) was bubbled into methanol (20 mL) for 10 minutes. Then 3-(6-Methoxy-1-oxo-2,3,4,9-tetrahydro-1H-beta-carbolin-3-yl)propanoic acid (600 mg, 2.1 mmol) was added and stirred for 30 minutes and then condensed to dryness. The solid was suspended in diethylether and filtered to give methyl 3-(6-methoxy-1-oxo-2,3,4,9-tetrahydro-1H-beta-carbolin-3-yl)propanoate as an off-white solid (505 mg, 80%): ¹HNMR (400 MHz, DMSO d₆) δ 11.38 (s, 1H), 7.53 (s, 1H), 7.23 (d, 1H), 7.01 (d, 1H), 6.82 (dd, 1H), 3.73 (m, 4H), 3.55 (s, 3H), 3.42 (bs, 2H), 3.01 (m, 1H), 2.64 (m, 1H), 1.90 (m, 1H), 1.77 (m, 1H). HRMS calculated for C₁₇H₂₂N₃O₃ (M+H) 303.1355, found 303.1339.

### Step D: Production of 3-(3-hydroxypropyl)-6-methoxy-2,3,4,9-tetrahydro-1H-beta-carbolin-1-one

A solution of methyl 3-(6-methoxy-1-oxo-2,3,4,9-tetrahydro-1H-beta-carbolin-3-yl)propanoate (445 mg, 1.47 mmol) in tetrahydrofuran (20 mL) cooled to 0° C. was slowly treated with lithium aluminum hydride (2.9 mL of a 1 M solution in tetrahydrofuran, 2.9 mmol) and allowed to warm to room temperature. The solution was then cooled to 15° C. and treated with water (0.27 mL), 15% NaOH solution (0.27 mL) and water (0.81 mL) then allowed to warm to room temperature. The solids were filtered off and the filterate condensed to yellow oil. The oil was purified by flash chromatography (100% ethyl actate→10% methanol/90% ethyl acetate) to give 3-(3-hydroxypropyl)-6-methoxy-2,3,4,9-tetrahydro-1H-beta-carbolin-1-one as an off white foam (150 mg, 37%): ¹HNMR (400 MHz, DMSO d₆) δ 11.36 (s, 1H), 7.44 (s, 1H), 7.23 (d, 1H), 7.03 (d, 1H), 6.82 (dd, 1H), 4.42 (t, 1H), 3.72 (m, 4H), 3.39 (m, 2H), 3.02 (m, 1H), 2.61 (m, 1H), 1.67 (m, 1H), 1.53 (m, 2H). HRMS calculated for C₁₇H₂₂N₃O₃ (M+H) 275.1362, found 275.1390.

### Step E: Production of 3-(6-methoxy-1-oxo-2,3,4,9-tetrahydro-1H-beta-carbolin-3-yl)propyl Methanesulfonate

Methanesulfonyl chloride (1.66 g, 14.6 mmol) was added to a solution of 3-(3-hydroxypropyl)-6-methoxy-2,3,4,9-tetrahydro-1H-beta-carbolin-1-one (1.6 g, 5.8 mmol) in pyridine (5 mL) and methylene chloride (10 mL) at 0° C. After 2 hours the solution was quenched with 1 N HCl, extracted with ethyl acetate, washed with brine, dried over magnesium sulfate and concentrated to afford 3-(6-methoxy-1-oxo-2,3,4,9-tetrahydro-1H-beta-carbolin-3-yl)propyl methanesulfonate as a foam (1.5 g, 74%): ¹HNMR (400 MHz, DMSO d₆) δ 11.38 (s, 1H), 7.52 (s, 1H), 7.23 (d, 1H), 7.01 (d, 1H), 6.82 (dd, 1H), 4.20 (m, 2H), 3.75 (s, 4H), 3.41 (m, 1H), 3.15 (s, 3H), 3.02 (m, 1H), 2.61 (m, 1H), 1.77 (m, 3H), 1.61 (m, 1H).

### Step F: Production of 3-(3-aminopropyl)-6-methoxy-2,3,4,9-tetrahydro-1H-beta-carbolin-1-one Trifluoroacetate

A solution of 3-(6-methoxy-1-oxo-2,3,4,9-tetrahydro-1H-beta-carbolin-3-yl)propyl methanesulfonate (105 mg, 0.30 mmol) in dimethylformamide (3.0 mL) was treated with sodium azide (29 mg, 0.45 mmol) and heated to 100° C. for 16 hours. Cooled to room temperature, poured into water, extracted with ethyl acetate, dried over magnesium sulfate, filtered and concentrated to give 3-(3-azidopropyl)-6-methoxy-2,3,4,9-tetrahydro-1H-beta-carbolin-1-one as an off-white foam (100 mg).

A solution of 3-(3-azidopropyl)-6-methoxy-2,3,4,9-tetrahydro-1H-beta-carbolin-1-one (100 mg, 0.33 mmol) in dimethylformamide (4 mL) was added 10% Pd(C) (50 mg) and stirred under an atmosphere of hydrogen (balloon) for 2 hours. The reaction was filtered, concentrated and purified by prep rpHPLC and condensed to give 3-(3-aminopropyl)-6-methoxy-2,3,4,9-tetrahydro-1H-beta-carbolin-1-one trifluoroacetate (53 mg, 65% overall yield): ¹HNMR (400 MHz, DMSO d₆) δ 11.38 (s, 1H), 7.67 (bs, 2H), 7.52 (s, 1H), 7.24 (d, 1H), 7.00 (d, 1H), 6.83 (dd, 1H), 3.74 (m, 4H), 3.03 (m, 1H), 2.78 (m, 2H), 2.65 (m, 1H), 1.63 (m, 4H). m/z 274.2 (M+H).

### EXAMPLE 47

This example illustrates the production of 3-[3-(benzylamino)propyl]-6-methoxy-2,3,4,9-tetrahydro-1H-beta-carbolin-1-one trifluoroacetate.

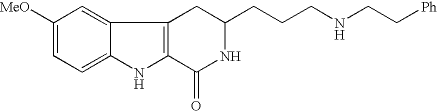

A solution of 3-(6-methoxy-1-oxo-2,3,4,9-tetrahydro-1H-beta-carbolin-3-yl)propyl methanesulfonate (product of Example 46, step E; 200 mg, 0.57 mmol) in dimethylformamide (4.0 mL) was treated with benzylamine (92 mg, 0.86 mmol) and stirred at 50° C. for 16 hours. Cooled to room temperature, filtered, purified by rpHPLC, and lypholized to give 3-[3-(benzylamino)propyl]-6-methoxy-2,3,4,9-tetrahydro-1H-beta-carbolin-1-one trifluoroacetate (75 mg, 27%): ¹HNMR (400 MHz, DMSO d₆) δ 11.40 (s, 1H), 8.78 (bs, 2H), 7.51 (s, 1H), 7.43 (m, 5H), 7.24 (d, 1H), 6.99 (s, 1H), 6.83 (dd, 1H), 4.12 (t, 2H), 3.73 (s, 3H), 3.02 (m, 1H), 2.91 (m, 2H), 2.65 (m, 1H), 1.65 (m, 4H). m/z 478.5 (M+H).

### EXAMPLE 48

This example illustrates the production of 8,9,10,11-tetrahydro-7H-pyrido[3′,4′:4,5]pyrrolo[2,3-f]isoquinolin-7-one trifluoroacetate.

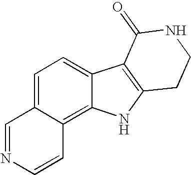

### Step A: Production of piperidine-2,4-dione

Sodium 3-(methoxycarbonyl)-4-oxo-1,4,5,6-tetrahydropyridin-2-olate (Degussa) (50 g, 259 mmol) was partitioned between 2N aqueous hydrogen chloride and dichloromethane. The aqueous layer was extracted two additional times with dichloromethane. The organic extracts were dried over sodium sulfate, filtered and evaporated. The residue was suspended in acetonitrile (500 mL) and water (100 mL) and heated to reflux for 3 hours. The reaction mixture was cooled and evaporated. The residue was recrystallized from 1:1 ethyl acetate:hexane to provide piperidine-2,4-dione (19.5 g, 67%) as a white solid: ¹HNMR (400 MHz, CDCl₃) δ 7.05 (s, 1H), 3.58 (td, 2H), 3.34 (s, 2H), 2.64 (t, 2H). m/z 114 (M+H).

### Step B: Production of 8,9,10,11-tetrahydro-7H-pyrido[3′,4′:4,5]pyrrolo[2,3-f]isoquinolin-7-one Trifluoroacetate

5-aminoisoquinoline (288 mg, 2 mmol) and piperdine-2,4-dione (248 mg, 2.2 mmol) were combined in a vial an heated at 120° C. for 1 hour. After cooling, a solution of Pd(OAc)₂ (90 mg) and Cu(OAc)₂ (725 mg) in DMF (5 mL) were added and the resulting suspension heated to 120° C. for 2 hours. After cooling the solids were removed by filtration and the product was isolated by reverse phase chromatography to yield the title compound as a yellow solid (29 mg, 6%): ¹H NMR (400 MHz, DMSO-d₆) δ 9.69 (br s, 1H), 8.68 (br s, 1H), 8.60 (d, 1H), 8.42 (d, 1H), 8.03 (d, 1H), 3.53 (t, 2H), 3.16 (t, 2H). m/z 238 (M+H).

### EXAMPLE 49

This example illustrates the production of methyl 1-oxo-2,3,4,5-tetrahydro-1H-pyrido[4,3-b]indole-6-carboxylate.

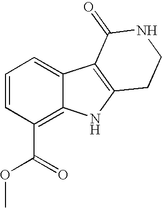

Methyl anthranilate (260 μL, 2 mmol) and piperdine-2,4-dione (248 mg, 2.2 mmol) were combined in a vial an heated at 120° C. for 1 hour. After cooling, a solution of Pd(OAc)₂ (90 mg) and Cu(OAc)₂ (725 mg) in DMF (5 mL) were added and the resulting suspension heated to 120° C. for 2 hours. After cooling the solids were removed by filtration and product was isolated by reverse phase chromatography to yield the title compound as a yellow solid (110 mg, 23%): ¹H NMR (400 MHz, DMSO-d₆) δ 10.99 (br s, 1H), 8.29 (d, 1H), 7.83 (d, 1H), 7.24 (t, 1H), 3.99 (s, 3H), 3.64 (t, 2H), 3.15 (t, 2H). m/z 245 (M+H).

### EXAMPLE 50

This example illustrates the production of 2-methoxy-7,8,9,10-tetrahydrocyclohepta[b]indol-6(5H)-one oxime.

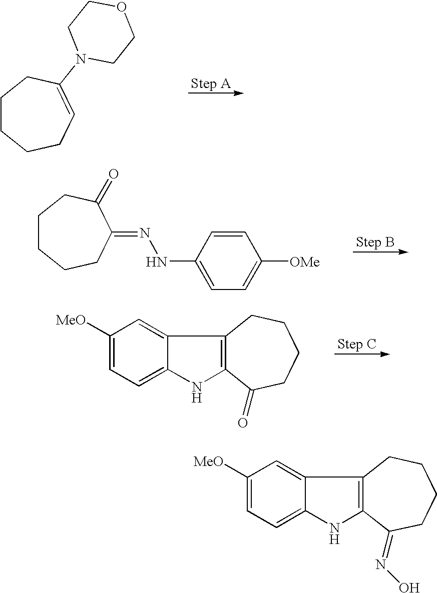

### Step A: Production of 1,2-cycloheptanedione, mono[(4-methoxyphenyl)hydrazone]

To a solution of p-anisidine (6.15 g, 50 mmol) in 4N HCl (25 mL) at 0° C. was added dropwise a solution of sodium nitrite (3.45 g, 50 mmol) in 12 ml water over 20 minutes. The mixture was stirred at 0° C. for for an additional 30 minutes and then a solution of 1-morpholino-1-cycloheptene (9.05 g, 50 mmol) in dimethoxyethane (25 ml) was added dropwise over 30 minutes. The mixture was allowed to warm to room temperature and stirred for additional 1.5 hours. The resultant dark red precipitate was collected by filtration and washed thoroughly with water. The solid was dissolved in ethyl acetate, dried (MgSO₄), and evaporated. The residue was triturated with hexane to give the title compound as a yellow solid (8.1 g, 66%): ¹HNMR (300 MHz, DMSO-d₆) δ 13.50 (s, 1H), 7.18 (d, 2H), 6.85 (d, 2H), 3.68 (s, 3H), 2.58 (m, 4H), 1.66 (m, 6H). m/z 247 (M+H).

### Step B: Production of 2-methoxy-7,8,9,10-tetrahydrocyclohepta[b]indol-6(5H)-one

To a solution of 1,2-cycloheptanedione, mono[(4-methoxyphenyl)hydrazone] (4.9 g, 20 mmol) in 50 ml ethanol was added concentrated H₂SO₄ (4 g) and the mixture was heated at reflux for 4 hours. After cooling to room temperature, the reaction mixture was stored at 0° C. for 2 hours. The resultant precipitate was collected by filtration, washed with water and air-dried. A solution of the above solid in ethyl acetate was treated with decolorizing carbon, warmed at 60° C. for 45 minutes and filtered. The filtrate was dried (MgSO₄), and evaporated to give the title compound as a pale yellow solid (1.15 g, 25%): ¹HNMR (300 MHz, DMSO-d₆) δ 11.18 (s, 1H), 7.29 (d, 1H), 7.10 (d, 1H), 6.93 (dd, 1H), 3.79 (s, 3H), 3.06 (t, 2H), 2.74 (t, 2H), 1.91 (m, 4H). m/z 230 (M+H).

### Step C: 2-Methoxy-7,8,9,10-tetrahydrocyclohepta[b]indol-6(5H)-one Oxime

A mixture of 2-methoxy-7,8,9,10-tetrahydrocyclohepta[b]indol-6(5H)-one (343 mg, 1.5 mmol), hydroxylamine hydrochloride (207 mg, 3 mmol) and sodium acetate (410 mg, 5 mmol) in 15 ml of ethanol-water (4:1 v/v) was heated at reflux for 15 hours. After cooling to room temperature, the reaction mixture was evaporated and the residue was diluted with ice water. The resultant white precipitate was collected by filtration, washed with water and dried under vacuum to give the title compound as a mixture of syn- and anti isomers (288 mg, 79%): ¹HNMR (300 MHz, CDCl₃) δ 8.71 (s, 1H), 6.87-7.27 (m, 3H), 3.86 (s, 3H), 2.93-2.98 (m, 4H), 1.89-2.05 (m, 4H). m/z 245 (M+H).

### EXAMPLE 51

This example illustrates the production of 6-methoxy-2,3,4,9-tetrahydro-1H-beta-carboline-1-thione.

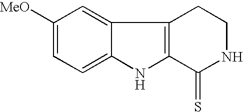

To a solution of 3-(2-isothiocyanatoethyl)-1H-Indole (2.32 g, 10 mmol) in dry methylene chloride (60 mL) at 0° C. was added 8 ml of trifluoroacetic acid in small portions. The mixture was warmed to room temperature and stirred for 60 hours. The reaction mixture was evaporated to dryness and the residue was triturated with ether-hexane to give a dark solid, which was collected by filtration, washed with ether-hexane and air-dried. A solution of the above solid in dimethoxy-ethane was treated with decolorizing carbon and filtered. The filtrate was dried (MgSO₄), and evaporated. Recrystallization of the residue from ethanol gave the title compound as a yellow solid (1.53 g, 66%): ¹HNMR (300 MHz, DMSO-d₆) δ 11.17 (s, 1H), 9.76 (s, 1H), 7.37 (d, 1H), 7.06 (d, 1H), 6.89 (dd, 1H), 3.78 (s, 3H), 3.54 (m, 2H), 2.95 (t, 2H). m/z 233 (M+H).

### EXAMPLE 52

This example illustrates the production of 6-methoxy-2,3,4,9-tetrahydro-1H-beta-carbolin-1-one oxime.

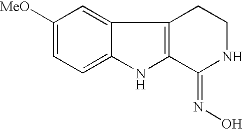

To a mixture of 6-methoxy-2,3,4,9-tetrahydro-1H-beta-carboline-1-thione (compound of Example 8; 0.58 g, 2.5 mmol) and anhydrous potassium carbonate (1.03 g, 7.5 mmol) in dry THF (50 mL) was added 2 ml of methyl iodide and the resultant suspension was stirred at room temperature overnight. The mixture was then filtered to remove the insoluble salts, the filtrate was evaporated and the residue was partitioned between ethyl acetate and water. The organic layer was dried (MgSO₄), and evaporated to give a yellow solid. To a solution of the above solid in methanol (50 ml) were added hydroxylamine hydrochloride (0.35 g, 5 mmol) and sodium bicarbonate (0.85 g, 10 mmol) and the mixture was heated at reflux for 24 hours. After cooling to room temperature, the solution was evaporated and the residue was treated with ice water. The resultant precipitate was collected by filtration and air-dried. Purification of the above solid by chromatography (silica gel, ethyl acetate) gave the title compound as a yellow crystalline solid (0.32 g, 55%): ¹HNMR (300 MHz, CDCl₃) δ9.17 (brs, 1H), 7.26 (s, 1H), 7.16 (d, 1H), 6.96 (d, 1H), 6.86 (dd, 1H), 5.45 (s, 1H), 3.85 (s, 3H), 3.56 (m, 2H), 2.96 (t, 2H). m/z 232 (M+H).

### EXAMPLE 53

This example illustrates that MK-2 knock-out mice (MK2 (−/−)) are resistant to the formation of K/BN serum-induced arthritis.

A strain of mice has been reported that develops symptoms similar to human rheumatoid arthritis. The mice were designated K/BxN mice. See, Wipke, B. T. and P. M. Allen, J. of Immunology, 167: 1601-1608 (2001). Serum from the mice can be injected into host animals to provoke a typical RA response. The progression of the RA symptoms in the mice is measured by measuring paw thickness as a function of time.

In the present example, host mice having normal MK-2 production (MK-2 (+/+)) were genetically altered by disabling the gene encoding MK-2 to produce mice having no capability of endogenous synthesis of active MK-2 (MK-2 (−/−)). Normal host mice (MK-2 (+/+)) and MK-2 knock-out mice (MK-2 (−/−), were separated into four groups with each group containing both male and female mice. All groups of mice were treated similarly, except that one group (Normal), composed of MK-2 (+/+) mice that served as the control group, was not injected with serum from K/BxN mice, while the other three groups were injected with K/BxN serum at day 0. The other three groups of mice were MK-2 (+/+), MK2 (−/−), and Anti-TNF. The Anti-TNF group was composed of MK-2 (+/+) mice which were also injected at day) with anti-TNF antibody. The paw thickness of all mice was measured immediately after the injections on day 0, and then on each successive day thereafter for 7 days.

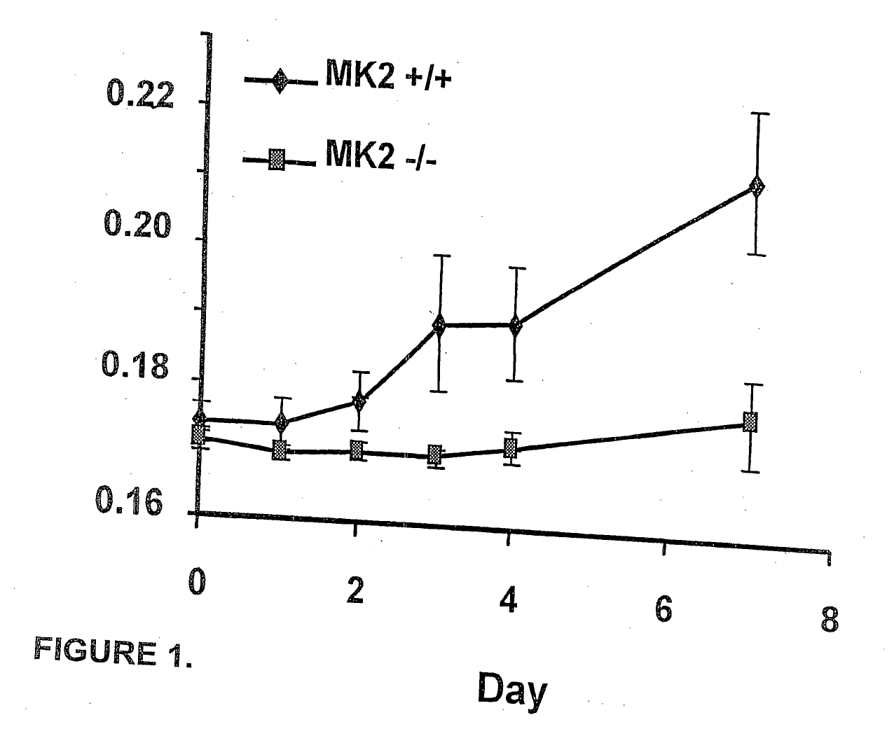

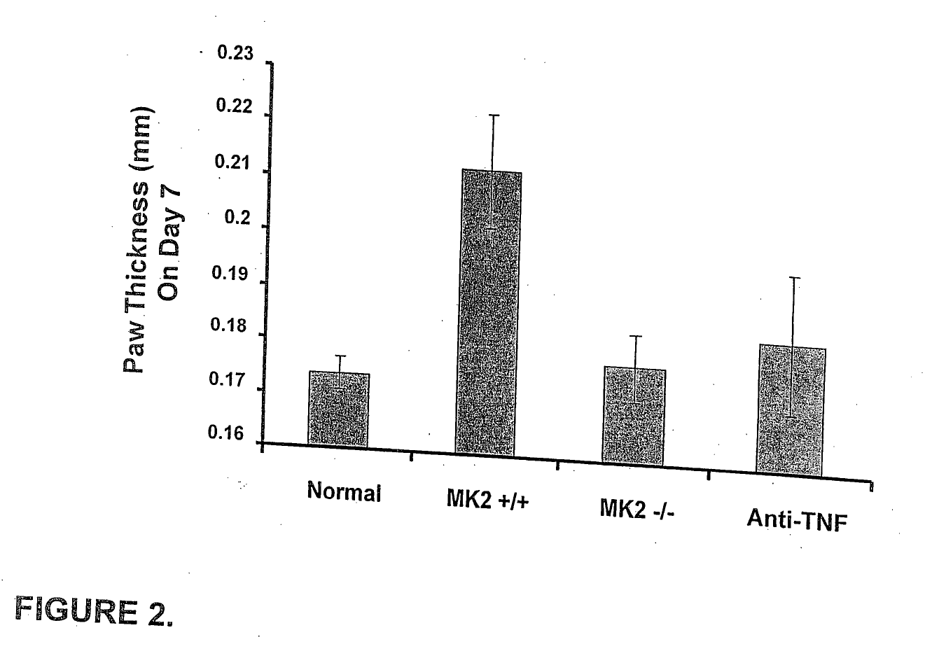

FIG. 1 is a graph that shows paw thickness as a function of time from day 0 to day 7 for MK-2 (+/+) and MK-2 (−/−) mice, which have received serum injection. It can be seen that paw thickness increased significantly for MK-2 (+/+) mice, whereas there was substantially no increase in paw thickness for MK-2 knock-out mice. This indicated the requirement for a functioning MK-2 regulatory system to the inflammatory response caused by the serum challenge. When anti-TNF antibody was administered to the MK-2 (+/+) mice along with the serum injection, the swelling response was significantly reduced. This can be seen in FIG. 2, which is a bar chart showing paw thickness at seven days after injection for normal mice, MK-2 (+/+) mice receiving serum, MK-2 (−/−) mice receiving serum, and MK2 (+/+) mice receiving serum and anti-TNF antibody.

This data shows that the MK-2 knock-out mice show no arthritic response to a serum challenge, whereas MK-2 (+/+) mice show a normal response. Treatment of MK-2 (+/+) mice that receive a serum challenge with anti-TNF antibody reduces the response back to near-normal levels. This illustrates the utility of the MK-2 regulatory system as a potential control point for the modulation of TNF production, and indicates that such regulation could serve as a treatment for inflammation—such as that caused by arthritis, for example. It further shows that MK-2 inhibition can have a beneficial effect on inflammation, and indicates that administration of an MK-2 inhibitor can be an effective method of preventing or treating TNF modulated diseases or disorders.

All references cited in this specification, including without limitation all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinency of the cited references.

In view of the above, it will be seen that the several advantages of the invention are achieved and other advantageous results obtained.

As various changes could be made in the above methods and compositions without departing from the scope of the invention, it is intended that all matter contained in the above description shall be interpreted as illustrative and not in a limiting sense. In addition, it should be understood that aspects of the various embodiments may be interchanged both in whole or in part.

1. A method for treating or preventing a disease or disorder in a subject, which disease or disorder is one that can be treated or prevented by inhibiting the activity of MK-2, the method comprising administering to the subject a compound, or a salt, isomer, or prodrug thereof, wherein the compound has the structure: 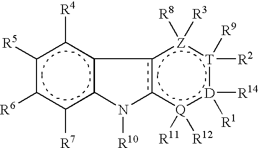
wherein:
Q, D, T and Z are selected from carbon, sulfur, or nitrogen;
when Q is sulfur, R¹² is oxo, and R¹¹ is optionally oxo or is absent;
when T is sulfur, R² is oxo, and R⁹ is optionally oxo or is absent;
when Z is nitrogen, D is nitrogen, and R² and R¹² are oxo;
R¹ is selected from H, alkyl, alkenyl, alkynyl, allyl, acetyl, amino, alkoxy, hydroxy, oxo, carboxy, allylalkyl, benzyl, 2-ethylalkyl, haloacetyl, t-butyldialkylsilyloxyalkyl, alkoxycarbonylaminoalkyl, haloalkylbenzyl, haloalkylcarbonyl, alkylcarbonyl, alkoxycarbonyl, arylcarbonyl, benzyloxycarbonyl, arylalkenylamino, alkoxyheterocyclylcarbonyl, haloarylcarbonyl, or substituted or unsubstituted alkyl, which, if substituted, has one or more substituent groups selected from H, halo, amino, aryl, or heteroaryl;
R² is selected from H, alkyl, alkoxy, hydroxy, carbonyl, oxo, benzyl, hydroxyalkyl, dialkylamino, carboxyl, amino, halo, alkoxycarbonyl, alkoxycarbonyl-R¹⁵, aminoalkyl, haloalkyl, hydroxyalkylbenzyl, haloalkylbenzyl, arylalkenylamino, haloalkoxybenzyl, cyanoalkyl, heterocyclylalkyl, arylalkyl-R¹⁵, alkyl-SO₂—R¹⁵, substituted or unsubstituted aryl, which, if substituted, has one or more substituent groups selected from H, alkyl, amino, nitro, halo, haloalkyl, or hydroxyalkyl, or substituted or unsubstituted alkyl, which, if substituted, has one or more substituent groups selected from H, halo, amino, aryl, or heteroaryl;
R³ is selected from H, oxo, alkyl, alkoxy, amino, halo, haloalkyl, hydroxy, carbonyl, alkoxycarbonyl, benzyl, aminoalkyl, hydroxyalkyl, dialkylamino, arylalkenylamino, carboxy, haloalkyl, hydroxyalkylbenzyl, haloalkylbenzyl, haloalkoxybenzyl, heterocyclylalkyl, substituted or unsubstituted aryl, which, if substituted, has one or more substituent groups selected from H, alkyl, amino, nitro, halo, haloalkyl, or hydroxyalkyl, or substituted or unsubstituted alkyl, which, if substituted, has one or more substituent groups selected from H, halo, amino, aryl, or heteroaryl, or R¹ and R³ optionally join to form an alkyl bridge between the ring atoms to which R¹ and R³ are attached, or R² and R³ optionally join to form a cycloalkyl ring;
R⁴, R⁶ and R⁷ are independently selected from H, alkyl, benzyl, halo, haloalkyl, alkoxy, haloalkoxy, carboxyl, CO₂—R¹⁵, carboxyl, nitro, amino, alkylamino, dialkylamino, benzyloxy, hydrazinocarbonyl, alkoxycarbonyl, alkoxycarbonylheterocyclylcarbonyl, hydroxy, or substituted or unsubstituted 6-membered heterocyclylcarbonyl, which, if substituted, has one or more substituent groups selected from H or alkoxycarbonyl, or R⁶ and R⁷ optionally join to form a heteroaryl ring;
R⁵ is selected from H, alkyl, alkenyl, alkylthio, hydroxy, carboxyl, halo, alkoxy, benzylalkyl, benzylalkoxy, arylalkyl, nitro, alkoxycarbonylalkoxy, alkylthio, benzyloxy, arylalkynyl, arylcarbonylalkoxy, haloalkoxy, haloalkyl, arylalkoxy, haloarylalkoxy, benzyloxoalkoxy, alkoxycarbonylbenzyloxy, alkoxycarbonyl, alkoxyoxoalkoxy, alkoxyoxoarylalkoxy, halobenzyloxy, heterocyclylalkoxy, dialkylaminooxoalkoxy, aminocarbonyl, arylalkynyl, hydrazinocarbonyl, carbonylalkoxy, alkylsulfonylbenzyloxy, CO₂—R¹⁵, carbamoyl, naphthylalkoxy, alkoxyaryloxy, alkoxycarbonylheterocyclylalkoxy, haloalkylbenzyloxy, alkoxyacetylaminoacetyloxy, acetoxy, cycloalkylalkoxy, acetyloxy, morpholinylcarbonyl, alkoxyalkoxy, or R⁴ and R⁵ optionally join to form a heterocyclic ring;
R⁸ and R⁹ are independently optionally present and if present, are independently selected from H, oxo, carbonitrile, alkyl, or is optionally absent, or R³ and R⁸ optionally join to form a cycloalkyl ring;
R¹⁰ is selected from H, alkyl, benzyl, aryl, or hydroxyalkyl;
R¹¹ is optionally present and if present, is selected from H, oxo, alkyl, halobenzyl, benzyl, haloalkyl, 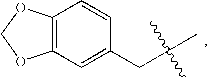
alkoxycarbonyl, carboxyalkyl, benzodioxinylalkyl, substituted or unsubstituted aryl or arylalkyl, which, if substituted, have one or more substituent groups selected from H, alkyl, or alkoxy;
R¹² is optionally present and if present, is selected from H, ═S, oxo, alkyl, carbonyl-R¹³, carboxyl-R¹³, ketone, cyano, cyanoalkyl, alkoxycarbonyl, aminoalkyl, alkoxycarbonylalkyl, carbamyl, hydroxycarbamyl, haloalkyl, heterocyclyl, alkylacetate, heterocyclylalkyl, alkoxycarbonylalkyl, hydroxyalkyl, dihydroxyalkyl, cyanoalkyl, ═N—OR¹⁵, ═N—N(R¹⁵)₂, or hydroxyamino, or R¹ and R¹² optionally join to form a substituted or unsubstituted 5 or 6-membered heteroaryl ring, which, if substituted, has one or more substituent groups selected from H, alkyl, oxo, or alkoxycarbonylalkyl, or R¹¹ and R¹² optionally join to form a ring system having the structure: 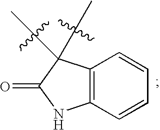
R¹³ is optionally present and if present, is selected from H, alkyl, alkoxy, amino, alkynyl, allyl, alkenyl, hydrazine, benzyl, glycine, allylalkyl, haloalkyl, hydroxyalkyl, hydroxyamino, dihydroxyalkyl, alkoxyalkyl, alkoxyamino, alkylthioalkyl, cycloalkyl, cyclohexylalkyl, amino, alkylamino, benzylalkyl, benzylamino, benzyloxycarbonylalkylamino, dialkylamino, adamantyl, haloalkoxybenzyl, alkoxyaminoalkyl, haloalkylarylalkyl, heterocyclylalkyl, alkoxyalkylamino, arylthioalkyl, substituted or unsubstituted aryl or arylalkyl, which, if substituted, have one or more substituent groups selected from H, alkyl, alkoxy, amino, nitro, halo, haloalkyl, or hydroxyalkyl;
R¹⁴ is optionally present and if present, is selected from H or alkyl; and
R¹⁵ is optionally present and if present, is selected from H, alkyl, alkoxy, or aminoalkyl.

2. The method according to claim 1, wherein:
Q, D, T and Z are selected from carbon, sulfur, or nitrogen;
when Q is sulfur, R¹² is oxo, and R¹¹ is optionally oxo or is absent;
when T is sulfur, R² is oxo, and R⁹ is optionally oxo or is absent;
when Z is nitrogen, D is nitrogen, and R² and R¹² are oxo;
R¹ is selected from H, alkyl, alkenyl, alkynyl, allyl, acetyl, amino, alkoxy, hydroxy, oxo, carboxy, allylalkyl, benzyl, 2-ethylalkyl, haloacetyl, alkoxycarbonylaminoalkyl, t-butyldialkylsilyloxyalkyl, alkoxycarbonylaminoalkyl, haloalkylbenzyl, haloalkylcarbonyl, alkylcarbonyl, alkoxycarbonyl, arylcarbonyl, benzyloxycarbonyl, alkoxycarbonyl, arylalkenylamino, alkoxyheterocyclylcarbonyl, haloarylcarbonyl, or substituted or unsubstituted alkyl, which, if substituted, has one or more substituent groups selected from H, halo, amino, aryl, or heteroaryl;
R² is selected from H, alkyl, alkoxy, hydroxy, carbonyl, oxo, benzyl, hydroxyalkyl, dialkylamino, carboxyl, amino, halo, alkoxycarbonyl, aminoalkyl, haloalkyl, hydroxyalkylbenzyl, haloalkylbenzyl, alkoxycarbonyl-R¹⁵, cyanoalkyl, arylalkyl-R¹⁵, alkyl-SO₂—R¹⁵, arylalkenylamino, haloalkoxybenzyl, heterocyclylalkyl, substituted or unsubstituted aryl, which, if substituted, has one or more substituent groups selected from H, alkyl, amino, nitro, halo, haloalkyl, or hydroxyalkyl, or substituted or unsubstituted alkyl, which, if substituted, has one or more substituent groups selected from H, halo, amino, aryl, or heteroaryl;
R³ is selected from H, oxo, alkyl, alkoxy, amino, halo, haloalkyl, hydroxy, carbonyl, alkoxycarbonyl, benzyl, aminoalkyl, hydroxyalkyl, dialkylamino, arylalkenylamino, carboxy, haloalkyl, hydroxyalkylbenzyl, haloalkylbenzyl, haloalkoxybenzyl, furylalkyl, substituted or unsubstituted aryl, which, if substituted, has one or more substituent groups selected from H, alkyl, amino, nitro, halo, haloalkyl, or hydroxyalkyl, or substituted or unsubstituted alkyl, which, if substituted, has one or more substituent groups selected from H, halo, amino, aryl, or heteroaryl, or R¹ and R³ optionally join to form an alkyl bridge between the ring atoms to which R¹ and R³ are attached, or R² and R³ optionally join to form a cycloalkyl ring;
R⁴, R⁶ and R⁷ are independently selected from H, alkyl, benzyl, halo, haloalkyl, alkoxy, haloalkoxy, CO₂—R¹⁵, carboxyl, nitro, amino, alkylamino, dialkylamino, benzyloxy, hydrazinocarbonyl, alkoxycarbonyl, alkoxycarbonylpiperazinylcarbonyl, hydroxy, morpholinylcarbonyl, or piperazinylcarbonyl, or R⁶ and R⁷ optionally join to form a heteroaryl ring;
R⁵ is selected from H, alkyl, alkenyl, alkylthio, hydroxy, halo, alkoxy, benzylalkyl, benzylalkoxy, arylalkyl, nitro, haloalkyl, alkoxycarbonylalkoxy, alkylthio, benzyloxy, phenylalkynyl, phenylcarbonylalkoxy, benzyloxoalkoxy, CO₂—R¹⁵, haloalkoxy, phenylalkoxy, haloarylalkoxy, alkoxycarbonylbenzyloxy, alkoxycarbonyl, alkoxyoxoalkoxy, alkoxyoxophenylalkoxy, halobenzyloxy, pyridinylalkoxy, dialkylaminooxoalkoxy, aminocarbonyl, phenylalkynyl, hydrazinocarbonyl, carbonylalkoxy, alkylsulfonylbenzyloxy, carboxyalkoxy, carbamoyl, naphthylalkoxy, alkoxycarbonylfurylalkoxy, haloalkylbenzyloxy, alkoxyacetylaminoacetyloxy, acetoxy, cyclohexylalkoxy, acetyloxy, morpholinylcarbonyl, alkoxyalkoxy, or R⁴ and R⁵ optionally join to form a pyranyl or 1,4 dioxinyl ring;
R⁸ and R⁹ are independently selected from H, oxo, carbonitrile, alkyl, or R³ and R⁸ optionally join to form a cycloalkyl ring;
R¹⁰ is selected from H, alkyl, benzyl, aryl, or hydroxyalkyl;
R¹¹ is selected from H, oxo, alkyl, halobenzyl, benzyl, haloalkyl, 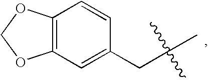
alkoxycarbonyl, carboxyalkyl, benzodioxinylalkyl, substituted or unsubstituted aryl or arylalkyl, which, if substituted, have one or more substituent groups selected from H, alkyl, or alkoxy;
R¹² is selected from H, oxo, alkyl, carbonyl-R¹³, carboxyl-R¹³, ═S, ═N—OR¹⁵, ═N—N(R¹⁵)₂, ketone, cyano, cyanoalkyl, alkoxycarbonyl, aminoalkyl, alkoxycarbonylalkyl, carbamyl, hydroxycarbamyl, haloalkyl, tetrazolyl, tetrazolylalkyl, alkylacetate, t-butoxycarbonylalkyl, hydroxyalkyl, dihydroxyalkyl, isoxazolyl, furanyl, pyrrolyl, pyrazolyl, imidazolyl, acetonitrile, or hydroxyamino, or R¹ and R¹² optionally join to form a ring system selected from: 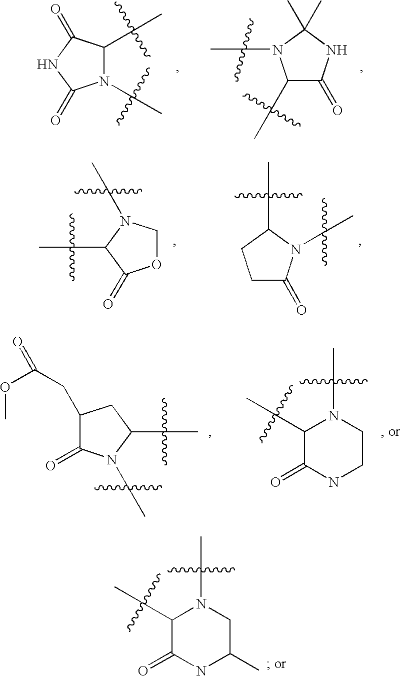
R¹¹ and R¹² optionally join to form a ring system having the structure: 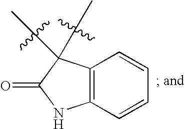
R¹³ is optionally present and if present, is selected from H, alkyl, alkoxy, amino, alkynyl, allyl, alkenyl, hydrazine, benzyl, glycine, allylalkyl, haloalkyl, hydroxyalkyl, hydroxyamino, dihydroxyalkyl, alkoxyalkyl, alkoxyamino, alkylthioalkyl, cyclohexyl, cyclohexylalkyl, amino, alkylamino, benzylalkyl, benzylamino, benzyloxycarbonylalkylamino, dialkylamino, adamantyl haloalkoxybenzyl, alkoxyaminoalkyl, haloalkylarylalkyl, pyridinylalkyl, alkoxyalkylamino, phenylthioalkyl, substituted or unsubstituted aryl or arylalkyl, which, if substituted, have one or more substituent groups selected from H, alkyl, alkoxy, amino, nitro, halo, haloalkyl, or hydroxyalkyl;
R¹⁴ is optionally present and if present, is selected from H or alkyl; and
R¹⁵ is optionally present and if present, is selected from H, alkyl, alkoxy, or aminoalkyl.

3. The method according to claim 1, wherein:
Q, D, T and Z are selected from carbon, sulfur, or nitrogen;
when Q is sulfur, R¹² is oxo, and R¹¹ is optionally oxo or is absent;
when T is sulfur, R² is oxo, and R⁹ is optionally oxo or is absent;
when Z is nitrogen, D is nitrogen, and R² and R¹² are oxo;
R¹ is selected from H, (C₁-C₆) alkyl, (C₁-C₄) alkenyl, (C₁-C₄) allyl, (C₁-C₄) alkynyl, acetyl, amino, (C₁-C₆) alkoxy, hydroxy, oxo, carboxy, allyl-(C₁-C₄) alkyl, benzyl, 2-ethyl-(C₁-C₄) alkyl, haloacetyl, (C₁-C₄) alkoxycarbonylamino-(C₁-C₄) alkyl, t-butyldimethylsilyloxy-(C₁-C₄) alkyl, t-butoxycarbonylamino-(C₁-C₄) alkyl, halo-(C₁-C₄) alkylbenzyl, halo-(C₁-C₄) alkylcarbonyl, (C₁-C₄) alkylcarbonyl, (C₁-C₄) alkoxycarbonyl, arylcarbonyl, benzyloxycarbonyl, t-butoxycarbonyl, phenylmethylideneamino, (C₁-C₄) alkoxyfuranylcarbonyl, or haloarylcarbonyl;
R² is selected from H, (C₁-C₆) alkyl, (C₁-C₆) alkoxy, hydroxy, carbonyl, oxo, benzyl, hydroxy-(C₁-C₄) alkyl, dimethylamino, carboxyl, amino, halo, (C₁-C₄) alkoxycarbonyl, amino-(C₁-C₄) alkyl, or halo-(C₁-C₄) alkyl, (C₁-C₆) alkoxycarbonyl-R¹⁵, cyano-(C₁-C₆) alkyl, aryl-(C₁-C₆) alkyl-R¹⁵, or (C₁-C₆) alkyl-SO₂—R¹⁵;
R³ is selected from H, (C₁-C₆) alkyl, oxo, halo-(C₁-C₄) alkyl, carboxy, oxo, hydroxy-(C₁-C₄) alkylbenzyl, halo-(C₁-C₄) alkylbenzyl, halo-(C₁-C₄) alkoxybenzyl, furyl-(C₁-C₄) alkyl, substituted or unsubstituted aryl, which, if substituted, has one or more substituent groups selected from H, (C₁-C₄) alkyl, amino, nitro, halo, halo-(C₁-C₄) alkyl, or hydroxy-(C₁-C₄) alkyl, or R¹ and R³ optionally join to form a (C₁-C₃) alkyl bridge between the ring atoms to which R¹ and R³ are attached, or R² and R³ optionally join to form a cyclohexyl ring;
R⁴, R⁶ and R⁷ are independently selected from H, (C₁-C₆) alkyl, benzyl, halo, halo-(C₁-C₄) alkyl, (C₁-C₆) alkoxy, CO₂—R¹⁵, halo-(C₁-C₄) alkoxy, carboxyl, nitro, benzyloxy, hydrazinocarbonyl, (C₁-C₄) alkoxycarbonyl, (C₁-C₄) alkoxycarbonylpiperazinylcarbonyl, hydroxy, morpholinylcarbonyl, or piperazinylcarbonyl, or R⁶ and R⁷ optionally join to form a pyridyl ring;
R⁵ is selected from H, (C₁-C₆) alkyl, (C₁-C₄) alkenyl, (C₁-C₄) alkylthio, hydroxy, halo, (C₁-C₆) alkoxy, benzyl-(C₁-C₄) alkyl, benzyl-(C₁-C₄) alkoxy, aryl-(C₁-C₄) alkyl, benzyloxo-(C₁-C₄) alkoxy, nitro, (C₁-C₄) alkoxycarbonylalkoxy, (C₁-C₄) alkylthio, benzyloxy, phenyl-(C₁-C₄) alkynyl, phenylcarbonyl-(C₁-C₄) alkoxy, halo-(C₁-C₆) alkyl, CO₂—R¹⁵, halo-(C₁-C₄) alkoxy, aryl-(C₁-C₄) alkoxy, arylcarbonyl-(C₁-C₄) alkoxy, haloaryl-(C₁-C₄) alkoxy, (C₁-C₄) alkoxycarbonylbenzyloxy, (C₁-C₄) alkoxycarbonyl, (C₁-C₄) alkoxyoxo-(C₁-C₄) alkoxy, -(C₁-C₄) alkoxyoxophenyl-(C₁-C₄) alkoxy, halobenzyloxy, pyridinyl-(C₁-C₄) alkoxy, dialkylaminooxo-(C₁-C₄) alkoxy, aminocarbonyl, phenyl-(C₁-C₄) alkynyl, hydrazinocarbonyl, carbonyl-(C₁-C₄) alkoxy, (C₁-C₄) alkylsulfonylbenzyloxy, carboxy-(C₁-C₄) alkoxy, carbamoyl, naphthyl-(C₁-C₄) alkoxy, (C₁-C₄) alkoxycarbonylfuryl-(C₁-C₄) alkoxy, halo-(C₁-C₄) alkylbenzyloxy, (C₁-C₄) alkoxyacetylaminoacetyloxy, acetoxy, cyclohexyl-(C₁-C₄) alkoxy, acetyloxy, morpholinylcarbonyl, (C₁-C₄) alkoxy-(C₁-C₄) alkoxy, or R⁴ and R⁵ optionally join to form a pyranyl or 1,4 dioxinyl ring;
R⁸ and R⁹ are independently selected from H, oxo, carbonitrile, (C₁-C₆) alkyl, or R³ and R⁸ optionally join to form a cycloalkyl ring;
R¹⁰ is selected from H, (C₁-C₆) alkyl, benzyl, phenyl, or hydroxy-(C₁-C₄) alkyl;
R¹¹ is selected from H, oxo, (C₁-C₆) alkyl, halobenzyl, benzyl, halo-(C₁-C₄) alkyl, C₁-C₄) alkoxycarbonyl, carboxy-(C₁-C₄) alkyl, 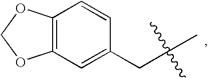
benzodioxinyl-(C₁-C₄) alkyl, substituted or unsubstituted aryl or aryl-(C₁-C₆) alkyl, which, if substituted, have one or more substituent groups selected from H, (C₁-C₆) alkyl, (C₁-C₆) alkoxy;
R¹² is selected from H, oxo, (C₁-C₆) alkyl, carbonyl-R¹³, carboxyl-R¹³, ═S, ═N—OR¹⁵, ═N—N(R¹⁵)₂, cyano, cyano-(C₁-C₄) alkyl, (C₁-C₄) alkoxycarbonyl, amino-(C₁-C₄) alkyl, (C₁-C₄) alkoxycarbonyl-(C₁-C₄) alkyl, carbamyl, hydroxycarbamyl, halo-(C₁-C₄) alkyl, tetrazolyl, tetrazolyl-(C₁-C₄) alkyl, (C₁-C₄) alkylacetate, t-butoxycarbonyl-(C₁-C₄) alkyl, hydroxy-(C₁-C₄) alkyl, dihydroxy-(C₁-C₄) alkyl, isoxazolyl, furanyl, pyrrolyl, pyrazolyl, imidazolyl, acetonitrile, or hydroxyamino, or R¹ and R¹² optionally join to form a ring system selected from: 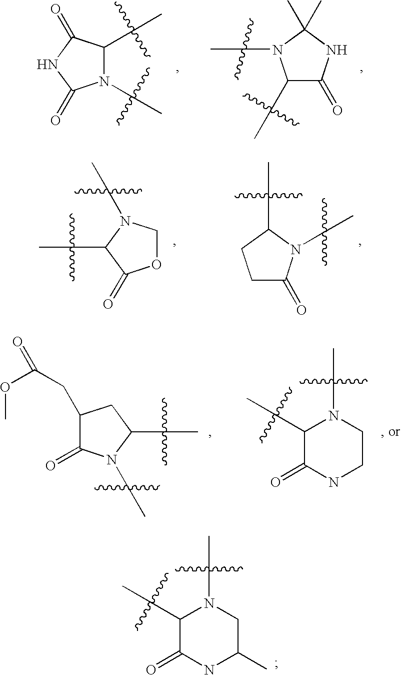
or R¹¹ and R¹² optionally join to form a ring system selected from: 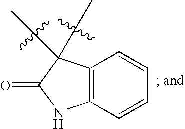
R¹³ is optionally present and if present, is selected from H, (C₁-C₆) alkyl, (C₁-C₆) alkoxy, amino, (C₁-C₆) allyl, (C₁-C₆) alkynyl, (C₁-C₆) alkenyl, hydrazine, benzyl, glycine, allyl-(C₁-C₄) alkyl, halo-(C₁-C₄) alkyl, hydroxy-(C₁-C₄) alkyl, hydroxyamino, dihydroxy-(C₁-C₄) alkyl, (C₁-C₄) alkoxy-(C₁-C₄) alkyl, (C₁-C₄) alkoxyamino, (C₁-C₄) alkylthio-(C₁-C₄) alkyl, cyclohexyl, cyclohexyl-(C₁-C₄) alkyl, amino, (C₁-C₄) alkylamino, benzyl-(C₁-C₄) alkyl, benzylamino, benzyloxycarbonyl-(C₁-C₄) alkylamino, dialkylamino, adamantyl halo-(C₁-C₄) alkoxybenzyl, (C₁-C₄) alkoxyamino-(C₁-C₄) alkyl, halo-(C₁-C₄) alkylaryl-(C₁-C₄) alkyl, pyridinyl-(C₁-C₄) alkyl, (C₁-C₄) alkoxy-(C₁-C₄) alkylamino, phenylthio-(C₁-C₄) alkyl, substituted or unsubstituted aryl or aryl-(C₁-C₄) alkyl, which, if substituted, have one or more substituent groups selected from H, (C₁-C₆) alkyl, (C₁-C₆) alkoxy, amino, nitro, halo, halo-(C₁-C₄) alkyl, or hydroxy-(C₁-C₄) alkyl;
R¹⁴ is optionally present and if present, is selected from H or (C₁-C₆) alkyl; and
R¹⁵ is optionally present and if present, is selected from H, (C₁-C₆) alkyl, (C₁-C₆) alkoxy, or amino-(C₁-C₆) alkyl.

4. The method according to claim 1, wherein:
Q, D, T and Z are selected from carbon, sulfur, or nitrogen;
when Q is sulfur, R¹² is oxo, and R¹¹ is optionally oxo or is absent;
when T is sulfur, R² is oxo, and R⁹ is optionally oxo or is absent;
when Z is nitrogen, D is nitrogen, and R² and R¹² are oxo;
R¹ is selected from H, methyl, ethyl, propyl, isopropyl, n-propyl, t-butyldimethylsilyloxypropyl, t-butoxycarbonylaminoethyl, 2-ethylbutyl, chlorophenylcarbonyl, trifluoroacetyl, methoxyfuranylcarbonyl, benzyloxycarbonyl, propene, acetyl, t-butoxycarbonyl, trifluoromethylbenzyl, benzyl, oxo, or phenylmethylideneamino.
R² is selected from H, methyl, ethyl, methoxy, propyl, n-propyl, amino-(C₁-C₄) alkyl, hydroxyl-(C₁-C₄) alkyl, dimethylaminomethyl, carboxyl, dimethylamino, benzyl, trifluoromethyl, methoxycarbonyl, oxo, methoxycarbonyl-R¹⁵, cyanomethyl, phenylalkyl-R⁵, or methyl-SO₂—R¹⁵;
R³ is selected from H, methyl, ethyl, propyl, n-propyl, carboxyl, trifluoromethyl, p-hydroxymethylbenzyl, m-trifluoromethylbenzyl, o-trifluoromethylbenzyl, trifluoromethoxybenzyl, phenyl, furylmethyl, or oxo, or R¹ and R³ optionally join to form a C₁-C₃ alkyl bridge between the ring atoms to which R¹ and R³ are attached, or R² and R³ optionally join to form a ring system consisting of: 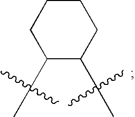
R⁴, R⁶ and R⁷ are independently selected from H, methyl, benzyl, methoxy, hydroxyl, methoxycarbonyl, benzyloxy, hydrazinocarbonyl, carbonxyl, fluoro, chloro, morpholinylcarbonyl, nitro, CO₂—R¹⁵, bromo, t-butoxycarbonylpiperazinylcarbonyl, piperazinylcarbonyl, or R⁶ and R⁷ optionally join to form a pyridyl ring;
R⁵ is selected from H, methyl, methoxy, acetoxy, carbamoyl, 2-oxo-2-phenylethoxy, hydroxyl, benzyloxy, benzyloxoethoxy, methoxycarbonyl, 2-ethoxy-2-oxoethoxy, 2-methoxy-2-oxo-1-phenylethoxy, butoxy, n-butoxy, 2-methoxy-2-oxoethoxy, p-chlorobenzyloxy, o-chlorobenzyloxy, m-chlorobenzyloxy, chloropropoxy, chlorobutoxy, chloro, 3-pyridylmethoxy, 4-pyridylmethoxy, N,N-dimethylcarbamylmethoxy, trifluoromethyl, CO₂—R¹⁵, 2-phenylethoxy, aminocarbonyl, bromo, fluoro, carboxymethoxy, p-(methylsulfonyl)benzyloxy, hydrazinocarbonyl, 2-naphthylmethoxy, 4-ethoxy-4-oxobutoxy, (5-ethoxycarbonyl-2-furyl)methoxy, 2-methoxyethoxy, p-(trifluoromethyl)benzyloxy, 3-phenylpropoxy, m-chlorobenzylmethoxy, 3-methoxycarbonylbenzyloxy, 2-pyridylmethoxy, cyclohexylmethoxy, ethoxycarbonylmethylaminocarbonylmethoxy, iodo, phenylethynyl, 2-phenylethyl, nitro, ethoxy, or methylthio, or R⁴ and R⁵ optionally join to form a pyranyl or 1,4 dioxinyl ring;
R⁸ and R⁹ are independently selected from H, oxo, carbonitrile, methyl, or R³ and R⁸ optionally join to form a cyclohexyl ring;
R¹⁰ is selected from H, benzyl, phenyl, methyl, ethyl, or hydroxymethyl;
R¹¹ is selected from H, oxo, methyl, benzyl, phenyl, chloromethyl, p-chlorobenzyl, methoxycarbonyl, ethoxycarbonyl, carboxyethyl, 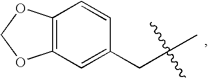
carboxypropyl, or trimethoxybenzyl;
R¹² is selected from H, oxo, methyl, ethyl, carbonyl-R¹³, carboxyl-R¹³, ═S, ═N—OR¹⁵, ═N—N(R¹⁵)₂, cyano, cyanomethyl, tetrazoyl, pyrrolyl, tetrazoylmethyl, methylacetate, t-butylacetate, hydroxymethyl, trifluoromethyl, aminomethyl, acetonitrile, ethane-1,2-diol, methoxycarbonyl, methoxycarbonylmethyl, carbamyl, hydroxycarbamyl, isoxazolyl, furanyl, pyrazolyl, imidazolyl, or hydroxyamino, or R¹ and R¹² optionally join to form a ring system selected from: 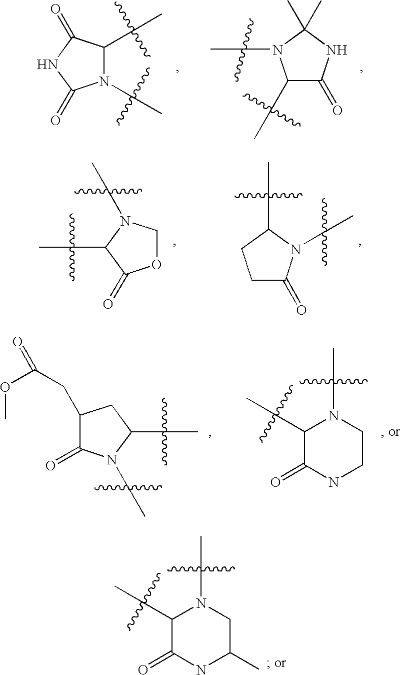
R¹¹ and R¹² optionally join to form a ring system having the structure: 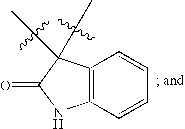
R¹³ is optionally present and if present, is selected from H, methyl, ethyl, i-propyl, n-propyl, propyl, benzyl, hydroxyamino, hydrazino, glycine, propene, benzyloxycarbonylmethylamino, 1-phenylethyl, 2,2,2-trifluoroethyl, 2-fluoroethyl, 2-hydroxyethyl, 2,3-dihydroxypropyl, 2-methoxyethyl, 2-(methylthio)ethyl, cyclohexyl, cyclohexylmethyl, t-butyl, neopentyl, 1-adamantyl, pentyl, amino, N,N-dimethylamino, benzylamino, 2-methoxyethylamino, n-hexyl, p-(trifluoromethylbenzyl), trifluoromethoxybenzyl, methylamino, pyridylmethyl, (N-methoxy-N-methyl)amino, 2,2,6,6-tetramethylcyclohexyl, or 2-(phenylthio)ethyl;
R¹⁴ is optionally present and if present, is selected from H or methyl; and
R¹⁵ is optionally present and if present, is selected from H, (C₁-C₄) alkyl, (C₁-C₄) alkoxy, or amino-(C₁-C₄) alkyl.

5. The method according to claim 1, wherein:
Q, D, T and Z are selected from carbon, sulfur, or nitrogen;
when Q is sulfur, R¹² is oxo, and R¹¹ is optionally oxo or is absent;
when T is sulfur, R² is oxo, and R⁹ is optionally oxo or is absent;
when Z is nitrogen, D is nitrogen, and R² and R¹² are oxo;
R¹ is selected from H, methyl, ethyl, i-propyl, propenyl, or trifluoromethylbenzyl;
R² is selected from H, methyl, ethyl, propyl, hydroxyl-(C₁-C₄) alkyl, methoxycarbonyl-R¹⁵, cyanomethyl, phenylalkyl-R¹⁵, methyl-SO₂—R¹⁵, or amino-(C₁-C₄) alkyl;
R³ is selected from H, oxo, methyl, ethyl, carboxyl, propyl, benzyl, hydroxymethylbenzyl, trifluoromethyl, trifluoromethylbenzyl, or R¹ and R³ optionally join to form a C₁-C₃ alkyl bridge between the ring atoms to which R¹ and R³ are attached;
R⁴, R⁶ and R⁷ are independently selected from H, methoxy, CO₂—R¹⁵, methoxycarbonyl, hydroxyl, or R⁶ and R⁷ optionally join to form a pyridyl ring;
R⁵ is selected from H, methyl, hydroxy, methoxy, butyl, butoxy, methylcarboxymethoxy, ethylcarboxymethoxy, chloropropoxy, chlorobutoxy, methoxycarbonyl, 2-methoxy-2-oxoethoxy, trifluoromethyl, CO₂—R⁵, methoxycarbonylbenzyloxy, 2-oxo-2-phenylethoxy, phenylmethoxy, phenylethoxy, aminocarbonyl, 2-methoxyethoxy, chlorobenzyloxy, 3-phenylpropoxy, phenylcarbonylmethoxy, chlorobenzyloxy, hydrazinocarbonyl, benzyloxy, acetyloxy, 2-methoxy-2-oxo-1-phenylethoxy, trifluoromethylbenzyloxy, methylcarboxyphenylmethoxy, methylcarboxy, fluoro, chloro, iodo, bromo, ethoxycarbonylmethylaminocarbonylmethoxy, methylthio, furylmethoxy, N,N-dimethylcarbamylmethoxy, p-(methylsulfonyl)benzyloxy, 2-ethoxy-2-oxoethoxy, carboxymethoxy, pyridyloxomethoxy, pyridylmethoxy, carbazoyl, methylpropoxy, carbamyl, naphthlylmethoxy, or R⁴ and R⁵ optionally join to form a pyran ring;
R⁸, R⁹ and R¹⁰ are independently selected from H, benzyl or methyl;
R¹¹ is selected from H, or hydroxamate;
R¹² is selected from H, oxo, tetrazolyl, carbonyl-R¹³, or carboxyl-R¹³, ═S, ═N—OR⁵, ═N—N(R¹⁵)₂, or R¹ and R¹² optionally join to form a ring system having the structure: 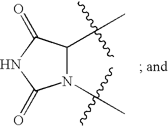
R¹³ is selected from H, methyl, ethyl, i-propyl, propyl, benzyl, 1-phenylethyl, 2-fluoroethyl, 2,2,2-trifluoroethyl, hydroxyethyl, 2,3-dihydroxypropyl, 3,5 hydroxypentyl, ethoxy, propoxy, methylthioethyl, cyclohexyl, cyclohexylmethyl, t-butyl, neopentyl, 1-adamantyl, 3-pentyl, 2-methoxyethylamino, methoxymethylamino, 2-methoxyethyl, hexyl, p-(trifluoromethylbenzyl), pyridylmethyl, 1-methoxy-1-methylamino, 2,2,6,6-tetramethylcyclohexyl, phenylthioethenyl, or allyl; and
R¹⁵ is optionally present and if present, is selected from H, alkyl, alkoxy, or aminoalkyl.

6. The method according to claim 1, wherein:
Q, D, T and Z are selected from carbon, sulfur, or nitrogen;
when Q is sulfur, R¹² is oxo, and R¹¹ is optionally oxo or is absent;
when T is sulfur, R² is oxo, and R⁹ is optionally oxo or is absent;
when Z is nitrogen, D is nitrogen, and R² and R¹² are oxo;
R¹ is selected from H, methyl, ethyl, propenyl, or trifluoromethylbenzyl;
R² is selected from H, methyl, ethyl, methoxycarbonyl-R¹⁵ cyanomethyl, phenylalkyl-R¹⁵, hydroxyl-(C₁-C₄) alkyl, or amino-(C₁-C₄) alkyl;
R³ is selected from H, oxo, methyl, ethyl, carboxyl, propyl, benzyl, hydroxymethylbenzyl, trifluoromethyl, trifluoromethylbenzyl, or R¹ and R³ optionally join to form a C₁-C₃ alkyl bridge between the ring atoms to which R¹ and R³ are attached;
R⁴, R⁶ and R⁷ are independently selected from H, CO₂—R¹⁵, methoxy, or hydroxyl, or R⁶ and R⁷ optionally join to form a pyridyl ring;
R⁵ is selected from H, CO₂—R¹⁵, methyl, hydroxy, methoxy, butyl, butoxy, methylcarboxymethoxy, ethylcarboxymethoxy, 2-oxo-2-phenylethoxy, 2-methoxy-2-oxo-1-phenylethoxy, 2-methoxy-2-oxoethoxy, chloropropoxy, chlorobutoxy, hydrazinocarbonyl, methoxycarbonyl, phenylmethoxy, phenylethoxy, phenylpropoxy, aminocarbonyl, phenylcarbonylmethoxy, chlorobenzyloxy, benzyloxy, methylcarboxyphenylmethoxy, methylcarboxy, fluoro, chloro, iodo, bromo, methylthio, furylmethoxy, N,N-dimethylcarbamylmethoxy, p-(methylsulfonyl)benzyloxy, 2-ethoxy-2-oxoethoxy, carboxymethoxy, pyridyloxomethoxy, pyridylmethoxy, carbazoyl, methylpropoxy, carbamyl, naphthlylmethoxy, or R⁴ and R⁵ optionally join to form a pyran ring;
R⁸, R⁹ and R¹⁰ are independently selected from H or methyl;
R¹¹ is selected from H, or hydroxamate;
R¹² is selected from H, oxo, carbonyl-R¹³, carboxyl-R³, ═S, or ═N—OR¹⁵;
R¹³ is selected from H, methyl, ethyl, i-propyl, propyl, benzyl, 1-phenylethyl, 2-fluoroethyl, 2,2,2-trifluoroethyl, hydroxyethyl, 2,3-dihydroxypropyl, 3,5 hydroxypentyl, ethoxy, propoxy, methylthioethyl, cyclohexyl, cyclohexylmethyl, t-butyl, neopentyl, 1-adamantyl, 3-pentyl, 2-methoxyethylamino, methoxymethylamino, 2-methoxyethyl, hexyl, p-(trifluoromethylbenzyl), pyridylmethyl, 1-methoxy-1-methylamino, 2,2,6,6-tetramethylcyclohexyl, phenylthioethenyl, or allyl; and
R¹⁵ is optionally present and if present, is selected from H, alkyl, alkoxy, or aminoalkyl.

7. The method according to claim 1, wherein:
T, Q and Z are carbon;
D is selected from carbon or nitrogen;
R¹ is selected from H, methyl or ethyl;
R² is selected from H, methyl, hydroxyl-(C₁-C₄) alkyl, amino-(C₁-C₄) alkyl, or phenylalkyl-R¹⁵,
R³ is selected from H, oxo, methyl, ethyl, propyl, benzyl, trifluoromethyl, or R¹ and R³ optionally join to form a C₁-C₃ alkyl bridge between the ring atoms to which R¹ and R³ are attached;
R⁴, R⁶ and R⁷ are independently selected from H, CO₂—R¹⁵ methoxy, or R⁶ and R⁷ optionally join to form a pyridyl ring;
R⁵ is selected from H, CO₂—R¹⁵, hydroxyl, methoxy, butoxy, 2-oxo-2-phenylethoxy, 2-methoxy-2-oxo-1-phenylethoxy, chloropropoxy, chlorobutoxy, methoxycarbonyl, phenylethoxy, aminocarbonyl, hydrazinocarbonyl, chlorobenzyloxy, benzyloxy, chloro, bromo, iodo, methylthio, N,N-dimethylcarbamylmethoxy, p-(methylsulfonyl)benzyloxy, 2-ethoxy-2-oxoethoxy, 2-methoxy-2-oxoethoxy, carboxymethoxy, pyridylmethoxy, or R⁴ and R⁵ optionally join to form a pyran ring;
R⁸, R⁹ and R¹⁰ are independently selected from H or methyl;
R¹¹ is selected from H, or hydroxamate;
R¹² is selected from H, oxo, carboxyl-R¹³, ═S, or ═N—OR¹⁵;
R¹³ is selected from H, methyl, ethyl, i-propyl, propyl, benzyl, 1-phenylethyl, 2-fluoroethyl, 2,2,2-trifluoroethyl, hydroxyethyl, 2,3-dihydroxypropyl, methylthioethyl, cyclohexyl, cyclohexylmethyl, t-butyl, neopentyl, 2-methoxyethyl, trifluoromethoxybenzyl, hexyl, pyridylmethyl, or allyl; and
R¹⁵ is optionally present and if present, is selected from H, alkyl, alkoxy, or aminoalkyl.

8. The method according to claim 1, wherein:
T, Q and Z are carbon;
D is selected from carbon or nitrogen;
R¹ is selected from H, methyl or ethyl;
R² is selected from H, methyl, hydroxymethyl, amino-(C₁-C₄) alkyl, methyl, or phenylalkyl-R¹⁵;
R³ is selected from H, oxo, methyl, ethyl, propyl, benzyl, trifluoromethyl, or R¹ and R³ optionally join to form a C₁-C₃ alkyl bridge between the ring atoms to which R¹ and R³ are attached;
R⁴, R⁶ and R⁷ are independently selected from H, CO₂—R¹⁵, methoxy, or R⁶ and R⁷ optionally join to form a pyridyl ring;
R⁵ is selected from H, CO₂—R¹⁵, hydroxyl, methoxy, butoxy, 2-oxo-2-phenylethoxy, 2-methoxy-2-oxo-1-phenylethoxy, chloropropoxy, chlorobutoxy, methoxycarbonyl, chlorobenzyloxy, benzyloxy, chloro, bromo, iodo, methylthio, 2-ethoxy-2-oxoethoxy, 2-methoxy-2-oxoethoxy, or R⁴ and R⁵ optionally join to form a pyran ring;
R⁸, R⁹ and R¹⁰ are independently selected from H or methyl;
R¹¹ is selected from H, or hydroxamate;
R¹² is selected from H, oxo, ═N—OR¹⁵, or carboxyl-R¹³;
R¹³ is selected from H, methyl, i-propyl, propyl, benzyl, 1-phenylethyl, 2-fluoroethyl, 2,2,2-trifluoroethyl, hydroxyethyl, 2,3-dihydroxypropyl, methylthioethyl, t-butyl, 2-methoxyethyl, trifluoromethoxybenzyl, pyridylmethyl, or allyl;
R¹⁵ is optionally present and if present, is selected from H, alkyl, alkoxy, or aminoalkyl.

9. The method according to claim 1, wherein:
T, Q and Z are carbon;
D is selected from carbon or nitrogen;
R¹ is selected from H or methyl;
R² is selected from H, methyl, amino-(C₁-C₄) alkyl, hydroxymethyl, or phenylalkyl-R¹⁵;
R³ is selected from H, oxo, methyl, ethyl, benzyl, or R¹ and R³ optionally join to form a C₁-C₃ alkyl bridge between the ring atoms to which R¹ and R³ are attached;
R⁴, R⁶ and R⁷ are independently selected from H, methoxy, CO₂—R¹⁵, or R⁶ and R⁷ optionally join to form a pyridyl ring;
R⁵ is selected from H, hydroxyl, methoxy, 2-oxo-2-phenylethoxy, methoxycarbonyl, chlorobenzyloxy, benzyloxy, chloro, methylthio, 2-ethoxy-2-oxoethoxy, or R⁴ and R⁵ optionally join to form a pyran ring;
R⁸, R⁹ and R¹⁰ are independently selected from H or methyl;
R¹¹ is selected from H, or hydroxamate;
R¹² is selected from H, oxo, ═N—OR¹⁵, or carboxyl-R¹³;
R¹³ is selected from H, methyl, 2-fluoroethyl, 2,2,2-trifluoroethyl, 2,3-dihydroxypropyl, methylthioethyl, t-butyl, 2-methoxyethyl, pyridylmethyl, or allyl; and
R¹⁵ is optionally present and if present, is selected from H, alkyl, alkoxy, or aminoalkyl.

10. The method according to claim 1, wherein the compound is chosen from:
7-methoxy-3,4,5,10-tetrahydro-1H-2,5-methanoazepino[3,4-b]indole-1-carboxylic acid,
7-hydroxy-3,4,5,10-tetrahydro-1H-2,5-methanoazepino[3,4-b]indole-1-carboxylic acid,
2,3,8,10,11,12-hexahydro-1H,7H-9,12-methanoazepino[3,4-b]pyrano[3,2-e]indole-8-carboxylic acid,
7-methoxy-3,4,5,10-tetrahydro-1H-2,5-methanoazepino[3,4-b]indole-1-carboxylic acid,
7-(methylthio)-3,4,5,10-tetrahydro-1H-2,5-methanoazepino[3,4-b]indole-1-carboxylic acid,
7-methoxy-3,4,5,10-tetrahydro-1H-2,5-methanoazepino[3,4-b]indole-1-carboxylic acid,
7-(benzyloxy)-3,4,5,10-tetrahydro-1H-2,5-methanoazepino[3,4-b]indole-1-carboxylic acid,
7-(methylthio)-3,4,5,10-tetrahydro-1H-2,5-methanoazepino[3,4-b]indole-1-carboxylic acid,
6-methoxy-2,3,4,9-tetrahydro-1H-beta-carboline-1-carboxylic acid,
6-(2-oxo-2-phenylethoxy)-2,3,4,9-tetrahydro-1H-beta-carboline-1-carboxylic acid,
6-methoxy-2-methyl-2,3,4,9-tetrahydro-1H-beta-carboline-1-carboxylic acid,
2,2,2-trifluoroethyl 7-methoxy-3,4,5,10-tetrahydro-1H-2,5-methanoazepino[3,4-b]indole-1-carboxylate,
6-methoxy-2,3,4,9-tetrahydro-1H-beta-carboline-1-carboxylic acid,
7-hydroxy-3,4,5,10-tetrahydro-1H-2,5-methanoazepino[3,4-b]indole-1-carboxylic acid,
6-hydroxy-2-methyl-2,3,4,9-tetrahydro-1H-beta-carboline-1-carboxylic acid,
7-methoxy-3,4,5,10-tetrahydro-1H-2,5-methanoazepino[3,4-b]indole-1-carboxylic acid,
6-methoxy-2-methyl-2,3,4,9-tetrahydro-1H-beta-carboline-1-carboxylic acid,
2,3-dihydroxypropyl 7-methoxy-3,4,5,10-tetrahydro-1H-2,5-methanoazepino[3,4-b]indole-1-carboxylate,
4-ethyl-6-methoxy-2,3,4,9-tetrahydro-1H-beta-carboline-1-carboxylic acid,
6-methoxy-4-methyl-2,3,4,9-tetrahydro-1H-beta-carboline-1-carboxylic acid,
8,9,10,11-tetrahydro-7H-pyrido[3′,4′:4,5]pyrrolo[2,3-f]isoquinolin-7-one trifluoroacetate,
3-(aminomethyl)-6-methoxy-2,3,4,9-tetrahydro-1H-beta-carbolin-1-one trifluoroacetate,
3-(aminomethyl)-6-methoxy-2,3,4,9-tetrahydro-1H-beta-carbolin-1-one hydrochloride, mixtures thereof, and pharmaceutically acceptable salts thereof.

11. The method according to claim 1, wherein the compound is chosen from:
7-methoxy-3,4,5,10-tetrahydro-1H-2,5-methanoazepino[3,4-b]indole-1-carboxylic acid,
7-hydroxy-3,4,5,10-tetrahydro-1H-2,5-methanoazepino[3,4-b]indole-1-carboxylic acid,
2,3,8,10,11,12-hexahydro-1H,7H-9,12-methanoazepino[3,4-b]pyrano[3,2-e]indole-8-carboxylic acid,
8,9,10,11-tetrahydro-7H-pyrido[3′,4′:4,5]pyrrolo[2,3-f]isoquinolin-7-one trifluoroacetate,
3-(aminomethyl)-6-methoxy-2,3,4,9-tetrahydro-1H-beta-carbolin-1-one trifluoroacetate,
3-(aminomethyl)-6-methoxy-2,3,4,9-tetrahydro-1H-beta-carbolin-1-one hydrochloride, mixtures thereof, and pharmaceutically acceptable salts thereof.

12. A method for treating or preventing a disease or disorder in a subject, which disease or disorder is one that can be treated or prevented by inhibiting the activity of MK-2, the method comprising administering to the subject a compound, or a pharmaceutically acceptable salt or prodrug thereof, wherein the compound has the structure: 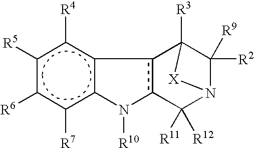
wherein:
X is alkyl;
R² is selected from H, alkyl, alkoxy, hydroxy, carbonyl, oxo, benzyl, hydroxyalkyl, dialkylamino, carboxyl, amino, halo, alkoxycarbonyl, aminoalkyl, haloalkyl, hydroxyalkylbenzyl, haloalkylbenzyl, haloalkoxybenzyl, heterocyclylalkyl, substituted or unsubstituted aryl, which, if substituted, has one or more substituent groups selected from H, alkyl, amino, nitro, halo, haloalkyl, or hydroxyalkyl;
R³ is selected from H, alkyl, alkoxy, hydroxy, carbonyl, benzyl, hydroxyalkyl, dialkylamino, carboxyl, amino, halo, alkoxycarbonyl, aminoalkyl, haloalkyl, hydroxyalkylbenzyl, haloalkylbenzyl, haloalkoxybenzyl, heterocyclylalkyl, substituted or unsubstituted aryl, which, if substituted, has one or more substituent groups selected from H, alkyl, amino, nitro, halo, haloalkyl, or hydroxyalkyl, or R² and R³ optionally join to form a cycloalkyl ring;
R⁴, R⁶ and R⁷ are independently selected from H, alkyl, benzyl, amino, alkylamino, dialkylamino, halo, haloalkyl, alkoxy, haloalkoxy, carboxyl, nitro, benzyloxy, hydrazinocarbonyl, alkoxycarbonyl, alkoxycarbonylheterocyclylcarbonyl, hydroxy, or heterocyclylcarbonyl;
R⁵ is selected from H, alkyl, hydroxy, alkenyl, alkylthio, hydroxy, halo, alkoxy, benzylalkyl, benzylalkoxy, arylalkyl, nitro, alkoxycarbonylalkoxy, alkylthio, benzyloxy, arylalkynyl, arylcarbonylalkoxy, haloalkoxy, arylalkoxy, arylcarbonylalkoxy, haloarylalkoxy, benzyloxoalkoxy, alkoxycarbonylbenzyloxy, alkoxycarbonyl, alkoxyoxoalkoxy, alkoxyoxoarylalkoxy, halobenzyloxy, heterocyclylalkoxy, dialkylaminooxoalkoxy, aminocarbonyl, arylalkynyl, hydrazinocarbonyl, carbonylalkoxy, alkylsulfonylbenzyloxy, carboxyalkoxy, carbamoyl, naphthylalkoxy, alkoxycarbonylheterocyclylalkoxy, haloalkylbenzyloxy, alkoxyacetylaminoacetyloxy, acetoxy, cycloalkylalkoxy, acetyloxy, morpholinylcarbonyl, alkoxyalkoxy, or R⁴ and R⁵ optionally join to form a heterocyclic ring;
R⁹ is selected from H, oxo, carbonitrile, or alkyl;
R¹⁰ is selected from H, alkyl, benzyl, aryl, or hydroxyalkyl;
R¹¹ is selected from H, oxo, alkyl, halobenzyl, benzyl, haloalkyl, 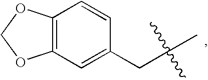
alkoxycarbonyl, carboxyalkyl, benzodioxinylalkyl, substituted or unsubstituted aryl or arylalkyl, which, if substituted, have one or more substituent groups selected from H, alkyl, or alkoxy;
R¹² is selected from H, oxo, alkyl, carbonyl-R¹³, carboxyl-R¹³, ketone, cyano, cyanoalkyl, alkoxycarbonyl, aminoalkyl, alkoxycarbonylalkyl, carbamyl, hydroxycarbamyl, haloalkyl, heterocyclyl, alkylacetate, heterocyclylalkyl, alkoxycarbonylalkyl, hydroxyalkyl, dihydroxyalkyl, cyanoalkyl, or hydroxyamino, substituted or unsubstituted 5 or 6-membered heterocyclic ring, or R¹¹ and R¹² optionally join to form a ring system having the structure: 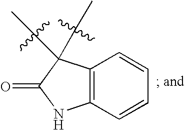
R¹³ is optionally present and if present, is selected from H, alkyl, alkoxy, amino, alkynyl, allyl, alkenyl, hydrazine, benzyl, glycine, allylalkyl, haloalkyl, hydroxyalkyl, hydroxyamino, dihydroxyalkyl, alkoxyalkyl, alkoxyamino, alkylthioalkyl, cycloalkyl, cyclohexylalkyl, amino, alkylamino, benzylalkyl, benzylamino, benzyloxycarbonylalkylamino, dialkylamino, adamantyl, haloalkoxybenzyl, alkoxyaminoalkyl, haloalkylarylalkyl, heterocyclylalkyl, alkoxyalkylamino, arylthioalkyl, substituted or unsubstituted aryl or arylalkyl, which, if substituted, have one or more substituent groups selected from H, alkyl, alkoxy, amino, nitro, halo, haloalkyl, or hydroxyalkyl.

13. The method according to claim 12, wherein:
X is C₁-C₃ alkyl;
R² is selected from H, (C₁-C₆) alkyl, carbonyl, (C₁-C₄) alkoxycarbonyl, amino-(C₁-C₄) alkyl, (C₁-C₆) alkoxy, amino-(C₁-C₄) alkyl, hydroxy, benzyl, hydroxyalkyl, dialkylamino, amino, halo, halo-(C₁-C₄) alkyl, carboxy, oxo, hydroxy-(C₁-C₄) alkylbenzyl, halo-(C₁-C₄) alkylbenzyl, halo-(C₁-C₄) alkoxybenzyl, furyl-(C₁-C₄) alkyl, substituted or unsubstituted aryl, which, if substituted, has one or more substituent groups selected from H, (C₁-C₄) alkyl, amino, nitro, halo, halo-(C₁-C₄) alkyl, or hydroxy-(C₁-C₄) alkyl;
R³ is selected from H, (C₁-C₆) alkyl, carbonyl, (C₁-C₄) alkoxycarbonyl, amino-(C₁-C₄) alkyl, (C₁-C₆) alkoxy, amino-(C₁-C₄) alkyl, hydroxy, benzyl, hydroxyalkyl, dialkylamino, amino, halo, halo-(C₁-C₄) alkyl, carboxy, hydroxy-(C₁-C₄) alkylbenzyl, halo-(C₁-C₄) alkylbenzyl, halo-(C₁-C₄) alkoxybenzyl, furyl-(C₁-C₄) alkyl, substituted or unsubstituted aryl, which, if substituted, has one or more substituent groups selected from H, (C₁-C₄) alkyl, amino, nitro, halo, halo-(C₁-C₄) alkyl, or hydroxy-(C₁-C₄) alkyl, or R² and R³ optionally join to form a cyclohexyl ring;
R⁴, R⁶ and R⁷ are independently selected from H, (C₁-C₆) alkyl, amino, (C₁-C₄) alkylamino, dialkylamino, benzyl, halo, halo-(C₁-C₄) alkyl, (C₁-C₆) alkoxy, halo-(C₁-C₄) alkoxy, carboxyl, nitro, benzyloxy, hydrazinocarbonyl, (C₁-C₄) alkoxycarbonyl, (C₁-C₄) alkoxycarbonylpiperazinylcarbonyl, hydroxy, morpholinylcarbonyl, or piperazinylcarbonyl;
R⁵ is selected from H, (C₁-C₆) alkyl, (C₁-C₄) alkenyl, (C₁-C₄) alkylthio, hydroxy, halo, (C₁-C₆) alkoxy, benzyl-(C₁-C₄) alkyl, benzyl-(C₁-C₄) alkoxy, aryl-(C₁-C₄) alkyl, benzyloxo-(C₁-C₄) alkoxy, nitro, (C₁-C₄) alkoxycarbonylalkoxy, (C₁-C₄) alkylthio, benzyloxy, phenyl-(C₁-C₄) alkynyl, phenylcarbonyl-(C₁-C₄) alkoxy, halo-(C₁-C₄) alkoxy, aryl-(C₁-C₄) alkoxy, arylcarbonyl-(C₁-C₄) alkoxy, haloaryl-(C₁-C₄) alkoxy, (C₁-C₄) alkoxycarbonylbenzyloxy, (C₁-C₄) alkoxycarbonyl, (C₁-C₄) alkoxyoxo-(C₁-C₄) alkoxy, (C₁-C₄) alkoxyoxophenyl-(C₁-C₄) alkoxy, halobenzyloxy, pyridinyl-(C₁-C₄) alkoxy, dialkylaminooxo-(C₁-C₄) alkoxy, aminocarbonyl, phenyl-(C₁-C₄) alkynyl, hydrazinocarbonyl, carbonyl-(C₁-C₄) alkoxy, (C₁-C₄) alkylsulfonylbenzyloxy, carboxy-(C₁-C₄) alkoxy, carbamoyl, naphthyl-(C₁-C₄) alkoxy, (C₁-C₄) alkoxycarbonylfuryl-(C₁-C₄) alkoxy, halo-(C₁-C₄) alkylbenzyloxy, (C₁-C₄) alkoxyacetylaminoacetyloxy, acetoxy, cyclohexyl-(C₁-C₄) alkoxy, acetyloxy, morpholinylcarbonyl, (C₁-C₄) alkoxy-(C₁-C₄) alkoxy, or R⁴ and R⁵ optionally join to form a pyranyl or 1,4 dioxinyl ring;
R⁹ is selected from H, oxo, carbonitrile, or (C₁-C₆) alkyl;
R¹⁰ is selected from H, (C₁-C₆) alkyl, benzyl, phenyl, or hydroxy-(C₁-C₄) alkyl;
R¹¹ is selected from H, oxo, (C₁-C₆) alkyl, halobenzyl, benzyl, halo-(C₁-C₄) alkyl, C₁-C₄) alkoxycarbonyl, carboxy-(C₁-C₄) alkyl, 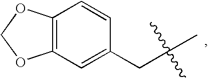
benzodioxinyl-(C₁-C₄) alkyl, substituted or unsubstituted aryl or aryl-(C₁-C₆) alkyl, which, if substituted, have one or more substituent groups selected from H, (C₁-C₆) alkyl, (C₁-C₆) alkoxy;
R¹² is selected from H, oxo, (C₁-C₆) alkyl, carbonyl-R¹³, carboxyl-R¹³, cyano, ketone, cyano-(C₁-C₄) alkyl, (C₁-C₄) alkoxycarbonyl, amino-(C₁-C₄) alkyl, (C₁-C₄) alkoxycarbonyl-(C₁-C₄) alkyl, carbamyl, hydroxycarbamyl, halo-(C₁-C₄) alkyl, tetrazolyl, tetrazolyl-(C₁-C₄) alkyl, (C₁-C₄) alkylacetate, t-butoxycarbonyl-(C₁-C₄) alkyl, hydroxy-(C₁-C₄) alkyl, dihydroxy-(C₁-C₄) alkyl, isoxazolyl, furanyl, pyrrolyl, pyrazolyl, imidazolyl, acetonitrile, or hydroxyamino, or R¹¹ and R¹² optionally join to form a ring system selected from: 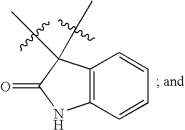
R¹³ is optionally present and if present, is selected from H, (C₁-C₆) alkyl, (C₁-C₆) alkoxy, amino, (C₁-C₆) allyl, (C₁-C₆) alkynyl, (C₁-C₆) alkenyl, hydrazine, benzyl, glycine, allyl-(C₁-C₄) alkyl, halo-(C₁-C₄) alkyl, hydroxy-(C₁-C₄) alkyl, hydroxyamino, dihydroxy-(C₁-C₄) alkyl, (C₁-C₄) alkoxy-(C₁-C₄) alkyl, (C₁-C₄) alkoxyamino, (C₁-C₄) alkylthio-(C₁-C₄) alkyl, cyclohexyl, cyclohexyl-(C₁-C₄) alkyl, amino, (C₁-C₄) alkylamino, benzyl-(C₁-C₄) alkyl, benzylamino, benzyloxycarbonyl-(C₁-C₄) alkylamino, dialkylamino, adamantyl halo-(C₁-C₄) alkoxybenzyl, (C₁-C₄) alkoxyamino-(C₁-C₄) alkyl, halo-(C₁-C₄) alkylaryl-(C₁-C₄) alkyl, pyridinyl-(C₁-C₄) alkyl, (C₁-C₄) alkoxy-(C₁-C₄) alkylamino, phenylthio-(C₁-C₄) alkyl, substituted or unsubstituted aryl or aryl-(C₁-C₄) alkyl, which, if substituted, have one or more substituent groups selected from H, (C₁-C₆) alkyl, (C₁-C₆) alkoxy, amino, nitro, halo, halo-(C₁-C₄) alkyl, or hydroxy-(C₁-C₄) alkyl.

14. The method according to claim 12, wherein:
X is C₁-C₃ alkyl;
R² is selected from H, (C₁-C₆) alkyl, carbonyl, (C₁-C₄) alkoxycarbonyl, amino-(C₁-C₄) alkyl, (C₁-C₆) alkoxy, amino-(C₁-C₄) alkyl, hydroxy, benzyl, hydroxyalkyl, dialkylamino, amino, halo, halo-(C₁-C₄) alkyl, carboxy, oxo, hydroxy-(C₁-C₄) alkylbenzyl, halo-(C₁-C₄) alkylbenzyl, halo-(C₁-C₄) alkoxybenzyl, furyl-(C₁-C₄) alkyl, substituted or unsubstituted aryl, which, if substituted, has one or more substituent groups selected from H, (C₁-C₄) alkyl, amino, nitro, halo, halo-(C₁-C₄) alkyl, or hydroxy-(C₁-C₄) alkyl;
R³ is selected from H, (C₁-C₆) alkyl, carbonyl, (C₁-C₄) alkoxycarbonyl, amino-(C₁-C₄) alkyl, (C₁-C₆) alkoxy, amino-(C₁-C₄) alkyl, hydroxy, benzyl, hydroxyalkyl, dialkylamino, amino, halo, halo-(C₁-C₄) alkyl, carboxy, hydroxy-(C₁-C₄) alkylbenzyl, halo-(C₁-C₄) alkylbenzyl, halo-(C₁-C₄) alkoxybenzyl, furyl-(C₁-C₄) alkyl, substituted or unsubstituted aryl, which, if substituted, has one or more substituent groups selected from H, (C₁-C₄) alkyl, amino, nitro, halo, halo-(C₁-C₄) alkyl, or hydroxy-(C₁-C₄) alkyl, or R² and R³ optionally join to form a cyclohexyl ring;
R⁴, R⁶ and R⁷ are independently selected from H, (C₁-C₆) alkyl, amino, (C₁-C₄) alkylamino, dialkylamino, benzyl, halo, halo-(C₁-C₄) alkyl, (C₁-C₆) alkoxy, halo-(C₁-C₄) alkoxy, carboxyl, nitro, benzyloxy, hydrazinocarbonyl, (C₁-C₄) alkoxycarbonyl, (C₁-C₄) alkoxycarbonylpiperazinylcarbonyl, hydroxy, morpholinylcarbonyl, or piperazinylcarbonyl;
R⁵ is selected from H, (C₁-C₆) alkyl, (C₁-C₄) alkenyl, (C₁-C₄) alkylthio, hydroxy, halo, (C₁-C₆) alkoxy, benzyl-(C₁-C₄) alkyl, benzyl-(C₁-C₄) alkoxy, aryl-(C₁-C₄) alkyl, benzyloxo-(C₁-C₄) alkoxy, nitro, (C₁-C₄) alkoxycarbonylalkoxy, (C₁-C₄) alkylthio, benzyloxy, phenyl-(C₁-C₄) alkynyl, phenylcarbonyl-(C₁-C₄) alkoxy, halo-(C₁-C₄) alkoxy, aryl-(C₁-C₄) alkoxy, arylcarbonyl-(C₁-C₄) alkoxy, haloaryl-(C₁-C₄) alkoxy, (C₁-C₄) alkoxycarbonylbenzyloxy, (C₁-C₄) alkoxycarbonyl, (C₁-C₄) alkoxyoxo-(C₁-C₄) alkoxy, -(C₁-C₄) alkoxyoxophenyl-(C₁-C₄) alkoxy, halobenzyloxy, pyridinyl-(C₁-C₄) alkoxy, dialkylaminooxo-(C₁-C₄) alkoxy, aminocarbonyl, phenyl-(C₁-C₄) alkynyl, hydrazinocarbonyl, carbonyl-(C₁-C₄) alkoxy, (C₁-C₄) alkylsulfonylbenzyloxy, carboxy-(C₁-C₄) alkoxy, carbamoyl, naphthyl-(C₁-C₄) alkoxy, (C₁-C₄) alkoxycarbonylfuryl-(C₁-C₄) alkoxy, halo-(C₁-C₄) alkylbenzyloxy, (C₁-C₄) alkoxyacetylaminoacetyloxy, acetoxy, cyclohexyl-(C₁-C₄) alkoxy, acetyloxy, morpholinylcarbonyl, (C₁-C₄) alkoxy-(C₁-C₄) alkoxy, or R⁴ and R⁵ optionally join to form a pyranyl or 1,4 dioxinyl ring;
R⁹ is selected from H, oxo, carbonitrile, or (C₁-C₆) alkyl;
R¹⁰ is selected from H, (C₁-C₆) alkyl, benzyl, phenyl, or hydroxy-(C₁-C₄) alkyl;
R¹¹ is selected from H, oxo, (C₁-C₆) alkyl, halobenzyl, benzyl, halo-(C₁-C₄) alkyl, C₁-C₄) alkoxycarbonyl, carboxy-(C₁-C₄) alkyl, 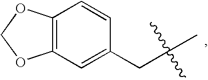
benzodioxinyl-(C₁-C₄) alkyl, substituted or unsubstituted aryl or aryl-(C₁-C₆) alkyl, which, if substituted, have one or more substituent groups selected from H, (C₁-C₆) alkyl, (C₁-C₆) alkoxy;
R¹² is selected from H, oxo, (C₁-C₆) alkyl, carbonyl-R¹³, carboxyl-R¹³, cyano, ketone, cyano-(C₁-C₄) alkyl, (C₁-C₄) alkoxycarbonyl, amino-(C₁-C₄) alkyl, (C₁-C₄) alkoxycarbonyl-(C₁-C₄) alkyl, carbamyl, hydroxycarbamyl, halo-(C₁-C₄) alkyl, tetrazolyl, tetrazolyl-(C₁-C₄) alkyl, (C₁-C₄) alkylacetate, t-butoxycarbonyl-(C₁-C₄) alkyl, hydroxy-(C₁-C₄) alkyl, dihydroxy-(C₁-C₄) alkyl, isoxazolyl, furanyl, pyrrolyl, pyrazolyl, imidazolyl, acetonitrile, or hydroxyamino, or R¹¹ and R¹² optionally join to form a ring system selected from: 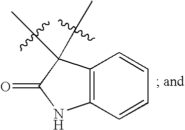
R¹³ is optionally present and if present, is selected from H, (C₁-C₆) alkyl, (C₁-C₆) alkoxy, amino, (C₁-C₆) allyl, (C₁-C₆) alkynyl, (C₁-C₆) alkenyl, hydrazine, benzyl, glycine, allyl-(C₁-C₄) alkyl, halo-(C₁-C₄) alkyl, hydroxy-(C₁-C₄) alkyl, hydroxyamino, dihydroxy-(C₁-C₄) alkyl, (C₁-C₄) alkoxy-(C₁-C₄) alkyl, (C₁-C₄) alkoxyamino, (C₁-C₄) alkylthio-(C₁-C₄) alkyl, cyclohexyl, cyclohexyl-(C₁-C₄) alkyl, amino, (C₁-C₄) alkylamino, benzyl-(C₁-C₄) alkyl, benzylamino, benzyloxycarbonyl-(C₁-C₄) alkylamino, dialkylamino, adamantyl halo-(C₁-C₄) alkoxybenzyl, (C₁-C₄) alkoxyamino-(C₁-C₄) alkyl, halo-(C₁-C₄) alkylaryl-(C₁-C₄) alkyl, pyridinyl-(C₁-C₄) alkyl, (C₁-C₄) alkoxy-(C₁-C₄) alkylamino, phenylthio-(C₁-C₄) alkyl, substituted or unsubstituted aryl or aryl-(C₁-C₄) alkyl, which, if substituted, have one or more substituent groups selected from H, (C₁-C₆) alkyl, (C₁-C₆) alkoxy, amino, nitro, halo, halo-(C₁-C₄) alkyl, or hydroxy-(C₁-C₄) alkyl.

15. The method according to claim 12, wherein:
X is C₁-C₃ alkyl;
R² is selected from H, methyl, ethyl, methoxy, propyl, n-propyl, aminomethyl, hydroxymethyl, dimethylaminomethyl, carboxy, dimethylamino, trifluoromethyl, methoxycarbonyl, oxo, carbonyl, alkoxycarbonyl, hydroxy, benzyl, amino, halo, trifluoromethyl, p-hydroxymethylbenzyl, m-trifluoromethylbenzyl, o-trifluoromethylbenzyl, trifluoromethoxybenzyl, phenyl, furylmethyl, or R² and R³ optionally join to form a ring system consisting of: 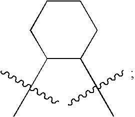
R³ is selected from H, methyl, ethyl, methoxy, propyl, n-propyl, aminomethyl, hydroxymethyl, dimethylaminomethyl, carboxy, dimethylamino, benzyl, trifluoromethyl, methoxycarbonyl, carbonyl, alkoxycarbonyl, hydroxy, amino, halo, trifluoromethyl, p-hydroxymethylbenzyl, m-trifluoromethylbenzyl, o-trifluoromethylbenzyl, trifluoromethoxybenzyl, phenyl, furylmethyl, or R² and R³ optionally join to form a ring system consisting of: 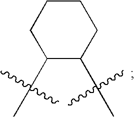
R⁴, R⁶ and R⁷ are independently selected from H, methyl, benzyl, methoxy, hydroxyl, methoxycarbonyl, benzyloxy, hydrazinocarbonyl, carbonxyl, fluoro, chloro, morpholinylcarbonyl, nitro, bromo, t-butoxycarbonylpiperazinylcarbonyl, or piperazinylcarbonyl;
R⁵ is selected from H, methyl, methoxy, acetoxy, carbamoyl, 2-oxo-2-phenylethoxy, hydroxyl, benzyloxy, benzyloxoethoxy, methoxycarbonyl, 2-ethoxy-2-oxoethoxy, 2-methoxy-2-oxo-1-phenylethoxy, butoxy, n-butoxy, 2-methoxy-2-oxoethoxy, p-chlorobenzyloxy, o-chlorobenzyloxy, m-chlorobenzyloxy, chloropropoxy, chlorobutoxy, chloro, 3-pyridylmethoxy, 4-pyridylmethoxy, N,N-dimethylcarbamylmethoxy, 2-phenylethoxy, aminocarbonyl, bromo, fluoro, carboxymethoxy, p-(methylsulfonyl)benzyloxy, hydrazinocarbonyl, 2-naphthylmethoxy, 4-ethoxy-4-oxobutoxy, (5-ethoxycarbonyl-2-furyl)methoxy, 2-methoxyethoxy, p-(trifluoromethyl)benzyloxy, 3-phenylpropoxy, m-chlorobenzylmethoxy, 3-methoxycarbonylbenzyloxy, 2-pyridylmethoxy, cyclohexylmethoxy, ethoxycarbonylmethylaminocarbonylmethoxy, iodo, phenylethynyl, 2-phenylethyl, nitro, ethoxy, or methylthio, or R⁴ and R⁵ optionally join to form a pyranyl or 1,4 dioxinyl ring;
R⁹ is selected from H, oxo, carbonitrile, or methyl
R¹⁰ is selected from H, benzyl, phenyl, methyl, ethyl, or hydroxymethyl;
R¹¹ is selected from H, oxo, methyl, benzyl, phenyl, chloromethyl, p-chlorobenzyl, methoxycarbonyl, ethoxycarbonyl, carboxyethyl, 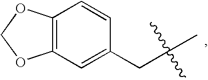
carboxypropyl, trimethoxybenzyl, or;
R¹² is selected from H, oxo, methyl, ethyl, carbonyl-R¹³, carboxyl-R¹³, ketone, cyano, cyanomethyl, tetrazoyl, pyrrolyl, tetrazoylmethyl, methylacetate, t-butylacetate, hydroxymethyl, trifluoromethyl, aminomethyl, acetonitrile, ethane-1,2-diol, methoxycarbonyl, methoxycarbonylmethyl, carbamyl, hydroxycarbamyl, isoxazolyl, furanyl, pyrazolyl, imidazolyl, or hydroxyamino, or R¹¹ and R¹² optionally join to form a ring system having the structure: 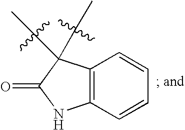
R¹³ is optionally present and if present, is selected from H, methyl, ethyl, i-propyl, n-propyl, propyl, benzyl, hydroxyamino, hydrazino, glycine, propene, benzyloxycarbonylmethylamino, 1-phenylethyl, 2,2,2-trifluoroethyl, 2-fluoroethyl, 2-hydroxyethyl, 2,3-dihydroxypropyl, 2-methoxyethyl, 2-(methylthio)ethyl, cyclohexyl, cyclohexylmethyl, t-butyl, neopentyl, 1-adamantyl, pentyl, amino, N,N-dimethylamino, benzylamino, 2-methoxyethylamino, n-hexyl, p-(trifluoromethylbenzyl), trifluoromethoxybenzyl, methylamino, pyridylmethyl, (N-methoxy-N-methyl)amino, 2,2,6,6-tetramethylcyclohexyl, or 2-(phenylthio)ethyl.

16. The method according to claim 12, wherein:
X is C₁-C₃ alkyl;
R² is selected from H, methyl, ethyl, methoxy, propyl, n-propyl, aminomethyl, hydroxymethyl, dimethylaminomethyl, carboxy, dimethylamino, trifluoromethyl, methoxycarbonyl, oxo, carbonyl, alkoxycarbonyl, hydroxy, benzyl, amino, halo, trifluoromethyl, p-hydroxymethylbenzyl, m-trifluoromethylbenzyl, o-trifluoromethylbenzyl, trifluoromethoxybenzyl, phenyl, furylmethyl, or R² and R³ optionally join to form a ring system consisting of: 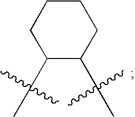
R³ is selected from H, methyl, ethyl, methoxy, propyl, n-propyl, aminomethyl, hydroxymethyl, dimethylaminomethyl, carboxy, dimethylamino, benzyl, trifluoromethyl, methoxycarbonyl, carbonyl, alkoxycarbonyl, hydroxy, amino, halo, trifluoromethyl, p-hydroxymethylbenzyl, m-trifluoromethylbenzyl, o-trifluoromethylbenzyl, trifluoromethoxybenzyl, phenyl, furylmethyl, or R² and R³ optionally join to form a ring system consisting of: 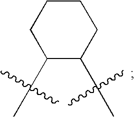
R⁴, R⁶ and R⁷ are independently selected from H, methyl, benzyl, methoxy, hydroxyl, methoxycarbonyl, benzyloxy, hydrazinocarbonyl, carbonxyl, fluoro, chloro, morpholinylcarbonyl, nitro, bromo, t-butoxycarbonylpiperazinylcarbonyl, or piperazinylcarbonyl;
R⁵ is selected from H, methyl, methoxy, acetoxy, carbamoyl, 2-oxo-2-phenylethoxy, hydroxyl, benzyloxy, benzyloxoethoxy, methoxycarbonyl, 2-ethoxy-2-oxoethoxy, 2-methoxy-2-oxo-1-phenylethoxy, butoxy, n-butoxy, 2-methoxy-2-oxoethoxy, p-chlorobenzyloxy, o-chlorobenzyloxy, m-chlorobenzyloxy, chloropropoxy, chlorobutoxy, chloro, 3-pyridylmethoxy, 4-pyridylmethoxy, N,N-dimethylcarbamylmethoxy, 2-phenylethoxy, aminocarbonyl, bromo, fluoro, carboxymethoxy, p-(methylsulfonyl)benzyloxy, hydrazinocarbonyl, 2-naphthylmethoxy, 4-ethoxy-4-oxobutoxy, (5-ethoxycarbonyl-2-furyl)methoxy, 2-methoxyethoxy, p-(trifluoromethyl)benzyloxy, 3-phenylpropoxy, m-chlorobenzylmethoxy, 3-methoxycarbonylbenzyloxy, 2-pyridylmethoxy, cyclohexylmethoxy, ethoxycarbonylmethylaminocarbonylmethoxy, iodo, phenylethynyl, 2-phenylethyl, nitro, ethoxy, or methylthio, or R⁴ and R⁵ optionally join to form a pyranyl or 1,4 dioxinyl ring;
R⁹ and R¹⁰ are H;
R¹¹ is selected from H, oxo, methyl, benzyl, phenyl, chloromethyl, p-chlorobenzyl, methoxycarbonyl, ethoxycarbonyl, carboxyethyl, 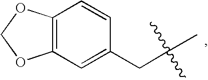
carboxypropyl, trimethoxybenzyl, or;
R¹² is selected from H, oxo, methyl, ethyl, carbonyl-R¹³, carboxyl-R¹³, ketone, cyano, cyanomethyl, tetrazoyl, pyrrolyl, tetrazoylmethyl, methylacetate, t-butylacetate, hydroxymethyl, trifluoromethyl, aminomethyl, acetonitrile, ethane-1,2-diol, methoxycarbonyl, methoxycarbonylmethyl, carbamyl, hydroxycarbamyl, isoxazolyl, furanyl, pyrazolyl, imidazolyl, or hydroxyamino, or R¹¹ and R¹² optionally join to form a ring system having the structure: 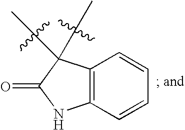
R¹³ is optionally present and if present, is selected from H, methyl, ethyl, i-propyl, n-propyl, propyl, benzyl, hydroxyamino, hydrazino, glycine, propene, benzyloxycarbonylmethylamino, 1-phenylethyl, 2,2,2-trifluoroethyl, 2-fluoroethyl, 2-hydroxyethyl, 2,3-dihydroxypropyl, 2-methoxyethyl, 2-(methylthio)ethyl, cyclohexyl, cyclohexylmethyl, t-butyl, neopentyl, 1-adamantyl, pentyl, amino, N,N-dimethylamino, benzylamino, 2-methoxyethylamino, n-hexyl, p-(trifluoromethylbenzyl), trifluoromethoxybenzyl, methylamino, pyridylmethyl, (N-methoxy-N-methyl)amino, 2,2,6,6-tetramethylcyclohexyl, or 2-(phenylthio)ethyl.

17. The method according to claim 12, wherein:
X is C₁-C₃ alkyl;
R², R³, R⁴, R⁶, R⁷, R⁹, R¹⁰ and R¹¹ are H;
R⁵ is selected from H, methoxy, hydroxy, benzyloxy, methylthio, or R⁴ and R⁵ optionally join to form a pyran ring;
R¹² is selected from carbonyl-R¹³, carboxyl-R¹³; and
R¹³ is optionally present and if present, is selected from H, methyl, ethyl, propyl, i-propyl, n-propyl, propenyl, trifluoromethyl, 4-(trifluoromethoxy)benzyl, trifluoroethyl, fluoroethyl, cyclohexylmethyl, 1-phenylethyl, cyclohexyl, 2-hydroxyethyl, benzyl, 2,3-dihydroxypropyl, 2-methoxyethyl, methylamino, benzylamino, 2-(methylthio)ethyl, hexyl, c-hexyl, n-hexyl, t-butyl, neopentyl, 1-adamantyl, 3-pentyl, amino, dimethylamino, benzylamino, methoxymethylamino, methoxyethylamino, pyridylmethyl, or 2,2,6,6-tetramethylcyclohexyl.

18. A method for treating or preventing a disease or disorder in a subject, which disease or disorder is one that can be treated or prevented by inhibiting the activity of MK-2, the method comprising administering to the subject a compound, or a pharmaceutically acceptable salt or prodrug thereof, wherein the compound has the structure: 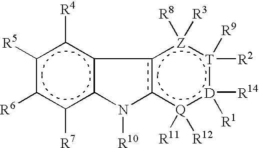
wherein:
Q, D, T and Z are selected from carbon, sulfur, or nitrogen;
when Q is sulfur, R¹² is oxo, and R¹¹ is optionally oxo or is absent;
when D is nitrogen, R¹² is selected from oxo, ═S, or ═N—OR¹⁵, and
R¹¹ is optionally oxo or is absent;
when T is sulfur, R² is oxo, and R⁹ is optionally oxo or is absent;
when Z is nitrogen, D is nitrogen, and R² and R¹² are oxo;
at least one of R¹, R², R³, R¹¹ and R¹² is oxo, ═S, ═N—OR¹⁵, or ═N—N(R¹⁵)₂;
R¹, R² and R³ are independently selected from H, alkoxy, oxo, arylalkenylamino, alkoxycarbonyl-R¹⁵, arylalkyl-R¹⁵, alkyl-SO₂—R¹⁵, amino, or substituted or unsubstituted alkyl, which, if substituted, has one or more substituent groups selected from H, halo, amino, cyano, hydroxyl, aryl, or heteroaryl;
R⁴, R⁶ and R⁷ are independently selected from H, alkyl, alkoxy, halo, carboxyl, nitro, CO₂—R¹⁵, substituted or unsubstituted 6-membered heteroarylcarbonyl, which, if substituted, have one or more substituent groups selected from H or alkoxycarbonyl, or R⁶ and R⁷ optionally join to form a heteroaryl ring;
R⁵ is selected from H, alkoxy, hydroxyl, halo, benzylalkyl, arylalkynyl, CO₂—R¹⁵, nitro, haloalkyl, alkoxycarbonyl or carboxyl, or R⁴ and R⁵ optionally join to form a heteroaryl ring;
R⁹ is independently selected from H and oxo or is optionally absent;
R¹⁰ is selected from H, alkyl, or aryl;
R¹¹ and R¹² are optionally present and if present, are independently selected from H, oxo, ═S, ═N—OR¹⁵, or ═N—N(R¹⁵)₂;
R¹⁴ is optionally present and if present, is selected from H or alkyl; and
R¹⁵ is optionally present and if present, is selected from H, alkyl, alkoxy, or aminoalkyl.

19. The method according to claim 18, wherein:
Q, D, T and Z are selected from carbon, sulfur, or nitrogen;
when Q is sulfur, R¹² is oxo, and R¹¹ is optionally oxo or is absent;
when D is nitrogen, R¹² is selected from oxo, ═S, or ═N—OR¹⁵, and R¹¹ is optionally oxo or is absent;
when T is sulfur, R² is oxo, and R⁹ is optionally oxo or is absent;
when Z is nitrogen, D is nitrogen, and R² and R¹² are oxo;
at least one of R¹, R², R³, R¹¹ and R¹² is oxo or oxime;
R¹, R² and R³ are independently selected from H, alkoxy, oxo, arylalkenylamino, alkoxycarbonyl-R¹⁵, arylalkyl-R¹⁵, alkyl-SO₂—R¹⁵, amino, or substituted or unsubstituted alkyl, which, if substituted, has one or more substituent groups selected from H, halo, amino, cyano, hydroxyl, aryl, or heteroaryl;
R⁴, R⁶ and R⁷ are independently selected from H, alkyl, alkoxy, halo, carboxyl, nitro, CO₂—R¹⁵, substituted or unsubstituted 6-membered heteroarylcarbonyl, which, if substituted, have one or more substituent groups selected from H or alkoxycarbonyl, or R⁶ and R⁷ optionally join to form a heteroaryl ring;
R⁵ is selected from H, alkoxy, hydroxyl, halo, benzylalkyl, arylalkynyl, CO₂—R¹⁵, nitro, haloalkyl, alkoxycarbonyl or carboxyl, or R⁴ and R⁵ optionally join to form a heteroaryl ring;
R⁹ is independently selected from H and oxo or is optionally absent;
R¹⁰ is selected from H, alkyl, or aryl;
R¹¹ and R¹² are optionally present and if present, are independently selected from H, oxo, or oxime;
R¹⁴ is optionally present and if present, is selected from H or alkyl; and
R¹⁵ is optionally present and if present, is selected from H, alkyl, alkoxy, or aminoalkyl.

20. The method according to claim 18, wherein:
Q, D, T and Z are selected from carbon, sulfur, or nitrogen;
when Q is sulfur, R¹² is oxo, and R¹¹ is optionally oxo or is absent;
when D is nitrogen, R¹² is selected from oxo, ═S, or ═N—OR¹⁵, and R¹¹ is optionally oxo or is absent;
when T is sulfur, R² is oxo, and R⁹ is optionally oxo or is absent;
when Z is nitrogen, D is nitrogen, and R² and R¹² are oxo;
at least one of R¹, R², R³, R¹¹ and R¹² is oxo or oxime;
R¹, R² and R³ are independently selected from H, (C₁-C₆) alkoxy, oxo, phenyl-(C₁-C₄) alkenylamino, amino, (C₁-C₄) alkoxycarbonyl-R¹⁵, cyano-(C₁-C₄) alkyl, aryl-(C₁-C₄) alkyl-R⁵, alkyl-SO₂—R⁵, or substituted or unsubstituted (C₁-C₆) alkyl, which, if substituted, has one or more substituent groups selected from H, halo, cyano, hydroxyl, amino, aryl, or heteroaryl;
R⁴, R⁶ and R⁷ are independently selected from H, (C₁-C₆) alkyl, (C₁-C₆) alkoxy, CO₂—R¹⁵, halo, carboxyl, nitro, or substituted or unsubstituted 6-membered heterocycliccarbonyl, which, if substituted, have one or more substituent groups selected from H or (C₁-C₄) alkoxycarbonyl, or R⁶ and R⁷ optionally join to form a heteroaryl ring;
R⁵ is selected from H, (C₁-C₆) alkoxy, hydroxyl, halo, benzyl-(C₁-C₄) alkyl, phenyl-(C₁-C₄) alkynyl, CO₂—R¹⁵, nitro, halo-(C₁-C₄) alkyl, (C₁-C₄) alkoxycarbonyl or carboxyl, or R⁴ and R⁵ optionally join to form a 6-membered heterocyclic ring;
R⁹ is independently selected from H and oxo or is optionally absent;
R¹⁰ is selected from H, (C₁-C₆) alkyl, or phenyl;
R¹¹ and R¹² are optionally present and if present, are independently selected from H, oxo, and oxime;
R¹⁴ is optionally present and if present, is selected from H or alkyl; and
R¹⁵ is optionally present and if present, is selected from H, (C₁-C₆) alkyl, (C₁-C₆) alkoxy, or amino-(C₁-C₄) alkyl.

21. The method according to claim 18, wherein:
Q, D, T and Z are selected from carbon, sulfur, or nitrogen;
when Q is sulfur, R¹² is oxo, and R¹¹ is optionally oxo or is absent;
when D is nitrogen, R¹² is selected from oxo, ═S, or ═N—OR¹⁵, and R¹¹ is optionally oxo or is absent;
when T is sulfur, R² is oxo, and R⁹ is optionally oxo or is absent;
when Z is nitrogen, D is nitrogen, and R² and R¹² are oxo;
at least one of R¹, R², R³, R¹¹ and R¹² is oxo or oxime;
R¹, R² and R³ are independently selected from H, methyl, oxo, phenylmethylideneamino, amino, hydroxy-(C₁-C₄) alkyl, cyano-(C₁-C₄) alkyl, amino-(C₁-C₄) alkyl, alkoxycarbonyl-R¹⁵, phenylalkyl-R¹⁵, or alkyl-SO₂—R¹⁵,
R⁴, R⁶ and R⁷ are independently selected from H, methyl, methoxy, chloro, bromo, fluoro, carboxyl, CO₂—R¹⁵, nitro, t-butoxycarbonylpiperazinylcarbonyl, or piperazinylcarbonyl, or R⁶ and R⁷ optionally join to form a pyridyl ring;
R⁵ is selected from H, methoxy, ethoxy, CO₂—R¹⁵, nitro, fluoro-(C₁-C₄) alkyl, hydroxyl, bromo, iodo, chloro, fluoro, 2-phenylethyl, phenylethynyl, nitro, methoxycarbonyl or carboxyl, or R⁴ and R⁵ optionally join to form a 1,4 dioxinyl ring;
R⁹ is independently selected from H and oxo or is optionally absent;
R¹⁰ is selected from H, methyl, or phenyl; and
R¹¹ and R¹² are optionally present and if present, are independently selected from H, oxo, and oxime;
R¹⁴ is optionally present and if present, is selected from H or methyl; and
R¹⁵ is optionally present and if present, is selected from H, (C₁-C₆) alkyl, (C₁-C₆) alkoxy, or amino-(C₁-C₄) alkyl.

22. The method according to claim 18, wherein:
Q, D, T and Z are selected from carbon, sulfur, or nitrogen, and at least one of Q, D, T and Z is nitrogen;
when Q is sulfur, R¹² is oxo, and R¹¹ is optionally oxo or is absent;
when D is nitrogen, R¹² is selected from oxo, ═S, or ═N—OR¹⁵, and R¹¹ is optionally oxo or is absent;
when T is sulfur, R² is oxo, and R⁹ is optionally oxo or is absent;
when Z is nitrogen, D is nitrogen, and R² and R¹² are oxo;
at least one of R¹, R², R³, R¹¹ and R¹² is oxo;
R¹, R², R³ and R¹⁰ are H;
R⁵ is selected from H, methoxy, hydroxyl, bromo, iodo, or chloro;
R⁹ is independently selected from H and oxo or is optionally absent;
R¹¹ and R¹² are independently selected from H and oxo, or are optionally absent; and
R¹⁴ is optionally present and if present, is selected from H or methyl.

23. The method according to claim 18, wherein:
Q, D, T and Z are selected from carbon, sulfur, or nitrogen, and at least one of Q, D, T and Z is nitrogen;
when Q is sulfur, R¹² is oxo, and R¹¹ is optionally oxo or is absent;
when D is nitrogen, R¹² is selected from oxo, ═S, or ═N—OR¹⁵, and R¹¹ is optionally oxo or is absent;
when T is sulfur, R² is oxo, and R⁹ is optionally oxo or is absent;
when Z is nitrogen, D is nitrogen, and R² and R¹² are oxo;
at least one of R¹, R², R³, R¹¹ and R¹² is oxo;
R¹, R², R³ and R¹⁰ are H;
R⁵ is selected from H, methoxy, hydroxyl;
R⁹ is independently selected from H and oxo or is optionally absent;
R¹¹ and R¹² are independently selected from H and oxo or are optionally absent; and
R¹⁴ is optionally present and if present, is selected from H or methyl.

24. The method according to claim 18, wherein:
Q, D, T and Z are selected from carbon, sulfur, or nitrogen, and at least one of Q, D, T and Z is nitrogen;
when Q is sulfur, R¹² is oxo, and R¹¹ is optionally oxo or is absent;
when D is nitrogen, R¹² is selected from oxo, ═S, or ═N—OR¹⁵, and R¹¹ is optionally oxo or is absent;
when T is sulfur, R² is oxo, and R⁹ is optionally oxo or is absent;
when Z is nitrogen, D is nitrogen, and R² and R¹² are oxo;
at least one of R¹, R², R³, R¹¹ and R¹² is oxo;
R¹, R², R³ and R¹⁰ are H;
R⁵ is selected from H or methoxy;
R⁹ is independently selected from H and oxo or is optionally absent;
R¹¹ and R¹² are independently selected from H and oxo or are optionally absent; and
R¹⁴ is optionally present and if present, is selected from H or methyl.

25. A method for treating or preventing a disease or disorder in a subject, which disease or disorder is one that can be treated or prevented by inhibiting the activity of MK-2, the method comprising administering to the subject a compound, or a pharmaceutically acceptable salt or prodrug thereof, wherein the compound has the structure: 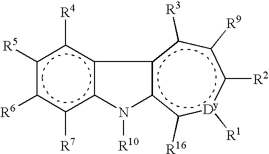
wherein:
D^{y} is selected from carbon or nitrogen;
R¹, R², R³ and R⁹ are independently selected from H, alkyl, alkoxy, carbonitrile, oxo, arylalkenylamino, or amino;
R⁴, R⁶ and R⁷ are independently selected from H, alkyl, alkoxy, halo, carboxyl, nitro, or substituted or unsubstituted 6-membered heteroarylcarbonyl, which, if substituted, have one or more substituent groups selected from H or alkoxycarbonyl;
R⁵ is selected from H or alkoxy;
R¹⁰ is selected from H, alkyl, or aryl; and
R¹⁶ is selected from H, oxo, CO₂H, or oxime.

26. The method according to claim 25, wherein:
D^{y} is selected from carbon or nitrogen;
R¹, R², R³ and R⁹ are independently selected from H, alkyl, alkoxy, carbonitrile, oxo, arylalkenylamino, or amino;
R⁴, R⁶ and R⁷ are independently selected from H, alkyl, alkoxy, halo, carboxyl, nitro, or substituted or unsubstituted 6-membered heteroarylcarbonyl, which, if substituted, have one or more substituent groups selected from H or alkoxycarbonyl;
R⁵ is selected from H or alkoxy;
R¹⁰ is selected from H, alkyl, or aryl; and
R¹⁶ is selected from H, oxo, CO₂H, or oxime.

27. The method according to claim 25, wherein:
D^{y} is selected from carbon or nitrogen;
when D^{y} is nitrogen, R¹⁶ is oxo;
when D^{y} is carbon, R¹⁶ oxime;
R¹, R², R³ and R⁹ are independently selected from H, (C₁-C₄) alkyl, methoxy, carbonitrile, oxo, phenylalkenylamino, or amino;
R⁴, R⁶ and R⁷ are independently selected from H, (C₁-C₄) alkyl, (C₁-C₄) alkoxy, halo, carboxyl, nitro, or substituted or unsubstituted 6-membered heteroarylcarbonyl, which, if substituted, have one or more substituent groups selected from H or alkoxycarbonyl;
R⁵ is selected from H or methoxy;
R¹⁰ is selected from H, (C₁-C₄) alkyl, or phenyl; and
R¹⁶ is selected from H, oxo, CO₂H, or oxime.

28. A method for treating or preventing a disease or disorder in a subject, which disease or disorder is one that can be treated or prevented by inhibiting the activity of MK-2, the method comprising administering to the subject a compound, or a pharmaceutically acceptable salt or prodrug thereof, wherein the compound has the structure: 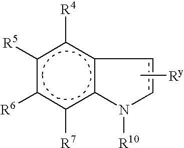
wherein:
R^{y} is optionally fused with the pyrrole ring and is selected from a substituted or unsubstituted 5-7 membered heterocyclic ring or a substituted or unsubstituted 5-7 membered cycloalkyl ring, which, if substituted, have one or more substituent groups selected from H, alkyl, alkoxy, carbonitrile, oxo, arylalkenylamino, halo, oxime, or amino;
R⁴, R⁶ and R⁷ are independently selected from H, alkyl, alkoxy, halo, carboxyl, nitro, or substituted or unsubstituted 6-membered heteroarylcarbonyl, which, if substituted, have one or more substituent groups selected from H or alkoxycarbonyl;
R⁵ is selected from H or alkoxy; and
R¹⁰ is selected from H, alkyl, or aryl.

29. The method according to claim 28, wherein:
R^{y} is fused with the pyrrole ring and is selected from a substituted or unsubstituted 5 or 7 membered heterocyclic ring or a substituted or unsubstituted 5 or 7 membered cycloalkyl ring, which, if substituted, have one or more substituent groups selected from H, alkyl, alkoxy, carbonitrile, oxo, arylalkenylamino, halo, oxime, or amino;
R⁴, R⁶ and R⁷ are independently selected from H, alkyl, alkoxy, halo, carboxyl, nitro, or substituted or unsubstituted 6-membered heteroarylcarbonyl, which, if substituted, have one or more substituent groups selected from H or alkoxycarbonyl;
R⁵ is selected from H or alkoxy; and
R¹⁰ is selected from H, alkyl, or aryl.

30. The method according to claim 28, wherein the compound has the structure:

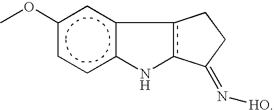

31. A method of modulating the activity of MK-2 in a subject in need of such modulation, the method comprising administering to the subject an MK-2 inhibiting compound, wherein the compound has the structure: 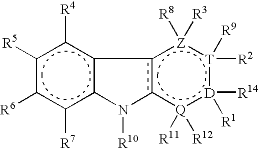
wherein:
Q, D, T and Z are selected from carbon, sulfur, or nitrogen;
when 0 is sulfur, R¹² is oxo, and R¹¹ is optionally oxo or is absent;
when T is sulfur, R² is oxo, and R⁹ is optionally oxo or is absent;
when Z is nitrogen, D is nitrogen, and R² and R¹² are oxo;
R¹ is selected from H, alkyl, alkenyl, alkynyl, allyl, acetyl, amino, alkoxy, hydroxy, oxo, carboxy, allylalkyl, benzyl, 2-ethylalkyl, haloacetyl, t-butyldialkylsilyloxyalkyl, alkoxycarbonylaminoalkyl, haloalkylbenzyl, haloalkylcarbonyl, alkylcarbonyl, alkoxycarbonyl, arylcarbonyl, benzyloxycarbonyl, arylalkenylamino, alkoxyheterocyclylcarbonyl, haloarylcarbonyl, or substituted or unsubstituted alkyl, which, if substituted, has one or more substituent groups selected from H, halo, amino, aryl, or heteroaryl;
R² is selected from H, alkyl, alkoxy, hydroxy, carbonyl, oxo, benzyl, hydroxyalkyl, dialkylamino, carboxyl, amino, halo, alkoxycarbonyl, alkoxycarbonyl-R¹⁵, aminoalkyl, haloalkyl, hydroxyalkylbenzyl, haloalkylbenzyl, arylalkenylamino, haloalkoxybenzyl, cyanoalkyl, heterocyclylalkyl, arylalkyl-R¹⁵, alkyl-SO₂—R¹⁵, substituted or unsubstituted aryl, which, if substituted, has one or more substituent groups selected from H, alkyl, amino, nitro, halo, haloalkyl, or hydroxyalkyl, or substituted or unsubstituted alkyl, which, if substituted, has one or more substituent groups selected from H, halo, amino, aryl, or heteroaryl;
R³ is selected from H, oxo, alkyl, alkoxy, amino, halo, haloalkyl, hydroxy, carbonyl, alkoxycarbonyl, benzyl, aminoalkyl, hydroxyalkyl, dialkylamino, arylalkenylamino, carboxy, haloalkyl, hydroxyalkylbenzyl, haloalkylbenzyl, haloalkoxybenzyl, heterocyclylalkyl, substituted or unsubstituted aryl, which, if substituted, has one or more substituent groups selected from H, alkyl, amino, nitro, halo, haloalkyl, or hydroxyalkyl, or substituted or unsubstituted alkyl, which, if substituted, has one or more substituent groups selected from H, halo, amino, aryl, or heteroaryl, or R¹ and R³ optionally join to form an alkyl bridge between the ring atoms to which R¹ and R³ are attached, or R² and R³ optionally join to form a cycloalkyl ring;
R⁴, R⁶ and R⁷ are independently selected from H, alkyl, benzyl, halo, haloalkyl, alkoxy, haloalkoxy, carboxyl, CO₂—R¹⁵, carboxyl, nitro, amino, alkylamino, dialkylamino, benzyloxy, hydrazinocarbonyl, alkoxycarbonyl, alkoxycarbonylheterocyclylcarbonyl, hydroxy, or substituted or unsubstituted 6-membered heterocyclylcarbonyl, which, if substituted, has one or more substituent groups selected from H or alkoxycarbonyl, or R⁶ and R⁷ optionally join to form a heteroaryl ring;
R⁵ is selected from H, alkyl, alkenyl, alkylthio, hydroxy, carboxyl, halo, alkoxy, benzylalkyl, benzylalkoxy, arylalkyl, nitro, alkoxycarbonylalkoxy, alkylthio, benzyloxy, arylalkynyl, arylcarbonylalkoxy, haloalkoxy, haloalkyl, arylalkoxy, haloarylalkoxy, benzyloxoalkoxy, alkoxycarbonylbenzyloxy, alkoxycarbonyl, alkoxyoxoalkoxy, alkoxyoxoarylalkoxy, halobenzyloxy, heterocyclylalkoxy, dialkylaminooxoalkoxy, aminocarbonyl, arylalkynyl, hydrazinocarbonyl, carbonylalkoxy, alkylsulfonylbenzyloxy, CO₂—R¹⁵, carbamoyl, naphthylalkoxy, alkoxyaryloxy, alkoxycarbonylheterocyclylalkoxy, haloalkylbenzyloxy, alkoxyacetylaminoacetyloxy, acetoxy, cycloalkylalkoxy, acetyloxy, morpholinylcarbonyl, alkoxyalkoxy, or R⁴ and R⁵ optionally join to form a heterocyclic ring;
R⁸ and R⁹ are independently optionally present and if present, are independently selected from H, oxo, carbonitrile, alkyl, or is optionally absent, or R³ and R⁸ optionally join to form a cycloalkyl ring;
R¹⁰ is selected from H, alkyl, benzyl, aryl, or hydroxyalkyl;
R¹¹ is optionally present and if present, is selected from H, oxo, alkyl, halobenzyl, benzyl, haloalkyl, 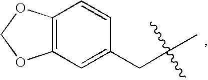
alkoxycarbonyl, carboxyalkyl, benzodioxinylalkyl, substituted or unsubstituted aryl or arylalkyl, which, if substituted, have one or more substituent groups selected from H, alkyl, or alkoxy;
R¹² is optionally present and if present, is selected from H, ═S, oxo, alkyl, carbonyl-R¹³, carboxyl-R¹³, ketone, cyano, cyanoalkyl, alkoxycarbonyl, aminoalkyl, alkoxycarbonylalkyl, carbamyl, hydroxycarbamyl, haloalkyl, heterocyclyl, alkylacetate, heterocyclylalkyl, alkoxycarbonylalkyl, hydroxyalkyl, dihydroxyalkyl, cyanoalkyl, ═N—OR¹⁵, ═N—N(R¹⁵)₂, or hydroxyamino, or R¹ and R¹² optionally join to form a substituted or unsubstituted 5 or 6-membered heteroaryl ring, which, if substituted, has one or more substituent groups selected from H, alkyl, oxo, or alkoxycarbonylalkyl, or R¹¹ and R¹² optionally join to form a ring system having the structure: 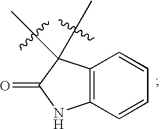
R¹³ is optionally present and if present, is selected from H, alkyl, alkoxy, amino, alkynyl, allyl, alkenyl, hydrazine, benzyl, glycine, allylalkyl, haloalkyl, hydroxyalkyl, hydroxyamino, dihydroxyalkyl, alkoxyalkyl, alkoxyamino, alkylthioalkyl, cycloalkyl, cyclohexylalkyl, amino, alkylamino, benzylalkyl, benzylamino, benzyloxycarbonylalkylamino, dialkylamino, adamantyl, haloalkoxybenzyl, alkoxyaminoalkyl, haloalkylarylalkyl, heterocyclylalkyl, alkoxyalkylamino, arylthioalkyl, substituted or unsubstituted aryl or arylalkyl, which, if substituted, have one or more substituent groups selected from H, alkyl, alkoxy, amino, nitro, halo, haloalkyl, or hydroxyalkyl;
R¹⁴ is optionally present and if present, is selected from H or alkyl; and
R¹⁵ is optionally present and if present, is selected from H, alkyl, alkoxy, or aminoalkyl.

32. A method of inhibiting MK-2 activity in a cell, the method comprising contacting the cell with a MK-2 inhibiting compound, wherein the compound has the structure: 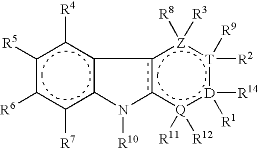
wherein:
Q, D, T and Z are selected from carbon, sulfur, or nitrogen;
when Q is sulfur, R¹² is oxo, and R¹¹ is optionally oxo or is absent;
when T is sulfur, R² is oxo, and R⁹ is optionally oxo or is absent;
when Z is nitrogen, D is nitrogen, and R² and R¹² are oxo;
R¹ is selected from H, alkyl, alkenyl, alkynyl, allyl, acetyl, amino, alkoxy, hydroxy, oxo, carboxy, allylalkyl, benzyl, 2-ethylalkyl, haloacetyl, t-butyldialkylsilyloxyalkyl, alkoxycarbonylaminoalkyl, haloalkylbenzyl, haloalkylcarbonyl, alkylcarbonyl, alkoxycarbonyl, arylcarbonyl, benzyloxycarbonyl, arylalkenylamino, alkoxyheterocyclylcarbonyl, haloarylcarbonyl, or substituted or unsubstituted alkyl, which, if substituted, has one or more substituent groups selected from H, halo, amino, aryl, or heteroaryl;
R² is selected from H, alkyl, alkoxy, hydroxy, carbonyl, oxo, benzyl, hydroxyalkyl, dialkylamino, carboxyl, amino, halo, alkoxycarbonyl, alkoxycarbonyl-R¹⁵, aminoalkyl, haloalkyl, hydroxyalkylbenzyl, haloalkylbenzyl, arylalkenylamino, haloalkoxybenzyl, cyanoalkyl, heterocyclylalkyl, arylalkyl-R¹⁵, alkyl-SO₂—R¹⁵, substituted or unsubstituted aryl, which, if substituted, has one or more substituent groups selected from H, alkyl, amino, nitro, halo, haloalkyl, or hydroxyalkyl, or substituted or unsubstituted alkyl, which, if substituted, has one or more substituent groups selected from H, halo, amino, aryl, or heteroaryl;
R³ is selected from H, oxo, alkyl, alkoxy, amino, halo, haloalkyl, hydroxy, carbonyl, alkoxycarbonyl, benzyl, aminoalkyl, hydroxyalkyl, dialkylamino, arylalkenylamino, carboxy, haloalkyl, hydroxyalkylbenzyl, haloalkylbenzyl, haloalkoxybenzyl, heterocyclylalkyl, substituted or unsubstituted aryl, which, if substituted, has one or more substituent groups selected from H, alkyl, amino, nitro, halo, haloalkyl, or hydroxyalkyl, or substituted or unsubstituted alkyl, which, if substituted, has one or more substituent groups selected from H, halo, amino, aryl, or heteroaryl, or R¹ and R³ optionally join to form an alkyl bridge between the ring atoms to which R¹ and R³ are attached, or R² and R³ optionally join to form a cycloalkyl ring;
R⁴, R⁶ and R⁷ are independently selected from H, alkyl, benzyl, halo, haloalkyl, alkoxy, haloalkoxy, carboxyl, CO₂—R¹⁵, carboxyl, nitro, amino, alkylamino, dialkylamino, benzyloxy, hydrazinocarbonyl, alkoxycarbonyl, alkoxycarbonylheterocyclylcarbonyl, hydroxy, or substituted or unsubstituted 6-membered heterocyclylcarbonyl, which, if substituted, has one or more substituent groups selected from H or alkoxycarbonyl, or R⁶ and R⁷ optionally join to form a heteroaryl ring;
R⁵ is selected from H, alkyl, alkenyl, alkylthio, hydroxy, carboxyl, halo, alkoxy, benzylalkyl, benzylalkoxy, arylalkyl, nitro, alkoxycarbonylalkoxy, alkylthio, benzyloxy, arylalkynyl, arylcarbonylalkoxy, haloalkoxy, haloalkyl, arylalkoxy, haloarylalkoxy, benzyloxoalkoxy, alkoxycarbonylbenzyloxy, alkoxycarbonyl, alkoxyoxoalkoxy, alkoxyoxoarylalkoxy, halobenzyloxy, heterocyclylalkoxy, dialkylaminooxoalkoxy, aminocarbonyl, arylalkynyl, hydrazinocarbonyl, carbonylalkoxy, alkylsulfonylbenzyloxy, CO₂—R¹⁵, carbamoyl, naphthylalkoxy, alkoxyaryloxy, alkoxycarbonylheterocyclylalkoxy, haloalkylbenzyloxy, alkoxyacetylaminoacetyloxy, acetoxy, cycloalkylalkoxy, acetyloxy, morpholinylcarbonyl, alkoxyalkoxy, or R⁴ and R⁵ optionally join to form a heterocyclic ring;
R⁵ and R⁹ are independently optionally present and if present, are independently selected from H, oxo, carbonitrile, alkyl, or is optionally absent, or R³ and R⁸ optionally join to form a cycloalkyl ring;
R¹⁰ is selected from H, alkyl, benzyl, aryl, or hydroxyalkyl;
R¹¹ is optionally present and if present, is selected from H, oxo, alkyl, halobenzyl, benzyl, haloalkyl, 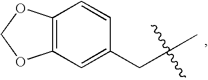
alkoxycarbonyl, carboxyalkyl, benzodioxinylalkyl, substituted or unsubstituted aryl or arylalkyl, which, if substituted, have one or more substituent groups selected from H, alkyl, or alkoxy;
R¹² is optionally present and if present, is selected from H, ═S, oxo, alkyl, carbonyl-R¹³, carboxyl-R¹³, ketone, cyano, cyanoalkyl, alkoxycarbonyl, aminoalkyl, alkoxycarbonylalkyl, carbamyl, hydroxycarbamyl, haloalkyl, heterocyclyl, alkylacetate, heterocyclylalkyl, alkoxycarbonylalkyl, hydroxyalkyl, dihydroxyalkyl, cyanoalkyl, ═N—OR¹⁵, ═N—N(R¹⁵)₂, or hydroxyamino, or R¹ and R¹² optionally join to form a substituted or unsubstituted 5 or 6-membered heteroaryl ring, which, if substituted, has one or more substituent groups selected from H, alkyl, oxo, or alkoxycarbonylalkyl, or R¹¹ and R¹² optionally join to form a ring system having the structure: 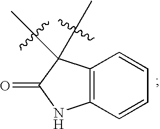
R¹³ is optionally present and if present, is selected from H, alkyl, alkoxy, amino, alkynyl, allyl, alkenyl, hydrazine, benzyl, glycine, allylalkyl, haloalkyl, hydroxyalkyl, hydroxyamino, dihydroxyalkyl, alkoxyalkyl, alkoxyamino, alkylthioalkyl, cycloalkyl, cyclohexylalkyl, amino, alkylamino, benzylalkyl, benzylamino, benzyloxycarbonylalkylamino, dialkylamino, adamantyl, haloalkoxybenzyl, alkoxyaminoalkyl, haloalkylarylalkyl, heterocyclylalkyl, alkoxyalkylamino, arylthioalkyl, substituted or unsubstituted aryl or arylalkyl, which, if substituted, have one or more substituent groups selected from H, alkyl, alkoxy, amino, nitro, halo, haloalkyl, or hydroxyalkyl;
R¹⁴ is optionally present and if present, is selected from H or alkyl; and
R¹⁵ is optionally present and if present, is selected from H, alkyl, alkoxy, or aminoalkyl.

33. The method according to claim 1, wherein the compound is administered to the subject in an MK-2 inhibitory amount.

34. The method according to claim 1, wherein the subject is administered an effective amount of the MK-2 inhibiting compound.

35. The method according to claim 31, wherein the effective amount comprises an amount within a range of from about 0.1 mg/kg/day to about 150 mg/kg/day.

36. The method according to claim 31, wherein the effective amount comprises an amount within a range of from about 0.1 mg/kg/day to about 10 mg/kg/day.

37. The method according to claim 1, wherein the MK-2 inhibiting compound provides a TNFα release IC₅₀ value of below 200 μM in an in vitro cell assay.

38. The method according to claim 1, wherein the MK-2 inhibiting compound provides a TNFα release IC₅₀ values of below 50 μM in an in vitro cell assay.

39. The method according to claim 1, wherein the MK-2 inhibiting compound provides a TNFα release IC₅₀ values of below 10 μM in an in vitro cell assay.

40. The method according to claim 1, wherein the MK-2 inhibiting compound provides a TNFα release IC₅₀ values of below 1 μM in an in vitro cell assay.

41. The method according to claim 1, wherein the MK-2 inhibiting compound provides a degree of inhibition of TNFα in a rat LPS assay of at least about 25%.

42. The method according to claim 1, wherein the MK-2 inhibiting compound provides a degree of inhibition of TNFα in a rat LPS assay of above 50%.

43. The method according to claim 1, wherein the MK-2 inhibiting compound provides a degree of inhibition of TNFα in a rat LPS assay of above 70%.

44. The method according to claim 1, wherein the MK-2 inhibiting compound provides a degree of inhibition of TNFα in a rat LPS assay of above 80%.

45. The method according to claim 1, wherein the MK-2 inhibiting compound provides an MK-2 inhibition IC₅₀ value of below 200 μM.

46. The method according to claim 1, wherein the MK-2 inhibiting compound provides an MK-2 inhibition IC₅₀ value of below 50 μM.

47. The method according to claim 1, wherein the MK-2 inhibiting compound provides an MK-2 inhibition IC₅₀ value of below 20 μM.

48. The method according to claim 1, wherein the MK-2 inhibiting compound provides an MK-2 inhibition IC₅₀ value of below 1 μM.

49. The method according to claim 1, wherein the subject is one that is in need of treatment or prevention of a disease or disorder that can be treated or prevented by inhibiting the activity of MK-2.

50. The method according to claim 1, wherein the disease or disorder that can be treated or prevented by inhibiting the activity of MK-2 is a disease or disorder that is mediated by TNFα.

51. The method according to claim 1, wherein the subject is one that is in need of treatment or prevention of a TNFα mediated disease or disorder.

52. The method according to claim 1, wherein the disease or disorder is pain, inflammation or an inflammation-related disease or disorder.

53. The method according to claim 1, wherein the subject is one that is in need of treatment or prevention of pain, inflammation, or inflammation-related disease or disorder.

54. The method according to claim 47, wherein the TNFα mediated disease or disorder is chosen from arthritis, rheumatoid arthritis, spondyloarthopathies, gouty arthritis, osteoarthritis, systemic lupus erythematosus, juvenile arthritis, asthma, bronchitis, menstrual cramps, tendinitis, bursitis, connective tissue injuries or disorders, skin related conditions, psoriasis, eczema, burns, dermatitis, gastrointestinal conditions, inflammatory bowel disease, gastric ulcer, gastric varices, Crohn's disease, gastritis, irritable bowel syndrome, ulcerative colitis, cancer, colorectal cancer, herpes simplex infections, HIV, pulmonary edema, kidney stones, minor injuries, wound healing, vaginitis, candidiasis, lumbar spondylanhrosis, lumbar spondylarthrosis, vascular diseases, migraine headaches, sinus headaches, tension headaches, dental pain, periarteritis nodosa, thyroiditis, aplastic anemia, Hodgkin's disease, sclerodoma, rheumatic fever, type I diabetes, myasthenia gravis, multiple sclerosis, sarcoidosis, nephrotic syndrome, Behcet's syndrome, polymyositis, gingivitis, hypersensitivity, swelling occurring after injury, myocardial ischemia, ophthalmic diseases, retinitis, retinopathies, conjunctivitis, uveitis, ocular photophobia, acute injury to the eye tissue, pulmonary inflammation, viral infections, cystic fibrosis, central nervous system disorders, cortical dementias, and Alzheimer's disease.

55. The method according to claim 1, wherein the compound is administered to the subject in an MK-2 inhibitory amount.

56. The method according to claim 1, wherein the subject is administered an effective amount of the MK-2 inhibiting compound.

57. A compound, or a pharmaceutically acceptable salt or isomer thereof, the compound having the structure: 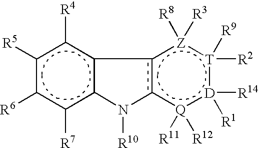
wherein:
Q, D, T and Z are selected from carbon, sulfur, or nitrogen;
when Q is sulfur, R¹² is oxo, and R¹¹ is optionally oxo or is absent;
when T is sulfur, R² is oxo, and R⁹ is optionally oxo or is absent;
when Z is nitrogen, D is nitrogen, and R² and R¹² are oxo;
R¹ is selected from H, alkyl, alkenyl, alkynyl, allyl, acetyl, amino, alkoxy, hydroxy, oxo, carboxy, allylalkyl, benzyl, 2-ethylalkyl, haloacetyl, t-butyldialkylsilyloxyalkyl, alkoxycarbonylaminoalkyl, haloalkylbenzyl, haloalkylcarbonyl, alkylcarbonyl, alkoxycarbonyl, arylcarbonyl, benzyloxycarbonyl, arylalkenylamino, alkoxyheterocyclylcarbonyl, haloarylcarbonyl, or substituted or unsubstituted alkyl, which, if substituted, has one or more substituent groups selected from H, halo, amino, aryl, or heteroaryl;
R² is selected from H, alkyl, alkoxy, hydroxy, carbonyl, oxo, benzyl, hydroxyalkyl, dialkylamino, carboxyl, amino, halo, alkoxycarbonyl, alkoxycarbonyl-R¹⁵, aminoalkyl, haloalkyl, hydroxyalkylbenzyl, haloalkylbenzyl, arylalkenylamino, haloalkoxybenzyl, cyanoalkyl, heterocyclylalkyl, arylalkyl-R¹⁵, alkyl-SO₂—R¹⁵, substituted or unsubstituted aryl, which, if substituted, has one or more substituent groups selected from H, alkyl, amino, nitro, halo, haloalkyl, or hydroxyalkyl, or substituted or unsubstituted alkyl, which, if substituted, has one or more substituent groups selected from H, halo, amino, aryl, or heteroaryl;
R³ is selected from H, oxo, alkyl, alkoxy, amino, halo, haloalkyl, hydroxy, carbonyl, alkoxycarbonyl, benzyl, aminoalkyl, hydroxyalkyl, dialkylamino, arylalkenylamino, carboxy, haloalkyl, hydroxyalkylbenzyl, haloalkylbenzyl, haloalkoxybenzyl, heterocyclylalkyl, substituted or unsubstituted aryl, which, if substituted, has one or more substituent groups selected from H, alkyl, amino, nitro, halo, haloalkyl, or hydroxyalkyl, or substituted or unsubstituted alkyl, which, if substituted, has one or more substituent groups selected from H, halo, amino, aryl, or heteroaryl, or R¹ and R³ optionally join to form an alkyl bridge between the ring atoms to which R¹ and R³ are attached, or R² and R³ optionally join to form a cycloalkyl ring;
R⁴, R⁶ and R⁷ are independently selected from H, alkyl, benzyl, halo, haloalkyl, alkoxy, haloalkoxy, carboxyl, CO₂—R¹⁵, carboxyl, nitro, amino, alkylamino, dialkylamino, benzyloxy, hydrazinocarbonyl, alkoxycarbonyl, alkoxycarbonylheterocyclylcarbonyl, hydroxy, or substituted or unsubstituted 6-membered heterocyclylcarbonyl, which, if substituted, has one or more substituent groups selected from H or alkoxycarbonyl, or R⁶ and R⁷ optionally join to form a heteroaryl ring;
R⁵ is selected from H, alkyl, alkenyl, alkylthio, hydroxy, carboxyl, halo, alkoxy, benzylalkyl, benzylalkoxy, arylalkyl, nitro, alkoxycarbonylalkoxy, alkylthio, benzyloxy, arylalkynyl, arylcarbonylalkoxy, haloalkoxy, haloalkyl, arylalkoxy, haloarylalkoxy, benzyloxoalkoxy, alkoxycarbonylbenzyloxy, alkoxycarbonyl, alkoxyoxoalkoxy, alkoxyoxoarylalkoxy, halobenzyloxy, heterocyclylalkoxy, dialkylaminooxoalkoxy, aminocarbonyl, arylalkynyl, hydrazinocarbonyl, carbonylalkoxy, alkylsulfonylbenzyloxy, CO₂—R¹⁵, carbamoyl, naphthylalkoxy, alkoxyaryloxy, alkoxycarbonylheterocyclylalkoxy, haloalkylbenzyloxy, alkoxyacetylaminoacetyloxy, acetoxy, cycloalkylalkoxy, acetyloxy, morpholinylcarbonyl, alkoxyalkoxy, or R⁴ and R⁵ optionally join to form a heterocyclic ring;
R⁸ and R⁹ are independently optionally present and if present, are independently selected from H, oxo, carbonitrile, alkyl, or is optionally absent, or R³ and R⁸ optionally join to form a cycloalkyl ring;
R¹⁰ is selected from H, alkyl, benzyl, aryl, or hydroxyalkyl;
R¹¹ is optionally present and if present, is selected from H, oxo, alkyl, halobenzyl, benzyl, haloalkyl, 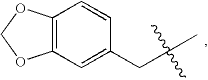
alkoxycarbonyl, carboxyalkyl, benzodioxinylalkyl, substituted or unsubstituted aryl or arylalkyl, which, if substituted, have one or more substituent groups selected from H, alkyl, or alkoxy;
R¹² is optionally present and if present, is selected from H, ═S, oxo, alkyl, carbonyl-R¹³, carboxyl-R¹³, ketone, cyano, cyanoalkyl, alkoxycarbonyl, aminoalkyl, alkoxycarbonylalkyl, carbamyl, hydroxycarbamyl, haloalkyl, heterocyclyl, alkylacetate, heterocyclylalkyl, alkoxycarbonylalkyl, hydroxyalkyl, dihydroxyalkyl, cyanoalkyl, ═N—OR¹⁵, ═N—N(R¹⁵)₂, or hydroxyamino, or R¹ and R¹² optionally join to form a substituted or unsubstituted 5 or 6-membered heteroaryl ring, which, if substituted, has one or more substituent groups selected from H, alkyl, oxo, or alkoxycarbonylalkyl, or R¹¹ and R¹² optionally join to form a ring system having the structure: 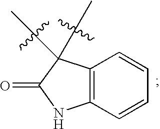
R¹³ is optionally present and if present, is selected from H, alkyl, alkoxy, amino, alkynyl, allyl, alkenyl, hydrazine, benzyl, glycine, allylalkyl, haloalkyl, hydroxyalkyl, hydroxyamino, dihydroxyalkyl, alkoxyalkyl, alkoxyamino, alkylthioalkyl, cycloalkyl, cyclohexylalkyl, amino, alkylamino, benzylalkyl, benzylamino, benzyloxycarbonylalkylamino, dialkylamino, adamantyl, haloalkoxybenzyl, alkoxyaminoalkyl, haloalkylarylalkyl, heterocyclylalkyl, alkoxyalkylamino, arylthioalkyl, substituted or unsubstituted aryl or arylalkyl, which, if substituted, have one or more substituent groups selected from H, alkyl, alkoxy, amino, nitro, halo, haloalkyl, or hydroxyalkyl;
R¹⁴ is optionally present and if present, is selected from H or alkyl; and
R¹⁵ is optionally present and if present, is selected from H, alkyl, alkoxy, or aminoalkyl.
